(19) United States
(12) Patent Application Publication
(10) Pub. No.: US 2026/0085325 A1
(43) Pub. Date: Mar. 26, 2026

(54) DISRUPTION OF GROOT1, GROOT2, AND GROOT3 GENE FUNCTION INCREASES ROOT AND SHOOT BIOMASS

(71) Applicant: SALK INSTITUTE FOR BIOLOGICAL STUDIES, LA JOLLA, CA (US)

(72) Inventors: Samsad Razzaque, San Diego, CA (US); Wolfgang Busch, San Diego, CA (US); Ashot Papikian, San Diego, CA (US); Ana Paez-Garcia, Las Rozas de Madrid (ES)

(73) Assignee: SALK INSTITUTE FOR BIOLOGICAL STUDIES, LA JOLLA, CA (US)

(21) Appl. No.: 19/337,581

(22) Filed: Sep. 23, 2025

Related U.S. Application Data
(60) Provisional application No. 63/698,475, filed on Sep. 24, 2024.

(51) Int. Cl. C12N 15/82; C12N 9/22; C12N 15/11

(52) U.S. Cl. CPC C12N 15/8262; C12N 9/226; C12N 15/11; C12N 15/8205; C12N 15/8213; C12N 2310/20

(57) ABSTRACT

Provided are methods and compositions for enhancing biomass, particularly shoot biomass and/or root biomass, and/or seed size in plants, by reducing expression and/or activity of GROOT1, GROOT2, and/or GROOT3 in plants. Also provided are plants and seeds generated using these methods. In some examples, reducing expression and/or activity includes introducing one or more exogenous nucleic acid molecules into a plant, plant part, or plant cell, thereby generating a transformed plant, plant part, or plant cell, wherein the one or more exogenous nucleic acid molecules reduce expression and/or activity of one or more of GROOT1, GROOT2, and GROOT3.

### CROSS REFERENCE TO RELATED APPLICATION

This application claims the benefit of U.S. Provisional Application No. 63/698,475, filed Sep. 24, 2024, which is hereby incorporated by reference in its entirety.

### FIELD

The present disclosure generally relates to the field of modulating root biomass, shoot biomass, and seed size in plants. More particularly, the present disclosure relates to compositions and methods for generating plants with increased root biomass, shoot biomass, and/or seed size, by reducing expression or activity of one or more of GROOT1, GROOT2, and GROOT3.

### INCORPORATION OF SEQUENCE LISTING

The Sequence Listing is submitted as an XML file in the form of the file named “sequencelisting_7158-112601-02.xml” (98,423 bytes), which was created on Sep. 20, 2025 which is incorporated by reference herein.

### BACKGROUND

Roots are essential for most land plants as they forage the soil for nutrients and water, which are critical for their growth and survival and provide anchorage for the plant body (Ogura et al. 2019; Maurel and Nacry 2020). Root systems are important for plant resilience, particularly in the face of climate change and temperature fluctuations (Mayjonade et al. 2019; Hancock et al. 2011; Karlova et al. 2021). Given the ever-changing environment, with significant fluctuations in various environmental variables, roots exhibit remarkable plasticity in their growth patterns, allowing plants to adapt and thrive in their natural habitats (Lorts and Lasky 2020; Fitz Gerald et al. 2006; Gruber et al. 2013). The extensiveness of the root system is determined by its growth rate. The cumulative growth of a root system can be measured as root biomass. Larger root systems promise to contribute significantly to carbon sequestration, as they store carbon captured through photosynthesis, with deeper, more extensive root systems enhancing soil carbon storage (Kumar et al. 2006; Panchal et al. 2022). Increased soil inputs via larger root systems also enhance soil health and fertility, fostering sustainable ecosystems (Oren et al. 2001; Sang et al. 2013).

Root biomass is a complex trait likely influenced by pleiotropic effects involving multiple genes and genetic pathways (Chen et al. 2021). While not well understood, the genetic basis for root biomass accumulation is shaped by various factors, including resource allocation strategies, which can create trade-offs between root and shoot growth (Lynch 2022). For instance, plants that invest more in root development might experience reduced above-ground growth. However, these trade-offs are not always straightforward and can be context dependent. Environmental conditions, such as soil fertility, water availability, and light intensity, can influence the extent and nature of these trade-offs. In nutrient-rich environments, plants may be able to support both robust root and shoot growth, whereas in nutrient-poor conditions, prioritizing root growth for better resource acquisition may come at the expense of shoot development. Moreover, environmental fluctuations and community assembly can alter this dynamic, as different conditions and community compositions may select for varying root and shoot traits (Lavorel and Garnier 2002; Garcia-Palacios et al. 2013; Gedroc et al. 1996). Additionally, genetic factors and the specific roles of different genes can modulate these trade-offs, indicating that the interplay between root and shoot growth is highly dynamic and influenced by a myriad of internal and external factors (He et al. 2022; Dwivedi et al. 2021; Smakowska et al. 2016; Lundgren and Des Marais 2020).

Simultaneously enhancing both shoot and root biomass is challenging. Overexpression of the CYTOKININ OXIDASE (CKX) gene generally accelerates and enlarges root growth but significantly reduces leaf production to just 3-4% of that in wild-type plants (Werner et al. 2001). This reduction may be due to CKX overexpression in both root and shoot tissues. However, tissue-specific expression of the CKX gene can modulate the trade-off between root and shoot growth. For example, transgenic maize with root-specific CKX expression showed up to 46% more root dry weight without affecting shoot growth (Ramireddy et al. 2021). Similarly, another study found that overexpressing the chickpea CKX gene (CaCKX6) under a root-specific promoter in both Arabidopsis thaliana and chickpea significantly increased lateral root number and root biomass, while leaving shoot growth unaffected (Khandal et al. 2020). A similar pattern of balancing shoot and root growth has also been observed in other genes, such as OsWOX11 in rice and TaNAC69-1 in wheat. OsWOX11 enhances root biomass by promoting the growth of crown roots without affecting shoot growth (Jiang et al. 2017). In contrast, TaNAC69-1 increases primary seminal root length and overall root biomass, as well as shoot biomass when overexpressed using specific promoters (Chen et al. 2016). The pattern of root to shoot growth under the same gene activity is inconsistent, and growth stimulation appears to be more dependent on the promoters driving the gene. Thus, there remains a need for identifying plant genes that can be manipulated to increase both shoot and root biomass without relying on tissue-specific promoters. Information on these genes will be highly valuable for enhancing plant performance, productivity, resilience, and carbon sequestration. Therefore, identifying genes and genetic pathways that limit growth and upon loss of function enhance root and shoot growth would be beneficial for optimizing overall plant performance and resilience.

### SUMMARY

Provided are methods for generating a plant with increased biomass, and/or increased seed size, comprising: reducing expression and/or activity of one or more of GROOT1, GROOT2, and GROOT3 in a plant, thereby generating the plant with increased biomass, and/or increased seed size. Also provided are methods for generating a plant with increased biomass, and/or increased seed size, comprising: reducing expression and/or activity of one or more of GROOT1, GROOT2, and GROOT3 in a plant cell or plant part, and growing the plant cell or plant part into a plant, thereby generating the plant with increased biomass, and/or increased seed size.

In some aspects, the reducing expression and/or activity comprises introducing one or more exogenous nucleic acid molecules into a plant, thereby generating a transformed plant, wherein the one or more exogenous nucleic acid molecules reduce expression of one or more of GROOT1, GROOT2, and GROOT3, and/or reduce activity of one or more proteins encoded by one or more of GROOT1, GROOT2, and GROOT3. In some aspects, the reducing expression and/or activity comprises introducing one or more exogenous nucleic acid molecules into a plant cell or plant part, thereby generating a gene-edited or transgenic plant cell or plant part; and the growing comprises growing the gene-edited or transgenic plant cell or plant part into a transformed plant, thereby generating the plant with increased biomass, and/or increased seed size; wherein the one or more exogenous nucleic acid molecules reduce expression of one or more of GROOT1, GROOT2, and GROOT3, and/or reduce activity of one or more proteins encoded by one or more of GROOT1, GROOT2, and GROOT3. In some aspects, the introducing one or more exogenous nucleic acid molecules generates one or more deletions of, or one or more loss-of-function mutations, in the one or more of GROOT1, GROOT2, and GROOT3. In some aspects, the method further comprises introducing one or more Cas proteins or one or more nucleic acid molecules encoding a Cas protein into the plant, plant cell, or plant part.

Also provided are transformed plants, gene-edited or transgenic plant cells or plant parts, transformed plant tissues, or transformed plantlets made by the methods provided herein.

Also provided are methods of producing a commodity plant product, comprising collecting or producing the commodity plant product from the transformed plant, gene-edited or transgenic plant cell or plant part, transformed plant tissue, transformed plantlet, or transformed progeny provided herein; optionally, wherein the commodity plant product comprises a non-native nucleic acid molecule or protein from the transformed plant, gene-edited or transgenic plant cell or plant part, transformed plant tissue, transformed plantlet, or transformed progeny; and optionally, wherein the commodity product comprises a protein concentrate, protein isolate, leaves, extract, oil, bean, and/or seed.

Also provided are methods of producing plant seed, comprising crossing the transformed plant, transformed plantlet, or transformed progeny provided herein with itself or a second plant.

Also provided are methods for breeding a plant with increased biomass, and/or increased seed size, comprising crossing the transformed plant provided herein with a second plant; obtaining seeds from the crossing; planting the seeds and growing the seeds to progeny plants; and selecting from the progeny plants those with increased biomass, and/or increased seed size when compared to a control plant. In some aspects, the method further comprising producing clones of the progeny plants, wherein the clones are selected based on increased biomass, and/or increased seed size when compared to a control plant.

Also provided are seeds that produces or are produced by the transformed plant provided herein, wherein said seed comprises one or more deletions of, or one or more loss-of-function mutations in the one or more of GROOT1, GROOT2, and GROOT3.

Also provided are gene-edited plants, plant parts, plant cells, or seeds, comprising one or more deletions of, or one or more loss-of-function mutations in one or more of GROOT1, GROOT2, and GROOT3.

In some aspects, the gene-edited plants, plant parts, plant cells, or seeds are transgene-free.

In some aspects, GROOT1, prior to the one or more deletions or loss-of-function mutations, comprises at least 60%, 65%, 70%, 75%, 80%, 85%, 90%, 95%, 96%, 97%, 98%, or 99%, or 100% sequence identity to any of SEQ ID NOs:1, 3-4 and 6; or encodes a coding sequence comprising at least 60%, 65%, 70%, 75%, 80%, 85%, 90%, 95%, 96%, 97%, 98%, or 99%, or 100% sequence identity to any of SEQ ID NOs: 2, 5 and 7; or encodes a protein sequence comprising at least 60%, 65%, 70%, 75%, 80%, 85%, 90%, 95%, 96%, 97%, 98%, or 99%, or 100% sequence identity to any of SEQ ID NOs: 8-13; GROOT2, prior to the one or more deletions or loss-of-function mutations, comprises at least 60%, 65%, 70%, 75%, 80%, 85%, 90%, 95%, 96%, 97%, 98%, or 99%, or 100% sequence identity to any of SEQ ID NOs: 14 and 16-17; or encodes a coding sequence comprising at least 60%, 65%, 70%, 75%, 80%, 85%, 90%, 95%, 96%, 97%, 98%, or 99%, or 100% sequence identity to any of SEQ ID NOs: 15 and 18; or encodes a protein sequence comprising at least 60%, 65%, 70%, 75%, 80%, 85%, 90%, 95%, 96%, 97%, 98%, or 99%, or 100% sequence identity to any of SEQ ID NOs: 19-22; and/or GROOT3, prior to the one or more deletions or loss-of-function mutations, comprise at least 60%, 65%, 70%, 75%, 80%, 85%, 90%, 95%, 96%, 97%, 98%, or 99%, or 100% sequence identity to any of SEQ ID NOs: 23 and 25-29; or encodes a coding sequence comprising at least 60%, 65%, 70%, 75%, 80%, 85%, 90%, 95%, 96%, 97%, 98%, or 99%, or 100% sequence identity to SEQ ID NO: 24; or encodes a protein sequence comprising at least 60%, 65%, 70%, 75%, 80%, 85%, 90%, 95%, 96%, 97%, 98%, or 99%, or 100% sequence identity to any of SEQ ID NOs: 30-35.

Also provided are ribonucleoprotein complexes, comprising: an isolated Cas protein; and a gRNA or sgRNA specific for one or more of GROOT1, GROOT2, and GROOT3.

### SEQUENCES

The nucleic and amino acid sequences listed herein are shown using standard letter abbreviations for nucleotide bases and amino acids. Only one strand of each nucleic acid sequence is shown, but the complementary strand is understood as included by any reference to the displayed strand.

### I. Exemplary Nucleic Acid and Protein Sequences of GROOT1

SEQ ID NO: 1 is an exemplary genomic sequence of GROOT1
in Arabidopsis thaliana (TAIR AT3G19440.1):
(SEQ ID NO: 1)
AATGACTATCGGTTCCATCTTTCCCAAGTTCCTCTGCTCCAAAACCTCTTAAACCCTAAT
TCGAATTTAGCCTAAACCCTAAAATGGCCAAATGGAGACTTGCGACTGCGACGCTCCGTC
GACAACTTCAATCCTCATCGCCAACTATCTCTACCTTCAAGAATCCAACCAAAGCATTGT
CTGCGGCGGCTCATCAGTCTACTCGCAGCTACAGTACAACTCAGACCGATGATTCGAGAG
GGAAATGGTTAACTTTACCTCCTTTTTCTCCCACCATCGATGGCACCGCCGTTGGAAAGG
ATCTCCTTTCCGACGGAGACTCTGTCAAATCTTCAACGGACAACTCAAAAACGACGGCGC
TTAGGTGGATTCTTCGTTGTCGTCCCGATTTACCCAGAACTCTCGTTCAGAAACTCTTCC
GCTTAAGACAGGTTCCTAGTCTCTTCCAATCTTGTATTCAACGAATTGTTGTCAGTTTCT
TACATTGTAATTGATTCAGGTTAGAAGAGAAATGTCTATGAGTGTTGATGGTGATGAACT
ACAAAGAAGCCAGCTTAAAAGGGTAATGTTATTTTGTTATCTATTGATTGGAACAAGAAC
TGTTTTGTTGAAAGGCTGCTCTTTTATCTGATGATGTTGATTTCTGACCCTTGAAAGGTG
GCAGCTAAGGAGTCCTTGAATGTAGGAGATAGAATTTACCTTCCTTTATCGGTTGACAAT
GATACGCCGCAGACGCCACCTGCTAAGAAAGAAAGCTTTCAATGTAGTGATGAAGAACGC
AAATTCGTTTGCAGTTTGGTGTTGTATAAGGTTTTGAAACTGTATACTATTGGTTTGCTT
TTTTGTTTTTTTATTGATGTTATATACTTATATAGATTGATAGATCTTAACTGTGTGTTA
ATGTGCCAGGATCCAGCCATTATTGTTTTGAATAAACCTCATGGTTTGGCTGTTCAAGTG
AGTCATTTATACTTCGCATTCTGTTGTATTGTGTATCATATTCTTGGAGCTTGTGAAATG
TGATGACATTTGCTCTTTAATAGGGTGGGAGTGGGATCAAAACCAGTATTGATGAACTCG
CTGCCTCTTGCTTGAAATTTGATAAATCAGAATCTCCCCGGCTGGTTAGTTGCGAATCAT
AGTAAAGCACTGATTAAAACTGTATCTAAACGAAAGTGTATGATTTATTATGTTGTTTTT
GTTAATTTGAAAAGTATGAATCTATTGGCTTATGCAAGTATGGGATATGTATTAGGTGCA
CAGACTTGACAGAGACTGTAGTGGACTTTTGGTGTTGGCAAGAACACAGACGGCTGCAAC
AGTTCTTCATTCCATATTCCGAGAGAAAACAACTGGTGCATCCGCATATGTAAATCCTCT
GCTTTCTCTCCGATCTTTCCATTGCTTGTCATGGATCGTGGGAAATAAACATAAGTTTCT
CTTCCAATGACTTTTATTTTACTTGGCAGGGTGTCAAAAAGAACGTAAAATCCTTGAAAA
GAAAATATATGGCACTTGTGATCGGGTGTCCACCACGTCAAAGGGGACAGATTTCAGCGC
CACTCAGAAAGGTCTTCACACTCTGTTTTAGCTAGAGAGTTTTATCCATCTGAGTTTTTA
GTCTATTTTGTTTTATCTAGGAGTTGCTTTGTTTGTTCGAATTCGGTCATTGCTTTTGCT
GCTTTACTGGAGTCAAATTTGAAGGTAAAATATATGTTAAATATCTGGGTAGGTGGTTGT
GGATGATGGAAAATCTGAACGTATCACTGTTAATGACAATGGAGAACTCGTTTCTACTCA
GCATGCTATCACCGAATACCGAGTGATTGAATCTTCACCACATGGTTAGTGAGACTGACT
TCCATTTCTATTCAGTTAAACTTAAAGCAAATGATTTTGCCTTGAGTTTTTAGCACATTG
TTGAATTGCAGGATACACATGGCTTGAGCTTCGCCCTTTAACCGGGAGAAAACATCAGGT
CTCTATAGATATTCAGTTTTTGTTTCAACTTTCTCTCTTTTTTATGTTCTCTTAATACTA
ATCTGTTTTCAACTGTTCTTCGATTGCCACAGCTTCGTGTACACTGCGCTGAAGTGCTAG
GAACACCGATAGTCGGGGACTACAAATACGGTTGGCAAGCTCATAAAGCCCGGGAACCTT
TTGTCTCTTCTGAAAACAACCCAACCAAGCAATCATCATCTCCTTTTGGATTGGATCTGG
ATGGTGGAGATGTCTCTTCGAAACAGCCACACCTTCATCTCCATTCAAAGCAAATCGATC
TGCCAAACATATCACAGCTCTTGGAGAAAATGCAGGTCTCTTCAGACTCTGATATTTCGG
ATCTCGATAGCCTTAAATTCGATGCTCCATTGCCTAGTCATATGCAACTAAGCTTTAATT
TGTTGAAATCTAGAGTCGAAACTTGTGACAAAAATTAGATTTTTTTTCTTACCGAGCTTT
CTTCTTTGTGTTCATTGAGGCCCAAGTATTTGTGTATTTGGACCTGAATATTCTCATACA
AAGATAAATAATTATAATTAAATGATTTTTCGCATATAATCATTATTGTGGTATGATTAA
CACAGTTGGTGTGATGACTGATTG
SEQ ID NO: 2 is an exemplary coding sequence of GROOT1
in Arabidopsis thaliana (GenBank NM_112831.5):
(SEQ ID NO: 2)
ATGGCCAAATGGAGACTTGCGACTGCGACGCTCCGTCGACAACTTCAATCCTCATCGCCA
ACTATCTCTACCTTCAAGAATCCAACCAAAGCATTGTCTGCGGCGGCTCATCAGTCTACT
CGCAGCTACAGTACAACTCAGACCGATGATTCGAGAGGGAAATGGTTAACTTTACCTCCT
TTTTCTCCCACCATCGATGGCACCGCCGTTGGAAAGGATCTCCTTTCCGACGGAGACTCT
GTCAAATCTTCAACGGACAACTCAAAAACGACGGCGCTTAGGTGGATTCTTCGTTGTCGT
CCCGATTTACCCAGAACTCTCGTTCAGAAACTCTTCCGCTTAAGACAGGTTAGAAGAGAA
ATGTCTATGAGTGTTGATGGTGATGAACTACAAAGAAGCCAGCTTAAAAGGGTGGCAGCT
AAGGAGTCCTTGAATGTAGGAGATAGAATTTACCTTCCTTTATCGGTTGACAATGATACG
CCGCAGACGCCACCTGCTAAGAAAGAAAGCTTTCAATGTAGTGATGAAGAACGCAAATTC
GTTTGCAGTTTGGTGTTGTATAAGGATCCAGCCATTATTGTTTTGAATAAACCTCATGGT
TTGGCTGTTCAAGGTGGGAGTGGGATCAAAACCAGTATTGATGAACTCGCTGCCTCTTGC
TTGAAATTTGATAAATCAGAATCTCCCCGGCTGGTGCACAGACTTGACAGAGACTGTAGT
GGACTTTTGGTGTTGGCAAGAACACAGACGGCTGCAACAGTTCTTCATTCCATATTCCGA
GAGAAAACAACTGGTGCATCCGCATATGGTGTCAAAAAGAACGTAAAATCCTTGAAAAGA
AAATATATGGCACTTGTGATCGGGTGTCCACCACGTCAAAGGGGACAGATTTCAGCGCCA
CTCAGAAAGGTGGTTGTGGATGATGGAAAATCTGAACGTATCACTGTTAATGACAATGGA
GAACTCGTTTCTACTCAGCATGCTATCACCGAATACCGAGTGATTGAATCTTCACCACAT
GGATACACATGGCTTGAGCTTCGCCCTTTAACCGGGAGAAAACATCAGCTTCGTGTACAC
TGCGCTGAAGTGCTAGGAACACCGATAGTCGGGGACTACAAATACGGTTGGCAAGCTCAT
AAAGCCCGGGAACCTTTTGTCTCTTCTGAAAACAACCCAACCAAGCAATCATCATCTCCT
TTTGGATTGGATCTGGATGGTGGAGATGTCTCTTCGAAACAGCCACACCTTCATCTCCAT
TCAAAGCAAATCGATCTGCCAAACATATCACAGCTCTTGGAGAAAATGCAGGTCTCTTCA
GACTCTGATATTTCGGATCTCGATAGCCTTAAATTCGATGCTCCATTGCCTAGTCATATG
CAACTAAGCTTTAATTTGTTGAAATCTAGAGTCGAAACTTGTGACAAAAATTAG
SEQ ID NO: 3 is an exemplary genomic sequence of GROOT1
in Glycine max (soybean) (Glyma.01G244300):
(SEQ ID NO: 3)
AAAAAAACACGAATGGGGGTGGTAAATTGAATTGGGTGAGATGAGGGCGGCACAATCCAT
GATGTTGAGAGCGTTGAGGAGTGGGCAGCGTCAGTTCTCGGTGGCGGTGACAAGGCCGTG
GGAGGACAAATGGCTTACTCTGCCTCCCGTCAGTGCGAGTTCGAGTGCGAGTGTGGAGCT
GAATCAACTCTCGTCCACTCCGACCACCGCACTCAAATGGGTTGTTCGGTGCTGCCCCCA
CCTTCCCAGAGCTCTGGTGCACAAGCTTTTCCGTCTAAGACAGGTTCGAATTCACCCTGC
CACTGTTATACAACAACAAACATTCAAAAGGGTAAGGGTATGTGTATGACTTTTCTCCCT
CTCCTCCTCCTCCCTCTTCTTCTAAACTAACTCATTAATGGAAGGTGGCAGCCAAGGACA
CCTTGAACACAGGAGACCGTATCTTACTTCCTCAATCTGTTAAAGTTAAACAAACGCCTA
CACATTCTCATCTCACTCCCCAACAAATCAACTTTATCCGTACTCTTGTTATCTATAAGG
TTTTCAACTTTTACTCCCTTCTTTTTTCTTTCCGCATTTCTCTTTCTCATTCTCATTTTG
TGCTTAGGATCCCGCCATTCTCGTCCTCAACAAACCTCCAGGAATGCCAGTGCAGGTACC
TCATCTCAATGTCCCCATTCTTATTCTCGGATGCTCTTCCATTCATTAATTCCTTCTCCT
GCTTTCTAATTAATTAGGGTGGCATTAATATCAAACGGAGTTTAGATGCTGTAGCTGCTG
CATCTTTAAATTATGGTTACTCTCAACCCCCTCGTCTGGTGAGCTCATTATTCACATTCA
CTATCTCATCTTTTAGTTCTGTGCCAATATGATTCGTAGTTTATTGTTTTTAGGTGCATA
GACTAGACAGAGACTGTTCTGGCATTCTGGTCATGGGAAGGACACACACCAGTACAACAG
TCCTGCATTCCATCTTCCGCGAGAAAACTTCCAGGGCGTCAGATGATGTGAGTGATAAAG
CATTCAGATCATTAGTCTATTATGGTCACATTCTCATATTGCTATATCCATTTCATTGTT
TTTCTTGATTTGGTGCCGTTAGATTGGCAAAGAGAAGAGAATACTACAAAGAAGGTACTG
GGCACTGGTCCTTGGATGTCCTAGACGTCCAAAGGGGTTGGTCACTGCTTCACTGGGTAA
GGTTGTATCCATATTGGCACTTGTTAACTATATTTACCAGAAAATTTCAGTTGGTTAATC
CTCCCTACTCCTGGCATATTATACATTGTGCATGCTTCATCCATTTCATACTCACAAATG
TCAAACTTATAAAACAGCAATTATTTGTATGTAATGTCTGCATAAATGAAAAAAGGTTTG
CACTGAATCATAGCTTTTTCTTAAAACTAAACCATCTAACATTTTAAACAACCAATGGTA
TATGCTGTCAGTAATCACTAAATTTACCATGTTCTTCACTATTTAAAAAAAAGTTTGAAC
AATTATATTACTCAGATGGTGGGGAATGTTATGTGTGGGGAATTTCCCTTAGTCCTTTAA
TACTTTGATGTTTCTTCTGGGTAGCTAGTCTTCTGATAAATCCTTTTTTCAGTTATGGTG
TTTCCTTTAATGCTTTACATGTCTTGTGAACCAACAAAACAAGTTCAAAACAAACGTGTT
AGTATCCATAAGATGCAGCCTCATACACACGAATTGCCACCAACTCTTTGGAATTGCGTG
ACATGAATGCTTAGGCCTTACAGTTACAATATAATAGCCCATAACCTAAATTAGCTTCCA
GCATTAAAGTAAATGGACATTGATTTGAGGGGCTTTCCTCATTTATTGCAAAATAATGTT
GTAAACTTGTAGTTCTTTATTTGATTTAAGGTGGTGGTTGACAATGGGAGATCTGATCGA
ATAACTATCGTTGACAATTCTACATTAATGTCATCACAGCATGCAATTACAGAGTACCGA
GTGATTGCATCATCATCACAAGGTTGGTAATGAAACTTTTAATTCTAAGATTAAATCTGA
TCAAATATCTTTTGAGAATGTTAAGAATAAATTTCATTATTTTCCCCATTTTGCATTTTA
TTTTGTGACATTTTGAACTTATTAGGACCCTAGTTTCAACCACTCTCACTCACACTAATT
GTTAATAGGATGGAGTCATTAAATGTCATTAGTGTTCTGTTCTAGAAGCAATATATCACC
ACTGCTTATAAATTTATTGCAATGTCAAAAAATTCATGCTTCCAAACTATGTTAGGTTAC
AAATTGTTTTATTAAATAATGTAGTTCAACATTTTCACTTGATACTGGAAAAGCTGAGAG
TCCTAGACAAAAAGAGCTAATAGTTACATATCAAGTATGACATCGACTTAGTTTCATGTT
TGGGAAATAGAATTTTACAGTCTGCCAAACAGCATAAGTACTCCTAGCAGCTGGAAGATG
TCTGATTTTTATCACAACAGGGCCTGTTTGCAATACGTTTGTGTAGAAAATTTGTCATGT
GTCTTTTTTCTCCCTCTTTGGCATGAGTTATCCTGCAGGTGCAAAGTTCTTCCCCTCAGC
TCTTCTTAATTGGCAACCCCTACTTATGTCATCTCAAATAGTGCAGGTTACACCTGGTTG
GAGCTGACCCCTTTAACTGGTAGAAAACACCAGGTAACAAGATGATATTTGTGTCACAAC
TTAAAATGTTAAATCATTTCATATGCCTCACCATATTTCTATTCTAACTGCGTTGTACTG
GTACCACAGCTTCGAGTCCACTGTGCTGAGGTGTTAGGTACACCAATAGTAGGAGACTAC
AAGTATGGATGGCAAGCTCACAGGAAGTGGGGACATTTTGATTTGCCTAATGTGGAGGAC
TCACGTGAAGAACTTCTCAATGAAGAAAAACTCCCCTTTGGCCTTAATTTGAATAAAGGG
AGCATCTCTGAGAATCATCCTCGTTTACATCTTCATTGCAAGCAAATGGTCTTGCCTAAT
ATATCTCAAGCACTGCAGAATGTGCAATCAGCTTCCAGTTGTGATCTTTCACTAGTTGAA
GAGCTTGAGTTGGTAGCGGATTTGCCTCCATACATGCAAAGAAGTTGGGATGTCACAAAT
TATTGAGTTAATGTCATCAAAGGCAATTTCTTCCGCTCGGATTTGCCTCTGTACTTTACT
TTTTCTATATAAAAAATGAATTAAATAAATTATTATTAAATATTCCATATATTACTATAG
CTCTCAGTATTACATTTGTTAACGATATCGTTCCTGATATATTGGAAACTAAAGGTTCAT
GTGATTTGTTTATGGACTGTACTTTTTTTTATGATCATTGACTGTATTTAGAATTAACGG
ACATGATCAAATTAATAGTATTGAAATTATTTTTCTTAATGAAAAAGCTATAGATATTTT
G
SEQ ID NO: 4 is an exemplary genomic sequence of GROOT1
in Thlaspi arvense (pennycress) (Ta1014.a04.6.g20490):
(SEQ ID NO: 4)
ATGGCCAAATGGCGACTTGCGACCGCGACGCTCCGTCGCCATCTCCGATCACCATCGCCT
ACTATCTCTTCCGTCTTCAGGGATCCGACCGGAGCCTTGTCTGCAGCTAATCAGCGTCGT
AGGTACAATACACCTGAAGATCCGAGAGGGAAATGGCTAACTCTACCTCCTTTTTCTCCC
ACCGTCGATGCCGCCGCAATCGGAAAGGAGCTATCTTCCGACAGAGACTCCGCAAAAGGT
TCAACGGATGGCTCAACGACGGCGATTAGGTGGATTCTTCGTTGCCGTCCCGATCTACCT
AGGAATCTCGTTCAGAAACTCTTTCGTCTAAGACAGGTCCTCTACTCTCTTCATCATCAG
CCAAGAGTGTATTTCAAAATTGAATCCAATCCCTCCTCAATGGTATTGTTGTTGTCAACT
TCTTACATTGTAAATGGATTCAGGTTAGAAGAGAAATGTCTCTGAGCTGTGATGGTGATG
AGCTACAAAGAAGCCAACTTAAAAGGGTAAATAATGTTCGTCTATTGATTTGAAACAAGA
ACCCTTTTTTTAGAAGCTGCTGTTATCTGATGTTCTTGATTCTGATTCTGACCATTGAAG
GTGTCAGCAAAGGAACCCTTAAACTTAGGTGATCGGATTTACCTTCCTATATCGGTAGAC
AACGATGCGCCGCCGCAGCCTGCTAAGAAAGAAAGCTTTCGTTGCAGTGAAGAAGAGCGC
AAATTCGTTTGCAGTTTGGTGTTGTACAAGGTTTTTAAGACTCTTCACACATGGCGATGT
TGTTAATATTGATTGGTAGATCTAAACTGTGAGTTAATATTGCTCCAGGATCCAGCCATT
ATCGTTTTGAATAAACCTCATGGCTTGGCTGTTCAAGTGAGTTCATTTATACTTTCTTGC
ATTGTAAATTGATCATGGTCTTAGAGCTTGTGACAGAAATGATTAGACTTTTTTGCTCTT
AAACAGGGTGGGACTGGGATCAAAACCAGTATCGATGAACTCGCTGCCACTTGCTTGACT
TTTGATAAATCAGAATCTCCTCGGCTGGTTAGTAGAGAATCATAGTATGATTTAATTAAG
ACTTTATTTTTTGTTAAAACTAGTTAATTTGAAAAAGTATGAATCTTTTGGCTAATGCAA
GTATGGTGTATGTATAATTAGGTGCACAGGCTTGACAGAGACTGTAGTGGACTTTTGGTG
TTGGGAAGAACACAAACGGCTGCAACAGTTCTTCATTCTCTATTCCGCGAGAAAACATCT
GGTGCATCCGCATATGTAAAATCCTCTCATTACTCTCACGCTTGTTTCTTACTTGAGATG
TTTGCTTGTCATGATTCAAGAGAAATCTTCTCTCCGAGTGACTTCTTTTTTTTTATCTCT
TTAGGGTGTAAAAAAGAACATAAAATCCCTGAAACGAAAATATTTGGCACTCGTGATCGG
GTTTCCAAGACGTCAACGTGGACAGATTTCAGCGCCACTCAGAAAGGTCAGAAAGCTTCA
CCAATCTGAGTTCTAGTCCATTTGGTTCGATCTAAGACTTGATTTGTTTGTTTACATTCA
GTCCATTGCTTTTGCTGCTTTAACATATGTGTGAATGTCTGAGTAGGTTGTTGTGGATGA
TGGAAAATCTGATCGTATCACGGTTAATGACAATGGAGAACTCGTTTCTACTCAGCATGC
TATCACTGAATACCGAGTGATTGAATCTTCTCCACATGGTTAGTGTGACAAATGACTTCG
TTTTTTTCTTTTCAGTCAAAACTTAAATCAAACGATTTGGCCTTGAGTAGCACATTGTTG
ATTTGTAGGATACACATGGCTTGAGCTTCGTCCTTTAACCGGGAGAAAACATCAGGTCTC
TATAAAAATCTTCAGTTTTTTTGTTTCAACTCTTGCTCTGTTTTCTCTTCTCTTATGAGT
TATAACAATGTTTTGTCCACTGTCTTCGATTGCGACAGCTTCGTGTACACTGCGCAGAAG
TTCTAGGAACACCGATACTCGGAGACTATAAATACGGTTGGCAAGCTCATAAAGCGCGGG
AACCATTTGTCTCATCCGGAAACAGTAACACCCCAACCAAGCCGTCATTGTCTCTTTTTG
GCTTGGATCTGGATGGTGGCGATGTGTCTTCAAAGCAGCCACACCTTCATCTCCATTCGA
AGCAGATTGATCTGCCAAACATCTCTCAGCTCTTGGAAAAGTTGCAGGTTTCTTCAGATT
CTGATATTTCGGATCTCGGTGGCCTCAAGTTCGATGCTCCGTTGCCTGCTCATATGCAGC
TAAGCTTTAACTTGTTAAAATCTAGAGTCGAAACTTGTGATATTGCTAAGCCCGAGTTGG
TATCTTTAACAGATAGTAATTGTTAG
SEQ ID NO: 5 is an exemplary coding sequence of GROOT1
in Sorghum bicolor (sorghum) (SbiRTX430.02G143400):
(SEQ ID NO: 5)
ATGGCGATCAGGACGGCAGCGCTCCTCTTCCGCCGCCGGGCCGTCGCCGCCGCCGCCGCC
GCCGCAGCTCCAAAGCCGCTACAACAGTACTTCGCTGGTCTGTCCAGCGCAGTGGGGCAC
GTTACTCTAGACGATGGCGGTCGCATTGGCGGTGGGGAGGAGAACAAGAAGCGGTGGGTG
GAGCTCCCTCCCTTCGCACCACTCGACGCCAACGCCGCCGCTCGAGCCATCTATCGGGGA
GATAATGGGGAAGGTTCACGCTCCAATTCCACGGCTATCAGGTGGGTCCGGCGCTGCTGC
CCACACCTGCCGGCGTCGCTCGTGCAGAAGCTGTTCCGCCTCCGCAAGGTGAAGAAGAAT
CTTGTGACTGCTGATACCTCTTCAACAGACAGTATTGCCCAGCAACTCCGGCTGAGAAGG
GTTTCAGCAAAAGATGAACTTGTGCCTGGTGATATTCTCTTTCTACCTGATAACATTCAA
GAATCTTCTGTTACTGAGAAGAAGAAATTTGGTAACAAGAATGAGATTGATTTTCTACGC
AGCCTTGAGATCTACAAGGATAGGGCCATCATCGTGCTCAACAAACCACCTGGAATGCCA
GTGCAAGGTGGTGTTGGCATAAAAAATAGTATTGATATACTGGCCCCAATGTTTGAGGAC
GGTTCTTCTGAAGCACCTCGGCTGGTCCACAGGCTTGATAGGGATTGCAGTGGCGTCCTA
GTTCTGGGAAGAACTCAACTTAGCACTTCCATTATGCATGCTATATTTCGTGAGAAAACT
GCTGATGCTTTAGGTGATGGTACTCAACAAGTACTGCAAAGAAAATATGTTGCACTTGTT
ATTGGAAGACCTAGGCATCCCAAGGGTTTATTGTCAGCTCCACTTGCAAAGGTTGTATTA
CAAGATGGCAGGTCGGAGCGTCTGACTGTTTGCGCTGGTCCAAATACCGCTTCTGTTCAA
GATGCTTTGACAGAGTACCGTGTGATTGAGTCTTGTCCTCAAGGGTACACTTGGTTAGAA
TTATTCCCTCGGACTGGGAGGAAGCATCAGCTCCGAGTCCATTGTGCGGAGGTTCTGGGA
ACACCAATCGTTGGGGATTACAAGTACGGACGGCAAGCACACCAGAACTGGACGCCTCTT
CCCATGCCGCAAACAATCGACGAGGAAATGCTCAAGAAAAGGAAGCTTCCCTTTGGGCTT
GCTTTGGGCGGTGGAAGCGTAGCTGAGCAGCAGCCACAGCTGCATCTACACTGCAAGCAG
ATGATCCTTCCTGACATCTCAGCAGCTATGCAGCAGCTGCAGTCTTCAGATGCTGACCAC
AATTTCTCTGATCTGGAGAAGCTGAGCTTTGTCGCCCCATTGCCATCGCACATGCGGTTG
AGCTGGAGGATTCTGATGTCCATAGGCAAGTAA
SEQ ID NO: 6 is an exemplary genomic sequence of GROOT1
in Brassica napus (canola) (BnaC05G0378600WE):
(SEQ ID NO: 6)
ATGGCGGCCAATTGGAGAGTTGCGACCGCGGCCCTCCGTCGTCATCTCCTATCCCCACCG
CCTATTATCTTTGCCGCCTTCAGGGAGTCCACCGGAGCCTTATCCGCGGCTAATCAGCGT
CGTAGCTACACGACAGCTCTCGCTGATGATGATCCGAGAGGGAAATGGCTCACGCTACCT
CCTTTCTCTCCCACCATCGACGCCGCCGCGGTAGGAAAGGAGCTCTCTTTCGACGACGGA
GACTCTGTCGTCAAAGGCTCAAATGATGGCTCAACGACGGCGCTGAGGTGGATTCTTCGT
TGCCGTCCTGACCTACCTAGAAACCTCGTACAGAAACTCTTTCGTTTAAGACAGGTCCGT
GTAAAGATTGTATTTTTTTGATGATTGAAGTTACAGGGGATAAGATATTTGTAAATGTTT
TCAGGTTAGAAGACAAGTAGTAGTAGTAGTGCCTATGAGTTGTGAACTACAAAGAAGCCA
ACTTAAAAGGGTAAATTGTTCTTCTGTCTATCTCTTGGTGCTGTTTTTTTTTTTTTTTTT
TTTTTTTTTTTGAGAAGCTGATGCTGTCTGATGGTACTGATTGAAGGTGGCAGCCAAGGA
GTCATTGAATGTGGGAGATAGAATTTACCTTCCTCTATCTGTTGGCAATGATGCTCCCCA
GCCTGCTAAGAAAGAACGCTTTCTTTGCAGTGAAGAAGAGCGCAAATTCGTTTGCAGTTT
AGTGTTGTACAAGGTTTGTTTGTTTGTTTTTTTTTTGCAAGAAAATGTTATATAGATTGA
TAGATTTGTAACTGTGGGTTAATTTGCCAGGATCCAGCCATTATTGTCTTGAATAAACCT
CATGGTATGGCTGTTCAAGTAAGTCATATATTCTCCATGCTTTCTGATGCATCGTATTCG
TGGAGCTGATAGACTTTTTCTCTTAAACAGGGAGGGACTGGGGTGAAAACCAGCATTGAT
GAACTCGCTGCCACTTGTTTGACTTTTGATAAGTCAGAATCTCCCCGGCTGGTTAGTAGC
GAAATCATATAGTAGTGCACCAAGGCTTTCTGTTAAGTCCTAGCTTATATGGAAGAAGTT
TATGAATCTCTTGGCTTATGCAAGTATGGTGGTGTATGTATTAGGTGCACAGACTTGACA
GAGACTGTAGTGGGCTTTTGGTGTTGGGAAGAACACAAACAGCTGCAACACTTCTTCATT
CTATATTCCGCGAGAAAACATCTGGTGCTTCCGCCTATGTAAGTCATATGCTTTCTCTTA
GAAGTTTTTCTTACTTTGAAATAAGTTTTCTTGTCATATGATTTCTGAGAAATCGTAAAA
AAATGTCTCTTTCGGTGACCTTTCCTTTATTTCTTTAGGGTGTCAAGAAGAACATAAAAT
CCTTGAAAAGAAAATATCTGGCACTCGTGATCGGGTGCCCAAGACGTCAAAGGGGACAGA
TCTCAGCGCCACTCAGAAAGGTGATTTTACACTCTGTTTTAGCTATCAGAATGTTTCACC
CGTCTAAGTTTTGTCCACTTTGGTTCGATCCAAGAGATGATTAGTCTGTGCACATTCAAT
CCATTGCTTATGTTGTTTTACTGGAGTAAAATTCTTTGCTAAAACATATGTGGAATGTCT
GTGTAGCTTGTTGTGGATGATGGAAAATCTGATCGCATCACAGTTAATGACAATGGAGAA
CTAGTTTCCACTCAGCACGCTATCACTGAATACCGAGTGGTCGAATCTTCACCACATGGT
TAGTGTACACTGTTGCTATCGTTTCACTTAACTTAGAATCAAATGGTTTGGCCTTTTATA
GCATACTGTTGCTTTATAGGATACACATGGCTGGAGCTTCGTCCTTTAACCGGCAGAAAA
CATCAGCTCCGTGTACACTGCGCAGAAGTGCTAGGAACACCGATACTCGGGGACTATAAA
TACGGTTGGCAAGCTCATAAAACCAGGGAACCTTTTGTGTCTCCAGAAAACACCCGGACC
AAGACATCATCTCCTTTTGGCCTCGATATGGAAGGTGGAGATGTATCTTCAAAACAGCCA
CACCTTCATCTCCATTCTAAGCAAATCGATCTGCCAAACATCTGTCAGCTCTTGGAGAAA
TTGGAGGTTTCTTCCGACTCTGATATCTCGGATCTCGATAGCCTTAAATTCGATGCTCCG
TTGCCTACTCATATGCAGCTAAGCTTCAACTTGTTGAAATCTAGAGTCGAAACTTGTGAC
TATTGTTAG
SEQ ID NO: 7 is an exemplary coding sequence of GROOT1
in Brassica napus (canola) (BnaA05G0316300):
(SEQ ID NO: 7)
ATGGCGGCCAATTGGAGAGTTGCGACCGCGGCCCTCCGTCGTCATCTCCGATCCCCACCG
CCTACTATCAGGGAGTCCACCGGAGCCTTATCCGCGGCTAATCAGCGTCGTAGCTACACG
ACAGCTCTCGCTGACGATGATGATGATCCGAGAGGGAAATGGCTCACTCTACCTCCTTTC
TCTCCCACCATCGACGCCGCCGCGGTAGGAAAGGAGCTCTCTTTCGGCGACGGAGACTCC
ATCGTCAAAGGCTCAACTGATGGCTCAACGACGGCGCTGAGGTGGATTCTTCGTTGCCGC
CCTGACCTACCTAGAAACCTCGTACAGAAACTCTTTCGTTTAAGACAGGTTAGAAGACAA
GTAGTAGTGCCTATGAGTTGTGAACTACAAAGAAGCCAACTTAAAAGGGTGGCAGCTAAG
GAGTCCTTGAATGTAGGAGATAGAATTTACCTTCCTTTGTCTGTAGGCAATGATGCGCCG
CCTCCTGCTAAGAAAGAACGCTTTCGTTGCAGTGAAGAAGAGCGCAAATTCGTTTGCAGT
TTGGTCTTGTACAAGGATCCAGCCATTATTGTCTTGAATAAACCTCATGGTATGGCTGTT
CAAGGAGGGACTGGGGTGAAAACTAGCATCGATGAACTCGCTGCCACTTGTTTGACTTTT
GATAAGTCAGAGTCTCCCCGACTGGTGCACAGACTTGACAGAGACTGTAGTGGGCTTTTG
GTGTTGGGAAGAACACAAACAGCTGCAACGCTTCTTCATTCTATATTCCGCGAGAAAACA
TCTGGTGCTTCCGCCTATGGTGTCAAGAAGAACATAAAATCCTTGAAAAGAAAATATCTG
GCACTCGTGATCGGGTGCCCAAGACGTCAAAGGGGACAGATCTCAGCGCCACTCAGAAAG
GTTGTTGTGGATGATGGAAAATCTGATCGTATCACAGTTAATGACAACGGAGAACTCGTT
TCCACTCAGCATGCTATCACCGAATACCGAGTGGTCGAATCTTCACCACATGGATACACA
TGGCTGGAGCTTCGTCCTTTAACCGGCAGAAAACATCAGCTCCGTGTACACTGCGCAGAA
GTGCTAGGAACACCGATACTCGGGGACTATAAATACGGTTGGCAAGCTCATAAAACCAGG
GAACCTTTTGTCTCTTCTGAAAACACCCCGACCAAGCCATCACCGTCTCCTTTTGGTCTG
GATATGGAAGGTGGAGATGTATCTTCAAAACAGCCACACCTTCATCTCCATTCTAAGCAA
ATCGATCTGCCAAACATCTGTCAGCTCTTGGAGAAATTGGAGGTTTCTCCGGACTCTGAT
ATCTCGGATCTCGATGGCCTTAAATTCGATGCTCCGTTGCCTACTCATATGCAGCTAAGC
TTCAACTTGTTGAAATCTAGAGTCGAAAGTAGTGACAATTGTTAG
SEQ ID NO: 8 is an exemplary protein sequence of GROOT1
in Arabidopsis thaliana:
(SEQ ID NO: 8)
MAKWRLATATLRRQLQSSSPTISTFKNPTKALSAAAHQSTRSYSTTQTDDSRGKWLTLPP
FSPTIDGTAVGKDLLSDGDSVKSSTDNSKTTALRWILRCRPDLPRTLVQKLFRLRQVRRE
MSMSVDGDELQRSQLKRVAAKESLNVGDRIYLPLSVDNDTPQTPPAKKESFQCSDEERKF
VCSLVLYKDPAIIVLNKPHGLAVQGGSGIKTSIDELAASCLKFDKSESPRLVHRLDRDCS
GLLVLARTQTAATVLHSIFREKTTGASAYGVKKNVKSLKRKYMALVIGCPPRQRGQISAP
LRKVVVDDGKSERITVNDNGELVSTQHAITEYRVIESSPHGYTWLELRPLTGRKHQLRVH
CAEVLGTPIVGDYKYGWQAHKAREPFVSSENNPTKQSSSPFGLDLDGGDVSSKQPHLHLH
SKQIDLPNISQLLEKMQVSSDSDISDLDSLKFDAPLPSHMQLSFNLLKSRVETCDKN
SEQ ID NO: 9 is an exemplary protein sequence of GROOT1
in Glycine max (soybean) (Glyma.01G244300):
(SEQ ID NO: 9)
MRAAQSMMLRALRSGQRQFSVAVTRPWEDKWLTLPPVSASSSASVELNQLSSTPTTALKW
VVRCCPHLPRALVHKLFRLRQVRIHPATVIQQQTFKRVRVAAKDTLNTGDRILLPQSVKV
KQTPTHSHLTPQQINFIRTLVIYKDPAILVLNKPPGMPVQGGINIKRSLDAVAAASLNYG
YSQPPRLVHRLDRDCSGILVMGRTHTSTTVLHSIFREKTSRASDDIGKEKRILQRRYWAL
VLGCPRRPKGLVTASLGKVVVDNGRSDRITIVDNSTLMSSQHAITEYRVIASSSQGYTWL
ELTPLTGRKHQLRVHCAEVLGTPIVGDYKYGWQAHRKWGHFDLPNVEDSREELLNEEKLP
FGLNLNKGSISENHPRLHLHCKQMVLPNISQALQNVQSASSCDLSLVEELELVADLPPYM
QRSWDVTNY
SEQ ID NO: 10 is an exemplary protein sequence of GROOT1
in Thlaspi arvense (pennycress) (Ta1014.a04.6.g20490):
(SEQ ID NO: 10)
MAKWRLATATLRRHLRSPSPTISSVFRDPTGALSAANQRRRYNTPEDPRGKWLTLPPFSP
TVDAAAIGKELSSDRDSAKGSTDGSTTAIRWILRCRPDLPRNLVQKLFRLRQVRREMSLS
CDGDELQRSQLKRVSAKEPLNLGDRIYLPISVDNDAPPQPAKKESFRCSEEERKFVCSLV
LYKDPAIIVLNKPHGLAVQGGTGIKTSIDELAATCLTFDKSESPRLVHRLDRDCSGLLVL
GRTQTAATVLHSLFREKTSGASAYGVKKNIKSLKRKYLALVIGFPRRQRGQISAPLRKVV
VDDGKSDRITVNDNGELVSTQHAITEYRVIESSPHGYTWLELRPLTGRKHQLRVHCAEVL
GTPILGDYKYGWQAHKAREPFVSSGNSNTPTKPSLSLFGLDLDGGDVSSKQPHLHLHSKQ
IDLPNISQLLEKLQVSSDSDISDLGGLKFDAPLPAHMQLSFNLLKSRVETCDIAKPELVS
LTDSNC
SEQ ID NO: 11 is an exemplary protein sequence of GROOT1
in Sorghum bicolor (sorghum) (SbiRTX430.02G143400):
(SEQ ID NO: 11)
MAIRTAALLFRRRAVAAAAAAAAPKPLQQYFAGLSSAVGHVTLDDGGRIGGGEENKKRWV
ELPPFAPLDANAAARAIYRGDNGEGSRSNSTAIRWVRRCCPHLPASLVQKLFRLRKVKKN
LVTADTSSTDSIAQQLRLRRVSAKDELVPGDILFLPDNIQESSVTEKKKFGNKNEIDFLR
SLEIYKDRAIIVLNKPPGMPVQGGVGIKNSIDILAPMFEDGSSEAPRLVHRLDRDCSGVL
VLGRTQLSTSIMHAIFREKTADALGDGTQQVLQRKYVALVIGRPRHPKGLLSAPLAKVVL
QDGRSERLTVCAGPNTASVQDALTEYRVIESCPQGYTWLELFPRTGRKHQLRVHCAEVLG
TPIVGDYKYGRQAHQNWTPLPMPQTIDEEMLKKRKLPFGLALGGGSVAEQQPQLHLHCKQ
MILPDISAAMQQLQSSDADHNFSDLEKLSFVAPLPSHMRLSWRILMSIGK
SEQ ID NO: 12 is an exemplary protein sequence of GROOT1
in Brassica napus (canola) (BnaC05G0378600WE):
(SEQ ID NO: 12)
MAANWRVATAALRRHLLSPPPIIFAAFRESTGALSAANQRRSYTTALADDDPRGKWLTLP
PFSPTIDAAAVGKELSFDDGDSVVKGSNDGSTTALRWILRCRPDLPRNLVQKLFRLRQVR
RQVVVVVPMSCELQRSQLKRVAAKESLNVGDRIYLPLSVGNDAPQPAKKERFLCSEEERK
FVCSLVLYKDPAIIVLNKPHGMAVQGGTGVKTSIDELAATCLTFDKSESPRLVHRLDRDC
SGLLVLGRTQTAATLLHSIFREKTSGASAYGVKKNIKSLKRKYLALVIGCPRRQRGQISA
PLRKLVVDDGKSDRITVNDNGELVSTQHAITEYRVVESSPHGYTWLELRPLTGRKHQLRV
HCAEVLGTPILGDYKYGWQAHKTREPFVSPENTRTKTSSPFGLDMEGGDVSSKQPHLHLH
SKQIDLPNICQLLEKLEVSSDSDISDLDSLKFDAPLPTHMQLSFNLLKSRVETCDYC
SEQ ID NO: 13 is an exemplary protein sequence of GROOT1
in Brassica napus (canola) (BnaA05G0316300):
(SEQ ID NO: 13)
MAANWRVATAALRRHLRSPPPTIRESTGALSAANQRRSYTTALADDDDDPRGKWLTLPPF
SPTIDAAAVGKELSFGDGDSIVKGSTDGSTTALRWILRCRPDLPRNLVQKLFRLRQVRRQ
VVVPMSCELQRSQLKRVAAKESLNVGDRIYLPLSVGNDAPPPAKKERFRCSEEERKFVCS
LVLYKDPAIIVLNKPHGMAVQGGTGVKTSIDELAATCLTFDKSESPRLVHRLDRDCSGLL
VLGRTQTAATLLHSIFREKTSGASAYGVKKNIKSLKRKYLALVIGCPRRQRGQISAPLRK
VVVDDGKSDRITVNDNGELVSTQHAITEYRVVESSPHGYTWLELRPLTGRKHQLRVHCAE
VLGTPILGDYKYGWQAHKTREPFVSSENTPTKPSPSPFGLDMEGGDVSSKQPHLHLHSKQ
IDLPNICQLLEKLEVSPDSDISDLDGLKFDAPLPTHMQLSFNLLKSRVESSDNC

### II. Exemplary Nucleic Acid and Protein Sequences of GROOT2

SEQ ID NO: 14 is an exemplary genomic sequence of
GROOT2 in Arabidopsis thaliana (TAIR AT3G19590):
(SEQ ID NO: 14)
TTCGAATTCAAATAAAATAAAATTAGTTTTTCTGTTTGGATATGTAGAGTAGATCTAATC
GACCCTTGTACTTCTGCAATTTGAATTCAAAATTTAAACCACTCTACTTTTGAATCTCTC
TCTCACTAAACCTAGAAAGTAGAAAACCCTAGCATTTGTGATCTTCAACGGGAAAAATGA
CGACTGTGACTCCGTCCGCCGGTCGTGAGCTCTCGAATCCGCCGTCCGACGGCATTTCTA
ATCTCCGATTTTCTAATAACAGTGATCATCTCCTCGTTTCTTCATGGGATAAGGTTAGTG
AAACACCTCATTTGCTCTTTGACTGATTTGATTCGAGTCCTCTCTTTCCTTCATCTGAAG
TTTTTTTTTCTGTTGTTCTGCGTTTGTTGAATCTAAGCGTGTGAGATTGTATGATGTGAG
CACCAATTCGTTGAAAGGAGAGTTCTTACATGGCGGAGCAGTACTCGATTGCTGTTTTCA
CGATGACTTCTCCGGCTTCAGTGTTGGCGCTGATTACAAAGTCCGACGGTATTGTCTCTC
TTTTCTCTCAGTCATGTGAAGAGACTGTTGGCATAATAATGTTTCCCCTCAGGCTAATTT
AACATTGGCTGCTCAGAAGTTGTTGATTTTACTGCGATTTTTTCTTCATATTTTGTCACT
GTGACGGCTATGGAATCTTTCTACATTTAGCAAATGCCTTGAATCCGTTGTATAATTTCC
TTAATTTATAAGGTTTCATCTGGTTATACTGCAGGATTGTATTCAATGTCGGCAAAGAGG
ACATTTTGGGGACACATGACAAAGCAGTGCGATGTGTTGAGTATTCTTACGCTGCGGGTA
GGTAGAGTGGTACTCTTTATGCTAGTTTGAACTTCAACACTGACGATTTCTGGTGCTTTT
AAGTTCTAGAAAACTCATAGCTAACCAGTTTTTGTTATATGACTCTGATGATGGATGATT
TAATACCTTAGCTGTCTTGGACAGTGAACTTTGTTCCCTAATCTTTTGTAATGTAACTAT
ATCTGGAACTCAGATATTTTGATTTCAGCATTTTGTTTTTTTACACATTATGTTTATTTT
GTGATGTTACAGGACAAGTGATCACTGGATCTTGGGATAAAACAGTTAAATGTTGGGATC
CAAGAGGCGCTAGTGGGCCTGAACGCACCCAAGTGGGAACATATTTGCAACCAGAACGTG
TTTACTCTATGTCTCTTGTTGGACATCGTTTGGTAGTGGCTACAGCAGGAAGGCATGTAA
ACATCTATGATCTCAGAAATATGTCTCAGCCTGAGCAAAGAAGGGAGTCTTCACTGAAAT
ACCAGACGAGATGTGTGCGTTGTTATCCTAATGGAACAGGTTAGATGACTGACATATTGT
TTCTGTCCTTGCTAATTTTTTCTTCCAATTCCAAAGTATACTCTCAGTTCCTCTTCTTCC
ATACCAATGAATTTCGGAAATTGAAAATTTACAGGCTATGCTCTTAGCTCTGTTGAAGGA
AGGGTTGCAATGGAGTTTTTTGATCTGTCAGAGGCTGCTCAAGCTAAAAAGTATATTCCT
TGTGTTCTTCTTTTCTCTTTTCCATGACTTTGACTGACAAATGTATTTGAACTTATTCTC
AGATATGCTTTCAAATGTCATCGGAAATCAGAGGCTGGAAGGGACATTGTTTACCCTGTA
AATTCCATTGCCTTCCATCCAATGTGAGTTCTTGTCTTAGAAGGAGCCCAACTGTTTCAT
AGATTTCTATATTTACTACTTTTTCAAAACACAAGAAGGTTAAGATAATACTCCAACTAA
ATAAGAAAGTAGGAAAAATATTTTCGTGTAAAGTTGTTCAGCCCTTGTCTGCTGATTGAT
ATGTTAAAGATGAAATTCTAATGATGAAGAGAACAAATCGACTTGGAAAGCTGCTTGGAT
GCTTGGTCTGAGAACTTATGGGATATAAGAGCAAATGAACTAATGCTAATATGCTGTGTC
TGTAAACAGTTTCTGTAATTAGACAATATGTTAGCTTATGCTTTGTCTGAGAACTAATGG
GAAAAATACAATGCTGAGAAATATCTCCTGTATAGCTACTTTCCTGTGTAATCATCTGAG
ATTTTTAATTGCAGCTATGGCACCTTTGCAACTGGAGGCTGTGATGGTTTCGTCAACATT
TGGGATGGTAACAACAAGAAGAGGCTATATCAGGTTAGTAATGAGCAAGATCTTTTGGTT
ATTATTCTTTCAATGTTAGTGTGGCACAAAATTTATTTGCCTCCTTGTTCCTTTTGTAGT
ACTCAAAGTATCCAACGAGTATCTCGGCACTGTCATTCAGTCGAGATGGTCAGCTGCTGG
CTGTTGCTTCAAGTTACACATTTGAAGAGGGAGAGAAATCGTAAGTAACTTGTTCCCTTT
CTAATCCTTTGAAGTTTTAGAATAGTATGTCTTTGAAATCTCTGATGATTTGTGCATTGG
TTTGATTGGTTGCTTATTGAGATAACATAGCAGAGAGTAACCCTGTGCCTTAATCTTACA
GAGATTATCACAAACAAATTAATGCTTTAAAGGCACCCTTTTTGTCTTTGTCACAAAACC
GCACACTTAAGGATAGTAATCTCTTTGTATTTGGTTTCAAGTTAGAATTCTCTTTGATTG
GGTCTCTGTGTGAGAGGTTTTGCAGGCAAGAACCGGAGGCCATCTTTGTAAGAAGCGTGA
ATGAAATCGAAGTGAAACCAAAACCGAAAGTATACCCGAATCCTGCGGCGTAGAAAGGAA
GAAACACAAGTTATTTCCTATGTTGGTTGTTTTTGTATTTGCTAGGAGTGTTCCAAAGAT
TGAATCAACGTATGTTTGAGCTTTTAAATAACTAGTGAAATATCCACATTCACCGTATAT
GTTTAAATTTCTGTAGTCAAACTCTCTTGTCAAACATATAATATAAGATGACAAATTTCC
AGTGTTTAAAAGATAGAAAGAGATCATTCTAC
SEQ ID NO: 15 is an exemplary coding sequence of GROOT2
in Arabidopsis thaliana (GenBank NM_112849.5):
(SEQ ID NO: 15)
ATGACGACTGTGACTCCGTCCGCCGGTCGTGAGCTCTCGAATCCGCCGTCCGACGGCATT
TCTAATCTCCGATTTTCTAATAACAGTGATCATCTCCTCGTTTCTTCATGGGATAAGCGT
GTGAGATTGTATGATGTGAGCACCAATTCGTTGAAAGGAGAGTTCTTACATGGCGGAGCA
GTACTCGATTGCTGTTTTCACGATGACTTCTCCGGCTTCAGTGTTGGCGCTGATTACAAA
GTCCGACGGATTGTATTCAATGTCGGCAAAGAGGACATTTTGGGGACACATGACAAAGCA
GTGCGATGTGTTGAGTATTCTTACGCTGCGGGACAAGTGATCACTGGATCTTGGGATAAA
ACAGTTAAATGTTGGGATCCAAGAGGCGCTAGTGGGCCTGAACGCACCCAAGTGGGAACA
TATTTGCAACCAGAACGTGTTTACTCTATGTCTCTTGTTGGACATCGTTTGGTAGTGGCT
ACAGCAGGAAGGCATGTAAACATCTATGATCTCAGAAATATGTCTCAGCCTGAGCAAAGA
AGGGAGTCTTCACTGAAATACCAGACGAGATGTGTGCGTTGTTATCCTAATGGAACAGGC
TATGCTCTTAGCTCTGTTGAAGGAAGGGTTGCAATGGAGTTTTTTGATCTGTCAGAGGCT
GCTCAAGCTAAAAAATATGCTTTCAAATGTCATCGGAAATCAGAGGCTGGAAGGGACATT
GTTTACCCTGTAAATTCCATTGCCTTCCATCCAATCTATGGCACCTTTGCAACTGGAGGC
TGTGATGGTTTCGTCAACATTTGGGATGGTAACAACAAGAAGAGGCTATATCAGTACTCA
AAGTATCCAACGAGTATCTCGGCACTGTCATTCAGTCGAGATGGTCAGCTGCTGGCTGTT
GCTTCAAGTTACACATTTGAAGAGGGAGAGAAATCGCAAGAACCGGAGGCCATCTTTGTA
AGAAGCGTGAATGAAATCGAAGTGAAACCAAAACCGAAAGTATACCCGAATCCTGCGGCG
TAG
SEQ ID NO: 16 is an exemplary genomic sequence of GROOT2
in Thlaspi arvense (pennycress) (Ta1014.a04.6.g20630):
(SEQ ID NO: 16)
ATGAATCTCGAACTCTCGAATCCACCTTCCGACGGAATTTCCAATCTCCGATTTTCCAAT
GCTAGTGATCATCTCCTCGTTTCTTCATGGGATAAGGTTAGAGACAAAACTTCTCTACAT
TTGTCTTGTTTTCCGACTGATGTGATTAAAGTCCTCTTACTGTATCTGAAGCTTGTGAGG
TTGTATGATGTGAGCACCAATTCGTTGAAAGGGGAGTTCTTACATGGCGGGCCAGTACTG
GATTGCTGTTTTCACGATGATTCCTCTGGCTTCAGTGTTGGCGCCGACAACAAAGTCAGA
CGGTAATGTCTCTTTTTCCTTTCTCTACTTCATACTTAATGTTTAGAAGTAAAATTTGAA
GTAGGCCTGCATAAAAGAATCTCAATCTCGTGAAGATCATACGAATAATTTCACTGAGGC
TTAACATGAAATTGGCTTAGAAGTTCTAAGTTTCTGTTGGCTTCTATCTCGGTTTTTCTA
CTCTTTTAGCAGAAAGTGCTCAAAAATGCGTTGTAATTTTTCTTTATATTCACGAGATTT
TCTCTCTGGTTATGTGCTTCAGGATCGTTTTCAATGTCGGCAAAGAGGATATCTTGGGGA
TGCATGAATCTCCAGTGCGATGTGTTGAGTATTCTTATGCTACAGGTAGGTGGAGTGGCC
ATCATGCTCATGATGCTAGTTCAACAGTAACATCTCTGCAAGTATTTGCTTTCAGCATTT
TGATTCTTTATTATTGCCTTCAAAGCTATACGGACACATTATGTTTTATGTGATGTGATG
TTGCAGGGCAAGTGATCACTGGATCTTGGGATAAAACGGTTAAATGCTGGGATCCAAGAG
GCGCGAGTGGGCCGGATCGAACTCAGGTGGGAACGTACTTGCAACCAGAGCGTGTTTACT
CTCTGTCACTTGTTGGGAACCGTTTGGTTGTGGCAACAGCAGGAAGACATGTGAACATCT
ATGATCTCAGAAACATGTCTCAGCCTGAGCAAAGAAGGGAGTCTTCACTTAAATACCAGA
CGAGATGTGTTCGTAGTTATCCTAATGGAACAGGTTTAGTGACTGACATATTGCTCTCAC
TCTGGTTTTTAACCTCAGTTCCTCTTCCACAGTTTTGAGTTTCTGATATTGAAATTTTTA
CAGGGTATGCTCTTAGCTCTGTTGAAGGAAGGGTTGCAATGGAGTTTTTCGATCTGTCAG
AGGCTGCTCAAGCTAAAAAGTATTCTTCCCTGTGTTATTTTTTTCTTTTCTCTTTTCCTT
GGCTTTGACTTACAGATGGATTTGAACTTGTTCTCAGATATGCTTTCAAATGTCATCGGA
AATCAGAGGCTGGAAGGGACATTGTTTACTCCGTAAATACCATTGCATATCATCCGATGT
GAGTTCTTATCTTTAGAAGGAGTCCAACTGTTTTCTTGATTAATCACTTTTCAGAGAACA
CTAGTAGGTTAATACTATCGTATAACTAAAGAAGGGAGGAGGCAACATATTATAGTCTAA
GGTGTTAATTACTGATTGAAATGGTGAAGATGAAATTCTAATGATGAAGAGAACAAATGG
GCTTTGAAGGCTAGATCGAAGTTTCTTGGATGCTAATATGCTATGTTTATAAATTTCTGT
AACTGTTTTGTTTTTGTGTTTATATATTCAACTAGACAAAGGTTGACTTCTGCTTGGTCA
TGAGAACGAATGAGAAAATTATGATGCTGAGAAAGTCTTCCCCTGAATCATCTGAAATTT
TTAATTGCAGTTATGGCACCTTTGCAACCGGAGGTTGTGATGGTTTTGTTAATATTTGGG
ATGGTAACAACAAGAAGAGGCTATATCAGGTTCAGTAAATAACAAGAGCTATATTGTTTT
CATTTTCTCAGTGTATCTTAGTGTGACACAGAAGTGTATTATTTGGTCCTTGTTTCGTTT
GCAGTATTCAAAGTATCCAACAAGTATCTCAGCACTTTCATTCAGTCGAGATGGTCAGCT
ACTAGCTGTTGCTTCGAGTTACACATATGAAGAGGGGGAGAAATCGTAAGTATCTTTCCC
CTTTATCATCATACACAGAATGATTTAGGATTGTTTCTGGCTGAAATGCTTATAGGGAAT
AATCACATAGCATTTCTCGTATAGGACAAAACTAAACTTTCGGTCTATATGAATCTTTAT
GTTCCTTAGTCATAAACAAATAAATGCTTAAACTGCACACTTGAGGATAGTAGTCTTGTA
ACGCAAACCCGAGAGTTTCATAACTAAACCACCTTCGAAAAAAGACTGCTGTAGAGATAT
CTTTTGTATTTGGGTTAAAAATGATCTTTAACTTGCTCTGTGTATGAGGTTTTACAGGCA
CGAACCTGATGCCATCTTTGTGAGAAGCGTTAATGAAATCGAAGTGAAACCTAAACCCAA
AGCATATCCAAATCATGCAGCGTAG
SEQ ID NO: 17 is an exemplary genomic sequence of GROOT2
in Brassica napus (canola) (BnaC01G0327800WE):
(SEQ ID NO: 17)
ATGAGTCTGCCTCCTCCGTCCGCCGGTCGTGAGCTAGCGAATCCACCGTCCGACGGCATT
TCGAATCTCAGATTTTCCAACACGAGCGACCATCTCCTCGTCTCTTCATGGGATAAGGTT
AGAAACACTGATTTGATCGAACTCCCTTTTCCTTTTAATCTGAATCGTTTCTTCTTCTTA
TGTGTCTAAGCGTGTGAGATTGTACGATGTGAGCACCAATTCGTTGAAGGGCGAGTTCTT
ACACGGCGGCGCAGTTCTCGATTGCTGTTTCCACGACGATTCCTCTGGTTTCAGCGTTGG
CAGCGACAACAAAGTCAGACGGTATTTGTCTCTTCTGTGAATCTGGGGTTGGACTCAACT
CTGTGAAGAAAAATAAGAAAGTTTTGGTTATATATGTTCAGGATTGTTTTCAATGTTGGC
AAAGAGGATGTTCTGGGGATGCATGAAAAGCCAGTGCGTTGTGTTGAGTATTCTTATGCT
GCAGGTATTGTTATAGTTTTGATCATCATCATGCTCTTTCATGCTAGCTTTGAAACAGTG
ACATTGACTAGTTCTGGTGCTTTTCTTTGTCTAGAAAACTCATCATAGTTAACCAATTTA
TGTTATAATTGAACATTAGGATAGTGAACCTTGATTGTTAATCATGCTCTTTGATGCTAG
CTTTGAACAGACCATTTCTGGTGCTTTTCATCTATAGAAAACTCTTAGTTAAGCAATTAC
TATGGACACTGAACTCTTGTTATTGAAGTAACGATTTCTTTTTAGAAGTAACTTATCATG
TTTCTCACTGCTCCAAGCCTCCAAAGTAATGCTGATATGTTATGTTTTTTTTTTGTTCTT
ATGTGATGTTACAGGGCAAGTGATTACTGGATCTTGGGATAAAACAGTTAAGTGTTGGGA
TCCAAGAGGTGCAAGTGGGCCCGAACGCACCCAGGTGGGGACATACTTGCAACCAGAGCG
TGTTTACTCTCTGTCTCTTGTTGGAAACCGTCTCGTTGTGGCAACAGCAGGAAGGCACGT
CAACATCTACGATCTCAGGAATATGTCTCAGCCTGAGCAAAGAAGGGAGTCTTCACTCAA
ATACCAGACCAGATGTGTTCGTAGTTATCCTAATGGAACAGGTTATTTGACTGACTGGCA
TATATTGCGTTCTGTGGTTGCTAGTTAATTTGTCTTTTTAAGTTCCCAAGTATCCCTCTT
TCCTCAGTTTTGAGTATTTGATATTGAAATTTTGCAGGTTATGCTCTAAGCTCTGTTGAA
GGAAGAGTTGCGATGGAGTTCTTTGATCTGTCAGAGGCTGCTCAGGCCAAGAAGTATTTT
TCCTCAGTGTTTTTTTTTTAATTCTACTCGTCTCTTTTCCACTACTTTGGTTACAAATAG
ATTTAAACTTGTTCTCAGATATGCTTTCAAATGCCATCGGAAATCAGAGGCTGGAAGAGA
CATTGTTTACCCTGTAAATGCCATTGCATTCCATCCAATGTGAGTTCCTATCTTAGCAAG
ATTCCAACTGTTTCTTAGATATCTAGATTTATTACATTTTTACACAACACGAGAAGGTTT
TAAAATAATAGTCCAACTAAAATTGTTTAGTGTAAGTTGAAGGCTAGTTCGAAGTTACTG
AGATGCTAATTTGCTATGTTCGGATGTTATACTATAAATGCTTTTTGAGTCTAACATCTT
AGATACATCTAAGACAAAGGGTTTCTTGTGCCTCGTATGATAATTAGTGGGAACAATTAT
AAACCTCCGCGTAATCATCTGATATTTTCAATTTTGCAGTTATGGCACATTTGCAACGGG
AGGCTGTGATGGTTTTGTCAACATATGGGATGGGAACAACAAGAAGAGGCTGTATCAGGT
TTAGCAAATGACTTGAGATCTTTTTGATTTTCATCTTCTGAGAATGTTCCCTAGTTTGGG
ACAAGAATGTATTATAATTTGGTCCTTGTTTCTTTTGCAGTATTCAAAATACCCATCAAG
CATCGCAGCACTGTCATTCAGCCGAGATGGTCAGCTACTAGCTGTTGCATCAAGCTACAC
GTTTGAAGAGGGGGAGAAATCGTAAGTATCTTTCCCTTTTATAGCTCTCTGAAGTTTGGA
TTGGAGTAG
SEQ ID NO: 18 is an exemplary coding sequence of GROOT2
in Brassica napus (canola) (BnaA01G0234400WE):
(SEQ ID NO: 18)
ATGAGTCAGCCTCCTCCGTCCGCCGGTCGTGAGCTCGCGAATCCACCGTCCGACGGCATC
TCCAATCTCAGGTTTTCCAACACGAGTGACCATCTCCTCGTTTCTTCATGGGATAAGCGT
GTGAGATTGTACGATGTGAGCACCAATTCGTTGAAAGGCGAGTTCTTACACGGCGGCGCT
GTTCTCGATTGCTGTTTCCACGACGATTCCTCTGGTTTCAGCGTTGGCAGCGACAACAAA
GTCAGACGGATTGTTTTCAATGTTGGCAAAGAGGATGTTCTGGGGATGCATGAAAAGCCT
GTGCGTTGTGTTGAGTATTCTTATGCTGCAGGGCAAGTGATTACGGTATCATGGGACAAA
ACAGTTAAGTGTTGGGATCCAAGAGGTGCAAGTGGGCCTGAGCGGACTCAGGTGGGGACA
TACGTGCAACCGGAGCGTGTTTACTCTCTGTCTCTTGTTGGAAACCGTCTCGTTGTGGCA
ACAGCAGGAAGGCACGTCAACATCTACGATCTCAGGAATATGTCTCAGCCTGAGCAAAGA
AGGGAGTCTTCACTCAAATACCAGACCAGATGTGTTCGTAGTTATCCTAATGGAACAGGT
TATGCTCTTAGCTCTGTTGAAGGAAGAGTTGCGATGGAGTTCTTTGATCTGTCAGAGGCT
GCTCAGGCTAAGAAATATGCTTTCAAATGCCATCGGAAATCAGAGGCTGGAAGAGACATT
GTTTACCCTGTGAATGCCATTGCTTTCCATCCAATTTATGGCACATTTGCAACGGGAGGC
TGTGATGGTTTTGTCAACATATGGGATGGGAACAACAAGAAGAGGCTGTATCAGTACTCA
AAATATCCATCAAGCATCGCAGCACTGTCATTCAGCCGAGATGGTCAGCTACTAGCTGTT
GCATCAAGCTACACATTTGAAGAGGGAGAGAAATCGCACGAGCCAGAAGCCATCTTTGTA
AGAAACGTCAATGAAATCGAAGTGAAGCCCAAACCCAAGGCATATCCAAATCCTGCGGCA
TAG
SEQ ID NO: 19 is an exemplary protein sequence of GROOT2
in Arabidopsis thaliana:
(SEQ ID NO: 19)
MTTVTPSAGRELSNPPSDGISNLRFSNNSDHLLVSSWDKRVRLYDVSTNSLKGEFLHGGA
VLDCCFHDDFSGFSVGADYKVRRIVFNVGKEDILGTHDKAVRCVEYSYAAGQVITGSWDK
TVKCWDPRGASGPERTQVGTYLQPERVYSMSLVGHRLVVATAGRHVNIYDLRNMSQPEQR
RESSLKYQTRCVRCYPNGTGYALSSVEGRVAMEFFDLSEAAQAKKYAFKCHRKSEAGRDI
VYPVNSIAFHPIYGTFATGGCDGFVNIWDGNNKKRLYQYSKYPTSISALSFSRDGQLLAV
ASSYTFEEGEKSQEPEAIFVRSVNEIEVKPKPKVYPNPAA
SEQ ID NO: 20 is an exemplary protein sequence of GROOT2
in Thlaspi arvense (pennycress) (Ta1014.a04.6.g20630):
(SEQ ID NO: 20)
MNLELSNPPSDGISNLRFSNASDHLLVSSWDKLVRLYDVSTNSLKGEFLHGGPVLDCCFH
DDSSGFSVGADNKVRRIVFNVGKEDILGMHESPVRCVEYSYATGQVITGSWDKTVKCWDP
RGASGPDRTQVGTYLQPERVYSLSLVGNRLVVATAGRHVNIYDLRNMSQPEQRRESSLKY
QTRCVRSYPNGTGYALSSVEGRVAMEFFDLSEAAQAKKYAFKCHRKSEAGRDIVYSVNTI
AYHPIYGTFATGGCDGFVNIWDGNNKKRLYQYSKYPTSISALSFSRDGQLLAVASSYTYE
EGEKSHEPDAIFVRSVNEIEVKPKPKAYPNHAA
SEQ ID NO: 21 is an exemplary protein sequence of GROOT2
in Brassica napus (canola)(BnaC01G0327800WE):
(SEQ ID NO: 21)
MSLPPPSAGRELANPPSDGISNLRFSNTSDHLLVSSWDKRVRLYDVSTNSLKGEFLHGGA
VLDCCFHDDSSGFSVGSDNKVRRIVFNVGKEDVLGMHEKPVRCVEYSYAAGQVITGSWDK
TVKCWDPRGASGPERTQVGTYLQPERVYSLSLVGNRLVVATAGRHVNIYDLRNMSQPEQR
RESSLKYQTRCVRSYPNGTGYALSSVEGRVAMEFFDLSEAAQAKKYAFKCHRKSEAGRDI
VYPVNAIAFHPIYGTFATGGCDGFVNIWDGNNKKRLYQYSKYPSSIAALSFSRDGQLLAV
ASSYTFEEGEKSSLKFGLE
SEQ ID NO: 22 is an exemplary protein sequence of GROOT2
in Brassica napus (canola)(BnaA01G0234400WE):
(SEQ ID NO: 22)
MSQPPPSAGRELANPPSDGISNLRFSNTSDHLLVSSWDKRVRLYDVSTNSLKGEFLHGGA
VLDCCFHDDSSGFSVGSDNKVRRIVFNVGKEDVLGMHEKPVRCVEYSYAAGQVITVSWDK
TVKCWDPRGASGPERTQVGTYVQPERVYSLSLVGNRLVVATAGRHVNIYDLRNMSQPEQR
RESSLKYQTRCVRSYPNGTGYALSSVEGRVAMEFFDLSEAAQAKKYAFKCHRKSEAGRDI
VYPVNAIAFHPIYGTFATGGCDGFVNIWDGNNKKRLYQYSKYPSSIAALSFSRDGQLLAV
ASSYTFEEGEKSHEPEAIFVRNVNEIEVKPKPKAYPNPAA
III. Exemplary nucleic acid and protein sequences of GROOT3
SEQ ID NO: 23 is an exemplary genomic sequence of GROOT3
in Arabidopsis thaliana (TAIR AT3G19630):
(SEQ ID NO: 23)
TTTTTGTTGTGACGGAGGGGATTTGCTTTAAAAGTTTAGGGCTTTTAAAATCGGCGACGG
CGAGCGGAGAATTCCCAAGGAAGAGTCGATGAAGTTGAAATCGGTGTTCGATGCTTCGGA
AATCAAATCGGAATTTGAATCAGCGGGAATAAACCCTAAATTCGCGATTCAAATCTGGAA
GTATGTAATTCAGAATCCTGATTGCGTTTGGGACGAGATTCCTTCATTGCCTTCCGCTGC
ATACTCTCTTCTCCATTCCAAGTTCAAGACTCTTACTTCGTCTCTTCACTCGCTATTTCA
TTCCTCCGATGGCACCACCTCAAAGCTTCTCATCAAGCTCCAGGTACTCTATACTCTGTT
ACTGTATACAACACATTGCTTTTATGGATTCTGAAAAACTTGTAATGTCTCTGTTTCCTG
ACGAACTCATATTGGATTCTTGCTTTTGGTTTTGAAATTGAAGAATGGAGCTTTTGTGGA
AGCTGTGGTAATGAGATACGATACTCGGTTGGGGATGTTAGGAGGGAAACCACGTCCTGG
AGGTATAAGATCTACGTTATGTATTTCATCTCAGGTATTGATTTTTATCACGCAATGTTA
TCTTGGTTTTATCGAATGAAAGACATGTGATGTGAAAGTATTTATCAATAGGTTGGTTGT
AAGATGGGTTGCACATTCTGTGCTACTGGTACCATGGGATTTAAAAGCAATTTAACATCT
GGAGAAATTGTGGAGCAGCTTGTTCACGCCTCTCGCATTGCTGATATACGCAACATCGTT
TTCATGGTATATTTCTGCTTCTGTTATTGCATTTTATCTACACTATGTGCACCTTGCCTT
ATGGTTATTTATTTTATATACTCTGCATTGATGAATTTTATTCACCAGTTGTTGCCTTGG
TTTGATGATTGTGATCAGGGAATGGGAGAACCTTTAAATAACTACAATGCTGTTGTTGAA
GCTGTTCGTGTCATGTTAAACCAACCATTICAGCTGTCGCCCAAAAGAATTACCATATCA
ACTGTAAGTACTCAGAAAGCTACCTTGAAACATTCGTAAAACAGAGAAACTTATTCACTG
AATTTTGTAGGAATACTTTAGATTATGGAATTTGATAAAGGGAACTGATAGATAAACTTC
AATCCATTAAACCTCGCAGTATGCAGCTTCCAAGACATCTTTCAGCTTTTTTTGTCTAGT
TGGTTACTTTTAAATTTTGGTGTTTTGGGATTTTCTTCTGATTTCCTTGTCCTTCCGTAA
TGCTTGTACCGTATATTCCAGGCATAAATCTAAATCATGCAATGCTGCCTGTGTTGACAC
TTTTTTTTACTAGTTAGCAAATAAATTTCCCTGTATCCTAATGTCATTTGCATAGGTCGG
AATTGTTCACGCAATTAACAAGCTACACAATGATCTACCCGGTGTAAGTTTAGCAGTATC
CCTCCATGCACCAGTTCAAGAAATCCGCTGCCAGATCATGCCAGCAGCTAGAGCCTTTCC
TTTACAAAAGCTTATGGATGCACTTCAAACTTTCCAAAAGAACAGGTAGTTCATGTACTC
TTGACTGGTTCGTTGCTGTTAAATATTAAAACCACAATCCCTTCAGTTACATTGACGATT
ATAACATTATGCCACATTGTCATTGGATTTAACAGTCAACAAAAGATCTTCATTGAGTAC
ATAATGCTTGATGGAGTAAATGATCAAGAGCAGCACGCTCATCTACTAGGCGAATTGCTA
AAGACATTTCAAGTGGTAAATCTTCGATGTCTTAATACTCTTAAAGGTTCTGCAATTATC
TGATGATATTTTTTATGGAAACTCTGTGCTCTTTTCTTGAACAGGTCATAAATTTGATAC
CATTTAATCCAATTGGATCCACAAGCCAATTCGAAACAAGCAGCATACAAGGCGTGTCAA
GATTCCAGAAAATCCTGAGGGAAACATACAAGATCCGGACTACAATTCGCAAAGAAATGG
GTCAGGATATTAGCGGCGCTTGCGGTCAGCTAGTGGTGAACCAACCAGACATCAAAAAGA
CTCCTGGAACTGTGGAACTTAGAGACATAGAAGATCTGCTTCTCTAACTTTAGGACCAGA
GACATGAAACAGACATTTGGAATGGTTGTGTAGTATCTTTGAAGCCACTCTTCAAAACCC
TTTTCTTAAACTTCACATTTTGTCCTCTCATCACTTTGTGGAAAACACTTGGCTCACTCT
TATATGCAAGACAAACAGAGATGGTCCTTGGTCCTTCCACACTATCACATGGAGGTTTAA
CTTCTCTTATAAAATATTCACCAACAAGTGATGTTATGTCATCGTTAAAGTTGGAAGTAA
ATGTTAAACTTTAATTTTAATGAAGTAAATAGTAGC
SEQ ID NO: 24 is an exemplary coding sequence of GROOT3
in Arabidopsis thaliana (GenBank NM_112853.5):
(SEQ ID NO: 24)
ATGAAGTTGAAATCGGTGTTCGATGCTTCGGAAATCAAATCGGAATTTGAATCAGCGGGA
ATAAACCCTAAATTCGCGATTCAAATCTGGAAGTATGTAATTCAGAATCCTGATTGCGTT
TGGGACGAGATTCCTTCATTGCCTTCCGCTGCATACTCTCTTCTCCATTCCAAGTTCAAG
ACTCTTACTTCGTCTCTTCACTCGCTATTTCATTCCTCCGATGGCACCACCTCAAAGCTT
CTCATCAAGCTCCAGAATGGAGCTTTTGTGGAAGCTGTGGTAATGAGATACGATACTCGG
TTGGGGATGTTAGGAGGGAAACCACGTCCTGGAGGTATAAGATCTACGTTATGTATTTCA
TCTCAGGTTGGTTGTAAGATGGGTTGCACATTCTGTGCTACTGGTACCATGGGATTTAAA
AGCAATTTAACATCTGGAGAAATTGTGGAGCAGCTTGTTCACGCCTCTCGCATTGCTGAT
ATACGCAACATCGTTTTCATGGGAATGGGAGAACCTTTAAATAACTACAATGCTGTTGTT
GAAGCTGTTCGTGTCATGTTAAACCAACCATTTCAGCTGTCGCCCAAAAGAATTACCATA
TCAACTGTCGGAATTGTTCACGCAATTAACAAGCTACACAATGATCTACCCGGTGTAAGT
TTAGCAGTATCCCTCCATGCACCAGTTCAAGAAATCCGCTGCCAGATCATGCCAGCAGCT
AGAGCCTTTCCTTTACAAAAGCTTATGGATGCACTTCAAACTTTCCAAAAGAACAGTCAA
CAAAAGATCTTCATTGAGTACATAATGCTTGATGGAGTAAATGATCAAGAGCAGCACGCT
CATCTACTAGGCGAATTGCTAAAGACATTTCAAGTGGTCATAAATTTGATACCATTTAAT
CCAATTGGATCCACAAGCCAATTCGAAACAAGCAGCATACAAGGCGTGTCAAGATTCCAG
AAAATCCTGAGGGAAACATACAAGATCCGGACTACAATTCGCAAAGAAATGGGTCAGGAT
ATTAGCGGCGCTTGCGGTCAGCTAGTGGTGAACCAACCAGACATCAAAAAGACTCCTGGA
ACTGTGGAACTTAGAGACATAGAAGATCTGCTTCTCTAA
SEQ ID NO: 25 is an exemplary genomic sequence of GROOT3
in Thlaspi arvense (pennycress) (Ta1014.a04.6.g20690):
(SEQ ID NO: 25)
ATGAAGTTGAAATCGGTGTTTGACGCTTCGGAAATCAGATCGGAATTCGAGTCAGCGGGA
ATAAACCCTAATTTCGTGATTCCCATCTGGAAGTATGTAATTCAGAATCCTGATTGCGTT
TGGGATGAGATTCCTTCATTGCCCTCCGCTGCATACACGCTCCTCCATTCCAAGTTCAAG
ACTCTCACTTCGTCTCTTCACTCCCTCTTCCACTCCTCCGATGGCACCACCTCAAAACTC
CTCATCAAGCTCCAGGTTCCTCTCCACTCACTCTGTTTCTTAGTTTGTCATGTAAATATT
CTGGGCATGATCTGAATGTCTATACAAATACGATATGAGTCTGTTTTTGACTTTTACGAT
TGTAAGGAAGATTAGTCTTGTAGATAAAAAAATGTACAAGAAGCAACCATTTTGTAAATC
GATTAGAATACATAGGAGATTTGTCTGCTGAAAAGGTTTACTTTTTGATTACAACTCATT
GGTTAATACACTACGAGACCTCGTAATTAGCCTTGGCTTTGTAACCTCTGTGTGAGTTGT
CTCTATCATCTGATGAACTCATATTCCATTCTTGTTTTTGTATTTGACATTTAAGAATGG
AGCTTTTGTGGAAGCTGTGATAATGCGATACGATACTCGGTTGGGGATGTGTGGAGGGAA
ACCACGTCCAGGAGGTGTAAGGTCTACATTATGCATTTCATCCCAGGTATTGATTTTTAT
CACCCAGTGTCATCTTTGTTTCATTGAATCGCTTTTTTGGAGTGAGAAGAGCGTCTGATG
TAAAAGTATTTATCAACAGGTTGGCTGCAAAATGGGCTGCACATTCTGTGCAACTGGAAG
CATGGGATTCAAAAGCAATTTAACATCTGGAGAAATTGTGGAGCAGCTCGTTCACGCCTC
TCGCCTAGCTGATATACGCAACATCGTTTTCATGGTACATTCCCTATTAAATGCATTTCA
CCCAAAGTTCTTTGTTTTTGCTGCGTTTTCGGTTGTTGTTGTTGTTCATGTGCACTTTTC
CCTGTGGTAATGGTAAGGTGTCTTTCTATACTCTCTCTATCGATGAAGTTCATTCCTTGG
TTTGCTGATTGTGATCAGGGAATGGGAGAACCGTTAAATAACTACAATGCTGTTGTTGAA
GCTGTCCGCGTCATGTTAAAACAGCCTTTTCAGCTTTCACCCAAAAGAATCACCATATCA
ACTGTAAGTACAGAGAAACCCACCTTGAAACATTTAGAAAACAGAGAAATAGTTACTGAA
ACTGGTAGTAATTATCCTTTAGATTATGGGATTCCTTAGGGTATCGGTGTGAAACAGAGA
AATGATAAACTTCATCCTATAAACCTCTCAATTTGCAGGGCTTTCATGGTTTAGTTGGTT
ACGCTTAGCATTTGGGACTATAGTAGGATTTACTTTGTGATTTTCTTTTTCTCTCCACTA
ACCCACATTCTAATTTATTTTGCATATAGGTTGGAGTTATTCATGCGATTAACAAGCTTC
ACAATGATCTACCAGGTGTAAGTTTGGCGGTATCTCTTCATGCGCCAGTTCAAGAAATTC
GCTGTCAGATCATGCCAGCAGCTAGAGCCTTTCCTCTTCAAAAACTTATGGATGCACTTC
AAGCTTTCCAGAAAAACAGGTAGCTCAAAATTGCTTCTTTGCTACCAAAATGTTAAAACC
ATAATTTATTAAAGATCATATAACATTTTGCCACATTTTCCCTGGATTTATAATAGTCAA
CAGAAGATCTTCATTGAGTACATCATGCTTGATGGAGTAAATGATCAAGAGGAGAACGCT
CATCAACTCGGCGAATTGCTAAAGACATTTCAAGTGGTAAAGTCTTCGATTTCTTAATAC
TACAAGAGTTTCTGGGACTATCTGATGGAAACTCTGTGCCGTTTTTCTTTAACCAGGTGA
TAAATTTGATACCGTTTAACCCAATCGGATCCACAAGCCAATTCAAGACCAGCACCAAAC
AAAGCGTCTCAAGCTTCCAGAAAATCCTGAGGGAAACATACAAAATCCGAACCACAATTC
GCAAAGAAATGGGTCAGGATATTAGCGGTGCTTGTGGTCAGCTAGTCGTGAATCAACCGG
ACAGCAAGAGACCTCCTGGAACTGTGGAACCACTTAGAGACATTGAAGACCTGCATCTTT
AA
SEQ ID NO: 26 is an exemplary genomic sequence of GROOT3
in Sorghum bicolor (sorghum) (Sobic.001G465500):
(SEQ ID NO: 26)
AGTACTCCCCACTCGAGCCGACGCAATTTCGCACCCGAAGTTCCAAACCCCGCCGGCCGC
CGCGTCCCTTCCACTTCCTCCTTACCCCAATGGCGTCGTCATCGAGGGCGACGTCGTCGC
GCCGTTCCGTCTTCGACGCCGCCTACATCCGCTCGGAGTTTTCTGCGGCTGGCATCTCCG
GCCACTTCATCCCTCTCATCTGGAAGTACGCCTCTTTACGCCAAATCACAGGCGCATGCC
ACAATCCCCGCCCGCCCGTCTCCCTCCCTTTCTCCAATTCCCAAGTTTTTTTTATATATA
TTTCTGTTTAACCTGAGGAGTGATTCTCTCTGCGTGCGCAGGTACGTACTTCAGAACCCT
AGGTGCAGCGACCTGGATGGCGTCCCGTCGCTGCCGGCGGCCGCGTACGCGCTCCTCCGG
CAGAAGTTCCGGCCGACCACGTCGACGCTAACCGCTGCCGCGGACTCCAAGGACCGCACC
ACGACCAAGCTCCTCATCTCCCTGCAGGTAATTTGCACATTCACAAATGTGGCATGTGGC
GTGCGAGTCCACTGGTGTTTGGGGATTATGCGAAATTGGGAATATGTTGTAGTCTCCCCG
GTTATTTCGGTAGCTACTTTTGCATTTGGTTCATTGTCCGTGGACATTAGAAATTTTCTC
TTGGTGATGTGAGCTTATGAAACATATGCTTGTTTGATTACTCCGTCAGGAATGGAATGG
TTGACACATGCATAATTGATTAGCCTCATGGGCACTATTTTAGGGATTGAATTCACCCTG
ATTGTCTCTGATAGAGTAGGCATTAGAAAACAGTACTTTTGTTTCGTGTTTATGTGGTTT
TCCAATGTTTGTGGAAGATAATAGCATACCTTGAAATACCTAATCTGTGTAGTTTTATGT
TTTTTTTAAGATCAATTCATCATTATTGTTGTCTTGGATGTAATAGAGCTGCAATACAAA
TAGGGAGCACCAAGATACAGATATGCACTATCTTGTACTAACTTCTACAGATTCAACCTG
CAGCACCTGAACCATCCAGTTATTGAATATTTATAGCAAAGACAAGTATAACTAACAAGC
AGGACCTTTTGCCCCTCCTTCCGCGAATAAAAAAGAAAAAAAGAGAGAAGTATAACTTAA
ATGCTTGACTTGTTCCTAATATTAACTCTACCAACTGTTGTGGAGGAGCATTTATTTTCT
GTTGCTATTTGGCTAGATAGTAGGAGGTTCGAGTCATCTTAAAAAATGTACAGCTTAAGC
CATATATATTTGATATTTCATTCTCGTTATTTGGATTTTATATTTCACACTTGTATCCTT
TAGTTTCTTTGCCCTTTGGCTCTATATCTGTAGTTTTTCAGTCATCTTCAAAATGAATAG
ATAAAGATATAATGGTTTCAGTCCTTATAATATGTTATATGACTTATGGTAGCATCTTCT
GTATTAGATAGCCTGTAACTTTGCGATTTAAATTCTACTCCCTTTTTTTTGCATAGTGAA
CTCCTTACAGGTGGACATCATTTCTTGCAAGCTACAATTCTATTTCAGACCGCCTCCTTT
TCTTTTTTAACTTGGCTGTTGTTTATCTAATTATTTTATGTCTGTGCATTGAAGAAGTTT
TTAACCACAAATTTGCCATTTTTTTGTTGTAAGAGTTTTTTCTTTTCAGATTTTGTATTT
TTTAACATTGAAGAACAACTTATTGGACTTCAAATTTGTGGTGTAGAATGGAGAGTCTGT
AGAGGCAGTGGTCATGCGATATGACACACGACTGGGGAAGTATGATGGAAAGCCTCGGCC
TGGTGGACTGCGCTCAACCCTTTGTGTGTCATCACAGGTGAATATATTTCTATATTGGAT
GTCAGGTGCTCAAGTCTGGATGAACTTGTTTCTAATGGTTTAACACCTTTTACCAGAAGA
CCTGTTCATAACTTAATTCTCACCAGTGTGTTATGATTCTTGGTAAACTTGTTACTCTAT
TGTTATTTGGTGACTTTGGCTCGGTAGATAGCCCAGCCGGGAACGAATCCCAAGCAATGA
GACCAAGAGAATGCAGAATGTCTACTAAAGAGCTCTCTCAAGTCTTTCTTTCTACAATAC
TAAACAGGTTGGGTGCAAGATGGGCTGTAGATTCTGTGCTACTGGAACAATGGGCTTCAA
AAGCAATCTGTCTTCTGGAGAAATTATAGAGCAGCTGGTCCATGCATCCCGCTATTCTCA
GATCCGAAATGTTGTTTTCATGGTAATTTTCACTCAAGTAGTAGCAATTGCCTGAAATTT
CTATGTAAGAAAGAAAAATTGTGGCATTTGAATGCGATGATTATGCATCTTTTGTGATCC
CTTGTAGTGTTGTTTTCATCTTGATTATGCTGATCAGATTTAACTTGTTACTTGGTTTTG
TCATTGATTCTGTGAACAAATAAGCTCAGTAAGATCCATATATTGGAAAATGACATCCAT
TACTTCATTCTTTTGCCAACAAGTATCACATGCCATCCTCACATTAGATTGTATTCCTAA
CTTGACCATTGTGACGATACTGAGCATTATTACCCTGGTAGTTGTATATACTTTACATTG
GGGGCAATTTACTTACAGACAAAATGCTTCATTTTGCAAGTATCACCACTGCAGAAATGG
GAAATTATTCCTGCAAAGTTTACATGAATAGAATGAATGCCAGAATTTTCTTAGCTAGTT
TGCTTGATTTATGGATTGAGCTGGCAATATGTTGATCTGTTTTTCTTTTGCTGGTGAGTG
GTGACAATGGTTCTTGTACAAATTCCTGCTGGTATTACAGTTATCCCTTGTTACCTATAA
TTCTCAAAAACTAGTGGTATGCTGTTTGAGTTAGTTTCTAAATTATTATAGGTTTATTCT
TTCATGTTCATGTTCTCTGGCACTTATAGCTTATGTGCTTTGCTGATTCTTTGATATAGA
AATTAGAAACACTGCTTTGCTGATTGCTAAAAAATCGTGGGTCGCTAAATGTTTGTGCAT
GTTTAACTTTAAAATTGCAAATTATTATCTACATATGTGAACATCCTTGGAACTGGGTGG
TGTCATGTCATTATTGAGAAGAACCATGATAAATGAATTCTATTTGATGTTTGTTATAAC
AGTATTAGTGTCCTCCAGAGGTTAGCGAGGTGTTATTTATATGCAGGGAATGGGGGAGCC
GATGAACAACTATAATGCTTTGGTTGAAGCAATCGGAGTGTTTACAGGATCTCCTTTCCA
GCTTTCACCTAAGAGAATTACTGTATCTACTGTAAGCATACTTAGTCACCTTTGCATGTG
AAATCACATTCTCAAGATTTTTGTTGAAGCACTATATTTTGTTAGTAGTGAAATATTCTT
ATAAACTATATGTTGTTTAGTGGCGGCTGTTACACTTCTGAATGACCTAATGAATTTCTG
CTGTGATAAACCACACAATGGTGAATCCGTCAAAAGCTTTTTATTTAATTGTGCAAATAT
AACTTGATAAATTGTGTAGCAGGCCAGTTGTATATTCTTTGGATGAACTGTTCTATTCCC
CCTTGTTGCTAGCAAGTATATCTTGTTTTAGCACTTGTAGGATTTAAACGTTAACATTTA
TTATAAAATACAGTTATTACATAAGATATGTGGTCTGATGCCAAACCTTTTTTTTCACTT
ACGTTATTGCTGCTTCTAGGTTTATAATAGAATTCATTGTCATGTTGTGTGGTAAAAACT
TTTCTATGCATGTTAATTTTATTTATATGTTTGCTCATTTATCAAGCCAAATTTTCCATA
TTAGCTTTGAAGCCATGGTGTGTTTTGCCACTGTATTTTTTGCTAAAAGTTTGTTTTAAG
ATTGAACTTTTGAATTAGAGAAGCTGATAGATTGATTTTTCTTAATAATTACTGCAGGTC
GGAATCATTCATGGAATCAACAAGTTCAATGCAGATCTTCCAAAGGTGAATTTAGCTGTG
TCATTGCATGCTCCTGACCAAGATATACGCTGTCAGATAATGCCTGCTGCACGTGCCTTT
CCTTTAGTAAAGTTAATGAACGCGCTGCAGTCCTATCAAAATGAGAGGTGAGATTACAAT
AACGCATCTCTTACCAGTGTCAAAATGTTAGCATGGAGATTTGATTTCTGATTACCCTTT
CATGTTTTATCACAGCAAACAGACCATCTTTATTGAGTACATTATGCTTGATGGAGTTAA
CGATCAGGAGGAGCATGCTCATCAGCTTGGTAAACTGCTTGAAACGTTCAAAGCGGTGAG
GCACTATTACATTACTTCATTTTATCATTATTACTTTGCTCTAGTTCCTTTGTAGTCACT
TCTTTTTGCAAGATGTCTAACGAGGCAGTTTTGTGTCGTTAACTTCTTGTGTTCATTATT
CAATCCATTTTGTATTGCCTCCACCAGTCTTACTTTTCCATATTTCCCTATCTGTTTGCT
TTTGACAATTGCCATTTCTGTTTAGTAATCCTGGTACTCCAAATTTCTTCTAGGTGTTTG
TCGTCCACTCCTGTTTTTATGTGTTTCAAAATTTTTGCAATAACTTACTAGTGTAATTTT
TGTACACTGAGCCTATTAATACCAAGTGATGGTCAAGACATATGTAGGTAGCAGTACTTG
TACATTCACCTTTGTGTATGGAAATGGAGTGATCAATATGTTCTCTAAACTACCAACAGG
TTGTCAATCTAATACCATTCAATCCAATTGGGTCATTAAGCAATTTCAAGACAAGCAGTG
ACCAAAATGTGAAGAAGTTCCAAAAGGTTCTAAAAGGCATCTACCACATCAGAACCACTG
TTCGCCAGCAGATGGGTCAAGACATAGCTGGTGCTTGTGGCCAGTTGGTGGTTAGCCTCC
CAGATGAAAGATCAGCTGGTGGAGCAACCCTGTTGTCAGACATCGAAGACCTTCGGATCT
GACCTCTCCCAATTGTTACTTCTATCTTCTCAAAACCAGATCGTCTTACTAATTAGCCTT
ACATGATATGGTAACTAGTGCTATAGTTTCAGTCAAGCTTTAAAACATGGAGATGTTTAG
TTTGACCATTGTTGTGTTTCCCTTTTGTTCTGGGATGCACAATTTTGCTGTAAACACAAT
GGTGAAGACTCCTGCTCGTGTGGATAGGTGGCCACTGAAACGGTTGGTAACTTGGTATTG
TAAATGGGACAATGTTATCGCAACAATGCCATGGTCAGCTATAATGATATGGTATCTTCT
GTCTAAGACGACTTTCCTGTCAGATCTCAACGAATATCTGATGTTTTTCCTCTCTTATTT
GCAGCTTTTCATTGCCATTAGCAAGTTGGTCGGGTTGGAATTGTGCCTTCGTTTTATGCT
TTATTCAGATTCCGTGTCGGTGGGCTGTATGTAATGGTGGTGGCATGGGCTTGGTGCTGC
TGCAGCGTGTTATCTTTGAAAATGTGAACCCAATTGCTGGAAATCATGTGAGATTGTCAG
GAGCGGGACAAGGCGAAACATATGAGCTGTGTTGTTTGGTTCTTATGCCCTCTTTATATC
CACCTAGCTAACTATTAGCTGTGTTAGCTGGGAAAAGGCAACTAATAGCTTATTGTCGAG
AGAAATCAGCTAATAACTGCTAACTGTTATCTAGGTGGTAGGTAAGAC
SEQ ID NO: 27 is an exemplary genomic sequence of GROOT3
in Brassica napus (canola) (BnaA03G0370900WE):
(SEQ ID NO: 27)
ATGAAGATGAAATCTGTATTCGACGCTCCGGAGATGAAATCGGAGTTCGAGTCAGCTGGA
ATAAACCCCAATTTCATGATCCCGATCTGGAAGTATGTAATTCAGAATCCCGATTGCGTT
TGGGACGAGATCCCTTCGTTGCCCACCGCCGCATACACTCTCCTCCATTCAAAGTTCAAG
ACTTTCACTTCCTCTCTTCACTCCCTCTTCCACTCCTCCGATGGCACCACCTCTAAACTC
CTCATCAAGCTCCAGGTAACTCTGCTCTCTCTCTCGCTCTCTCTGTTTCTGGGTTTTGAT
GTGAGTGTGTATACAATGAGAGTATGTTTTTGTTATTAAGATTGTATAGATGAGATTAGT
CTTTGTAGATGAGAAGCTGTCCTTTTAATAATACATAAAAGAGATTTGGCTGCTGAACAT
TGCTTGTAGTAAGTCCTTTGTTATAGTGTTATCTGATGAACTCGTACTTGATTCTTGATT
TTAAAGAATGGAGCTTTTGTGGAAGCTGTGATAATGAGATACGATACTCGTTTGGGGATG
TGTGGAGGGAAGCCACGCCCTGGAGGTGTGAGATCTACTCTATGCATTTCATCCCAGGTA
TTGATTTTTTTCATCACGCTGTTTCACTGAATCACTGTGGAGCATCTGATACAAGTGGTT
TGTCAATAGGTTGGTTGCAAAATGGGCTGCACGTTCTGTGCAACTGGTAGTATGGGATTC
AAAAGCAATTTAACATCTGGAGAAATCGTGGAGCAGCTAGTCCACGCCTCTCGCCTAGCT
GATATACGCAACATCGTATTCATGGTAACGCATCTCATCTAAGTTTCTTTTTGCCTGTTG
TTGTTGTTACTACATTATGTATCCGTGTCTTCACTTCTTGCCTTGTGGTAATTGCTAGAT
GTGGCTTTCTATAGTTTCTGTATCGATGAAGTAAACATTCACCAGTTGTTGGCTTGATTT
TGCTGATTGTGATCAGGGAATGGGAGAGCCTTTAAATAACTACAATGCTGTTGTTGAATC
TGTTCGTGCCATGTTAAAGCAGCCTTTTCAGCTCTCACCCAAGAGAATTACCATTTCAAC
CGTAAGTACTGAGAAAACTACCTTGAAACATTCATGAATCAGAGAAGAAAATATTCTCTT
TCTTATTTTTTTTTTTCTCCACTAACCCATATCGTTTGCACATAGGTTGGAGTTGTTCAT
GCGATTAACAAGCTTCACAATGATCTACCAGGTATAAGTTTGGCGGTATCTCTTCATGCA
CCAGTTCAAGAAATCCGCTGCCAGATCATGCCAGCAGCTAGAGCCTTTCCTCTTCAAAAG
CTCATGGATGCACTTCAAACTTTCCAGAAAAACAGGTAACTTTGCTACTGCATGTTCAAA
CCATAATCTCAAAAGTTGATAAAGATCATAACATACTTCGATTTTCAAACAGTCAGCAGA
AAATCTTCATTGAGTACATCATGCTTGATGGAGTAAATGATCAAGAGGAGAACGCTCATC
AACTAGGCGAATTGCTAAAGACATTTCAAGTGGTAAAAATCTTCTGTTTCTTTATACTCT
TAATAGTTTGAGCAATTATCTGATGGAAATTCTCTGCTGTTTTCTCAAATAATAGGTGAT
AAATTTGATACCATTCAATCCAATTGGATCCACAAGCCAGTTCAAAACCAGCACCAAAGA
AAGCGTCTCAAGTTTCCAGAAAATCCTGAGAGAAACCTACAATATACGAACCACGGTTCG
CAAAGAAATGGGTCAGGATATTAGTGGGGCTTGCGGACAGCTAGTCGTGAACCAACCTGA
CAACAAGAGACCTGCAGAACCACTTAGAGACATTGAAGATCTGCATCTTTAA
SEQ ID NO: 28 is an exemplary genomic sequence of GROOT3
in Brassica napus (canola)(BnaC03G0368100WE):
(SEQ ID NO: 28)
TAGCGACGGCTACGGCGAGGTGAGAGTGTTCAAGGAAGATCGATGAAGATGAAATCTGTA
TTCGATGCTCCGGAGATCAAATCGGAGTTCGAATCAGCGGGAATAAACCCCAATTTCATG
ATTCCGATCTGGAAGTATGTAATTCAGAATCCCGATTGCGTTTGGGACGAGATCCCTTCA
TTGCCCACCGCCGCATACACTCTCCTCCATTCAAAGTTCAAGACTTTCACTTCGTCTCTC
CACTCCCTCTTCCACTCCTCCGATGGCACCACCTCTAAACTCCTCATCAAGCTCCAGGTA
CTCCGCTCTCTCTCTCTGTTTCTGGGTTTTGATATGAGTGTGTATACAAACGCGATATGA
GTATGTGATTGCCTTTTCAATTGTATAGGAGATTAGTCTTTGTAGATGAGAAGCTGTCCT
TTTAATAATACATAAAAGAGATTTGGCTGCTGAACATTTGCTTGTAGTAAGTCCTTTGTT
ATAGTGTTATCTGATGAACTCGTACTTGATTCTTGATTTAAAGAATGGAGCTTTTGTGGA
AGCTGTGATAATGAGATACGATACTCGGTTGGGGATGTGTGGAGGGAAGCCACGCCCTGG
AGGTGTGAGATCTACTCTATGCATTTCATCCCAGGTTTTGATTTTTTCATCACTCGTGTT
TCATTGAGTTACTGTAGAGTGATGAGAGCATCTGATTTACAAGTATTTATCAATAGGTTG
GTTGCAAAATGGGCTGCACCTTCTGTGCAACTGGTAGCATGGGATTCAAAAGCAATTTAA
CATCAGGAGAAATTGTGGAGCAGCTTGTCCACGCCTCTCGCCTAGCTGATATACGCAACA
TCGTATTCATGGTAACGCATCTCGTCTAAAGTTTCTTTTTGGCTGTTGTTGTTGTTACTA
CATTATGTATCCGTGTCTTCACTTCTTGCCTTGCGGTGATTGCTATAGGTGTCTTTCTAT
ATAGTTTATGTATCGATGAAGTAAACGTTCACCGGTTGTTGGCTTGATTTTACTGATTGT
GATTAGGGAATGGGAGAGCCTTTAAATAACTACAATGCTGTTGTTGAATCTGTGCGTGCC
ATGTTAAAGCAGCCTTTTCAGCTCTCACCCAAGAGAATTACCATTTCAACTGTAAGTACT
GAGAAAACTACCTTGAAACATTCATGAATCAGAGCAGAAACATTCTCTTTCTTATTTTCT
TTTTCTCCACTAACCCATATCGTTTGCACATAGGTTGGAGTTGTTCATGCGATAAACAAG
CTTCACAATGATCTACCAGGTATAAGTTTAGCGGTATCTCTTCACGCACCAGTTCAAGAA
ATCCGCTGCCAGATCATGCCAGCGGCTAGAGCCTTTCCTCTTCAAAAGCTCATGGATGCA
CTTCAAGCTTTCCAGAAAAACAGGTAACTTTGCTACTGCATGTTCAAACCATAATCTCAG
AAGTTGATAATTATAATAACATAGTGACATATCTTCCTTTAATTTTAAACAGTCAGCAGA
AGATCTTCATTGAGTACATCATGCTTGATGGAGTAAATGATCAAGAGGAGAACGCTCATC
AACTAGGCGAATTGCTAAAGACATTTCAAGTGGTAAAAATCTTCTGTTTCTTAATACTCT
TAATAGTTTCAGCAATTATCTGATGTGAATCTCTGTGGCGTTTTCTCAAAAAAAACAGGT
GATAAATTTGATACCGTTCAACCCAATTGGGTCCACAAGCCAGTTCAAAACCAGCACCAA
ACAAAGCGTCTCGATCTTCCAGAAGATCCTGAGGGAAACCTACAATATACGAACCACGGT
TCGCAAAGAAATGGGTCAGGATATTAGCGGTGCTTGCGGCCAGCTAGTCGTGAACCAACC
TGACAACAAGAGACCAGCAGAACCACTTAGAGACATTGAAGATCTGCATCTCTAACTTTA
GGACCAGGCATCTCTGAAATTTTCCTAAAGAGACCAAAGATATATGGTTTGTATTATCTT
CGAAGTCACTCTTGAAAACCTTTTTCTTAGTTACTTGACATATTGTTCCTCATTACTCTG
GTGAAAACACTT
SEQ ID NO: 29 is an exemplary genomic sequence of
GROOT3 in Glycine max (soybean)(Glyma.11G001700):
(SEQ ID NO: 29)
GTTGAGAAGAAAGAGAGAGAGAGGGAGAGGGCATGGGAATCCGATCGGTATTCGATGGCG
GCGAGCTGAGAAGGGAAGTGGAGAAAAGTGGAATTGACCCAAAATTCATTCCAAAGATAT
GGAAGCATATCCTCATCTCTGCCAAAGATGAAGATTGGGATTGGGAGAAACAAGTTCCCT
CCTTGCCCTCTTCGGCCTACTCTCTCCTTCGTTCCAACTTCAAAACCCCTCTCTCTTCCT
CTATTCACTCCGTTTTCCACTCTGCCGACAACCTCACCACCAAGCTCCTCATCCAGCTCC
ACCACAATCATGGACCTTTCGTCGAGGCTGTCATTATGAGGTACGATACTCGTTTGGGCA
AATATGCCGGCCAACCTCGCCCCGGTGGTCTCAGAGCTACTTTGTGTATTTCTTCTCAGG
TAACCATATCTTTCTTTTCTTTTCTTTTCTTTTCTTTTCTTTTTCTATTGCTAACGCTTC
CTTTTCTCTTCTGCAGGTTGGATGCAAAATGGGTTGCAATTTCTGTGCCACTGGATCCAT
GGGATTCAAAAACAACCTATCATCCGGTGAAATTGTGGAACAGCTCGTTCATGCCTCTAC
CTTCTCACAAATCCGTAATGTTGTCTTCATGGGCATGGGAGAGCCTCTCAACAACTATTC
TGCTGTGGTAGAAGCCGTTCGCATCATGACTGGGTTGCCATTTCAATTGTCATCCAAAAG
GATTACCATCTCAACGGTATAAACATAAACACACAAATTTATGATAGCCGTTTTTGAGTA
AAATCTTAGAATCTGTTTGTTACATTTCTCTAATAGGTTGGCATCATTCATGCTATCAAC
AAGCTTCATGATGACCTGCCTGGTTTGAACTTGGCTGTCTCACTGCATGCGCCGGCCCAA
GACATCCGTTGCCAGATAATGCCTGCTGCTCGTGCTTTTCCTTTGGGAAAACTCATGGAT
TCACTGCAAGTCTATCAAAGGAAAAGGTCATTCATACTTTCTCTCAACAATTTCTCTCAT
TTTTTTAAGCCATTAATATTAATATTTCATCCTTAATTATAGTCTGCAGAAAATATTTAT
TGAATACATAATGCTTGATGGGGTGAACGACGAAGAGCACCATGCCCACCTATTGGGAAA
ACTGTTGGAGACATTTCAAGTGGTAAATACTCAACTGCTTCTTCCTTTCTCTATTCAATA
TTCAATGTCTCTACCAAACATTTATGCTCTGCCCCCATAATTCCAATGCCACTCTTCTAC
TCTAATAAAATGTAGGCAATCGTGAATGAATAGTGTTGTTTCTCGATAATTCAGTGACTC
TCAGGAAAATGTTCTTAAAACTTGATCGTCATTCTCTGCTCAGTTACACAATGCTGTTAA
GTACTAACATTTTGCATATGGTCTGAATTTTGAAACAGGTTGTGAACTTAATACCTTTCA
ACTCTATTGGTACCTTGAGTCAATTCAAACCTACCAGTGAGCAGAAAGTCTCAAATTTTC
AGAAAATTCTTAGGGGTACCTATAATATTCGAACAACAGTTCGGAAGCAGATGGGTCAGG
ACATAAGTGGTGCTTGTGGACAATTGCTGGTGAACATATCTGACAAGTCCCTTGGCACTG
CAGTTCCCCTAACGGACATAGAAGATATTGTTATCTGATATCAATAGCTTCAATTTAATT
CTTGCCCTCAATTTTAATTTCGTTTCCTAGTTTTTCTATTGTTTTTATGTTTGGTTCTAC
AGAAATTAGAAGTAGAACTGTAGAAGACAAGGTGCACTGGCTGAAATGCAACCAAAATGC
TTTATATGAATGTGTGATGATCTTTTAGATTTTGATAAAATTGAATTTCAAAGAATTGAA
GAAAAAAAAATCATTTGTGAG
SEQ ID NO: 30 is an exemplary protein sequence of GROOT3
in Arabidopsis thaliana:
(SEQ ID NO: 30)
MKLKSVFDASEIKSEFESAGINPKFAIQIWKYVIQNPDCVWDEIPSLPSAAYSLLHSKFK
TLTSSLHSLFHSSDGTTSKLLIKLQNGAFVEAVVMRYDTRLGMLGGKPRPGGIRSTLCIS
SQVGCKMGCTFCATGTMGFKSNLTSGEIVEQLVHASRIADIRNIVFMGMGEPLNNYNAVV
EAVRVMLNQPFQLSPKRITISTVGIVHAINKLHNDLPGVSLAVSLHAPVQEIRCQIMPAA
RAFPLQKLMDALQTFQKNSQQKIFIEYIMLDGVNDQEQHAHLLGELLKTFQVVINLIPFN
PIGSTSQFETSSIQGVSRFQKILRETYKIRTTIRKEMGQDISGACGQLVVNQPDIKKTPG
TVELRDIEDLLL
SEQ ID NO: 31 is an exemplary protein sequence of GROOT3
in Thlaspi arvense (pennycress)(Ta1014.a04.6.g20690):
(SEQ ID NO: 31)
MKLKSVFDASEIRSEFESAGINPNFVIPIWKYVIQNPDCVWDEIPSLPSAAYTLLHSKFK
TLTSSLHSLFHSSDGTTSKLLIKLONGAFVEAVIMRYDTRLGMCGGKPRPGGVRSTLCIS
SQVGCKMGCTFCATGSMGFKSNLTSGEIVEQLVHASRLADIRNIVFMGMGEPLNNYNAVV
EAVRVMLKQPFQLSPKRITISTVGVIHAINKLHNDLPGVSLAVSLHAPVQEIRCQIMPAA
RAFPLQKLMDALQAFQKNSQQKIFIEYIMLDGVNDQEENAHQLGELLKTFQVVINLIPFN
PIGSTSQFKTSTKQSVSSFQKILRETYKIRTTIRKEMGQDISGACGQLVVNQPDSKRPPG
TVEPLRDIEDLHL
SEQ ID NO: 32 is an exemplary protein sequence of GROOT3
in Sorghum bicolor (sorghum)(Sobic.001G465500):
(SEQ ID NO: 32)
MASSSRATSSRRSVFDAAYIRSEFSAAGISGHFIPLIWKYVLQNPRCSDLDGVPSLPAAA
YALLRQKFRPTTSTLTAAADSKDRTTTKLLISLONGESVEAVVMRYDTRLGKYDGKPRPG
GLRSTLCVSSQVGCKMGCRFCATGTMGFKSNLSSGEIIEQLVHASRYSQIRNVVFMGMGE
PMNNYNALVEAIGVFTGSPFQLSPKRITVSTVGIIHGINKFNADLPKVNLAVSLHAPDQD
IRCQIMPAARAFPLVKLMNALQSYQNESKQTIFIEYIMLDGVNDQEEHAHQLGKLLETFK
AVVNLIPFNPIGSLSNFKTSSDQNVKKFQKVLKGIYHIRTTVRQQMGQDIAGACGQLVVS
LPDERSAGGATLLSDIEDLRI
SEQ ID NO: 33 is an exemplary protein sequence of
GROOT3 in Brassica napus (canola)(BnaA03G0370900WE):
(SEQ ID NO: 33)
MKMKSVFDAPEMKSEFESAGINPNFMIPIWKYVIQNPDCVWDEIPSLPTAAYTLLHSKFK
TFTSSLHSLFHSSDGTTSKLLIKLQNGAFVEAVIMRYDTRLGMCGGKPRPGGVRSTLCI
SSQVGCKMGCTFCATGSMGFKSNLTSGEIVEQLVHASRLADIRNIVFMGMGEPLNNYNAV
VESVRAMLKQPFQLSPKRITISTVGVVHAINKLHNDLPGISLAVSLHAPVQEIRCQIMPA
ARAFPLQKLMDALQTFQKNSQQKIFIEYIMLDGVNDQEENAHQLGELLKTFQVVINLIPF
NPIGSTSQFKTSTKESVSSFQKILRETYNIRTTVRKEMGQDISGACGQLVVNQPDNKRPA
EPLRDIEDLHL
SEQ ID NO: 34 is an exemplary protein sequence of GROOT3
in Brassica napus (canola)
(BnaC03G0368100WE):
(SEQ ID NO: 34)
MKMKSVFDAPEIKSEFESAGINPNFMIPIWKYVIQNPDCVWDEIPSLPTAAYTLLHSKFK
TFTSSLHSLFHSSDGTTSKLLIKLQNGAFVEAVIMRYDTRLGMCGGKPRPGGVRSTLCI
SSQVGCKMGCTFCATGSMGFKSNLTSGEIVEQLVHASRLADIRNIVFMGMGEPLNNYNAV
VESVRAMLKOPFQLSPKRITISTVGVVHAINKLHNDLPGISLAVSLHAPVQEIRCQIMPA
ARAFPLQKLMDALQAFQKNSQQKIFIEYIMLDGVNDQEENAHQLGELLKTFQVVINLIPF
NPIGSTSQFKTSTKQSVSIFQKILRETYNIRTTVRKEMGQDISGACGQLVVNQPDNKRPA
EPLRDIEDLHL
SEQ ID NO: 35 is an exemplary protein sequence of GROOT3
in Glycine max (soybean):
(SEQ ID NO: 35)
MGIRSVFDGGELRREVEKSGIDPKFIPKIWKHILISAKDEDWDWEKQVPSLPSSAYSLLR
SNFKTPLSSSIHSVFHSADNLTTKLLIQLHHNHGPFVEAVIMRYDTRLGKYAGQPRPGGL
RATLCISSQVGCKMGCNFCATGSMGFKNNLSSGEIVEQLVHASTFSQIRNVVFMGMGEPL
NNYSAVVEAVRIMTGLPFQLSSKRITISTVGIIHAINKLHDDLPGLNLAVSLHAPAQDIR
CQIMPAARAFPLGKLMDSLQVYQRKSLQKIFIEYIMLDGVNDEEHHAHLLGKLLETFQVV
VNLIPFNSIGTLSQFKPTSEQKVSNFQKILRGTYNIRTTVRKQMGQDISGACGQLLVNIS
DKSLGTAVPLTDIEDIVI
IV. Exemplary promoter sequences
SEQ ID NO: 36 is a DNA sequence of an exemplary UBQ10
promoter from Arabidopsis thaliana:
(SEQ ID NO: 36)
CTAGTCTAGCTCAACAGAGCTTTTAACCCAAATTGGTACAATAGAATACAACTTTAGATC
ATAATTCTCAAAAGAAAGAGATTCCTTAGCTATTCTATCTGCCACTCCATTTCCTTCTCG
GCTTGTATGCACAAGCATAAAATCCTCAAACTTGCTAAGTAGATACTTTATGTCTTGGAT
AATTGGATTGAGACTTGACAAGCATAACTTTCATGTAACCAAAGACACAAGTTGCTGAGA
ATCCACCTCAAAAATGATCTTCCTATAATTGAATCGGGATAATGACAGCACAGCCCATCT
AAGAGCCTCCACTTCTACTTCCAGCACGCTTCTTACTTTTACCACAGCTCTTGCACCTAA
CCATAACACCTTCCCTGTATGATCGCGAAGCACCCACCCTAAGCCACATTTTAATCCTTC
TGTTGGCCATGCCCCATCAAAGTTGCACTTAACCCAAGATTGTGGTGGAGCTTCCCATGT
TTCTCGTCTGTCCCGACGGTGTTGTGGTTGGTGCTTTCCTTACATTCTGAGCCTCTTTCC
TTCTAATCCACTCATCTGCATCTTCTTGTGTCCTTACTAATACCTCATTGGTTCCAAATT
CCCTCCCTTTAAGCACCAGCTCGTTTCTGTTCTTCCACAGCCTCCCAAGTATCCAAGGGA
CTAAAGCCTCCACATTCTTCAGATCAGGATATTCTTGTTTAAGATGTTGAACTCTATGGA
GGTTTGTATGAACTGATGATCTAGGACCGGATAAGTTCCCTTCTTCATAGCGAACTTATT
CAAAGAATGTTTTGTGTATCATTCTTGTTACATTGTTATTAATGAAAAAATATTATTGGT
CATTGGACTGAACACGAGTGTTAAATATGGACCAGGCCCCAAATAAGATCCATTGATATA
TGAATTAAATAACAAGAATAAATCGAGTCACCAAACCACTTGCCTTTTTTAACGAGACTT
GTTCACCAACTTGATACAAAAGTCATTATCCTATGCAAATCAATAATCATACAAAAATAT
CCAATAACACTAAAAAATTAAAAGAAATGGATAATTTCACAATATGTTATACGATAAAGA
AGTTACTTTTCCAAGAAATTCACTGATTTTATAAGCCCACTTGCATTAGATAAATGGCAA
AAAAAAACAAAAAGGAAAAGAAATAAAGCACGAAGAATTCTAGAAAATACGAAATACGCT
TCAATGCAGTGGGACCCACGGTTCAATTATTGCCAATTTTCAGCTCCACCGTATATTTAA
AAAATAAAACGATAATGCTAAAAAAATATAAATCGTAACGATCGTTAAATCTCAACGGCT
GGATCTTATGACGACCGTTAGAAATTGTGGTTGTCGACGAGTCAGTAATAAACGGCGTCA
AAGTGGTTGCAGCCGGCACACACGAGTCGTGTTTATCAACTCAAAGCACAAATACTTTTC
CTCAACCTAAAAATAAGGCAATTAGCCAAAAACAACTTTGCGTGTAAACAACGCTCAATA
CACGTGTCATTTTATTATTAGCTATTGCTTCACCGCCTTAGCTTTCTCGTGACCTAGTCG
TCCTCGTCTTTTCTTCTTCTTCTTCTATAAAACAATACCCAAAGAGCTCTTCTTCTTCAC
AATTCAGATTTCAATTTCTCAAAATCTTAAAAACTTTCTCTCAATTCTCTCTACCGTGAT
CAAGGTAAATTTCTGTGTTCCTTATTCTCTCAAAATCTTCGATTTTGTTTTCGTTCGATC
CCAATTTCGTATATGTTCTTTGGTTTAGATTCTGTTAATCTTAGATCGAAGACGATTTTC
TGGGTTTGATCGTTAGATATCATCTTAATTCTCGATTAGGGTTTCATAGATATCATCCGA
TTTGTTCAAATAATTTGAGTTTTGTCGAATAATTACTCTTCGATTTGTGATTTCTATCTA
GATCTGGTGTTAGTTTCTAGTTTGTGCGATCGAATTTGTCGATTAATCTGAGTTTTTCTG
ATTAACAGG
SEQ ID NO: 37 is a DNA sequence of an exemplary Ubil
promoter from maize:
(SEQ ID NO: 37)
CTGCAGTGCAGCGTGACCCGGTCGTGCCCCTCTCTAGAGATAATGAGCATTGCATGTCTA
AGTTATAAAAAATTACCACATATTTTTTTTGTCACACTTGTTTGAAGTGCAGTTTATCTA
TCTTTATACATATATTTAAACTTTACTCTACGAATAATATAATCTATAGTACTACAATAA
TATCAGTGTTTTAGAGAATCATATAAATGAACAGTTAGACATGGTCTAAAGGACAATTGA
GTATTTTGACAACAGGACTCTACAGTTTTATCTTTTTAGTGTGCATGTGTTCTCCTTTTT
TTTTGCAAATAGCTTCACCTATATAATACTTCATCCATTTTATTAGTACATCCATTTAGG
GTTTAGGGTTAATGGTTTTTATAGACTAATTTTTTTAGTACATCTATTTTATTCTATTTT
AGCCTCTAAATTAAGAAAACTAAAACTCTATTTTAGTTTTTTTATTTAATAATTTAGATA
TAAAATAGAATAAAATAAAGTGACTAAAAATTAAACAAATACCCTTTAAGAAATTAAAAA
AACTAAGGAAACATTTTTCTTGTTTCGAGTAGATAATGCCAGCCTGTTAAACGCCGTCGA
CGAGTCTAACGGACACCAACCAGCGAACCAGCAGCGTCGCGTCGGGCCAAGCGAAGCAGA
CGGCACGGCATCTCTGTCGCTGCCTCTGGACCCCTCTCGAGAGTTCCGCTCCACCGTTGG
ACTTGCTCCGCTGTCGGCATCCAGAAATTGCGTGGCGGAGCGGCAGACGTGAGCCGGCAC
GGCAGGCGGCCTCCTCCTCCTCTCACGGCACGGCAGCTACGGGGGATTCCTTTCCCACCG
CTCCTTCGCTTTCCCTTCCTCGCCCGCCGTAATAAATAGACACCCCCTCCACACCCTCTT
TCCCCAACCTCGTGTTGTTCGGAGCGCACACACACACAACCAGATCTCCCCCAAATCCAC
CCGTCGGCACCTCCGCTTCAAGGTACGCCGCTCGTCCTCCCCCCCCCCCCCTCTCTACCT
TCTCTAGATCGGCGTTCCGGTCCATGGTTAGGGCCCGGTAGTTCTACTTCTGTTCATGTT
TGTGTTAGATCCGTGTTTGTGTTAGATCCGTGCTGCTAGCGTTCGTACACGGATGCGACC
TGTACGTCAGACACGTTCTGATTGCTAACTTGCCAGTGTTTCTCTTTGGGGAATCCTGGG
ATGGCTCTAGCCGTTCCGCAGACGGGATCGATTTCATGATTTTTTTTGTTTCGTTGCATA
GGGTTTGGTTTGCCCTTTTCCTTTATTTCAATATATGCCGTGCACTTGTTTGTCGGGTCA
TCTTTTCATGCTTTTTTTTGTCTTGGTTGTGATGATGTGGTCTGGTTGGGCGGTCGTTCT
AGATCGGAGTAGAATTCTGTTTCAAACTACCTGGTGGATTTATTAATTTTGGATCTGTAT
GTGTGTGCCATACATATTCATAGTTACGAATTGAAGATGATGGATGGAAATATCGATCTA
GGATAGGTATACATGTTGATGCGGGTTTTACTGATGCATATACAGAGATGCTTTTTGTTC
GCTTGGTTGTGATGATGTGGTGTGGTTGGGCGGTCGTTCATTCGTTCTAGATCGGAGTAG
AATACTGTTTCAAACTACCTGGTGTATTTATTAATTTTGGAACTGTATGTGTGTGTCATA
CATCTTCATAGTTACGAGTTTAAGATGGATGGAAATATCGATCTAGGATAGGTATACATG
TTGATGTGGGTTTTACTGATGCATATACATGATGGCATATGCAGCATCTATTCATATGCT
CTAACCTTGAGTACCTATCTATTATAATAAACAAGTATGTTTTATAATTATTTTGATCTT
GATATACTTGGATGATGGCATATGCAGCAGCTATATGTGGATTTTTTTAGCCCTGCCTTC
ATACGCTATTTATTTGCTTGGTACTGTTTCTTTTGTCGATGCTCACCCTGTTGTTTGGTG
TTACTTCTGCAG
SEQ ID NO: 38 is a DNA sequence of an exemplary 35S promoter:
(SEQ ID NO: 38)
TCGACGAATTAATTCCAATCCCACAAAAATCTGAGCTTAACAGCACAGTTGCTCCTCTCA
GAGCAGAATCGGGTATTCAACACCCTCATATCAACTACTACGTTGTGTATAACGGTCCAC
ATGCCGGTATATACGATGACTGGGGTTGTACAAAGGCGGCAACAAACGGCGTTCCCGGAG
TTGCACACAAGAAATTTGCCACTATTACAGAGGCAAGAGCAGCAGCTGACGCGTACACAA
CAAGTCAGCAAACAGACAGGTTGAACTTCATCCCCAAAGGAGAAGCTCAACTCAAGCCCA
AGAGCTTTGCTAAGGCCCTAACAAGCCCACCAAAGCAAAAAGCCCACTGGCTCACGCTAG
GAACCAAAAGGCCCAGCAGTGATCCAGCCCCAAAAGAGATCTCCTTTGCCCCGGAGATTA
CAATGGACGATTTCCTCTATCTTTACGATCTAGGAAGGAAGTTCGAAGGTGAAGGTGACG
ACACTATGTTCACCACTGATAATGAGAAGGTTAGCCTCTTCAATTTCAGAAAGAATGCTG
ACCCACAGATGGTTAGAGAGGCCTACGCAGCAGGTCTCATCAAGACGATCTACCCGAGTA
ACAATCTCCAGGAGATCAAATACCTTCCCAAGAAGGTTAAAGATGCAGTCAAAAGATTCA
GGACTAATTGCATCAAGAACACAGAGAAAGACATATTTCTCAAGATCAGAAGTACTATTC
CAGTATGGACGATTCAAGGCTTGCTTCATAAACCAAGGCAAGTAATAGAGATTGGAGTCT
CTAAAAAGGTAGTTCCTACTGAATCTAAGGCCATGCATGGAGTCTAAGATTCAAATCGAG
GATCTAACAGAACTCGCCGTGAAGACTGGCGAACAGTTCATACAGAGTCTTTTACGACTC
AATGACAAGAAGAAAATCTTCGTCAACATGGTGGAGCACGACACTCTGGTCTACTCCAAA
AATGTCAAAGATACAGTCTCAGAAGACCAAAGGGCTATTGAGACTTTTCAACAAAGGATA
ATTTCGGGAAACCTCCTCGGATTCCATTGCCCAGCTATCTGTCACTTCATCGAAAGGACA
GTAGAAAAGGAAGGTGGCTCCTACAAATGCCATCATTGCGATAAAGGAAAGGCTATCATT
CAAGATCTCTCTGCCGACAGTGGTCCCAAAGATGGACCCCCACCCACGAGGAGCATCGTG
GAAAAAGAAGACGTTCCAACCACGTCTTCAAAGCAAGTGGATTGATGTGACATCTCCACT
GACGTAAGGGATGACGCACAATCCCACTATCCTTCGCAAGACCCTTCCTCTATATAAGGA
AGTTCATTTCATTTGGAGAGGACACG

### IV. Exemplary Guide RNA Sequences

SEQ ID NOs: 39 and 40 are exemplary guide
sequences for targeting pennycress GROOT1
(Ta1014.a04.6.g20490):
(SEQ ID NO: 39)
ACGCUGAUUAGCUGCAGACA
(SEQ ID NO: 40)
UUCAACGGAUGGCUCAACGA
SEQ ID NOs: 41 and 42 are exemplary guide
sequences for targeting pennycress GROOT2
(Ta1014.a04.6.g20630):
(SEQ ID NO: 41)
CCUUAUCCCAUGAAGAAACG
(SEQ ID NO: 42)
UGAAAACAGCAAUCCAGUAC
SEQ ID NOs: 43 and 44 are exemplary guide
sequences for targeting pennycress GROOT3
(Ta1014.a04.6.g20690):
(SEQ ID NO: 43)
AUGGAGGAGCGUGUAUGCAG
(SEQ ID NO: 44)
GAUACUCGGUUGGGGAUGUG
SEQ ID NOs: 45 and 46 are exemplary guide
sequences for targeting canola GROOT1,
and can target both BnaC05G0378600WE
and BnaA05G0316300:
(SEQ ID NO: 45)
ACGCUGAUUAGCCGCGGAUA
(SEQ ID NO: 46)
UCCCACCAUCGACGCCGCCG
SEQ ID NOs: 47 and 48 are exemplary guide
sequences for targeting canola GROOT2,
and can target both BnaC01G0327800WE
and BnaA01G0234400WE:
(SEQ ID NO: 47)
GCAGCGACAACAAAGUCAGA
(SEQ ID NO: 48)
UGUUGGCAAAGAGGAUGUUC
SEQ ID NOs: 49 and 50 are exemplary guide
sequences for targeting canola GROOT3,
and can target both BnaA03G0370900WE
and BnaC03G0368100WE:
(SEQ ID NO: 49)
AGGAGAGUGUAUGCGGCGGU
(SEQ ID NO: 50)
UACUCUAUGCAUUUCAUCCC
SEQ ID NO: 51 is an exemplary scaffold
sequence that can be joined to the 3′
end of any of the guide sequences
provided herein to form a sgRNA:
(SEQ ID NO: 51)
GUUUUAGAGCUAGAAAUAGCAAGUUAAAAUAAGGCUAGU
CCGUUAUCAACUUGAAAAAGUGGCACCGAGUCGGUGC

The foregoing and other objects and features of the disclosure will become more apparent from the following detailed description, which proceeds with reference to the accompanying figures.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D: Natural variations of Arabidopsis thaliana, its quantification pipeline, and validation for root biomass trait. 1A: Images of growth patterns of Arabidopsis thaliana accessions with varying biomass production after 21 days of growth on % MS media under long day conditions (16 h/8 h). The images show representative accessions with low, intermediate, or high root biomass. 1B: Schematic representation of the Total Root Pixel (TRP) pipeline for estimating biomass directly from root images by calculating total pixel counts. 1C: A TRP distribution graph for all the Arabidopsis thaliana natural accessions studied herein; x-axis: total root pixel number; y-axis: frequency of the total root pixel number. The distribution is centered around the mean value. The three arrows indicate the positions of the accessions shown in 1A. 1D: A correlation plot between fresh weight (g) and TRP data obtained from the TRP pipeline. Each dot represents the mean root fresh weight value of a natural accession from 72 randomly selected accessions used for validation purposes. Major population groups are indicated by different colors.

FIGS. 2A-2D: Genome-wide association study (GWAS) identifies loci associated with biomass and reveals patterns linked to local climates. 2A: A Manhattan plot of GWA mapping for root biomass (measured by TRP) after 21 days of growth on ½ MS media. Horizontal blue line: 5% false discovery rate threshold; horizontal red line: Bonferroni 5% threshold. Top five biomass-associated SNPs are indicated by arrows. Colors indicate different chromosomes. 2B: Box plots of TRP for accessions containing a reference (Col-0) allele and accessions containing a non-reference (non-Col-0) allele at one of the top five SNPs, for each of the top five SNPs. Non-Col-0 allele accessions exhibit significantly higher TRP values. The horizontal line within each box indicates the mean value; the lower and upper edges represent the 25th and 75th percentiles: the whiskers extend to the minimum and maximum values. 2C: A map showing accession collection sites. Each dot represents an accession; colors indicate SNP variants for the top five SNPs. 2D: A plot showing correlation between TRP (y-axis) and Bio24 (radiation of the wettest quarter in W m⁻²; x-axis) using accessions having a non-Col-0 allele for at least one of the top five SNPs. Correlation coefficients (r) and significance levels (P-values) are indicated.

FIGS. 3A-3E: GROOT mutants show increased plant biomass and seed area. 3A: Images of growth patterns of wild-type (WT) Col-0 plants and T-DNA lines for the three GROOT genes after 21 days on ½ MS plates. 3B-3D: Box plots of root dry weight (B), root fresh weight (C), and shoot dry weight (D) of WT plants and T-DNA lines for GROOT1, GROOT2, and GROOT3 at 21 days after plating. Color-coded by gene; statistically significant (T-test) indicated by asterisks. 3E: Plots showing correlations between seed area and root dry weight, root fresh weight, or shoot dry weight; between shoot dry weight and root dry weight or root fresh weight; between root fresh weight and root dry weight. Dots represent individual lines, including WT lines, and GROOT1, GROOT2, and GROOT3 T-DNA lines. Correlation coefficients (r) and P-values are shown in figure.

FIGS. 4A-4H: GROOT gene expression patterns and coregulated genes indicate diverse biological functions. 4A-4C: Illustrations showing expression of GROOT1, GROOT2, and GROOT3 across different organs and developmental stages of Arabidopsis thaliana according to Klepikova et al. (2016). 4D-4E: Single cell RNAseq root expression data according to Shahan et al. (2022). 4D: A plot showing expression levels of GROOT1, GROOT2, GROOT3 in different types of root cells; y-axis: cell types; x-axis: genes; dot color indicates intensity of average expression level, and dot size indicates percentage of cells within the type of cells expressing the gene. 4E: UMAP plots for GROOT genes. 4F-4H: Bar graphs showing gene ontology (GO) enrichment for the 200 co-expressed genes of GROOT genes according to AttedII database (https://atted.jp/). Y-axis: GO categories for biological processes; x-axis: fold enrichment, with the bar length indicating the significance of enrichment as measured by −log₁₀(FDR) values.

FIGS. 5A-5D: GROOT gene SNPs show a robust association with biomass accumulation. 5A-5C: Box plots of TRP for GROOT gene variants; y-axis: TRP value; x-axis: types of variants (Col-0 allele and non-Col-0 allele); SNP position: (A) 6745394 for GROOT1, (B) 6810046 for GROOT2, (C) 6815838 for GROOT3. Significant differences (P<0.05) were determined using a T-test and are indicated by letters above the error bars. 5D: A box plot showing TRP for accessions grouped by different combinations of alleles of GROOT1, GROOT2, and GROOT3; x-axis: unique allele combination; y-axis: TRP of accessions in the corresponding group. Letters denote significance levels.

FIGS. 6A-6F: Root biomass accumulation under elevated temperatures in natural accessions. 6A: A graph showing distribution of TRP values of natural Arabidopsis thaliana accessions, with the positions of accessions used for elevated temperature experiments shown in red; y-axis: frequency; x-axis: TRP. 6B: Images showing growth differences between two accessions with different SNP types at chromosome 3 position 6772287 at 22° C. and 28° C. Bå5-1 contains Col-0 allele; IP-Pie-0 contains non-Col-0 allele. Both lines show higher root mass at elevated temperatures. 6C-6F: Line graphs showing phenotypes of Col-0 and non-Col-0 allele accessions at 22° C. and 28° C.; 6C: Root fresh weight; 6D: root dry weight; 6E: shoot fresh weight; 6F: shoot dry weight. Significance levels are shown in the figures (G: genotype; E: environment; GxE: genotype-by-environment interaction). Significance was determined using a two-way ANOVA followed by Tukey's HSD test (p<0.05). This experiment involved 22 accessions (12 with the Col-0 allele and 10 with the Non-Col-0 allele), with data collected from root and shoot tissues at 21 days after planting (DAP).

FIGS. 7A-7G: Plant biomass in GROOT mutants shows a significant genotype-by-environment (GxE) interactions. 7A-7B: Images showing root growth patterns of WT and GROOT1, GROOT2, and GROOT3 T-DNA lines, grown under 22° C. and 28° C., for 7 days (A) and 14 days (B) after plating. 7C-7D: Bar graphs showing primary root length (cm) of WT and GROOT1, GROOT2, and GROOT3 T-DNA lines, grown under 22° C. and 28° C., for 7 days (C) and 14 days (D) after plating. Significance levels are shown in the figures (G: genotype; E: environment; GxE: genotype-by-environment interaction). 7E: Images showing root growth patterns of WT and GROOT1, GROOT2, and GROOT3 T-DNA lines, grown under 28° C. for 21 days after plating. 7F-7G: Bar graphs showing root dry weight (F) or shoot dry weight (G) biomass of WT and GROOT1, GROOT2, and GROOT3 T-DNA lines, grown under 22° C. and 28° C., for 21 days after plating. Significance levels are shown in the figures (G: genotype; E: environment; GxE: genotype-by-environment interaction). Significant GxE interactions drive biomass accumulation. Significance was determined using a two-way ANOVA followed by Tukey's HSD test (p<0.05). The number of replicates for each WT and T-DNA line is indicated at the top of each bar.

FIG. 8: Early growth differences between wildtype and GROOT mutants. Representative images of four-week-old Arabidopsis thaliana plants grown on Turface under greenhouse conditions. Wildtype (Col-0) plants are shown alongside GROOT mutant lines. GROOT mutants exhibited more vigorous shoot development compared to wildtype plants at this stage.

FIGS. 9A-9C: Biomass accumulation and flowering time in nine-week-old GROOT mutant plants. 9A: Representative images of nine-week-old Col-0 wildtype and GROOT mutant plants grown on Turface. Mutants show visibly larger above-ground biomass compared to wildtype controls. 9B: Quantification of root dry weight after separation and drying at 50° C. for four days. 9C: Quantification of shoot dry weight under the same conditions. Mutant lines exhibited significantly higher biomass relative to wildtype controls. Error bars represent ±standard error of the mean (SEM); statistical significance was determined by Student's t-test.

### DETAILED DESCRIPTION

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “a cell” includes singular or plural cells and can be considered equivalent to the phrase “at least one cell.” As used herein, the term “comprises” means “includes.” For example, reference to “comprising a plant” includes one or a plurality of such plants. It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting.

In some examples, the numbers expressing quantities of ingredients, properties such as molecular weight, reaction conditions, and so forth, used to describe and claim certain embodiments are to be understood as being modified in some instances by the term “about” or “approximately.” For example, “about” or “approximately” can indicate +/−10% variation of the value it describes. Accordingly, in some aspects, the numerical parameters set forth herein are approximations that can vary depending upon the desired properties sought to be obtained by a particular embodiment. Notwithstanding that the numerical ranges and parameters setting forth the broad scope of some examples are approximations, the numerical values set forth in the specific examples are reported as precisely as practicable. The recitation of ranges of values herein is merely intended to serve as a shorthand method of referring individually to each separate value falling within the range.

To facilitate review of the various aspects, the following explanations of terms are provided:

Backcross: The mating of a hybrid to one of its parents. For example hybrid progeny, for example a first generation hybrid (F₁), can be crossed back one or more times to one of its parents. Backcrossing can be used to introduce one or more single locus conversions (such as one or more desirable traits) from one genetic background into another.

Biomass: As used herein includes biomass of an entire plant, above-ground biomass, and below-ground biomass. Above-ground biomass, synonymous with shoot biomass, refers to all plant material found above the soil surface, including stems, leaves, buds, flowers, and seeds. Below-ground biomass refers to all plant material found below the soil surface, primarily roots and sometimes underground stems (including bulbs, corms, rhizomes, stolons, and tubers). Below-ground biomass is synonymous with root biomass when the plant only has roots below the soil surface.

Cell: Cell as used herein includes a plant cell, whether isolated, in tissue culture, or being part of a plant or plant part. In some examples a cell is gene-edited, e.g., it has a nucleic acid and/or protein sequence not found in nature (e.g., a mutated GROOT1, GROOT2, and/or GROOT3). In some examples a cell is recombinant/transformed/transgenic, e.g., it includes an exogenous nucleic acid molecule.

Codon optimization: Referring to adapting the codon usage of nucleic acid (such as an exogenous or recombinant nucleic acid) to that of a cell or organism of interest (such as a plant cell or a plant) to improve the transcription rate of the nucleic acid in the cell or organism. Due to codon degeneracy, codon optimization does not alter the amino acid sequence of the encoded protein. Codon optimization can be based on codons that are differentially utilized in genes highly expressed within the cell or organism of interest.

Complementarity: The ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will form hydrogen bonds with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

Control: Refers to a plant, plant part, or plant cell that has a similar (or the same) genetic makeup and/or phenotypic traits as a treated plant, plant part, or plant cell before receiving the treatment. The treatment, for example, can include gene editing resulting in one or more modifications in one or more genes, expression of an exogenous gene, or RNAi treatment that reduces targeted mRNA translation. In some aspects, the control plant, plant part, or plant cell is wild-type with respect to the gene(s) being modified by the treatment, or is wild-type.

Clustered regularly interspaced short palindromic repeats (CRISPR)-CRISPR associated (Cas) system: CRISPR-Cas is an adaptive immune system existing in most bacteria and archaea, preventing them from being infected by phages, viruses and other foreign genetic elements. It includes CRISPR repeat-spacer arrays, which upon transcription generates CRISPR RNA (crRNA) and optionally trans-activating CRISPR RNA (tracrRNA), and a set of Cas genes which encode Cas proteins with endonuclease activity. CRISPR-Cas systems can be classified into two classes (Class 1 and Class 2), six types (I to VI), and several subtypes. Class 1 systems (Type I, III, and IV) utilize a multi-protein effector complex, whereas Class 2 systems (Type II, V, and VI) utilizes a single effector protein. CRISPR/Cas systems can be used for nucleic acid (DNA and RNA) targeting or editing, for example to detect a target nucleic acid, or cut or modify a target nucleic acid at any desired location.

The CRISPR repeat-spacer array (or CRISPR array) is a defining feature of CRISPR-Cas systems. The term “CRISPR” refers to the architecture of the array which includes constant direct repeats (DRs) interspaced with the variable spacers. In some examples, a CRISPR array includes at least a DR-spacer-DR-spacer. CRISPR spacer sequences are transcribed into short RNA sequences (“CRISPR RNAs” or “crRNAs”) capable of guiding Cas proteins to matching sequences of DNA.

Cas proteins provide the enzymatic machinery required for acquiring new spacers targeting invading elements and cleaving these elements upon subsequent encountering. Cas proteins that have endonuclease activity include Cas9, Cas12 (Cpf1), and Cas13.

Cas9 cleaves DNA and possesses two nuclease domain (HNH and RuvC), each cleaving one strand of the target double-stranded DNA. Cas9 nucleic acid and protein sequences are publicly available. For example, GenBank® Accession Nos. nucleotides 796693 . . . 800799 of CP012045.1 and nucleotides 1100046 . . . 1104152 of CP014139.1 disclose Cas9 nucleic acids, and GenBank® Accession Nos. AMA70685.1 and AKP81606.1 disclose Cas9 proteins. In some examples, Cas9 comprises at least 80% sequence identity, for example at least 85%, at least 90%, at least 95%, at least 96%, at least 97%, at least 98%, or at least 99% sequence identity to such sequences, and retains the ability to cut DNA.

In some examples, Cas9 can be catalytically inactive or deactivated (dCas9), such as one that is nuclease deficient. In some examples, dCas9 includes one or more of the following point mutations: D10A, 5 H840A, and N863A. In some examples, dCas9 comprises a sequence as shown in GenBank® Accession Nos. AKA60242.1 and KR011748.1, or comprises at least 80% sequence identity, for example at least 85%, at least 90%, at least 95%, at least 96%, at least 97%, at least 98%, or at least 99% sequence identity to such sequences.

Cross: Synonymous with hybridize or crossbreed; includes the mating of genetically different individual plants, such as the mating of two parent plants.

Cross-pollination: Fertilization by the union of two gametes from different plants.

Deletion: Elimination of a nucleic acid sequence from an organism's genome. Deletions can vary in size, ranging from small deletions involving a few nucleotides to large deletions encompassing one or more entire genes.

Endogenous: With reference to a nucleic acid and/or protein, referring to the nucleic acid and/or protein as found in a plant in its natural form (without any human intervention). “Endogenous” is synonymous with “native” as used herein. Endogenous genes include any naturally occurring alleles, and include those that have been modified at some point by traditional plant breeding methods and/or next generation plant breeding methods. Endogenous genes can be edited or mutated according to any methods known or described herein.

Exogenous: With reference to a nucleic acid molecule, protein, vector, plasmid, and/or construct, referring to any such substance that does not naturally occur in a cell or plant but is introduced into the cell or plant through human intervention. An exogenous nucleic acid can be identical to a nucleic acid found in a plant in its natural form, but not integrated within the same natural genetic environment. In some examples, an exogenous nucleic acid may be a guide nucleic acid (such as one specific for a region of a GROOT1, GROOT2, and/or GROOT3 gene). In some examples, an exogenous nucleic acid may be a gene carried by a vector for expression in a cell or plant to which it is introduced (optionally integrated into the genome of the cell or plant), wherein the gene can be a copy or variant of a gene naturally occurring in the cell or plant, or can be a gene not naturally occurring in the cell or plant. In some examples, an exogenous nucleic acid or vector or plasmid may be a CRISPR/Cas construct (such as a CRISPR/Cas9 construct) encoding the components of a CRISPR/Cas system, such as one specific for a GROOT1, GROOT2, and/or GROOT3 gene. In some examples, an exogenous construct may be a preassembled Cas protein (such as Cas9)-gRNA ribonucleoproteins.

Expression: Refers to the production of a functional gene product, e.g., an mRNA or a protein (precursor or mature).

F₁ hybrid: The first-generation progeny of the cross of two stable parents that are nonisogenic or isogenic plants.

Fragment: The terms “at least a portion” or “fragment” of a nucleic acid or protein refers to a portion having the minimal size characteristics of the molecule, or any larger fragment of the full-length molecule, up to and including the full-length molecule. A fragment may be a C-terminal fragment, N-terminal fragment, or an internal fragment that lies anywhere between the C-terminal and N-terminal amino acids. In some aspects, a fragment of a gene (e.g., GROOT1, GROOT2, and/or GROOT3) includes no more than 1500, 1400, 1300, 1200, 1100, 1000, 950, 900, 850, 800, 750, 700, 650, 600, 550, 500, 450, 400, 350, 300, 250, 200, 150, 140, 130, 120, 110, or 100, and/or no less than 30, 40, 50, 60, 70, 80, 90, 100, 110, 120, 130, 140, 150, 200, 250, or 300 contiguous nucleic acids of a full-length gene (such as one that includes a genomic sequence set forth in any of SEQ ID NOs: 1, 3-4, 6, 14, 16-17, 23 and 25-29, or includes (e.g., after introns are removed) a protein coding sequence set forth in any of SEQ ID NOs: 2, 5, 7, 15, 18 and 24). In some aspects, a fragment of a gene is a N-terminal fragment including the N-terminal nucleic acid of a full-length gene. In some aspects, a fragment of a gene is a C-terminal fragment including the C-terminal nucleic acid of a full-length gene. In some aspects, a fragment of a gene is an internal fragment including neither the C-terminal nor N-terminal nucleic acid of a full-length gene. In some aspects, a fragment of a gene may encode a biologically active portion of a full-length protein (such as any of those set forth in SEQ ID NOs: 8-13, 19-22, and 30-35). In some aspects, a fragment of a gene encodes no more than 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, or 30 and/or no less than 10, 15, 20, 25, 30, 35, 40, 45, 50, 55, 60, 65, 70, or 80 contiguous amino acids of a full-length protein. In some aspects, a biologically active portion of a full-length protein encoded by a fragment of a gene is a C-terminal, N-terminal, or internal fragment of the full-length protein. A functional fragment is a fragment that retains one or more functions or activities of the corresponding full-length nucleic acid or protein at a desirable level (e.g., at least 60%, 70%, 80%, 90%, 95%, 99%, or 100% of the level provided by the full-length molecule).

Gene editing: Modifying a genome of an organism, including mutating one or more genomic nucleotides, deleting one or more genomic nucleotides, adding one or more nucleotides into the genome, replacing a genomic sequence with an exogenous sequence, inserting an exogenous sequence into the genome, and any combination thereof. Gene editing can be achieved, for example, by using engineered nucleases, which create site-specific double-strand breaks (DSBs) at desired locations in the genome, and whose improper repair by endogenous natural mechanisms results in an altered/non-native genomic sequence. The induced DSBs may be repaired through nonhomologous end-joining (NHEJ) or homologous recombination (HR), resulting in targeted mutations or deletions of a genomic sequence, or insertion of an exogenous sequence into the genome. Thus, the resulting genome is one that does not occur in nature. Gene editing can also be achieved by, for example, Agrobacterium-mediated plant transformation.

In some examples, gene editing results in the introduction of an exogenous transgene into the genome of a plant cell, for example, one or more T-DNA sequences integrated into one or more of GROOT1, GROOT2, and GROOT3; or sequences that encode RNAi molecules specific for mRNAs transcribed from GROOT1, GROOT2, and/or GROOT3.

In other examples, a plant cell is edited using an exogenous nucleic acid molecule (e.g., a CRISPR/Cas vector) specific for an endogenous gene (e.g., one or more of GROOT1, GROOT2, and GROOT3), thereby altering the endogenous sequence of the gene, such as generating a loss-of-function mutation in the gene, but the exogenous nucleic acid molecule is not integrated into the genome of the gene-edited plant, plant part, or plant cell.

In either case, such edited plants, plant parts, and plant cells are referred to as gene-edited plants, gene-edited plant parts, and gene-edited plant cells, respectively. In some examples, the gene-edited plants, plant parts or plant cells are transgene-free. Gene editing in a plant can be used, for example, to confer a desirable trait to the plant, such as drought resistance, flooding resistance, erosion resistance, resistance to pests, increased root biomass, increased shoot biomass, increased seed size, increased carbon sequestration, etc.

Gene inactivation/down-regulation: When used in reference to the expression of a gene (e.g., one or more of GROOT1, GROOT2, and GROOT3), refers to any process which results in a decrease in production of a gene product and/or a decrease in its biological activity, such as a decrease of at least about 10%, 20%, 30%, 40%, 50%, 60%, 70%, 75%, 80%, or 85%. A gene product can be an RNA or a protein. A gene is “silenced” or “knocked out” when its expression, or activity of its gene product is significantly reduced (e.g., a reduction of at least about 80%, 85%, 90%, 95%, or 99%) or even prevented. A gene is “knocked down” when its expression or activity of its gene product is reduced but not completely eliminated. Gene inactivation, down-regulation, or silencing includes processes that decrease transcription of a gene, stability or translation of mRNA, and/or activity of protein. In some examples, a mutation, such as a substitution, partial or complete deletion, insertion, or other variation, can be made to a gene sequence that reduces or significantly reduces (and in some cases eliminates) production of a gene product, or renders a gene product partially, substantially, or completely non-functional.

In some aspects, the target of gene inactivation, down-regulation, or silencing is genomic DNA, such as a coding or regulatory DNA region for one or more of GROOT1, GROOT2, and GROOT3. In some examples, CRISPR/Cas systems may be used to introduce the desired mutation or deletion to genomic DNA. For example, to generate a knockout or knockdown line, CRISPR/Cas9 can be used to homozygously introduce DSBs in target genomic regions. NHEJ fixes these double-strand breaks, but results in insertions and/or deletions (INDELs) during the process. INDELs in target exons generate a premature termination codon (PTC) in mRNA by changes in the reading-frame, which induces degradation of nascent mRNAs with a PTC by the nonsense-mediated decay (NMD) system. In some examples, Agrobacterium-mediated plant transformation may be used, where one or more T-DNA sequences are used to disrupt one or more of GROOT1, GROOT2, and GROOT3 through Agrobacterium infection, such that expression of the one or more genes are reduced or eliminated.

In some aspects, the target of gene inactivation, down-regulation, or silencing is mRNA. Translation and/or stability of mRNA can be reduced to decrease the level of protein, for example, by RNAi technology.

In some examples, gene inactivation, down-regulation, or silencing reduces a detectable level or activity of a mRNA or protein encoded by a target gene (e.g., one or more of GROOT1, GROOT2, and GROOT3) in a plant, plant part, or plant cell by at least about 10%, at least about 20%, at least about 30%, at least about 40%, at least about 50%, at least about 60%, at least about 70%, at least about 75%, at least about 80%, at least about 90%, at least about 95%, at least about 96%, at least about 97%, at least about 98%, or at least about 99% (such as a decrease of 40% to 90%, 40% to 80% or 50% to 95%), or 100% as compared to that of a control.

Genome: All genetic material of an organism (such as a plant), including nuclear genome and organelle genome and excluding artificially introduced nucleic acid molecules not integrated into a chromosome.

Genotype: The genetic constitution (e.g., the specific allele makeup) of a cell or an organism (e.g., a plant) usually with reference to a specific character under consideration.

GROOT1: Includes both GROOT1 genes and proteins encoded by GROOT1 genes (GROOT1 proteins). GROOT1 genes (or GROOT1) include the GROOT1 gene found in Arabidopsis thaliana, as well as any orthologs thereof found in any other plants. GROOT1 genes also include any functional fragments of full-length genes. GROOT1 proteins include the GROOT1 protein found in Arabidopsis thaliana, as well as any orthologs thereof found in any other plants. GROOT1 proteins also include any functional fragments of full-length proteins. GROOT1 proteins are members of the pseudouridine synthase family. Pseudouridine (Ψ), the isomer of uridine (U), is the most abundant type of RNA modification, which is crucial for gene regulation in various cellular processes. Pseudouridine synthases are the key enzymes for the U-to-Ψ conversion. It is shown herein that reducing GROOT1 expression and/or activity increases plant biomass, particularly root biomass and/or shoot biomass, and/or seed size. Thus, reducing GROOT1 expression and/or activity can be used to increase plant biomass, particularly root biomass and/or shoot biomass, and/or seed size.

Exemplary GROOT1 protein sequences are provided in SEQ ID NOs: 8-13. In some aspects, GROOT1 proteins include any protein that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to any of the sequences set forth in SEQ ID NOs: 8-13, and retains the one or more functions or activities of any of SEQ ID NOs: 8-13 (e.g., pseudouridine synthase activity; regulating plant biomass, particularly root biomass and/or shoot biomass, and/or seed size).

GROOT1 genes include any gene that encodes any of the GROOT1 proteins provided herein, or any gene that (e.g., when introns are removed) includes a GROOT1 protein coding sequence provided herein. Exemplary GROOT1 genomic sequences are provided in SEQ ID NOs: 1, 3-4 and 6. In some aspects, GROOT1 genes include any nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to any of SEQ ID NOs: 1, 3-4 and 6, and encodes a GROOT1 protein (which is a pseudouridine synthase, that can regulate plant biomass, particularly root biomass and/or shoot biomass, and/or seed size).

Exemplary GROOT1 protein coding sequences are provided in SEQ ID NOs: 2, 5 and 7. In some aspects, GROOT1 protein coding sequences include any nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to any sequence set forth in SEQ ID NOs: 2, 5 and 7, and encodes a GROOT1 protein (which is a pseudouridine synthase, that can regulate plant biomass, particularly root biomass and/or shoot biomass, and/or seed size).

GROOT2: Includes both GROOT2 genes and proteins encoded by GROOT2 genes (GROOT2 proteins). GROOT2 genes (or GROOT2) include the GROOT2 gene found in Arabidopsis thaliana, as well as any orthologs thereof found in any other plants. GROOT2 genes also include any functional fragments of full-length genes. GROOT2 proteins include the GROOT2 protein found in Arabidopsis thaliana, as well as any orthologs thereof found in any other plants. GROOT2 proteins also include any functional fragments of full-length proteins. GROOT2 proteins are mitotic checkpoint proteins, and in Arabidopsis thaliana is BUB3.1 (budding uninhibited by benzymidazol 3.1) and function in spindle assembly checkpoint (SAC) signaling, promoting the establishment of correct kinetochore-microtubule (K-MT) attachments and the formation of stable end-on bipolar attachments. The mitotic checkpoint or spindle checkpoint is a cell cycle checkpoint that delays the segregation until all chromosomes are correctly attached to the mitotic spindle. For proper segregation, kinetochores on sister chromatids must be connected to opposite poles of the spindle (bipolar orientation). This checkpoint operates by inhibiting the activity of an anaphase-promoting complex/cyclosome (APC/C). It is shown herein that reducing GROOT2 expression and/or activity increases plant biomass, particularly root biomass and/or shoot biomass, and/or seed size. Thus, reducing GROOT2 expression and/or activity can be used to increase plant biomass, particularly root biomass and/or shoot biomass, and/or seed size.

Exemplary GROOT2 protein sequences are provided in SEQ ID NOs: 19-22. In some aspects, GROOT2 proteins include any protein that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to any of the sequences set forth in SEQ ID NOs: 19-22, and retains the one or more functions or activities of any of SEQ ID NOs: 19-22 (e.g., SAC signaling; promoting the establishment of correct K-MT attachments; promoting the formation of stable end-on bipolar attachments; regulating plant biomass, particularly root biomass and/or shoot biomass, and/or seed size).

GROOT2 genes include any gene that encodes any of the GROOT2 proteins provided herein, or any gene that (e.g., when introns are removed) includes a GROOT2 protein coding sequence provided herein. Exemplary GROOT2 genomic sequences are provided in SEQ ID NOs: 14 and 16-17. In some aspects, GROOT2 genes include any nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to any of SEQ ID NOs: 14 and 16-17, and encodes a GROOT2 protein (which is a mitotic checkpoint protein, that can regulate plant biomass, particularly root biomass and/or shoot biomass, and/or seed size).

Exemplary GROOT2 protein coding sequences are provided in SEQ ID NOs: 15 and 18. In some aspects, GROOT2 protein coding sequences include any nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to any sequence set forth in SEQ ID NOs: 15 and 18, and encodes a GROOT2 protein (which is a mitotic checkpoint protein, that can regulate plant biomass, particularly root biomass and/or shoot biomass, and/or seed size).

GROOT3: Includes both GROOT3 genes and proteins encoded by GROOT3 genes (GROOT3 proteins). GROOT3 genes (or GROOT3) include the GROOT3 gene found in Arabidopsis thaliana, as well as any orthologs thereof found in any other plants. GROOT3 genes also include any functional fragments of full-length genes. GROOT3 proteins include the GROOT3 protein found in Arabidopsis thaliana, as well as any orthologs thereof found in any other plants. GROOT3 proteins also include any functional fragments of full-length proteins. GROOT3 proteins are radical S-adenosyl-L-methionine (SAM) proteins. Radical SAM proteins use an iron-sulfur cluster (4Fe-4S) to reductively cleave SAM to generate a radical, usually a 5′-deoxyadenosyl radical (5′-dAdo⋅), as a critical intermediate. Radical SAM proteins then utilize this radical intermediate to perform diverse functions, including methylating unreactive carbon and phosphorus centers, catalyzing methylthiolation on tRNA nucleotides, catalyzing biotin synthesis and lipoic acid metabolism, catalyzing carbon insertion reactions, catalyzing post-translational modification, etc. Radical SAM proteins have a cysteine-rich motif that matches or resembles CX₃CX₂C, where X represents any amino acid. It is shown herein that reducing GROOT3 expression and/or activity increases plant biomass, particularly root biomass and/or shoot biomass, and/or seed size. Thus, reducing GROOT3 expression and/or activity can be used to increase plant biomass, particularly root biomass and/or shoot biomass, and/or seed size.

Exemplary GROOT3 protein sequences are provided in SEQ ID NOs: 30-35. In some aspects, GROOT3 proteins include any protein that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to any of the sequences set forth in SEQ ID NOs: 30-35, and retains the one or more functions or activities of any of SEQ ID NOs: 30-35 (e.g., using iron-sulfur cluster (4Fe-4S) to reductively cleave SAM to generate a radical; regulating plant biomass, particularly root biomass and/or shoot biomass, and/or seed size).

GROOT3 genes include any gene that encodes any of the GROOT3 proteins provided herein, or any gene that (e.g., when introns are removed) includes a GROOT3 protein coding sequence provided herein. Exemplary GROOT3 genomic sequences are provided in SEQ ID NOs: 23 and 25-29. In some aspects, GROOT2 genes include any nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to any of SEQ ID NOs: 23 and 25-29, and encodes a GROOT3 protein (which is a radical SAM protein, that can regulate plant biomass, particularly root biomass and/or shoot biomass, and/or seed size).

Exemplary GROOT3 protein coding sequence is provided in SEQ ID NO: 24. In some aspects, GROOT3 protein coding sequences include any nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 91.5%, at least about 92%, at least about 92.5%, at least about 93%, at least about 93.5%, at least about 94%, at least about 94.5%, at least about 95%, at least about 95.5%, at least about 96%, at least about 96.5%, at least about 97%, at least about 97.5%, at least about 98%, at least about 98.5%, at least about 99%, or at least about 99.5% sequence identity, or 100% sequence identity to the sequence set forth in SEQ ID NO: 24, and encodes a GROOT3 protein (which is a radical SAM protein, that can regulate plant biomass, particularly root biomass and/or shoot biomass, and/or seed size).

Growing or regeneration: Growing a whole, differentiated plant from a seed, a plant cell, a protoplast, a group of plant cells, callus, a plant part, a plant tissue, etc. In some examples, regeneration refers to the development of a plant from tissue culture. The cells may or may not have been genetically modified. Plant tissue culture relies on the fact that plant cells have the ability to generate a whole plant (totipotency). Single cells (protoplasts), pieces of leaves, or roots can often be used to generate a new plant on culture media given the required nutrients and plant hormones.

Guide nucleic acid: Including guide RNAs (including single guide RNAs), any nucleic acid intermediates/precursors that can be processed into guide RNAs, and/or DNA molecules from which the guide RNAs or the intermediates/precursors can be transcribed. A guide nucleic acid can include modified bases or chemical modifications (e.g., see Latorre et al., Angewandte Chemie 55:3548-50, 2016).

Guide RNA (gRNA): RNA molecules including a guide sequence, and a sequence that assists binding of a nuclease (such as a DNA endonuclease, such as a Cas protein). “Guide sequence,” also known as “CRSPR RNA (crRNA),” refers to an RNA sequence that has sufficient complementarity with a target polynucleotide sequence to hybridize with the target sequence. In some aspects, guide RNA is used to refer to a final component of a CRSPR/Cas complex, that binds with a Cas protein and hybridizes to a target sequence.

A single guide RNA (sgRNA) refers to a single RNA molecule, typically including two parts, a guide sequence or crRNA, and a trans-activating CRISPR RNA (tracrRNA), serving as a binding scaffold for a nuclease (e.g., Cas9). A sgRNA also includes a single crRNA that functions together with a Cpf1.

In some examples, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some examples, a guide sequence is about, or at least about, 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some examples, a guide sequence is less than about 75, 50, 45, 40, 35, 30, 25, 20, 15, 12, or fewer nucleotides in length. In some examples, a guide sequence is 15-25 nucleotides (such as 18-22, or 18 nucleotides) in length.

The ability of a guide sequence to direct sequence-specific binding of a CRISPR/Cas complex to a target sequence may be assessed by a suitable assay. For example, the components of a CRISPR/Cas system sufficient to form a CRISPR/Cas complex, including the guide sequence to be tested, may be provided to a host cell having the corresponding target sequence, followed by an assessment of preferential cleavage within the target sequence. Additionally, cleavage of a target sequence may be evaluated in a test tube by providing the target sequence, and components of a CRISPR/Cas complex, including the guide sequence to be tested in the test tube, and comparing binding or rate of cleavage at the target sequence of the test complex to a control.

Heterologous: A substance coming from some source or location other than its native source or location. A heterologous nucleic acid sequence can refer to a sequence that is not naturally found in the particular organism. Two nucleic acid sequences are heterologous to one another if the sequences are derived from separate organisms, whether or not such organisms are of different species, as long as the sequences do not naturally occur together in the same arrangement in the same organism. In some examples, a heterologous promoter refers to a promoter that has been taken from one source organism and utilized in another organism, in which the promoter is not naturally found. In some examples, a heterologous promoter refers to a promoter that is from within the same source organism, but is used at a novel location, in which the promoter is not normally located. Heterologous gene sequences can be introduced into a cell (such as a plant cell) by using an expression vector, which can be a eukaryotic expression vector, for example a plant expression vector. Methods used to construct vectors are known and described in various publications. In particular, techniques for constructing suitable vectors, including selecting and organizing the functional components such as promoters, enhancers, termination and polyadenylation signals, selection markers, origins of replication, and splicing signals, are known. Heterologous gene sequences can also be introduced into a cell (such as a plant cell) using an integration vector, such as the Ti plasmid from Agrobacterium tumefaciens.

Homologs: With reference to a gene or gene product, nucleic acids, and proteins thought, believed, or known to be functionally related. A functional relationship may be indicated by, for example (a) degree of sequence identity and/or (b) the same or similar biological function. Homology can be determined using software programs readily available in the art, such as those discussed in Current Protocols in Molecular Biology (F. M. Ausubel et al., eds., 1987) Supplement 30, section 7.718, Table 7.71. Some alignment programs are MacVector (Oxford Molecular Ltd, Oxford, U.K.) and ALIGN Plus (Scientific and Educational Software, Pennsylvania). Other non-limiting alignment programs include Sequencher (Gene Codes, Ann Arbor, Michigan), AlignX, and Vector NTI (Invitrogen, Carlsbad, CA). Homologous genes/proteins arise in evolution in two possible ways: separation of two populations with the ancestral gene into two species, and duplication of the ancestral gene within a lineage. Homologous genes/proteins separated by speciation are also called orthologs. Homologous genes/proteins separated by speciation and brought back together in a single species by allopolyploidization are also called homeologs. Homologous genes/proteins arise from gene duplication events within a species are also called paralogs. Homologs include orthologs, homeologs, and paralogs.

Increase or decrease: A statistically significant positive or negative change, respectively, in quantity from a control value. An increase is a positive change, such as an increase of at least about 50%, at least about 100%, at least about 200%, at least about 300%, at least about 400%, or at least about 500% as compared to the control value. A decrease is a negative change, such as a decrease of at least about 20%, at least about 25%, at least about 50%, at least about 75%, at least about 80%, at least about 90%, at least about 95%, at least about 98%, at least about 99%, or at least about 100% decrease as compared to a control value. In some examples, the control value is a value or range of values expected for a similar plant that is not gene-edited (e.g., a wild-type plant), or not gene-edited with respect to the gene in question (e.g., a wild-type plant with respect to a particular gene).

Isolated: An “isolated” biological substance (such as a protein, nucleic acid, protein-nucleic acid complex, vector cell) has been substantially separated, produced apart from, or purified away from other biological components in a cell or biological agent in which the substance naturally occurs, such as other cells, chromosomal and extrachromosomal DNAs and RNAs, and proteins. Isolated nucleic acids and proteins include nucleic acids and proteins purified by standard purification methods. The term also embraces artificially synthesized nucleic acids, proteins, and complexes thereof, including nucleic acids generated by PCR, and in vitro and in vivo synthesis or replication or transcription (e.g., in vitro transcription to synthesize RNA), and including proteins prepared by recombinant expression in a host cell or otherwise artificially synthesized (e.g., chemical synthesis, cell-free synthesis, etc.). Isolated substance, in some examples, are at least about 50% pure, such as at least about 75%, at least about 80%, at least about 90%, at least about 95%, at least about 98%, or 100% pure.

Loss-of-function mutation: A genetic mutation (including substitution, insertion, deletion, inversion, etc.) that leads to a reduction or complete loss of the normal function of the gene in which the mutation occurs. The normal function of a gene includes being transcribed into mRNAs, that can be translated into proteins with normal activity. In some examples, a loss-of-function mutation reduces or eliminates transcription of the gene, for example, by causing a loss of interaction between the gene (e.g., regulatory elements upstream of a coding sequence) and the transcriptional machinery. In some examples, a loss-of-function introduces a premature stop codon or frameshift mutation, which leads to the production of truncated mRNA transcripts, which are subject to degradation by cellular mechanism. In some examples, a loss-of-function mutation results in the production of a completely nonfunctional protein, or a protein with a reduced function, compared to the protein encoded by the gene before the mutation.

Next generation plant breeding: Refers to plant breeding tools and methodologies that are available to a plant breeder. One distinguishing feature of next generation plant breeding is that the breeder is no longer confined to relying upon observed phenotypic variation, in order to infer underlying genetic causes for a given trait. Rather, next generation plant breeding can include the utilization of molecular markers and marker assisted selection (MAS), such that the breeder can directly observe movement of alleles and genetic elements of interest from one plant in the breeding population to another, and is not confined to merely observing phenotypes. Further, next generation plant breeding methods are not confined to utilizing natural genetic variation found within a plant population. Rather, the breeder utilizing next generation plant breeding methodology can access modern genetic engineering tools that directly alter/change/edit the plant's underlying genetic architecture in a targeted manner, in order to bring about a phenotypic trait of interest. In some aspects, the plants bred with a next generation plant breeding methodology are indistinguishable from a plant that was bred in a traditional manner, as the resulting end product plant could theoretically be developed by either method. In particular aspects, a next generation plant breeding methodology may result in a plant that comprises a genetic modification (e.g., a deletion or insertion of any size; a substitution of one or more base pairs; an introduction of nucleic acid sequences from within the plant's natural gene pool (e.g. any plant that could be crossed or bred with a plant of interest) or from editing of nucleic acid sequences in a plant to correspond to a sequence known to occur in the plant's natural gene pool); and offspring of the plant.

Naturally occurring: As applied to a substance (e.g., nucleic acid, polypeptide/protein, etc.), cell, or organism, refers to a substance, cell, or organism that is found in nature, without any intentional human intervention in its existence or evolvement.

Non-naturally occurring or engineered: Indicating involvement of the hand of human. In some examples, the terms, when referring to a substance (e.g., a nucleic acid molecule, or a polypeptide/protein) or a cell, indicate that the substance or cell is at least substantially free from at least one other substances or cells with which it is naturally associated or found together in nature. In some examples, a non-naturally occurring or engineered sequence (e.g., of a nucleic acid molecule, polypeptide/protein, etc.) refers to a sequence that is at least partially different from naturally occurring sequences, and the difference is achieved by synthesis, recombinant technology, gene editing, or any other production or intervention means that are developed by human.

Nucleotide change/modification: Refers to nucleotide substitution, deletion, and/or insertion to a reference sequence. In some examples, nucleotide changes/modifications can be “silent,” meaning that the changes/modifications do not alter the properties or activities of the encoded protein and/or how the protein is made. In some examples, the changes/modifications are not silent.

Offspring: Refers to any plant resulting as progeny from a vegetative or sexual reproduction from one or more parent plants or descendants thereof. For instance, an offspring plant may be obtained by cloning or selfing of a parent plant (or a plant of F1, F2, or still further generations), or by crossing two parent plants (or a plant of F1, F2, or still further generations). An offspring of F1 generation is a first-generation offspring produced from parents. Subsequent generations, denoted as F2, F3, and so forth, arise from selfing or crossing within the preceding generation. In some examples, an F1 may be (and usually is) a hybrid resulting from a cross between two true breeding parents (true breeding referring to homozygous for a trait), while an F2 may be (and usually is) an offspring resulting from self-pollination of the F1 hybrids.

Operably linked: Two nucleic acid sequences are operably linked if the nature of the linkage does not interfere with the normal functions of the sequences. A first nucleic acid sequence is operably linked with a second nucleic acid sequence when the first nucleic acid sequence is placed in a functional relationship with the second nucleic acid sequence. In some examples, a promoter is operably linked to a nucleic acid sequence (such as a guide nucleic acid sequence or a coding sequence) if the promoter controls the transcription or expression of the nucleic acid sequence. In some examples, operably linked DNA sequences are contiguous and, where necessary join two protein-coding regions in the same reading frame. In some examples, coding sequences can be operably linked to regulatory sequences in a sense or antisense orientation.

Ortholog: Refers to genes or proteins in different species that evolved from a common ancestral gene or protein by speciation. Normally, orthologs retain the same or similar function in the course of evolution. Identification of orthologs is useful for reliable prediction of gene function in newly sequenced genomes. In some aspects, orthologs to the GROOT1 gene in Arabidopsis thaliana includes a nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 90.5%, at least about 91%, at least about 92%, at least about 93%, at least about 94%, or at least about 95% sequence identity to any of SEQ ID NO: 1, or to SEQ ID NO: 2 (e.g., after introns are removed). In some aspects, orthologs to the GROOT2 gene in Arabidopsis thaliana includes a nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95% sequence identity to SEQ ID NO: 14, or to SEQ ID NO: 15 (e.g., after introns are removed). In some aspects, orthologs to the GROOT3 gene in Arabidopsis thaliana includes a nucleic acid sequence that includes or has at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or at least about 95% sequence identity to SEQ ID NO: 23, or to SEQ ID NO: 24 (e.g., after introns are removed).

Plant: Includes reference to an immature or mature whole plant, including seedlings and plantlets, including a plant from which seed, roots, or leaves have been removed. Seeds or embryos that will produce a plant is also considered to be the plant. In some examples, the plants (including seeds and embryos) can include one or more exogenous nucleic acid molecules (such as a construct encoding a CRISPR/Cas complex). In some examples, the plants (including seeds and embryos) can include one or more loss-of-function mutations in one or more of GROOT1, GROOT2, and GROOT3.

Any commercially or scientifically valuable plant can be used in accordance with this disclosure. Exemplary plants include plants belonging to the super family Viridiplantae, such as monocotyledonous and dicotyledonous plants including a fodder or forage legume, ornamental plant, food crop, tree, or shrub, such as Acacia spp., Acer spp., Actinidia spp., Aesculus spp., Agathis australis, Albizia amara, Alsophila tricolor, Andropogon spp., Arachis spp, Areca catechu, Astelia fragrans, Astragalus cicer, Baikiaea plurijuga, Betula spp., Brassica spp., Bruguiera gymnorrhiza, Burkea africana, Butea frondosa, Cadaba farinosa, Calliandra spp, Camellia sinensis, Canna indica, Capsicum spp., Cassia spp., Centroema pubescens, Chacoomeles spp., Cinnamomum cassia, Coffea arabica, Colophospermum mopane, Coronillia varia, Cotoneaster serotina, Crataegus spp., Cucumis spp., Cupressus spp., Cyathea dealbata, Cydonia oblonga, Cryptomeria japonica, Cymbopogon spp., Cynthea dealbata, Cydonia oblonga, Dalbergia monetaria, Davallia divaricata, Desmodium spp., Dicksonia squarosa, Dibeteropogon amplectens, Dioclea spp, Dolichos spp., Dorycnium rectum, Echinochloa pyramidalis, Ehraffia spp., Eleusine coracana, Eragrestis spp., Erythrina spp., Eucalyptus spp., Euclea schimperi, Eulalia villosa, Pagopyrum spp., Feijoa sellowlana, Fragaria spp., Flemingia spp, Freycinetia banksli, Geranium thunbergii, GinAgo biloba, Glycine javanica, Gliricidia spp, Gossypium hirsutum, Grevillea spp., Guibourtia coleosperma, Hedysarum spp., Hemaffhia altissima, Heteropogon contoffus, Hordeum vulgare, Hyparrhenia rufa, Hypericum erectum, Hypeffhelia dissolute, Indigo incamata, Iris spp., Leptarrhena pyrolifolia, Lespediza spp., Lettuca spp., Leucaena leucocephala, Loudetia simplex, Lo tonus bainesli, Lotus spp., Macro tyloma axillare, Malus spp., Manihot esculenta, Medicago saliva, Metasequoia glyptostroboides, Musa sapientum, Nicotianum spp., Onobrychis spp., Ornithopus spp., Oryza spp., Peltophorum africanum, Pennisetum spp., Persea gratissima, Petunia spp., Phaseolus spp., Phoenix canadensis, Phormium cookianum, Photinia spp., Picea glauca, Pinus spp., Pisum sativum, Podocarpus totara, Pogonarthria fleckii, Pogonaffhria squarrosa, Populus spp., Prosopis cineraria, Pseudotsuga menziesii, Pterolobium stellatum, Pyrus communis, Quercus spp., Rhaphiolepsis umbellata, Rhopalostylis sapida, Rhus natalensis, Ribes grossularia, Ribes spp., Robinia pseudoacacia, Rosa spp., Rubus spp., Salix spp., Schyzachyrium sanguineum, Sciadopitys vefficillata, Sequoia sempervirens, Sequoiadendron giganteum, Sorghum bicolor, Spinacia spp., Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia spp., Vitis vinifera, Watsonia pyramidata, Zantedeschia aethiopica, Zea mays, amaranth, artichoke, asparagus, broccoli, Brussels sprouts, cabbage, canola, carrot, cauliflower, celery, collard greens, flax, kale, lentil, oilseed rape, okra, onion, potato, rice, soybean, straw, sugar beet, sugar cane, sunflower, tomato, squash tea, maize, wheat, barley, rye, oat, peanut, pea, lentil and alfalfa, cotton, rapeseed, canola, pepper, sunflower, tobacco, eggplant, switchgrass, Miscanthus, Setaria, fescue, eucalyptus, a tree, an ornamental plant, a perennial grass and a forage crop. In some aspects, the plant is a pennycress plant, such as Thlaspi arvense. In some aspects, the plant is a soybean plant, such as Glycine max. In some aspects, the plant is a canola plant, such as Brassica napus. In some aspects, the plant is a cereal grass. A cereal grass is a member of the Poaceae family that is cultivated for its edible grain. A cereal grass includes barley, corn/maize, goat grass, millet, oat, rice, rye, sorghum, and wheat. In one aspect, the plant is a rice plant, such as a plant of the genus Oryza, or such as Oryza sativa. In another aspect, the plant is a sorghum or great millet plant, such as Sorghum bicolor.

Plant cell: Includes a single plant cell or a plurality of plant cells; includes any cell that constitutes a plant; includes protoplasts, gamete producing cells, and cells that can regenerate into a whole plant, embryos, and callus tissue; includes cells from seeds, suspension cultures, embryos, meristematic regions, callus tissue, leaves, roots, shoots, gametophytes, sporophytes, pollen and microspores.

Plant part: Includes protoplasts, leaves, stems, roots, root tips, anthers, pistils, seeds, embryos, pollens, stamens, ovules, microspores, sporophytes, gametophytes, cotyledons, hypocotyls, flowers, shoots, fruits, tissues, petioles, cells, meristematic cells, and the like; includes differentiated and undifferentiated tissues (which may be in a plant, a plant organ, or a tissue or cell culture); includes plant cells of a tissue culture from which plants can be regenerated. In some examples, a plant part is one or more plant cells (e.g., single cells, protoplasts, embryos, and callus tissue).

Polynucleotide/nucleic acid molecule/nucleotide sequence: These terms are used interchangeably herein and refer to a polymeric form of nucleotides of any length, either ribonucleotides or deoxyribonucleotides, or analogs thereof. Includes double-stranded (such as sense and antisense) and single-stranded (such as sense or antisense) DNA, double- and single-stranded RNA, as well as multi-stranded DNA or RNA. Includes genomic DNA, cDNA, DNA-RNA hybrids, or a polymer comprising purine and pyrimidine bases or other natural, chemically or biochemically modified, non-natural, or derivatized nucleotide bases. Also includes modified nucleic acids such as methylated and/or capped nucleic acids, nucleic acids containing modified bases, backbone modifications, and the like. Oligonucleotide generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art.

Progeny: Offspring; descendants.

Promoter: A nucleic acid sequence, or an array of nucleic acid sequences, that direct or control transcription of a nucleic acid (e.g., a coding sequence). A promoter includes a necessary nucleic acid sequence near the start site of transcription. A promoter also optionally includes distal enhancer or repressor elements. In some examples, a promoter used for recombinant expression of a nucleic acid molecule is not naturally occurring in the cell into which it is introduced, is not native to the nucleic acid molecule to which it is attached, or both. In one example, a promoter used is not endogenous (i.e., is exogenous) to the plant in which it is introduced. In some examples, promoter is about 80-120 base pairs long and located upstream of the initiation site of a gene, to which RNA polymerase may bind and initiate correct transcription. There can be associated additional transcription regulatory sequences which provide on/off regulation of transcription and/or which enhance (increase) expression of the downstream coding sequence.

Protein/peptide/polypeptide: These terms are used interchangeably herein, and refer to a polymeric form of amino acids of any length, which can include coded and non-coded amino acids, chemically or biochemically modified or derivatized amino acids, and polypeptides having modified peptide backbones.

Recombinant: Of or resulting from new combinations of genetic material.

A recombinant protein may refer to a protein produced by the use of recombinant DNA technology, which involves the combination of genetic material from different sources to create a new (non-naturally occurring) DNA sequence, which is then introduced into a host organism (such as bacteria, yeast, or mammalian cells) to produce the desired protein.

A recombinant nucleic acid or a recombinant construct may refer to an artificial combination of nucleic acid sequences, e.g., regulatory and coding sequences that are not found together in nature. For example, a recombinant construct may include regulatory sequences and coding sequences that are derived from different sources, or regulatory sequences and coding sequences derived from the same source, but arranged in a manner different than that found in nature. Such a construct may be used by itself or may be incorporated into a vector or plasmid to form a recombinant vector or plasmid. Different independent transformation events of a recombinant construct or vector can result in different levels and patterns of expression (Jones et al., (1985) EMBO J. 4:2411-2418; De Almeida et al., (1989) Mol. Gen. Genetics 218:78-86). Lines displaying the desired expression level and pattern can be screened. Such screening may be accomplished, for example, by Southern analysis of DNA, Northern analysis of mRNA expression, immunoblotting analysis of protein expression, or phenotypic analysis, among others.

A recombinant or host cell may refer to a cell that has been genetically altered, or is capable of being genetically altered, by introduction of an exogenous polynucleotide, such as a recombinant construct, plasmid or vector. In some examples, the exogenous polynucleotide may express a protein complex (e.g., a CRSPR/Cas complex) or RNAi molecule that leads to reduced expression of one or more of GROOT1, GROOT2, and GROOT3. Typically, a host cell is a cell in which a vector can be propagated and its nucleic acid expressed. In specific examples, such cells are plant cells, such as from a monocot or dicot. The term also includes any progeny of the subject host cell. It is understood that all progenies may not be identical to the parental cell since there may be mutations that occur during replication. However, such progenies are included when the term “host cell” is used.

Ribonucleoprotein (RNP): A complex of RNA(s) and protein(s). In some aspects, the RNP is a CRSPR/Cas complex, which includes Cas protein(s) (such as a native or mutant Cas9 protein), and guide RNA(s). In some examples, the RNP includes one or more, such as two, three, four, or five different RNAs, such as guide RNAs specific for different targets, such as one or more regions of one or more of GROOT1, GROOT2, and GROOT3.

Self-pollination: The transfer of pollen from the anther to the stigma of the same plant.

Sequence identity/similarity: The similarity between proteins, or between nucleic acid molecules can be characterized by similarity between the amino acid sequences or nucleotide sequences, otherwise referred to as sequence identity. Sequence identity is frequently measured in terms of percentage identity (or similarity or homology); the higher the percentage, the more similar the two sequences are.

Methods of alignment of sequences for comparison are well known. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, MD) and on the internet, for use in connection with the sequence analysis programs blastp, blastn, blastx, tblastn and tblastx. A description of how to determine sequence identity using this program is available on the NCBI website on the internet.

Variants of protein sequences known and disclosed herein are typically characterized by possession of at least about 80%, at least 90%, at least 95%, at least 96%, at least 97%, at least 98% or at least 99% sequence identity counted over the full-length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. These sequence identity ranges are provided for guidance only; it is entirely possible that strongly significant homologs could be obtained that fall outside of the ranges provided.

Variants of the disclosed nucleic acid sequences are typically characterized by possession of at least about 80%, at least about 90%, at least about 95%, at least about 96%, at least about 97%, at least about 98% or at least about 99% sequence identity counted over the full-length alignment with the nucleic acid sequence using the NCBI Blast 2.0, gapped blastn set to default parameters. One of skill in the art will appreciate that these sequence identity ranges are provided for guidance only; it is possible that sequences coding for the disclosed proteins (e.g., one or more of GROOT1, GROOT2, GROOT3 proteins) could be obtained that fall outside of the ranges provided.

Tissue culture: A composition that includes isolated cells of the same or a different type or a collection of such cells organized into parts of a plant. In some examples, the tissue culture includes a homogenous population of plant cells. In some examples, the tissue culture includes a callus tissue. In some examples, the tissue culture includes an anther culture or apical stem tip meristem culture. In some examples, the tissue culture includes a hairy root culture.

Traditional plant breeding: Refers to the utilization of natural variation found within a plant population as a source for alleles and genetic variants that impart a trait of interest to a given plant. Traditional breeding methods make use of crossing procedures that rely largely upon observed phenotypic variation to infer causative allele association. That is, traditional plant breeding relies upon observations of expressed phenotype of a given plant to infer underlying genetic cause. These observations are utilized to inform the breeding procedure in order to move allelic variation into germplasm of interest. Further, traditional plant breeding has also been characterized as comprising random mutagenesis techniques, which can be used to introduce genetic variation into a given germplasm. These random mutagenesis techniques may include chemical and/or radiation-based mutagenesis procedures. Consequently, one feature of traditional plant breeding is that the breeder does not utilize a genetic engineering tool that directly alters/changes/edits the plant's underlying genetic architecture in a targeted manner, in order to introduce genetic diversity and bring about a phenotypic trait of interest.

Transformation: The introduction of exogenous material (e.g., vectors encoding gene editing machineries; RNAi molecules or antisense RNAs or vectors providing for such; vectors including non-coding or random sequences intended for disrupting a genomic region; guide RNAs; RNPs) into cells, for example a plant cell. Exemplary mechanisms for introducing nucleic acids into plant cells include (but are not limited to) electroporation, microprojectile bombardment, Agrobacterium-mediated transformation, and direct DNA uptake by protoplasts.

Transformed: A transformed plant, plant part or plant cell is a plant, plant part or plant cell that has taken up an exogenous nucleic acid (including a linear or circular DNA, a vector, a plasmid, an RNAi molecule (e.g., siRNA and miRNA), a guide RNA molecule, etc.), regardless of whether the exogenous nucleic acid is integrated into the genome of the plant, plant part or plant cell, and regardless of whether the exogenous nucleic acid alters the genome of the plant, plant part or plant cell. Transformed plant, plant parts or plant cells can also be made of cells, entirely or partially, that have a genomic change due to the exogenous nucleic acid, but do not include the exogenous nucleic acid. Thus, transformed plants, plant parts or plant cells include transgenic plants, plant parts or plant cells; gene-edited plants, plant parts or plant cells; as well as plants, plant parts or plant cells that have taken up the exogenous nucleic acid but with an unaltered genome.

Transgene: An exogenous gene or other nucleic acid material that has been integrated into the genome of a plant, plant part or plant cell, for example by transformation or genetic engineering methods. In some examples, a transgene describes a segment of DNA containing a gene sequence or a random sequence and is integrated into the genome of a plant cell. This non-native segment of DNA may retain the ability to produce RNA or protein in the transgenic plant, or it may alter the normal function of the transgenic plant's genetic code. In some examples, a transgene is incorporated into the plant's germ line.

Transgene-free: Not containing any transgene. Many strategies have been developed to remove or prevent the integration of a transgene (such as a gene editing construct), thereby generating a transgene-free plant, plant part, plant cell, or plant seed. Such strategies include elimination of a transgene via genetic segregation; transient expression by DNA vectors; and DNA-independent editor delivery, such as co-delivery of mRNA encoding a Cas protein, and guide RNA; or delivery of preassembled Cas protein-guide RNA ribonucleoproteins (Gu, Xiaoyong et al. “Transgene-free Genome Editing in Plants.” Frontiers in genome editing vol. 3 805317. 2 Dec. 2021, doi:10.3389/fgeed.2021.805317).

Under conditions sufficient for: Referring to any combinations of factors or environmental conditions that permit a desired activity. In some examples, the desired activity is expression of a nucleic acid, leading to specifically reduced expression or activity of one or more of GROOT1, GROOT2, and GROOT3 in a plant cell, which in combination with other necessary elements, leading to increased biomass, particularly shoot biomass and/or root biomass, and/or seed size in a plant.

Vector: A nucleic acid molecule capable of carrying a nucleic acid molecule of interest and permitting its expression and/or integration in a host cell. A vector may also be capable of replicating in a host cell (e.g., along with or independent of the host genome replication during cell division), for example, by including a nucleic acid sequence (such as an origin of replication) that permits its replication. A vector may also include one or more selectable marker genes and other genetic elements known in the art. An integration vector is capable of integrating itself or the nucleic acid molecule of interest it carries into a host nucleic acid. An expression vector is a vector that contains necessary regulatory sequences to allow transcription and translation of the nucleic acid molecule of interest (e.g., one or more genes encoding a protein), without integration with a host nucleic acid. Vectors include, but are not limited to, nucleic acid molecules that are single-stranded, double-stranded, or partially double-stranded; nucleic acid molecules that comprise one or more free ends, or no free ends (e.g., circular); nucleic acid molecules that include DNA, RNA, other varieties of polynucleotides known in the art, or any combination thereof.

In some examples, a vector is not native to the cell into which it is introduced. In some examples, the vector includes coding sequences for proteins participating in gene editing (e.g., a Cas protein, such as a naturally occurring or engineered Cas9 protein), operably linked to a promoter sequence, which can be non-native (e.g., promoter that does not occur naturally in the plant into which the vector is introduced) or native (e.g., a promoter found in the plant). In some examples, a vector includes a guide nucleic acid (e.g., specific for one or more of GROOT1, GROOT2, and GROOT3) operably linked to a promoter sequence, which can be non-native or native to the plant into which the vector is introduced.

One type of vector is a “plasmid,” which refers to a circular double stranded DNA loop into which additional DNA segments can be inserted, such as by standard molecular cloning techniques.

One type of vector is a “plasmid,” which refers to a circular double stranded DNA loop into which additional DNA segments can be inserted, such as by standard molecular cloning techniques.

Another type of vector is a viral vector, wherein virally derived DNA or RNA sequences are present in the vector for packaging into a virus (e.g., retroviruses, replication defective retroviruses, adenoviruses, herpes simplex viruses, baculoviruses, replication defective adenoviruses, and adeno-associated viruses). Viral vectors also include recombinant plant viruses, such as TMV-mediated (transient) transfection into tobacco (Tuipe, T-H et al (1993), J. Virology Meth, 42: 227-239), ssDNA genomes viruses (e.g., family Geminiviridae), reverse transcribing viruses (e.g., families Caulimoviridae, Pseudoviridae, and Metaviridae), dsNRA viruses (e.g., families Reoviridae and Partitiviridae), (−) ssRNA viruses (e.g., families Rhabdoviridae and Bunyaviridae), (+) ssRNA viruses (e.g., families Bromoviridae, Closteroviridae, Comoviridae, Luteoviridae, Potyviridae, Sequiviridae and Tombusviridae) and viroids (e.g., families Pospiviroldae and Avsunviroidae). Detailed classification information of plant viruses can be found in Fauquet et al. (2008, Geminivirus strain demarcation and nomenclature. Archives of Virology 153:783-821, incorporated herein by reference in its entirety), and Khan et al. (Plant viruses as molecular pathogens; Publisher Routledge, 2002, ISBN 1560228954, 9781560228950).

Vectors also include phagemids, cosmids, artificial/mini-chromosomes (e.g., ACE), bacteriophages, pro-viruses, phagemids, transposons, artificial chromosomes, polyamine derivatives of DNA, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating.

Eukaryotic expression vectors in some examples also contain prokaryotic sequences that facilitate the propagation of the vector in bacteria such as an origin of replication and antibiotic resistance genes for selection in bacteria. A variety of eukaryotic expression vectors, containing a cloning site into which a polynucleotide can be operatively linked, are well known and some are commercially available from companies such as Stratagene, La Jolla, Calif.; Invitrogen, Carlsbad, Calif.; Promega, Madison, Wis. or BD Biosciences Clontech, Palo Alto, Calif.

### II. Overview

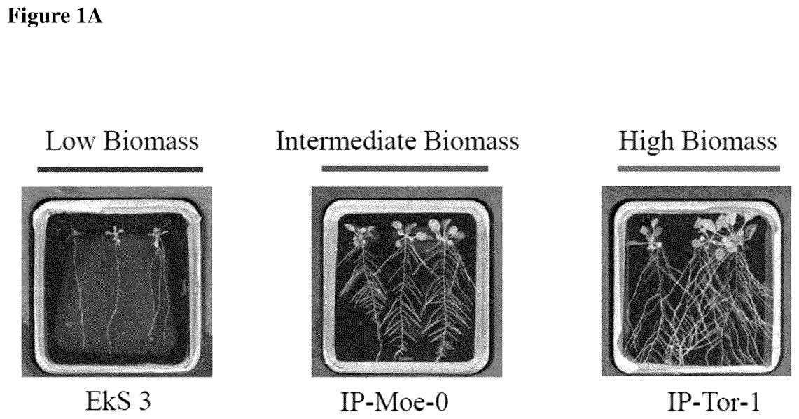

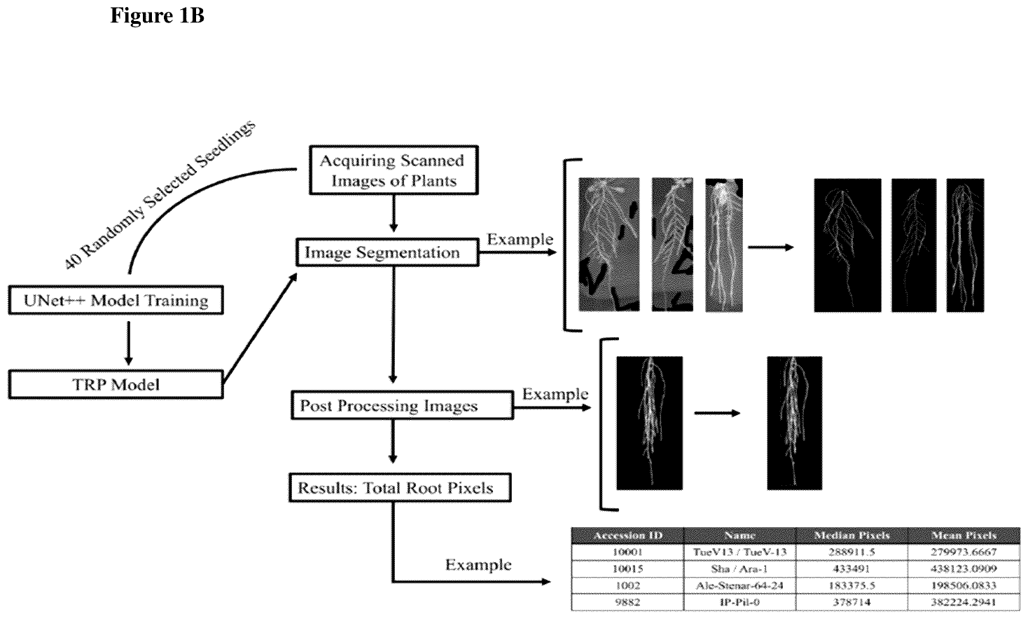

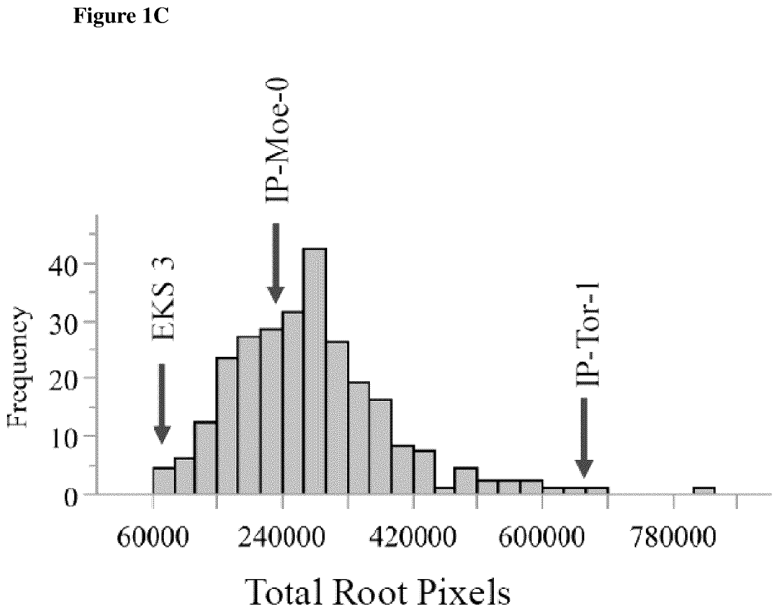

A high-throughput, deep learning-based technique called the Total Root Pixel (TRP) method, was developed to accurately estimate root biomass, particularly focusing on fine roots, which are often challenging for existing tools. This method simplifies the traditionally labor-intensive process by providing a non-destructive, image-based alternative, with a strong correlation observed between TRP estimates and actual biomass measurements (FIG. 1D). Using the UNet⁺⁺ architecture, the TRP method effectively segments and quantifies root pixels from images of seedlings grown on MS plates over 21 days. When applied to GWAS, the TRP method identified several candidate loci, underscoring the polygenic nature of complex traits such as root biomass (Weigel and Nordborg 2005; Rockman 2012; Courtois et al. 2013; Zurek et al. 2015). This tool represents a significant advancement in root phenotyping, offering a scalable solution for accelerating genetic research and breeding programs aimed at improving crop resilience.

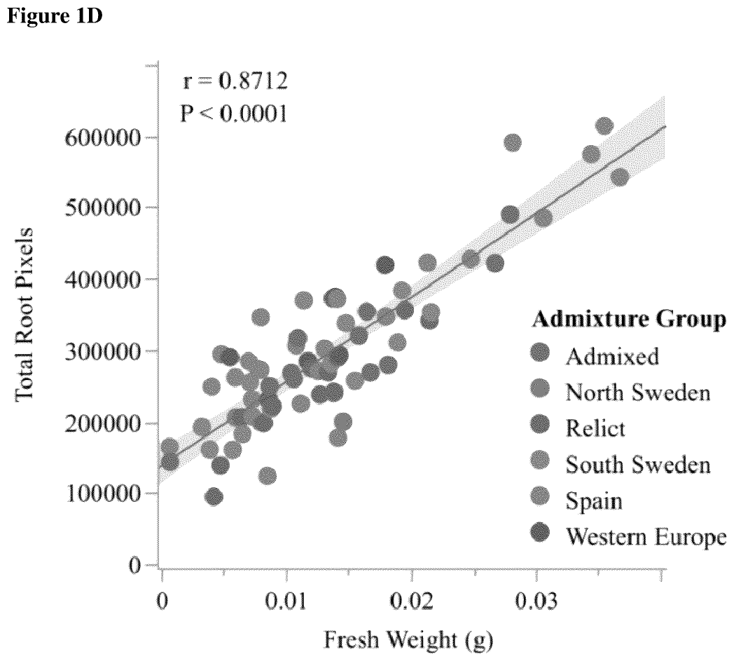

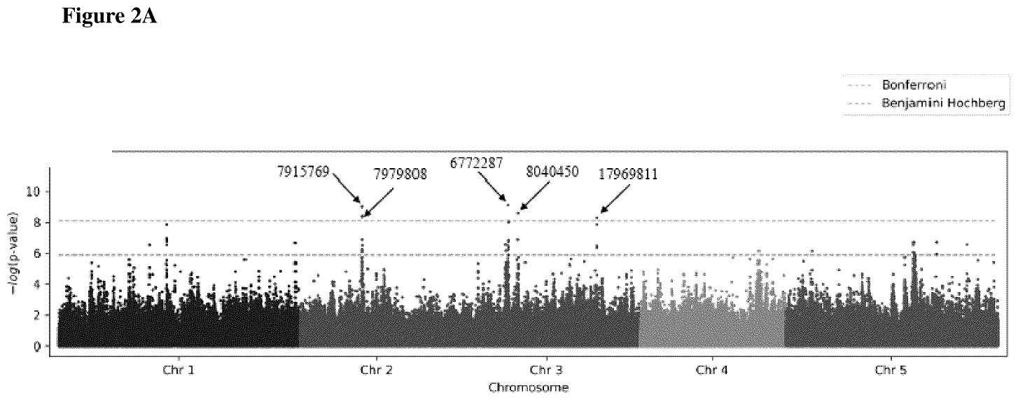

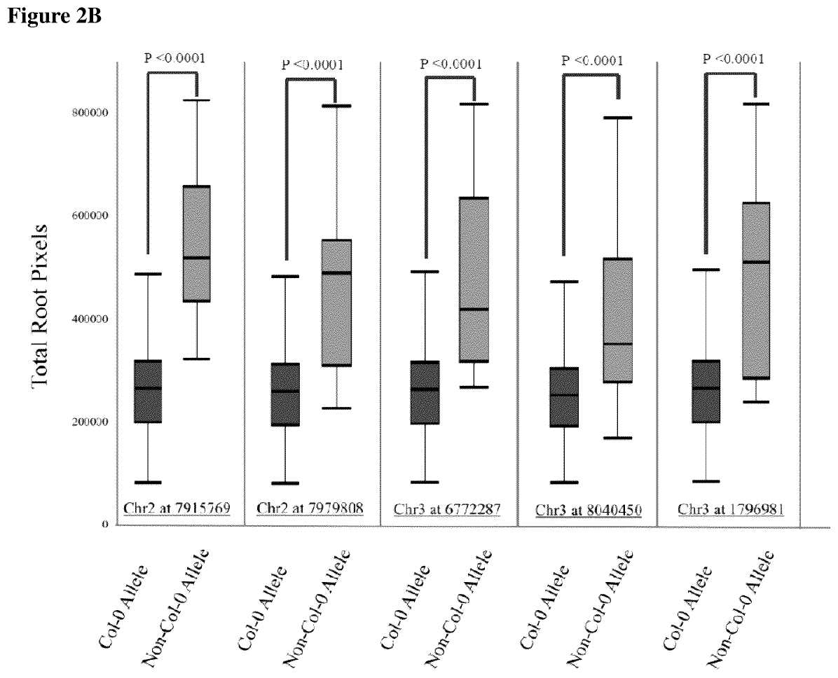

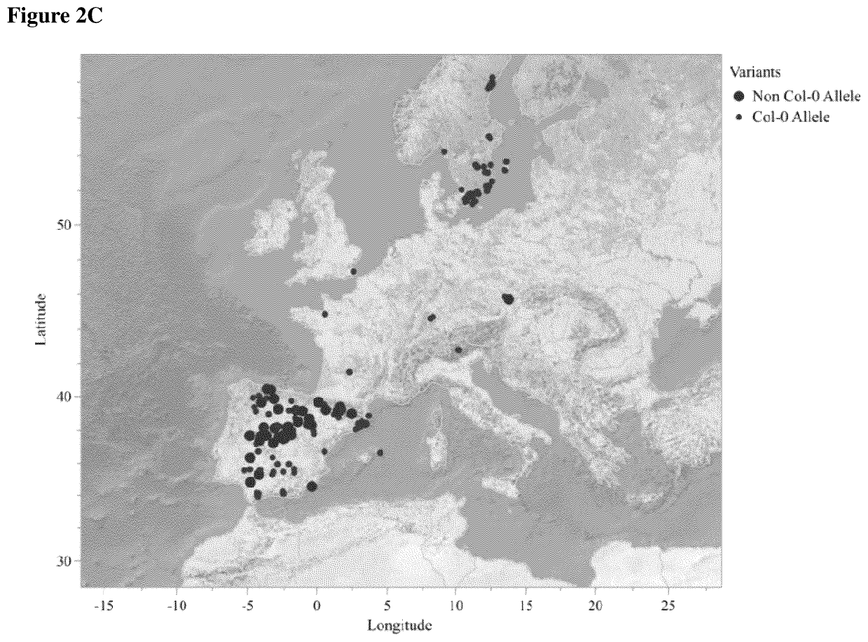

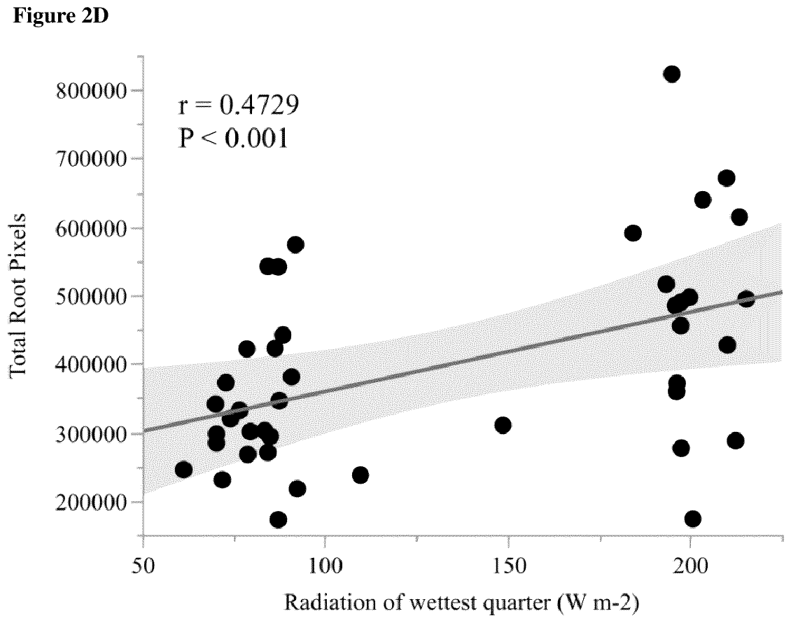

This study leveraged the TRP-based root biomass trait in a GWAS to investigate the genetic architecture of root biomass, identifying multiple loci associated with complex traits such as root biomass across various plant species. For example, in Arabidopsis thaliana, comprehensive GWAS analyses have pinpointed several loci significantly influencing several life history traits (Atwell et al. 2010; Ristova and Busch 2014). The findings herein also showed that the top five significant GWAS loci were predominantly associated with accessions from a specific geographical region. These accessions, carrying mutations at the highest GWAS peaks, consistently exhibited higher root biomass (FIGS. 2A-2C), indicating that selection pressures in this region may favor these loci, potentially as an adaptation to local environmental conditions. This study adds to the growing body of evidence emphasizing the need to consider both genetic and environmental factors when investigating complex traits.

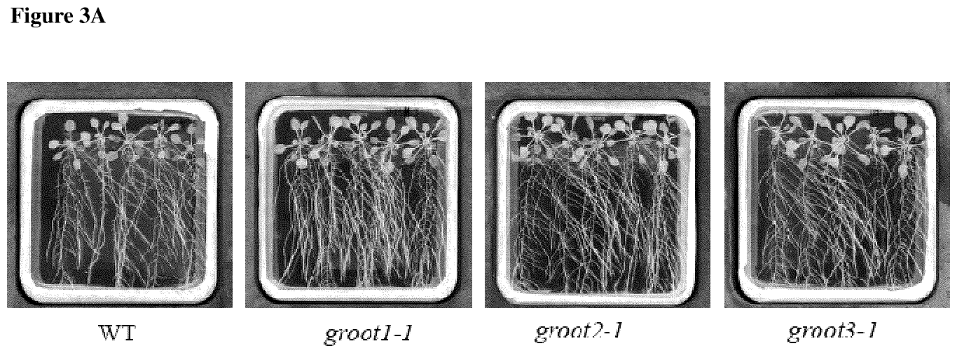

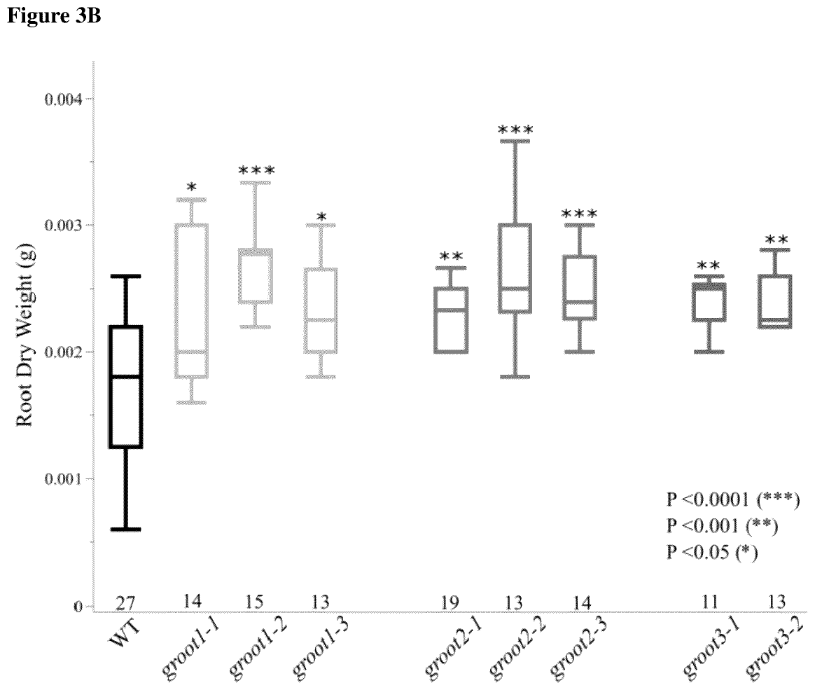

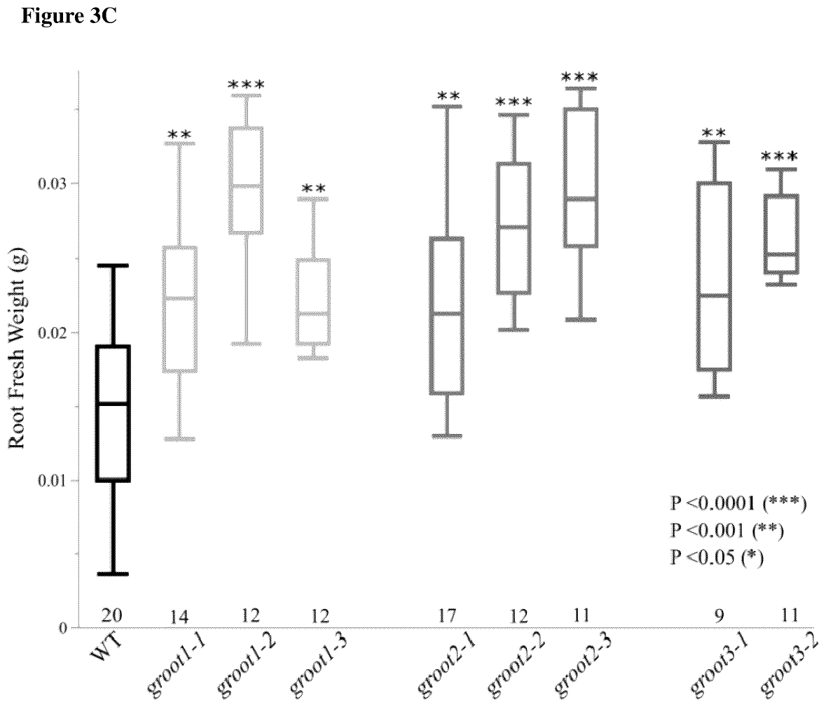

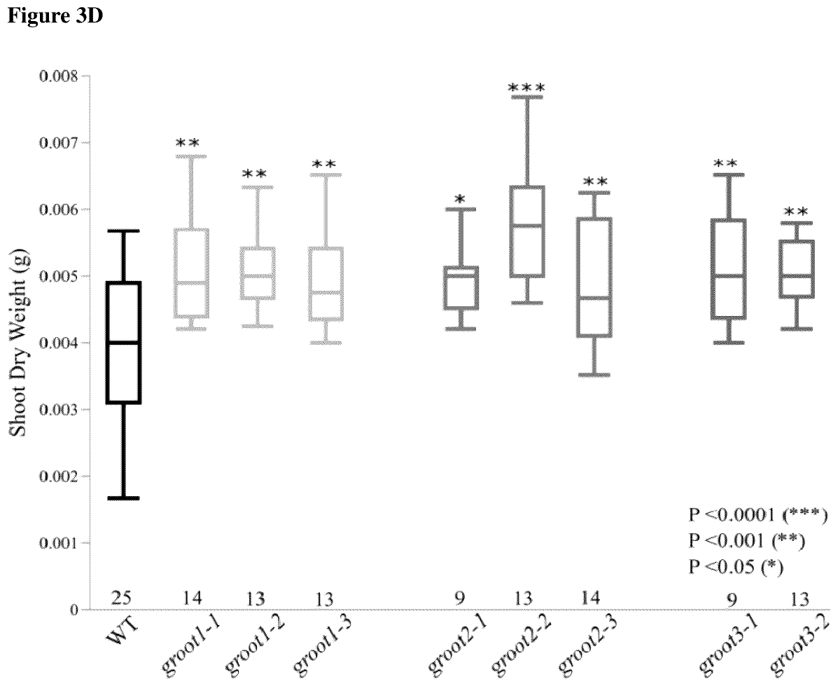

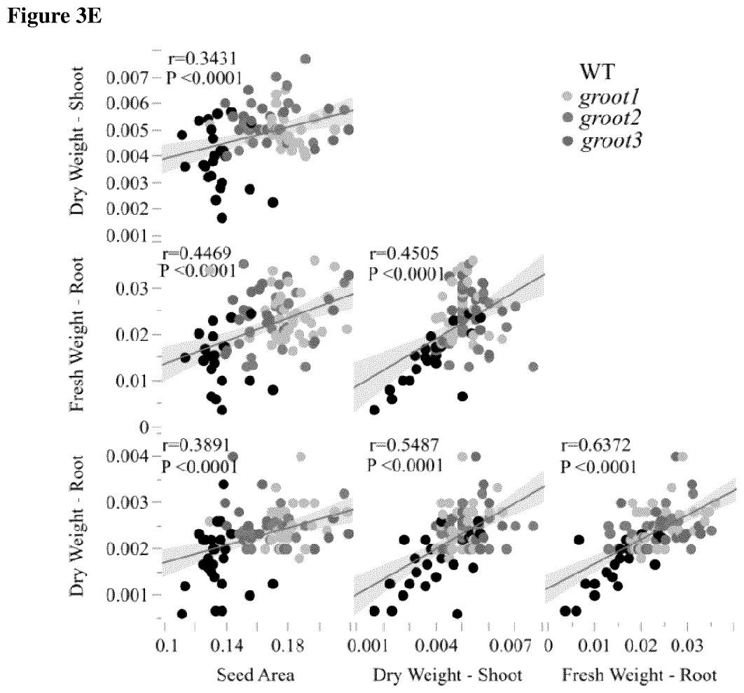

Three closely linked genes—GROOT1, GROOT2, and GROOT3—were identified and characterized, which significantly regulate plant biomass accumulation (FIGS. 3A-3E). These genes, located in close proximity on chromosome 3, exhibit strong linkage disequilibrium with SNPs identified in our GWAS, indicating a coordinated influence on root and shoot biomass traits. Disruption of these genes in multiple T-DNA mutant lines resulted in substantial increases in both root and shoot biomass, with no observable trade-offs in other critical life history traits (FIGS. 3A-3E). The absence of trade-offs, along with additive effects across multiple traits, indicates that GROOT1, GROOT2, and GROOT3 function as general growth limiters. This finding indicates that these genes are not simply reallocating resources between different growth processes but instead broadly restricting growth potential across the plant. Such a mechanism could provide a selective advantage by enabling plants to optimize resource allocation and maximize overall fitness in environments where competition for resources is intense (Grime and Pierce 2012; Smith 1978). Additionally, the close physical proximity of these genes, coupled with high linkage disequilibrium, indicates that they may operate as a coordinated cluster, contributing to a unified regulatory mechanism that limits growth across multiple traits. This clustering and strong linkage disequilibrium might reflect an evolutionary strategy, where these genes have been selected together to ensure robust control over developmental processes, allowing plants to thrive under diverse environmental conditions (Weigel and Nordborg 2005; Anderson et al. 2011). Overall, these findings provide new insights into the genetic regulation of plant growth and indicate that these genes play a critical role in optimizing resource allocation and overall plant fitness, particularly in response to varying environmental conditions.

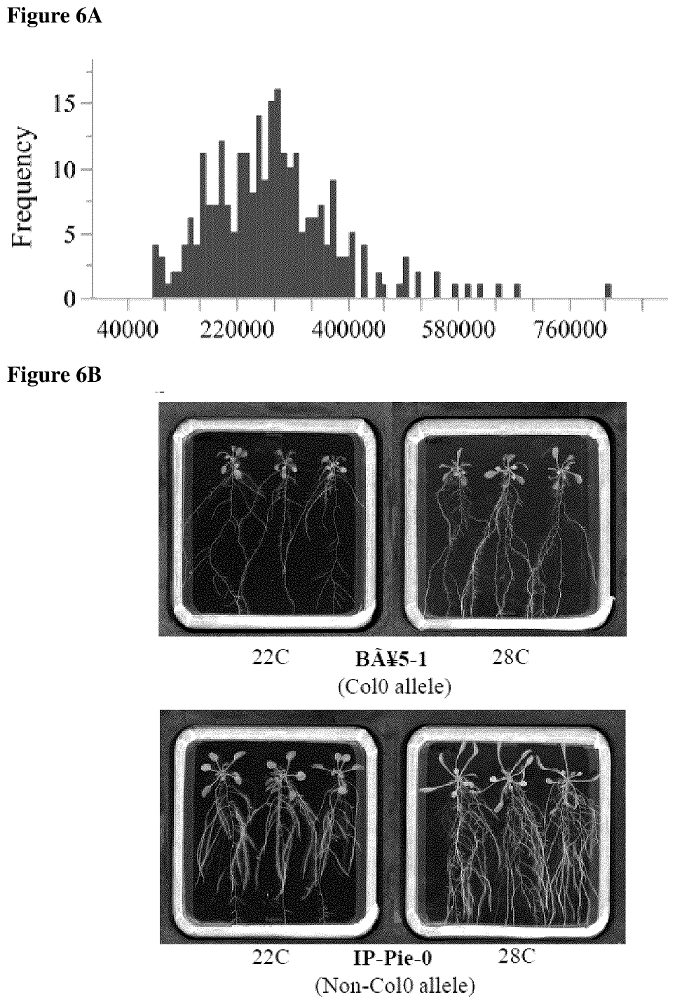

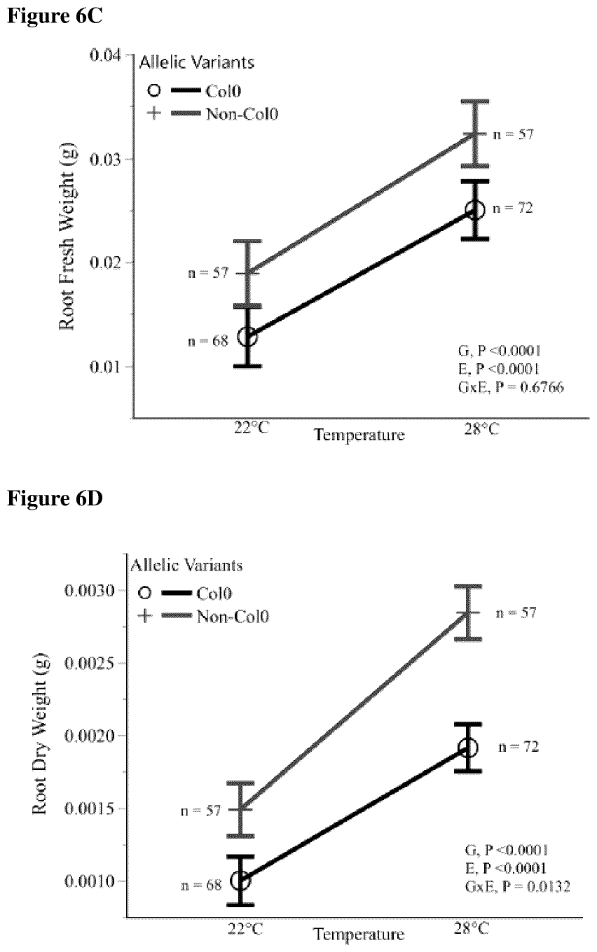

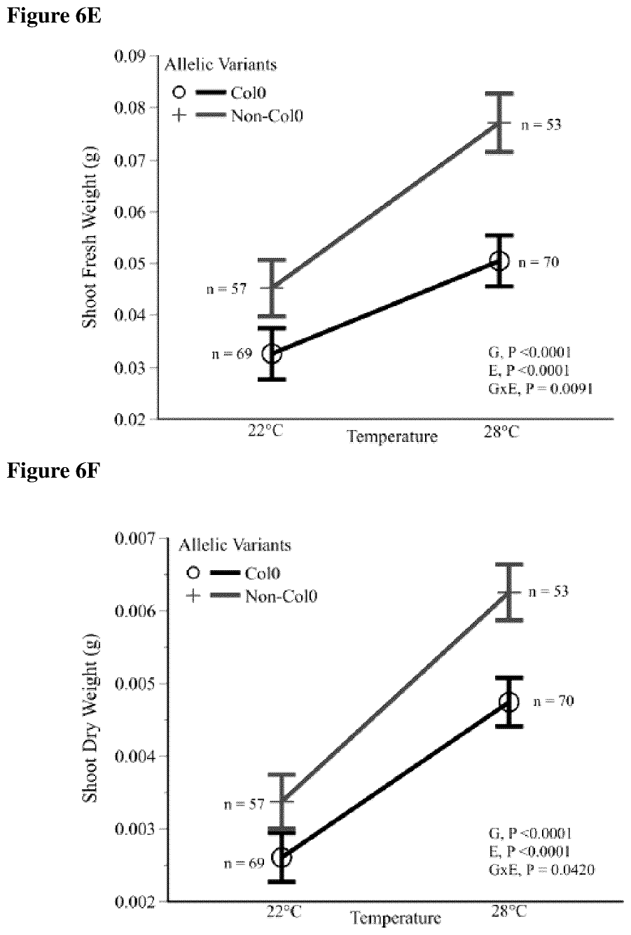

SNPs identified in the GWAS are associated with a significant response to elevated temperatures. Specifically, accessions carrying the SNP at position 6772287 on chromosome 3 showed markedly higher shoot and root biomass under elevated temperatures (28° C.), both in fresh and dry weight, compared to those grown under standard conditions (22° C.; FIGS. 6A-6F). Thus, accessions with this SNP might be regularly exposed to temperature fluctuations in their native environments, leading to the selection of this allele for improved adaptation to higher temperatures. The data herein indicates that the SNP at position 6772287 on chromosome 3 may facilitate temperature adaptation. GROOT mutant lines revealed a similar adaptive response, with increased primary root length observed under elevated temperatures on days 7 and 14. However, by day 21, regular temperature conditions resulted in greater dry mass accumulation, likely due to early flowering triggered by elevated temperatures, as described by Blázquez et al. (2003) and Balasubramanian et al. (2006). This early flowering may have limited vegetative growth, contrasting with the sustained growth seen in SNP-containing accessions. These findings demonstrate that the GROOT genes play a role in temperature adaptation, and may play a role in root development in warmer climates.

In summary, the data herein indicate that the SNP at chromosome 3 position 6772287 is an important genetic marker for temperature adaptation in plants. The differential growth responses observed between SNP-containing accessions and GROOT mutant lines under elevated temperatures further underscore the complexity of plant responses to different environments and the potential for specific genetic loci to confer adaptive advantages. In addition, the data herein highlight genetic strategies to increase plant biomass by mutating the orthologues of GROOT genes in crop species. In contrast to previous genetic interventions that required transgenes, GROOT knockout are compatible with modern gene editing strategies.

### Overview of Several Aspects

Provided herein are methods for generating a plant with increased biomass, and/or increased seed size, comprising: reducing expression and/or activity of one or more of GROOT1, GROOT2, and GROOT3 in a plant, thereby generating the plant with increased biomass, and/or increased seed size.

Also provided are methods for generating a plant with increased biomass, and/or increased seed size, comprising: reducing expression and/or activity of one or more of GROOT1, GROOT2, and GROOT3 in a plant cell or plant part, and growing the plant cell or plant part into a plant, thereby generating the plant with increased biomass, and/or increased seed size.

In some aspects, the reducing expression and/or activity comprises introducing one or more exogenous nucleic acid molecules into a plant, thereby generating a transformed plant, wherein the one or more exogenous nucleic acid molecules reduce expression of one or more of GROOT1, GROOT2, and GROOT3, and/or reduce activity of one or more proteins encoded by one or more of GROOT1, GROOT2, and GROOT3.

In any or all of the above aspects, the reducing expression and/or activity comprises introducing one or more exogenous nucleic acid molecules into a plant cell or plant part, thereby generating a gene-edited or transgenic plant cell or plant part; and the growing comprises growing the gene-edited or transgenic plant cell or plant part into a transformed plant, thereby generating the plant with increased biomass, and/or increased seed size; wherein the one or more exogenous nucleic acid molecules reduce expression of one or more of GROOT1, GROOT2, and GROOT3, and/or reduce activity of one or more proteins encoded by one or more of GROOT1, GROOT2, and GROOT3.

In any or all of the above aspects, the biomass is below-ground biomass, above-ground biomass, or entire biomass.

In any or all of the above aspects, the biomass is root biomass, shoot biomass, or root and shoot biomass.

In any or all of the above aspects, the plant with increased biomass, and/or increased seed size has increased productivity, resilience, and/or carbon sequestration capacity.

In any or all of the above aspects, the introducing one or more exogenous nucleic acid molecules generates one or more deletions of, or one or more loss-of-function mutations, in the one or more of GROOT1, GROOT2, and GROOT3.

In any or all of the above aspects, the one or more exogenous nucleic acid molecules comprise one or more guide nucleic acid molecules that can delete or mutate the one or more of GROOT1, GROOT2, and GROOT3.

In any or all of the above aspects, the method further comprises introducing one or more Cas proteins or one or more nucleic acid molecules encoding a Cas protein into the plant, plant cell, or plant part.

In any or all of the above aspects, the transformed plant, or gene-edited or transgenic plant cell or plant part comprises one or more deletions of, or one or more loss-of-function mutations, in the one or more of GROOT1, GROOT2, and GROOT3.

In any or all of the above aspects, the one or more exogenous nucleic acid molecules are one or more RNAi molecules or one or more exogenous nucleic acid molecules that generate one or more RNAi molecules, wherein the one or more RNAi molecules target the mRNAs transcribed from the one or more of GROOT1, GROOT2, and GROOT3.

In any or all of the above aspects, GROOT1 comprises at least 60%, 65%, 70%, 75%, 80%, 85%, 90%, 95%, 96%, 97%, 98%, or 99%, or 100% sequence identity to any of SEQ ID NOs:1, 3-4 and 6; or encodes a coding sequence comprising at least 60%, 65%, 70%, 75%, 80%, 85%, 90%, 95%, 96%, 97%, 98%, or 99%, or 100% sequence identity to any of SEQ ID NOs: 2, 5 and 7; or encodes a protein sequence comprising at least 60%, 65%, 70%, 75%, 80%, 85%, 90%, 95%, 96%, 97%, 98%, or 99%, or 100% sequence identity to any of SEQ ID NOs: 8-13; GROOT2 comprises at least 60%, 65%, 70%, 75%, 80%, 85%, 90%, 95%, 96%, 97%, 98%, or 99%, or 100% sequence identity to any of SEQ ID NOs: 14 and 16-17; or encodes a coding sequence comprising at least 60%, 65%, 70%, 75%, 80%, 85%, 90%, 95%, 96%, 97%, 98%, or 99%, or 100% sequence identity to any of SEQ ID NOs: 15 and 18; or encodes a protein sequence comprising at least 60%, 65%, 70%, 75%, 80%, 85%, 90%, 95%, 96%, 97%, 98%, or 99%, or 100% sequence identity to any of SEQ ID NOs: 19-22; and/or GROOT3 comprise at least 60%, 65%, 70%, 75%, 80%, 85%, 90%, 95%, 96%, 97%, 98%, or 99%, or 100% sequence identity to any of SEQ ID NOs: 23 and 25-29; or encodes a coding sequence comprising at least 60%, 65%, 70%, 75%, 80%, 85%, 90%, 95%, 96%, 97%, 98%, or 99%, or 100% sequence identity to SEQ ID NO: 24; or encodes a protein sequence comprising at least 60%, 65%, 70%, 75%, 80%, 85%, 90%, 95%, 96%, 97%, 98%, or 99%, or 100% sequence identity to any of SEQ ID NOs: 30-35.

In any or all of the above aspects, the expression and/or activity of the one or more of GROOT1, GROOT2, and GROOT3 is reduced as compared to a control plant, plant cell, or plant part.

In any or all of the above aspects, the expression and/or activity of the one or more of GROOT1, GROOT2, and GROOT3 is reduced by at least 10%, 15%, 20%, 25%, 30%, 35%, 40%, 45%, 50%, 55%, 60%, 65%, 70%, 75%, 80%, 85%, 90%, 95%, or 100% as compared to a control plant, plant cell, or plant part.

In any or all of the above aspects, the one or more exogenous nucleic acid molecules are operably linked to a heterologous promoter.

In any or all of the above aspects, the heterologous promoter drives expression of the one or more exogenous nucleic acid molecules in a plant cell.

In any or all of the above aspects, the plant is, or the plant cell or plant part is from a pennycress, soybean, canola, rice, wheat, corn, or sorghum plant.

In any or all of the above aspects, the biomass, and/or seed size is increased by at least 10%, at least 20%, at least 30%, at least 40%, at least 50%, at least 75%, at least 90%, at least 100%, at least 200%, or at least 400% as compared to a control plant.

In any or all of the above aspects, the method further includes producing a transformed plant tissue from the gene-edited or transgenic plant cell.

In any or all of the above aspects, the method further includes producing a transformed plantlet from the gene-edited or transgenic plant cell or plant part, or from the transformed plant tissue, wherein the transformed plantlet has increased biomass, and/or increased seed size when compared to a control plantlet.

In any or all of the above aspects, the method further includes producing a transformed progeny from the transformed plantlet, wherein the transformed progeny has increased biomass, and/or increased seed size when compared to a control progeny.

In any or all of the above aspects, the method further includes growing the transformed plantlet or the transformed progeny into the transformed plant.

In any or all of the above aspects, the method further includes using the transformed plant or a clone of the transformed plant in a breeding method.

In any or all of the above aspects, the breeding method includes crossing the transformed plant or the clone of the transformed plant with itself, or with a second plant.

In any or all of the above aspects, the transformed plant, gene-edited or transgenic plant cell or plant part, transformed plant tissue, transformed plantlet, or transformed progeny further comprises one or more additional exogenous nucleic acid(s) encoding a protein(s) that confers upon the transformed plant, gene-edited or transgenic plant cell or plant part, transformed plant tissue, transformed plantlet, or transformed progeny a desired trait, wherein the desired trait is one or more of herbicide tolerance, drought tolerance, heat tolerance, low or high soil pH level tolerance, salt tolerance, resistance to an insect, resistance to a bacterial disease, resistance to a viral disease, resistance to a fungal disease, resistance to a nematode, resistance to a pest, male sterility, site-specific recombination, abiotic stress tolerance, modified phosphorus characteristics, modified antioxidant characteristics, modified essential seed amino acid characteristics, decreased phytate, modified fatty acid metabolism, and modified carbohydrate metabolism.

Also provided are transformed plants, gene-edited or transgenic plant cells or plant parts, transformed plant tissues, transformed plantlets, or transformed progenies made by the methods according to any or all of the above aspects.

Also provided are methods of producing a commodity plant product, comprising collecting or producing the commodity plant product from the transformed plant, gene-edited or transgenic plant cell or plant part, transformed plant tissue, transformed plantlet, or transformed progeny of any or all of the above aspects; optionally, wherein the commodity plant product comprises a non-native nucleic acid molecule or protein from the transformed plant, gene-edited or transgenic plant cell or plant part, transformed plant tissue, transformed plantlet, or transformed progeny; and optionally, wherein the commodity product comprises a protein concentrate, protein isolate, leaves, extract, oil, bean, and/or seed.

Also provided are methods of producing plant seed, comprising crossing the transformed plant, transformed plantlet, or transformed progeny of any one of the prior claims with itself or a second plant.

In any or all of the above aspects, the plant part is a protoplast, leaf, stem, root, root tips, anther, pistil, stamen, seed, embryo, pollen, ovule, microspore, sporophyte, gametophyte, cotyledon, hypocotyl, flower, shoot, tissue, petiole, or meristematic cell.

Also provided are methods for breeding a plant with increased biomass, and/or increased seed size, comprising: crossing the transformed plant of any or all of the above aspects with a second plant; obtaining seeds from the crossing; planting the seeds and growing the seeds to progeny plants; and selecting from the progeny plants those with increased biomass, and/or increased seed size when compared to a control plant.

In any or all of the above aspects, the method further comprising producing clones of the progeny plants, wherein the clones are selected based on increased biomass, and/or increased seed size when compared to a control plant.

Also provided are seeds that produce or are produced by the transformed plant of any or all of the above aspects, wherein the seed comprises one or more deletions of, or one or more loss-of-function mutations in the one or more of GROOT1, GROOT2, and GROOT3.

Also provided are gene-edited plants, plant parts, plant cells, or seeds, comprising one or more deletions of, or one or more loss-of-function mutations in one or more of GROOT1, GROOT2, and GROOT3.

In any or all of the above aspects, the gene-edited plants, plant parts, plant cells, or seeds do not comprise a transgene used to generate the one or more deletions or loss-of-function mutations.

In any or all of the above aspects, the gene-edited plants, plant parts, plant cells, or seeds are transgene-free.

In any or all of the above aspects, the gene-edited plants, plant parts, plant cells, or seeds comprises one or more transgenes.

In any or all of the above aspects, the one or more transgenes comprise an exogenous vector, an inhibitory RNA molecule, a guide nucleic acid, a Cas gene, or combinations thereof.

In any or all of the above aspects, GROOT1, prior to the one or more deletions or loss-of-function mutations, comprises at least 60%, 65%, 70%, 75%, 80%, 85%, 90%, 95%, 96%, 97%, 98%, or 99%, or 100% sequence identity to any of SEQ ID NOs:1, 3-4 and 6; or encodes a coding sequence comprising at least 60%, 65%, 70%, 75%, 80%, 85%, 90%, 95%, 96%, 97%, 98%, or 99%, or 100% sequence identity to any of SEQ ID NOs: 2, 5 and 7; or encodes a protein sequence comprising at least 60%, 65%, 70%, 75%, 80%, 85%, 90%, 95%, 96%, 97%, 98%, or 99%, or 100% sequence identity to any of SEQ ID NOs: 8-13; GROOT2, prior to the one or more deletions or loss-of-function mutations, comprises at least 60%, 65%, 70%, 75%, 80%, 85%, 90%, 95%, 96%, 97%, 98%, or 99%, or 100% sequence identity to any of SEQ ID NOs: 14 and 16-17; or encodes a coding sequence comprising at least 60%, 65%, 70%, 75%, 80%, 85%, 90%, 95%, 96%, 97%, 98%, or 99%, or 100% sequence identity to any of SEQ ID NOs: 15 and 18; or encodes a protein sequence comprising at least 60%, 65%, 70%, 75%, 80%, 85%, 90%, 95%, 96%, 97%, 98%, or 99%, or 100% sequence identity to any of SEQ ID NOs: 19-22; and/or GROOT3, prior to the one or more deletions or loss-of-function mutations, comprise at least 60%, 65%, 70%, 75%, 80%, 85%, 90%, 95%, 96%, 97%, 98%, or 99%, or 100% sequence identity to any of SEQ ID NOs: 23 and 25-29; or encodes a coding sequence comprising at least 60%, 65%, 70%, 75%, 80%, 85%, 90%, 95%, 96%, 97%, 98%, or 99%, or 100% sequence identity to SEQ ID NO: 24; or encodes a protein sequence comprising at least 60%, 65%, 70%, 75%, 80%, 85%, 90%, 95%, 96%, 97%, 98%, or 99%, or 100% sequence identity to any of SEQ ID NOs: 30-35.

In any or all of the above aspects, the plant is, or the plant cell, plant part, or seed is from a pennycress, soybean, canola, rice, wheat, corn, or sorghum plant; or the plant cell, plant part, or seed is from a pennycress, soybean, canola, rice, wheat, corn, or sorghum plant.

In any or all of the above aspects, biomass, and/or seed size of the plant, or seed size of the seed is increased by at least 10%, at least 20%, at least 30%, at least 40%, at least 50%, at least 75%, at least 90%, at least 100%, at least 200%, or at least 400% as compared to a control plant or control seed.

In any or all of the above aspects, biomass, and/or seed size of the plant when grown at an elevated temperature is increased by at least 10%, at least 20%, at least 30%, at least 40%, at least 50%, at least 75%, at least 90%, at least 100%, at least 200%, or at least 400% as compared to a control plant grown at the same elevated temperature.

In any or all of the above aspects, the biomass is below-ground biomass, above-ground biomass, or entire biomass.

In any or all of the above aspects, the biomass is root biomass, shoot biomass, or root and shoot biomass.

Also provided are gRNAs specific for one or more of GROOT1, GROOT2, and GROOT3, wherein GROOT1 comprises at least 60%, 65%, 70%, 75%, 80%, 85%, 90%, 95%, 96%, 97%, 98%, or 99%, or 100% sequence identity to any of SEQ ID NOs:1, 3-4 and 6; or encodes a coding sequence comprising at least 60%, 65%, 70%, 75%, 80%, 85%, 90%, 95%, 96%, 97%, 98%, or 99%, or 100% sequence identity to any of SEQ ID NOs: 2, 5 and 7; or encodes a protein sequence comprising at least 60%, 65%, 70%, 75%, 80%, 85%, 90%, 95%, 96%, 97%, 98%, or 99%, or 100% sequence identity to any of SEQ ID NOs: 8-13; GROOT2 comprises at least 60%, 65%, 70%, 75%, 80%, 85%, 90%, 95%, 96%, 97%, 98%, or 99%, or 100% sequence identity to any of SEQ ID NOs: 14 and 16-17; or encodes a coding sequence comprising at least 60%, 65%, 70%, 75%, 80%, 85%, 90%, 95%, 96%, 97%, 98%, or 99%, or 100% sequence identity to any of SEQ ID NOs: 15 and 18; or encodes a protein sequence comprising at least 60%, 65%, 70%, 75%, 80%, 85%, 90%, 95%, 96%, 97%, 98%, or 99%, or 100% sequence identity to any of SEQ ID NOs: 19-22; and/or GROOT3 comprise at least 60%, 65%, 70%, 75%, 80%, 85%, 90%, 95%, 96%, 97%, 98%, or 99%, or 100% sequence identity to any of SEQ ID NOs: 23 and 25-29; or encodes a coding sequence comprising at least 60%, 65%, 70%, 75%, 80%, 85%, 90%, 95%, 96%, 97%, 98%, or 99%, or 100% sequence identity to SEQ ID NO: 24; or encodes a protein sequence comprising at least 60%, 65%, 70%, 75%, 80%, 85%, 90%, 95%, 96%, 97%, 98%, or 99%, or 100% sequence identity to any of SEQ ID NOs: 30-35.

Also provided are gRNAs or sgRNAs comprising any of SEQ ID NOs: 39-51.

Also provided are ribonucleoprotein complexes, which include an isolated Cas9 protein; and a gRNA or sgRNA according to any or all of the above aspects.

### III. Nucleic Acids and Proteins of GROOT1, GROOT2, and GROOT3

In accordance with the present disclosure, decreasing expression of one or more of GROOT1, GROOT2, and GROOT3 genes or activity of proteins encoded by these genes, can be used to generate plants that have increased biomass, particularly root biomass and/or shoot biomass, and/or increased seed size, when comparted to appropriate control plants. In some examples, expression of at least 2, at least 3, at least 4, at least 5, at least 6, or at least 7 different GROOT1, GROOT2, and/or GROOT3 genes (such as 1, 2, 3, 4, 5, 6, 7, 8, 9, or 10 different GROOT1, GROOT2, and/or GROOT3 genes) is decreased in a plant, thereby generating plants that have increased biomass, particularly root biomass and/or shoot biomass, and/or increased seed size, when comparted to appropriate control plants. In some examples, activity of at least 2, at least 3, at least 4, at least 5, at least 6, or at least 7 different GROOT1, GROOT2, and/or GROOT3 proteins (such as 1, 2, 3, 4, 5, 6, 7, 8, 9, or 10 different GROOT1, GROOT2, and/or GROOT3 proteins) is decreased in a plant, thereby generating plants that have increased biomass, particularly root biomass and/or shoot biomass, and/or increased seed size, when comparted to appropriate control plants.

In some examples, expression of GROOT1 and GROOT2; GROOT1 and GROOT3; GROOT2 and GROOT3; or GROOT1, GROOT2, and GROOT3 is reduced. In some examples, activities of GROOT1 and GROOT2 proteins; GROOT1 and GROOT3 proteins; GROOT2 and GROOT3 proteins; or GROOT1, GROOT2, and GROOT3 proteins are reduced.

The present disclosure provides exemplary nucleic acid and protein sequences of GROOT1, GROOT2, and GROOT3 (including the ones found in Arabidopsis thaliana and any ortholog thereof). Thus, the provided sequences can be used in breeding programs, for example by designing appropriate inhibitory RNA molecules, guide nucleic acid molecules, or other nucleic acid molecules that mutate one or more of GROOT1, GROOT2, and GROOT3 genes. See, for example, Gentzbittel et al. (1998, Theor. Appl. Genet. 96:519-523). The provided sequences can thus be used to modulate plant biomass, particularly root biomass and/or shoot biomass, and/or increased seed size. See, generally, Sambrook et al. (1989) Molecular Cloning: A Laboratory Manual (2nd ed., Cold Spring Harbor Laboratory Press, Plainview, N.Y.).

The disclosure also encompasses isolated or substantially purified nucleic acid or protein. “Isolated” or “substantially purified” means substantially or essentially free from components that normally accompany or interact with the nucleic acid molecule or protein as found in its naturally occurring environment. Thus, an isolated or purified polynucleotide or polypeptide is substantially free of other cellular material, or culture medium when produced by recombinant techniques, or substantially free of chemical precursors or other chemicals when chemically synthesized. In one example, an isolated polynucleotide is free of sequences (especially protein encoding sequences) that naturally flank the polynucleotide (i.e., sequences located at the 5′ and 3′ ends of the polynucleotide) in the genomic DNA of the plant from which the polynucleotide was derived. In some embodiments, the isolated polynucleotide can contain less than about 5 kb 4 kb, 3 kb, 2 kb, 1 kb, 0.5 kb, or 0.1 kb of nucleotide sequences that naturally flank the polynucleotide in genomic DNA of the cell from which the polynucleotide was derived. A polypeptide that is substantially free of cellular material includes preparations of protein having less than about 30%, 20%, 10%, 5%, (by dry weight) of contaminating protein. When a protein (including its functional fragments) of the disclosure is recombinantly produced, in some examples the culture medium suitably represents less than about 30%, 20%, 10%, or 5% (by dry weight) of chemical precursors or non-protein-of-interest chemicals.

Exemplary GROOT1, GROOT2, and GROOT3 genomic sequences are provided in SEQ ID NOs: 1, 3-4, 6, 14, 16-17, 23 and 25-29. Exemplary GROOT1, GROOT2, and GROOT3 coding sequences are provided in SEQ ID NOs: 2, 5, 7, 15, 18, and 24. Exemplary GROOT1, GROOT2, and GROOT3 protein sequences are provided in SEQ ID NOs: 8-13, 19-22 and 30-35. Guide nucleic acids (or vectors providing for such), RNAi molecules or antisense RNAs (or vectors providing for such), hybridization probes, PCR primers, etc. can be generated based on these sequences or any fragment thereof, or sequences upstream or downstream of these sequences (such as regulatory sequences). These sequences can also be used to study protein-protein interactions and protein-DNA interactions, thereby identifying GROOT1, GROOT2, and/or GROOT3 regulators.

The disclosure also contemplates using variants of the disclosed nucleotide sequences. Nucleic acid variants can be naturally occurring, such as allelic variants (same locus), paralogous (different locus), and orthologues (different organism) or can be non-naturally occurring. Naturally occurring variants can be identified with the use of well-known molecular biology techniques, as, for example, with polymerase chain reaction (PCR) and hybridization techniques as known in the art. Non-naturally occurring variants can be made by mutagenesis techniques, including those applied to polynucleotides, cells, or organisms. The variants can contain nucleotide substitutions, deletions, inversions and insertions. Variation can occur in either or both the coding and non-coding regions. The variations can produce both conservative and non-conservative amino acid substitutions (as compared to the encoded product). For nucleotide sequences, conservative variants include those sequences that, because of the degeneracy of the genetic code, encode the amino acid sequence of a GROOT1, GROOT2, or GROOT3 protein of the disclosure. Variant nucleotide sequences also include synthetically derived nucleotide sequences, such as those generated, for example, by using site-directed mutagenesis but which still encode a GROOT1, GROOT2, and GROOT3 protein of the disclosure. Generally, variants of a particular nucleotide sequence of the disclosure have at least about 30%, at least about 40%, at least about 50%, at least about 55%, at least about 60%, at least about 65%, at least about 70%, at least about 75%, at least about 80%, at least about 85%, at least about 90%, at least about 95%, at least about 98%, at least about 99%, or 100% sequence identity to that particular nucleotide sequence as determined by sequence alignment programs described elsewhere herein using default parameters.

Variant nucleotide sequences also encompass sequences derived from mutagenic or recombinant procedures such as “DNA shuffling” which can be used for swapping domains in a polypeptide of interest with domains of other polypeptides. With DNA shuffling, one or more of different GROOT1, GROOT2, or GROOT3 coding sequences can be manipulated to create a new GROOT1, GROOT2, or GROOT3 sequence possessing desired properties. In this procedure, libraries of recombinant polynucleotides are generated from a population of related polynucleotides comprising sequence regions that have substantial sequence identity and can be homologously recombined in vitro or in vivo. For example, using this approach, sequence motifs encoding a domain of interest may be shuffled between different GROOT1, GROOT2, or GROOT3 genes or coding sequences, to obtain a new gene coding for a protein with an altered or reduced property or function, thereby increasing biomass and/or seed size. Strategies for DNA shuffling are known from e.g., Stemmer (1994, Proc. Natl. Acad. Sci. USA 91:10747-10751; 1994, Nature 370:389-391); Crameri et al. (1997, Nature Biotech. 15:436-438); Moore et al. (1997, J. Mol. Biol. 272:336-347); Zlang et al. (1997 Proc. Natl. Acad. Sci. USA 94:450-44509); Crameri et al. (1998, Nature 391:288-291); and U.S. Pat. Nos. 5,605,793 and 5,837,458.

The present disclosure provides nucleotide sequences for GROOT1, GROOT2, and GROOT3 genes or protein coding sequences, and fragments and variants thereof.

In some aspects, a GROOT1 gene or a variant or functional fragment thereof includes (e.g., prior to its mutation, functional deletion, or inactivation) a nucleotide sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 1, 3-4 and 6.

In some aspects, a GROOT1 gene (e.g., after introns are removed) or GROOT1 coding sequence, or a variant or functional fragment thereof includes (e.g., prior to its mutation, functional deletion, or inactivation) a nucleotide sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 2, 5 and 7.

In some aspects, a GROOT1 gene or GROOT1 coding sequence, or a variant or functional fragment thereof includes (e.g., prior to its mutation, functional deletion, or inactivation) a nucleotide sequence that encodes a protein that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 8-13.

In some aspects, a GROOT2 gene or a variant or functional fragment thereof includes (e.g., prior to its mutation, functional deletion, or inactivation) a nucleotide sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 14 and 16-17.

In some aspects, a GROOT2 gene (e.g., after introns are removed) or GROOT2 coding sequence, or a variant or functional fragment thereof includes (e.g., prior to its mutation, functional deletion, or inactivation) a nucleotide sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 15 and 18.

In some aspects, a GROOT2 gene or GROOT2 coding sequence, or a variant or functional fragment thereof includes (e.g., prior to its mutation, functional deletion, or inactivation) a nucleotide sequence that encodes a protein that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 19-22.

In some aspects, a GROOT3 gene or a variant or functional fragment thereof includes (e.g., prior to its mutation, functional deletion, or inactivation) a nucleotide sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 23 and 25-29.

In some aspects, a GROOT3 gene (e.g., after introns are removed) or GROOT3 coding sequence, or a variant or functional fragment thereof includes (e.g., prior to its mutation, functional deletion, or inactivation) a nucleotide sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to SEQ ID NO: 24.

In some aspects, a GROOT3 gene or GROOT3 coding sequence, or a variant or functional fragment thereof includes (e.g., prior to its mutation, functional deletion, or inactivation) a nucleotide sequence that encodes a protein that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 30-35.

In some aspects, nucleotide sequences for one or more of GROOT1, GROOT2, and GROOT3 genes (such as endogenous GROOT1, GROOT2, and/or GROOT3 genomic sequences) are mutated or deleted to decrease gene expression and/or activity of proteins, thereby increasing plant biomass, particularly shoot biomass and/or root biomass, and/or increasing seed size.

The present disclosure provides nucleotide sequences for GROOT1, GROOT2, and GROOT3 transcripts or mRNAs. These sequences are readily derivable from the GROOT1, GROOT2, and GROOT3 protein coding sequences provided herein.

In some aspects, one or more of GROOT1, GROOT2, and GROOT3 transcripts or mRNAs are targeted for degradation or prevented from translation, thereby plant biomass, particularly shoot biomass and/or root biomass, and/or increasing seed size.

The present disclosure provides amino acid sequences for GROOT1, GROOT2, and GROOT3 proteins, and fragments and variants thereof.

In some aspects, the present disclosure provides protein sequences encoded by the nucleotide sequences of GROOT1, GROOT2, and GROOT3 and functional fragments and variations thereof.

In some aspects, a GROOT1 protein or a variant or functional fragment thereof includes an amino acid sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 8-13 (for example prior to its mutation, functional deletion, or inactivation).

In some aspects, a GROOT2 protein or a variant or functional fragment thereof includes an amino acid sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 19-22 (for example prior to its mutation, functional deletion, or inactivation).

In some aspects, a GROOT3 protein or a variant or functional fragment thereof includes an amino acid sequence that shares at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to any of SEQ ID NOs: 30-35 (for example prior to its mutation, functional deletion, or inactivation).

Functional fragments and variants of a GROOT1, GROOT2, or GROOT3 protein include those fragments and variants that maintain one or more functions of the reference GROOT1, GROOT2, or GROOT3 protein. It is recognized that the gene or coding sequence encoding a protein can be mutated without materially altering one or more of the protein's functions. First, the genetic code is degenerate, and thus different codons encode the same amino acids. Second, even where an amino acid substitution is introduced, the mutation can be conservative and have no material impact on the essential function(s) of a protein. See, e.g., Stryer Biochemistry 3rd Ed., 1988. Third, part of a protein chain can be deleted without impairing or eliminating all of its functions. Fourth, insertions or additions can be made in the protein chain for example, adding epitope tags, without impairing or eliminating its functions (Ausubel et al. J. Immunol. 159(5): 2502-12, 1997). Other modifications that can be made without materially impairing one or more functions of a protein can include, for example, in vivo or in vitro chemical and biochemical modifications or the incorporation of unusual amino acids. Such modifications include, but are not limited to, for example, acetylation, carboxylation, phosphorylation, glycosylation, ubiquination, labelling, e.g., with radionucleotides, and various enzymatic modifications, as will be readily appreciated by those well skilled in the art. A variety of methods for labelling polypeptides, and labels useful for such purposes, are well known in the art, and include radioactive isotopes such as 32P, ligands which bind to or are bound by labelled specific binding partners (e.g., antibodies), fluorophores, chemiluminescent agents, enzymes, and anti-ligands. Functional fragments and variants can be of varying length. For example, some fragments have at least 10, 25, 50, 75, 100, 200, or even more amino acid residues.

Variants of GROOT1, GROOT2, or GROOT3 proteins can have “conservative” changes, or “nonconservative” changes as described above, such as an addition or deletion that does not alter a protein function significantly. Conservative amino acid substitutions are those substitutions that, when made, least interfere with the properties of the original protein, that is, the structure and especially the function of the protein is conserved and not significantly changed by such substitutions. Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain. Further information about conservative substitutions can be found, for instance, in Ben Bassat et al. (J. Bacteriol., 169:751 757, 1987), O'Regan et al. (Gene, 77:237 251, 1989), Sahin Toth et al. (Protein Sci., 3:240 247, 1994), Hochuli et al. (Bio/Technology, 6:1321 1325, 1988) and in widely used textbooks of genetics and molecular biology. The Blosum matrices are commonly used for determining the relatedness of polypeptide sequences. The Blosum matrices were created using a large database of trusted alignments (the BLOCKS database), in which pairwise sequence alignments related by less than some threshold percentage identity were counted (Henikoff et al., Proc. Natl. Acad. Sci. USA, 89:10915-10919, 1992). A threshold of 90% identity was used for the highly conserved target frequencies of the BLOSUM90 matrix. A threshold of 65% identity was used for the BLOSUM65 matrix. Scores of zero and above in the Blosum matrices are considered “conservative substitutions” at the percentage identity selected. Table 1 shows exemplary conservative amino acid substitutions.

TABLE 1
Exemplary conservative amino acid substitutions

| | | Highly Conserved | Conserved |
|---|---|---|---|
| | Very Highly - | Substitutions (from | Substitutions (from |
| Original | Conserved | the Blosum90 | the Blosum65 |
| Residue | Substitutions | Matrix) | Matrix) |
| Ala | Ser | Gly, Ser, Thr | Cys, Gly, Ser, Thr, |
| | | | Val |
| Arg | Lys | Gln, His, Lys | Asn, Gln, Glu, His, |
| | | | Lys |
| Asn | Gln; His | Asp, Gln, His, Lys, | Arg, Asp, Gln, Glu, |
| | | Ser, Thr | His, Lys, Ser, Thr |
| Asp | Glu | Asn, Glu | Asn, Gln, Glu, Ser |
| Cys | Ser | None | Ala |
| Gln | Asn | Arg, Asn, Glu, His, | Arg, Asn, Asp, Glu, |
| | | Lys, Met | His, Lys, Met, Ser |
| Glu | Asp | Asp, Gln, Lys | Arg, Asn, Asp, Gln, |
| | | | His, Lys, Ser |
| Gly | Pro | Ala | Ala, Ser |
| His | Asn; Gln | Arg, Asn, Gln, Tyr | Arg, Asn, Gln, Glu, |
| | | | Tyr |
| Ile | Leu; Val | Leu, Met, Val | Leu, Met, Phe, Val |
| Leu | Ile; Val | Ile, Met, Phe, Val | Ile, Met, Phe, Val |
| Lys | Arg; Gln; Glu | Arg, Asn, Gln, Glu | Arg, Asn, Gln, Glu, |
| | | | Ser, |
| Met | Leu; Ile | Gln, Ile, Leu, Val | Gln, Ile, Leu, Phe, |
| | | | Val |
| Phe | Met; Leu; Tyr | Leu, Trp, Tyr | Ile, Leu, Met, Trp, |
| | | | Tyr |
| Ser | Thr | Ala, Asn, Thr | Ala, Asn, Asp, Gln, |
| | | | Glu, Gly, Lys, Thr |
| Thr | Ser | Ala, Asn, Ser | Ala, Asn, Ser, Val |
| Trp | Tyr | Phe, Tyr | Phe, Tyr |
| Tyr | Trp; Phe | His, Phe, Trp | His, Phe, Trp |
| Val | Ile; Leu | Ile, Leu, Met | Ala, Ile, Leu, Met, |
| | | | Thr |

In some examples, variants can have no more than 3, 5, 10, 15, 20, 25, 30, 40, 50, or 100 conservative amino acid changes (such as very highly conserved or highly conserved amino acid substitutions). In other examples, one or several hydrophobic residues (such as Leu, Ile, Val, Met, Phe, or Trp) in a variant sequence can be replaced with a different hydrophobic residue (such as Leu, Ile, Val, Met, Phe, or Trp) to create a variant functionally similar to the disclosed amino acid sequences.

In some aspects, variants may differ from the disclosed sequences by alteration of the coding region to fit the codon usage bias of the particular organism into which the molecule is to be introduced. In other embodiments, the coding region may be altered by taking advantage of the degeneracy of the genetic code to ater the coding sequence such that, while the nucleotide sequence is substantially altered, it nevertheless encodes a protein having an amino acid sequence substantially similar to the disclosed amino acid sequences.

In some aspects, functional fragments derived from GROOT1, GROOT2, or GROOT3 proteins of the present disclosure are provided. Reducing expression or activity of a functional fragment in a plant can still confer the ability to increase biomass and/or seed size. In some examples, the functional fragments contain one or more conserved region shared by two or more GROOT1 genes, two or more GROOT2 genes, or two or more GROOT3 genes (including homologs, including paralogs and orthologs), for example, shared by two or more orthologs in the same plant genus, shared by two or more dicot GROOT1, GROOT2, or GROOT3 orthologs, or shared by two or more monocot from GROOT1, GROOT2, or GROOT3 orthologs. The conserved regions can be determined by any suitable computer program, such as NCBI protein BLAST program and NCBI Alignment program, or equivalent programs. In some examples, the functional fragments are 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acids shorter compared to a full-length from GROOT1, GROOT2, or GROOT3 protein of the present disclosure. In some examples, the functional fragments are made by deleting one or more amino acids of a full-length GROOT1, GROOT2, or GROOT3 protein of the present disclosure. In some examples, the functional fragments share at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to a full-length GROOT1, GROOT2, or GROOT3 protein of the present disclosure.

The present disclosure also provides conserved regions of GROOT1, GROOT2, or GROOT3 proteins or genes. The conserved regions can be determined by any suitable computer program, such as NCBI protein BLAST program and NCBI Alignment program, or equivalent programs. Sequences of conserved regions can be used to knock down or knock out the level of one or more of GROOT1, GROOT2, and GROOT3 genes (including homologs, including paralogs and orthologs). In some examples, sequences of conserved regions can be used to make gene silencing molecules to target one or more of GROOT1, GROOT2, and GROOT3 genes or gene products (e.g., mRNA). In some aspects, the gene silencing molecules are double-stranded polynucleotides, single-stranded polynucleotides or mixed duplex oligonucleotides. In some aspects, the gene silencing molecules include a DNA or RNA fragment of about 10 bp, 15 bp, 19 bp, 20 bp, 21 bp, 25 bp, 30 bp, 40 bp, 50 bp, 60 bp, 70 bp, 80 bp, 90 bp, 100 bp, 150 bp, 200 bp, 250 bp, 300 bp, 350 bp, 400 bp, 500 bp, 600 bp, 700 bp, 800 bp, 900 bp, 1000 bp, or more polynucleotides, wherein the DNA or RNA fragment share at least 90%, 95%, 99%, or more identity to a conserved region of GROOT1, GROOT2, or GROOT3 sequences of the present disclosure, or complementary sequences thereof.

The present disclosure also provides loss-of-function GROOT1, GROOT2, or GROOT3 gene variants. Such variants can occur in nature or result from human intervention (such as mutagenesis, for example the gene-editing methods provided herein or known in the art). In some examples, such variants are generated by CRISPR/Cas technologies to edit genomic DNA or RNA.

In some aspects, loss-of-function GROOT1, GROOT2, or GROOT3 gene variants have a transcription or expression level reduced by at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, compared to that of a corresponding functional or wild-type GROOT1, GROOT2, or GROOT3 gene. In some aspects, loss-of-function GROOT1, GROOT2, or GROOT3 gene variants encode a GROOT1, GROOT2, or GROOT3 protein that has an activity reduced by at least about 20%, at least about 30%, at least about 40%, at least about 50%, at least about 60%, at least about 70%, at least about 80%, at least about 90%, at least about 95%, at least about 96%, at least about 97%, at least about 98%, or at least about 99%, compared to that of a corresponding functional or wild-type GROOT1, GROOT2, or GROOT3 protein.

In some examples, loss-of-function GROOT1, GROOT2, or GROOT3 gene variants have an insertion of one or more nucleotides, such as an insertion of at least 1, at least 2, at least 3, at least 4, at least 5, at least 10, at least 25, at least 50, or at least 100 nucleotides, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 75, or 100 added nucleotides as compared to a corresponding functional or wild-type GROOT1, GROOT2, or GROOT3 gene. In some examples, loss-of-function GROOT1, GROOT2, or GROOT3 gene variants have a deletion of 5-40 nucleotides, such as 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides, as compared to a corresponding functional or wild-type GROOT1, GROOT2, or GROOT3 gene. In some examples, loss-of-function GROOT1, GROOT2, or GROOT3 gene variants have a combination of one or more nucleotide insertions, one or more nucleotide deletions, one or more nucleotide substitutions, or combinations thereof.

### IV. Constructs

The present disclosure provides constructs, that include expression cassettes, that include a promoter, an optional terminal signal, and a sequence for expression operably linked to the promoter, and optionally to the terminal signal. The expression cassettes may also include sequences required for proper translation of the sequence for expression.

In some aspects, the sequence for expression encodes one or more components of a gene editing complex, such as one or more guide RNAs targeting one or more of GROOT1, GROOT2, and GROOT3 genes; and/or an endonuclease such as a Cas protein, such as a Cas9 protein, naturally occurring or not. In some aspects, the sequence for expression encodes one or more RNAi molecule or antisense RNA, targeting one or more mRNAs transcribed from one or more of GROOT1, GROOT2, and GROOT3 genes.

When the expression cassettes are transformed into plants, they enable the plants to increase biomass, particularly shoot biomass and/or root biomass, and/or seed size, without negatively impacting plant health.

In some aspects, the expression cassette is chimeric so that at least one of its components is heterologous with respect to at least one of its other components.

In some aspects, the expression cassette is naturally occurring but has been obtained in a recombinant form useful for heterologous expression. The expression of the nucleotide sequence in the expression cassette can be under the control of a constitutive promoter, or an inducible promoter which initiates transcription only when the host cell is exposed to specific external stimuli. Also, the expression of the nucleotide sequence in the expression cassette can be under the control of a tissue-specific promoter. In addition, the promoter can also be specific to a particular stage of development in a plant.

### V. Increasing Biomass and Seed Size in Plants with Gene Editing

GROOT1, GROOT2, and/or GROOT3 expression or activity can be decreased or even eliminated, for example by methods of gene editing, mutagenesis, RNAi, or antisense RNA.

In some aspects, a gene editing system is used that includes one or more nucleic acid (e.g., DNA or RNA)-binding components, and one or more nucleic acid (e.g., DNA or RNA)-modifying components; or isolated nucleic acids, e.g., one or more vectors, that encode the one or more nucleic acid-binding components, and the one or more nucleic acid-modifying components. Gene editing systems can be used for modifying a coding sequence of a target gene and/or for modulating the expression of a target gene, e.g., by modifying a non-coding/regulatory sequence (e.g., promoter, operator, etc.) of the gene, or by modifying the coding sequence/expression of a regulator (e.g., repressor or activator) of the gene. In some examples, the nucleic acid binding components are associated with the nucleic acid-modifying components, such that the nucleic acid-binding components target the nucleic acid-modifying components to a specific site of a genome. Methods and compositions for enhancing gene editing is known. See, for example, U.S. Patent Application Publication No. 2018/0245065. The nucleic acid-binding domains can be protein domains or nucleic acids that are engineered to recognize target sequences.

Exemplary gene editing systems include but are not limited to, zinc finger nucleases (ZFNs), transcription activator-like effector nucleases (TALENs), CRISPR/Cas systems, meganuclease systems, Fok1 restriction endonuclease systems, and viral vector-mediated gene editing. In some aspects, CRISPR/Cas-based gene editing methods are used to genetically modify the genome of a plant, in order to increase biomass, particularly shoot biomass and/or root biomass, and/or seed size.

### A. CRISPR/Cas Systems

CRISPR and Cas were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers) (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. Al., Nat. Struct. Mol. Biol. 2001, 18:688; Jinek, M., et. Al., Science. 2012:337; 816-21). Target recognition by crRNAs occurs through complementary base pairing with target DNA, which directs cleavage of foreign sequences by means of Cas proteins (Jinek et. Al. 2012 “A Programmable dual-RNA-guided DNA endonuclease in adaptive bacterial immunity.” Science. 2012:337; 816-821).

There are at least five main CRISPR system types (Type I, II, III, IV and V) and at least 16 distinct subtypes (Makarova, K. S., et al., Nat Rev Microbiol. 2015. Nat. Rev. Microbiol. 13, 722-736). CRISPR systems are also classified based on their effector proteins. Class 1 systems possess multi-subunit crRNA-effector complexes, whereas in Class 2 systems, all functions of the effector complex are carried out by a single protein (e.g., Cas9 or Cpf1). In some examples, the present disclosure provides using type II and/or type V single-subunit effector systems.

As CRISPR systems occur in many different types of bacteria, the exact arrangements and structures of CRISPR, function and number of Cas genes and their product differ somewhat from species to species (Haft et al. (2005) PloS Comput. Biol. 1: e60; Kunin et al. (2007) Genome Biol. 8: R61; Mojica et al. (2005) J. Mol. Evol. 60: 174-182; Bolotin et al. (2005) Microbiol. 151: 2551-2561; Pourcel et al. (2005) Microbiol. 151: 653-663; and Stern et al. (2010) Trends. Genet. 28: 335-340.). For example, in E. coli K12, the CRISPR/cas system comprises eight cas genes: Cas3 (predicted HD-nuclease fused to a DEAD-box helicase), five genes designated casABCDE, cas1 (predicted integrase), and the endoribonuclease gene Cas2. After transcription of the CRISPR, a complex of Cas proteins (casABCDE) termed Cascade cleaves a CRISPR RNA precursor in each repeat and retains the cleavage products containing the virus-derived sequence. Assisted by the helicase Cas3, these mature CRISPR RNAs then serve as small guide RNAs that enable Cascade to interfere with virus proliferation (Brouns et al. (2008) Science 321: 960-964). In other prokaryotes, Cas6 processes the CRISPR transcript. The CRISPR-based phage inactivation in E. coli requires Cascade and Cas3, but not Cas1 or Cas2. The Cmr (Cas RAMP module) proteins in Pyrococcus furiosus and other prokaryotes form a functional complex with small CRISPR RNAs that recognizes and cleaves complementary target RNAs. A simpler CRISPR system relies on the protein Cas9, which is a nuclease with two active cutting sites, one for each strand of the double helix. Combining Cas9 and modified CRISPR locus RNA can be used in a system for gene editing (Pennisi (2013) Science 341: 833-836).

a. CRISPR/Cas9

Provided are methods of gene editing using a Type II CRISPR system. Type II systems rely on i) a single endonuclease protein, ii) a transactivating crRNA (tracrRNA), and iii) a crRNA wherein a ˜20-nucleotide portion of the 5′ end of the crRNA is complementary to a target nucleic acid. The region of a CRISPR crRNA strand that is complementary to its target DNA protospacer is referred to as “guide sequence.”

In some aspects, the tracrRNA and crRNA components of a Type II system can be replaced by a single guide RNA (sgRNA), also known as a guide RNA (gRNA). The sgRNA can include, for example, an at least 12-20 nucleotide sequence complementary to the target DNA sequence (guide sequence) and can include a common scaffold RNA sequence at its 3′ end. As used herein, “a common scaffold RNA” refers to any RNA sequence that mimics the tracrRNA sequence or any RNA sequences that function as a tracrRNA.

Cas9 endonucleases produce blunt end DNA breaks, and are recruited to target DNA by a combination of a crRNA oligo and a tracrRNA oligo, which tether the endonuclease via complementary hybridization of the RNA CRISPR complex. The HNH and RuvC nuclease domains of Cas9 in a type II CRISPR-Cas system are responsible for cleaving the DNA strand complementary to the guide sequence and the non-target strand, respectively, which creates a double-stranded break in the DNA utilized to introduce modifications by non-homologous end joining (NHEJ) or homology-directed repair (HDR) (Gao et al., 2017; Jiang & Doudna, 2017; Jinek et al., 2012; Symington & Gautier, 2011). HDR is more precise and requires a donor DNA template to repair the double-strand breaks, whereas NHEJ does not require a repair template (Puchta, 2005; Puchta et al., 1996). Due to its comparative simplicity, NHEJ is a more common method to disrupt genes in plants, especially in wheat (Li et al., 2021), by inducing small indels (insertions/deletions) in target genes, while HDR can precisely introduce specific point mutations and insert or replace sequences into the target DNA (Li et al., 2013a).

In some examples, DNA recognition by the crRNA/tracrRNA/endonuclease (or sgRNA/endonuclease) complex uses additional complementary base-pairing with a protospacer adjacent motif (PAM) (e.g., 5′-NGG-3′) located in a 3′ portion of the target DNA, downstream from the target protospacer (Jinek, M., et. Al., Science. 2012, 337:816-821). In some examples, the PAM motif recognized by a Cas9 varies for different Cas9 proteins.

Cas9 proteins that can be used in the methods and systems described herein include any naturally occurring and artificially obtained variants. In some examples, Cas9 can include one or more of the mutations described in the literature, including but not limited to the functional mutations described in: Fonfara et al. Nucleic Acids Res. 2014 February; 42(4):2577-90; Nishimasu H. et al. Cell. 2014 Feb. 27,156(5):935-49; Jinek M. et al. Science. 2012 337:816-21; and Jinek M. et al. Science. 2014 Mar. 14, 343(6176) (which are hereby incorporated by reference). See also U.S. Pat. Nos. 10,266,850; 8,697,359; 8,771,945; 8,795,965; 8,865,406; 8,871,445; 8,889,356; 8,895,308; 8,906,616; 8,932,814; 8,945,839; 8,993,233; and 8,999,641. Thus, in some examples, the systems and methods disclosed herein can be used with the wild type Cas9 protein having double-stranded nuclease activity, Cas9 mutants that act as single stranded nickases, or other mutants with modified nuclease activity.

Cas9 proteins that can be used in the methods and systems described herein include Cas9 proteins (or variant thereof) of a variety of species, e.g., S. pyogenes, S. thermophilus, Staphylococcus aureus, and Neisseria meningitidis. Additional Cas9 species include those from: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

Cas9 proteins that can be used in the methods and systems described herein also include SpyCas9, SaCas9, and St1Cas9. See for example, Song et al. (2016), The Crop Journal 4:75-82; Mali et al. (2013) Science 339: 823-826; Ran et al. (2015) Nature 520: 186-191; Esvelt et al. (2013) Nature methods 10(11): 1116-1121.

Editing a single base pair in the genome without introducing double-strand breaks can also be achieved by utilizing an engineered Cas9-based editors comprising a dead Cas9 domain fused to a cytidine deaminase enzyme, and a sgRNA, which can convert G to A and C to T (Komor et al., 2016). The same base conversions can also be achieved with a Cas9 fused with a transfer RNA adenosine deaminase (Gaudelli et al., 2017). The main benefit of these techniques is they induce point mutations without generating excess undesired editing by-products, such as off-target editing. These techniques have been used to edit genes in maize, rice, wheat, etc. (Rees & Liu, 2018; Zong et al., 2017).

b. CRISPR/Cpf1

In some aspects, a Type V CRISPR system is used to edit a plant genome. In some examples, the Cpf1 CRISPR system from Prevotella, Francisella, Acidaminococcus, Lachnospiraceae, or Moraxella is used.

The Cpf1 CRISPR systems can include i) a single endonuclease protein, and ii) a crRNA, wherein a portion of the 3′ end of crRNA contains the guide sequence complementary to a target nucleic acid. In this system, the Cpf1 nuclease is directly recruited to the target DNA by the crRNA. In some embodiments, guide sequences for Cpf1 are at least 12 nt, 13 nt, 14 nt, 15 nt, or 16 nt in order to achieve detectable DNA cleavage, and a minimum of 14 nt, 15 nt, 16 nt, 17 nt, or 18 nt to achieve efficient DNA cleavage.

The Cpf1 system differs from the Cas9 system in some ways. First, unlike Cas9, Cpf1 does not require a separate tracrRNA for cleavage. In some examples, Cpf1 crRNAs can be as short as about 42-44 nt long—of which about 23-25 nt is guide sequence and about 19 nt is the constitutive direct repeat sequence. In contrast, in some examples, the combined Cas9 tracrRNA and crRNA synthetic sequences can be about 100 nt long.

Second, certain Cpf1 systems prefer a “TTN” PAM motif that is located 5′ upstream of its target. This is in contrast to the “NGG” PAM motifs located on the 3′ of the target DNA for common Cas9 systems such as Streptococcus pyogenes Cas9 system. In some examples, the uracil base immediately preceding the guide sequence cannot be substituted (Zetsche, B. et al. 2015. “Cpf1 Is a Single RNA-Guided Endonuclease of a Class 2 CRISPR-Cas System” Cell 163, 759-771, which is hereby incorporated by reference in its entirety for all purposes).

Third, the cut sites for Cpf1 are staggered by about 3-5 nt, which create “sticky ends” (Kim et al., 2016. “Genome-wide analysis reveals specificities of Cpf1 endonucleases in human cells” published online Jun. 6, 2016). These sticky ends with 3-5 nt overhangs are thought to facilitate NHEJ-mediated-ligation, and improve gene editing of DNA fragments with matching ends. The cut sites are in the 3′ end of the target DNA, distal to the 5′ end where the PAM is. The cut positions usually follow the 18th nt on the non-hybridized strand and the corresponding 23rd nt on the complementary strand hybridized to the crRNA.

Fourth, in Cpf1 complexes, the “seed” region is located within the first 5 nt of the guide sequence. Cpf1 crRNA seed regions are highly sensitive to mutations, and even single base substitutions in this region can drastically reduce cleavage activity (see Zetsche B. et al. 2015 “Cpf1 Is a Single RNA-Guided Endonuclease of a Class 2 CRISPR-Cas System” Cell 163, 759-771). Critically, unlike the Cas9 CRISPR target, the cleavage sites and the seed region of Cpf1 systems do not overlap. Additional guidance on designing Cpf1 crRNA targeting oligos is available on Zetsche B. et al. 2015 (“Cpf1 Is a Single RNA-Guided Endonuclease of a Class 2 CRISPR-Cas System” Cell 163, 759-771).

c. Guide Nucleic Acids

In some examples, a guide nucleic acid (e.g., RNA or DNA) of the present disclosure includes two regions, being or encoding for crRNA and tracrRNA, respectively. The crRNA is complementary to a target, and the tracrRNA is responsible for binding with a Cas protein. In some examples, the two regions are provided as separate molecules. In some examples, the guide RNA is a single guide RNA (sgRNA) (a crRNA/tracrRNA hybrid). In some examples, the guide RNA is a crRNA for a Cpf1 endonuclease.

Guide nucleic acids are designed to recruit the CRISPR endonuclease to a target nucleic acid region. Such methods are known in the art. Software programs can be used to identify candidate CRISPR target sequences on both strands of an input DNA sequence based on desired guide sequence length and a CRISPR motif sequence (e.g., PAM) for a specified CRISPR enzyme. For example, target sites for Cpf1 from Francisella novicida U112, with PAM sequences TTN, may be identified by searching for 5′-TTN-3′ both on the input sequence and on the reverse-complement of the input. The target sites for Cpf1 from Lachnospiraceae bacterium and Acidaminococcus sp., with PAM sequences TTTN, may be identified by searching for 5′-TTTN-3′ both on the input sequence and on the reverse complement of the input. Likewise, target sites for Cas9 of S. thermophilus CRISPR, with PAM sequence NNAGAAW, may be identified by searching for 5′-Nx-NNAGAAW-3′ both on the input sequence and on the reverse-complement of the input. The PAM sequence for Cas9 of S. pyogenes is 5′-NGG-3′.

Since multiple occurrences in the genome of the DNA or RNA target site may lead to nonspecific genome editing, after identifying all potential sites, sequences may be filtered out based on the number of times they appear in the relevant reference genome or modular CRISPR construct. For those CRISPR enzymes for which sequence specificity is determined by a “seed” sequence (such as the first 5 nt of the guide sequence for Cpf1-mediated cleavage) the filtering step may also account for any seed sequence limitations.

In some aspects, algorithmic tools identify potential off target sites for a particular guide sequence. For example, Cas-Offinder can be used to identify potential off target sites for Cpf1 (see Kim et al., 2016. Nature Biotechnology 34, 863-868). Any other publicly available CRISPR design/identification tool may also be used, including for example the Zhang lab crispr.mit.edu tool (see Hsu, et al. 2013 “DNA targeting specificity of RNA guided Cas9 nucleases” Nature Biotech 31, 827-832).

In some aspects, the user can choose the length of the seed sequence. The user can specify the number of occurrences of the seed and PAM sequence in a genome for purposes of passing the filter. The default is to screen for unique sequences. Filtration level is altered by changing both the length of the seed sequence and the number of occurrences of the sequence in the genome. The program may in addition or alternatively provide the sequence of a guide sequence complementary to the reported target sequence(s) by providing the reverse complement of the identified target sequence(s).

In some aspects, the transgenic plant, plant part, plant cell, or plant tissue culture taught herein includes a recombinant construct, which includes at least one nucleic acid sequence encoding a guide RNA. In some examples, the nucleic acid is operably linked to a promoter. In some examples, a recombinant construct further comprises a nucleic acid sequence encoding a CRISPR endonuclease. In some examples, the guide RNA or DNA is capable of forming a complex with the CRISPR endonuclease, and the complex is capable of binding to and creating a double-strand break in a target nucleic acid sequence of the plant genome. In some examples, the CRISPR endonuclease is Cas9. In some examples, the CRISPR endonuclease is Cpf1. In some examples, the CRISPR endonuclease is Cas13d. In some aspects, the target sequence is a region within a GROOT1, GROOT2, or GROOT3 gene.

### B. Promoters

A nucleic acid sequence for expression (such as a coding sequence or a guide nucleic acid sequence) included in an expression cassette is typically operably linked to a regulatory element, such as a promoter. Exemplary promoters include a plant promoter, such as one from Arabidopsis or other plants (e.g., constitutive promoter from the Arabidopsis serine carboxypeptidase-like gene AtSCPL30, PD1 from Arabidopsis, HVA22E, PLDdelta, AtS1, and AtS3). In some examples, the promoter is heterologous to the plant into which it is introduced. In some examples, the promoter is heterologous to the sequence to which it is operably linked.

Promoter includes a region of DNA upstream from the start of transcription and involved in recognition and binding of RNA polymerase and other proteins to initiate transcription. A plant promoter is a promoter capable of initiating transcription in plant cells. Examples of promoters under developmental control include promoters that preferentially initiate transcription in certain tissues, such as leaves, roots, seeds, fibers, xylem vessels, tracheids, or sclerenchyma. Such promoters are referred to as “tissue preferred.” Promoters that initiate transcription only in a certain tissue are referred to as “tissue specific”. A “cell-type specific” promoter primarily drives expression in certain cell types in one or more organs, for example, vascular cells in roots or leaves. An “inducible” promoter is a promoter that is under environmental control. Examples of environmental conditions that may affect transcription by inducible promoters include anaerobic conditions or the presence of light. Tissue-specific, tissue-preferred, cell-type specific, and inducible promoters constitute the class of “non-constitutive” promoters. A “constitutive” promoter is a promoter that is active under most environmental conditions, and in most cell types. Exemplary promoter sequences are provided as SEQ ID NOs: 36-38. In some aspects, the promoter used in the presently disclosed methods includes at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to any of SEQ ID NOs: 36-38. In some aspects, a constitutive or inducible promoter that is transcribed in at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or all of the plant tissues or cells is used. In some aspects, a constitutive or inducible tissue specific or cell-type specific promoter is used.

a. Constitutive Promoters

Any suitable constitutive promoters can be used in the present methods and systems. Exemplary constitutive promoters include, but are not limited to, the promoters from plant viruses such as the 35S promoter from CaMV (Odell et al., Nature 313:810-812 (1985)) and the promoters from such genes as rice actin (McElroy et al., Plant Cell 2: 163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)) and maize H3 histone (Lepetit et al., Mol. Gen. Genetics 231:276-285 (1992) and Atanassova et al., Plant Journal 2 (3): 291-300 (1992)).

The ALS promoter, XbaI/NcoI fragment 5′ to the Brassica napus ALS3 structural gene (or a nucleotide sequence similarity to said XbaI/NcoI fragment), represents a particularly useful constitutive promoter. See WO 96/30530.

In a specific example the constitutive promoter is CaMV-35S, CaMV-35Somega, UBQ10 from Arabidopsis, Ubi1 from maize/rice, or barley leaf thionin BTH6 promoter.

b. Inducible Promoters

With an inducible promoter the rate of transcription increases in response to an inducing agent.

Any suitable inducible promoters can be used in the present methods and systems. See Ward et al., Plant Mol. Biol. 22:361-366 (1993). Exemplary inducible promoters include, but are not limited to, that from the ACEI system which responds to copper (Mett et al., PNAS 90:4567-4571 (1993)); In2 gene from maize which responds to benzenesulfonamide herbicide safeners (Hershey et al., Mol. Gen Genetics 227:229-237 (1991) and Gatz et al., Mol. Gen. Genetics 243:32-38 (1994)) or Tet repressor from Tn10 (Gatz et al., Mol. Gen. Genetics 227:229-237 (1991)). In some aspects, an inducible promoter is a promoter that responds to an inducing agent to which plants do not normally respond. An exemplary inducible promoter is the inducible promoter from a steroid hormone gene, the transcriptional activity of which is induced by a glucocorticosteroid hormone (Schena et al., Proc. Natl. Acad. Sci. USA 88:0421 (1991)).

c. Tissue Specific or Tissue Preferred Promoters

Any suitable tissue specific or tissue preferred promoter can be used in the present methods and systems. Exemplary tissue specific or tissue preferred promoters include, but are not limited to, a root-preferred promoter such as that from the phaseolin gene (Murai et al., Science 23:476-482 (1983) and Sengupta-Gopalan et al., Proc. Natl. Acad. Sci. USA 82:3320-3324 (1985)); a leaf specific and light induced promoter such as that from cab or rubisco (Simpson et al., EMBO J. 4(11):2723-2729 (1985) and Timko et al., Nature 318:579-582 (1985)); an anther-specific promoter such as that from LAT52 (Twell et al., Mol. Gen. Genetics 217:240-245 (1989)); a pollen-specific promoter such as that from Zm13 (Guerrero et al., Mol. Gen. Genetics 244:161-168 (1993)); or a microspore-preferred promoter such as that from anther-specific gene (apg) (Twell et al., Sex. Plant Reprod. 6:217-224 (1993)). In some examples, a tissue specific or tissue preferred promoter is a native promoter of FACT gene, HORST gene, ASFT gene, GPAT5 gene, RALPH gene, and/or MYB84 gene.

### C. Exemplary Methods of GROOT1, GROOT2, or GROOT3 Downregulation

The present disclosure provides gene editing methods of reducing the expression of one or more of GROOT1, GROOT2, and GROOT3 genes, or activity of one or more of GROOT1, GROOT2, and GROOT3 proteins by, for example, creating a loss-of-function mutation in one or more of GROOT1, GROOT2, and GROOT3 genes. Creating a loss-of-function mutation includes mutating a promoter or other regulatory regions of the genes, so that transcription cannot be initiated or is reduced; and/or mutating a coding region of the genes, so that transcription terminates prematurely, or translation cannot be initiated or completed, or the protein translated has a reduced function, compared to before the mutation is introduced.

The present disclosure also provides other methods that do not result in an altered plant genome, such as RNAi or antisense RNA methods targeting mRNA transcripts of one or more of GROOT1, GROOT2, and GROOT3 genes.

In some examples, gene edited plants are generated using gene editing technologies, for example, using a guide nucleic acid molecule specific for one or more of GROOT1, GROOT2, and GROOT3 genes, that can mutate the target, resulting in its decreased expression and/or activity of the protein encoded. In some examples, a CRISPR/Cas system is used. In some examples, gene edited plants provided herein include mutated GROOT1, GROOT2, and/or GROOT3 genes, and have increased biomass, particularly shoot biomass and/or root biomass, and/or seed size. In some aspects, the gene edited plants do not include exogenous nucleic acid molecules or transgenes.

Unlinked transgenic sequences (including the gRNA, Cas9, and selectable marker (Kan^{R}) expression cassettes) will naturally segregate away from any gene-edited site in ¼ of the T₁ generation. Thus, it is possible for plants to segregate out the gRNA and Cas9 transgenes in subsequent generations, thus producing transgene-free, gene-mutated plants. In one example, the use of recombinant DNA in the construction of gene-edited plants is avoided, and instead plant leaf tissues are transformed using pre-assembled gRNA and Cas9 RNP complexes. In one example, polyethylene glycol (PEG)-transformation of protoplasts (e.g., see Woo et al., Nat. Biotechnol., 2015. 33(11): 1162-4) or gene gun bombardment of immature embryos with RNPs (e.g., see Zhang et al., Nat Commun, 2016. 7:12617; Liang et al., Nat Commun, 2017. 8:14261) is used.

gRNAs can be produced using commercial kits, such as the Invitrogen GeneArt™ Precision gRNA Synthesis Kit. To produce more gRNAs, a DNA template can be assembled by PCR with forward and reverse overlapping oligonucleotides that contain the target DNA sequence, together with the T7 promoter and universal reverse primers supplied with the kit. In vitro transcripts can be produced by T7 RNA polymerase and purified by phenol/chloroform extraction and ethanol precipitation. The RNP complexes of 1-5 μg gRNA and 1 μg GeneArt™ Platinum™ Cas9 nuclease with nuclear-targeting signal (Invitrogen) can be assembled and incubated for 10 min at room temperature. The RNP complexes can be mixed with 1 mg 0.6 μm gold particles sterilized by 70% ethanol for gene gun bombardment. Seeds can be sterilized by 10% bleach for 15 min and rinsed three times with sterile water. Seeds can then be germinated. Leaf bases from the first true leaves of 3-week old young plants or callus generated from embryos can be used as explants for bombardment. The bombarded plant tissues can be cultured on MS medium supplemented with Gamborg vitamins, 3% sucrose and 16.8 μM thidiazuron (TDZ) until shoot formation. Regenerated shoots can be transferred onto MS medium without TDZ but containing 1 μg/l indole-3-butyric acid (IBA) to induce root formation. Fully regenerated plantlets can be transferred to soil and allowed to produce seeds under isolated conditions.

Since CRISPR-mediated gene editing occurs in T₀ plants, the integration of the gRNA/Cas cassettes into the plant genome can be examined by PCR on T₀ plants. Cas9 expression can be validated using a 3× FLAG antibody to detect the epitope-tagged Cas9 protein in Western blot analysis.

In one example, the gene editing method is free of recombinant technology and does not involve T-DNA, Ti-plasmids (or other plasmids), Agrobacterium or other pathogenic microbes. Once the gRNA/Cas9 RNP complex is delivered into leaf tissue, it can be rapidly degraded and lost from cells. Gene edited plants without any transgene can be produced immediately from edited plant cells.

In one example, editing of more than one gene at a time (e.g., two or more of GROOT1, GROOT2, GROOT3, and an unrelated gene) can be achieved by bombarding leaf tissues with two or more gRNA/Cas9 RNP complexes. Since there is no selectable marker delivered into leaf tissues, regenerated plantlets can be screened for gene-editing individually.

In some examples, gene edited plants are generated using Agrobacterium-mediated transformation, which stably integrates a single copy of an exogenous nucleic acid into plant genomes (e.g., see Deschamps and Simon, Plant Cell Rep., 2002. 21:359-364; Phippen and Simon, Cell. Dev. Biol., 2000. 36: 250-4) to produce gene-edited plants. Seeds can be germinated and the leaf tissues taken as explant for Agrobacterium inoculation for 30 min. The EHA105 strain of Agrobacterium can be transformed with a CRISPR-editing vector. The infected plant tissues can be cultured on MS medium supplemented with Gamborg vitamins, 3% sucrose and 16.8 μM thidiazuron (TDZ) for 3 days, after which plant tissues can be transferred to the same medium containing 300 μg/ml cefotaxime to inhibit the further growth of Agrobacterium and 50 μg/ml kanamycin to select transformed tissues and regenerate transgenic shoots. Regenerated transgenic shoots can be transferred onto MS medium without TDZ but containing 25 μg/ml kanamycin and 1 μg/l indole-3-butyric acid (IBA) to induce root formation. Fully regenerated transgenic plantlets can be transferred to soil and allowed to produce seeds. To transgenic plants can be examined for the integration of the transgenes by PCR analysis.

In some examples, antisense or inhibitory RNA (RNAi) technology is used to reduce or eliminate the activity of one or more of GROOT1, GROOT2, and GROOT3 genes. For example, a plant or plant cell can be engineered to contain a cDNA that encodes an antisense molecule that reduces or prevents one or more of GROOT1, GROOT2, and GROOT3 genes from being translated. The term “antisense molecule” encompasses any nucleic acid molecule or nucleic acid analog (e.g., peptide nucleic acids) that contains a sequence that corresponds to the coding strand of a GROOT1, GROOT2, or GROOT3 gene. Antisense molecules can also have flanking sequences (e.g., regulatory sequences). Antisense molecules can be ribozymes or antisense oligonucleotides. A ribozyme can have any general structure including, without limitation, hairpin, hammerhead, or axehead structures, provided the molecule cleaves RNA.

### D. Exemplary Methods of Screening Gene Edited and Transgenic Plants

Gene-edited and transgenic plants generated using the provided methods, such as those generated to contain non-native GROOT1, GROOT2, and/or GROOT3 sequence, can be screened to identify or confirm the presence of a mutation introduced.

PCR primers can be used to amplify all or a portion of a GROOT1, GROOT2, or GROOT3 sequence (or a fragment thereof), such as genomic DNA fragments spanning the selected gene target sites. Restriction enzyme digestion can be carried out on the PCR products. In some examples, restriction enzyme sites are included at the target sites (before editing occurs), and undigested PCR products in the presence of the restriction enzyme can thus indicate a gene-edited plant. The undigested PCR fragments can also be sequenced to confirm the presence and nature of any mutations or added sequences. RFLP methods can be used to screening large numbers of candidate mutant plants.

A T7E1 assay can be used to screen regenerated mutant plants. This assay allows mutated, edited sites to be detected based on their incomplete hybridization to the WT sequence (due to a mismatch between the WT and edited hybridized DNA strands at the edited site). PCR fragments spanning the mutation sites can be denatured at 95° C. and cooled down to 22° C. slowly using a thermal cycler. Annealed PCR products can be incubated with T7 endonuclease 1 (NEB) at 37° C. for 20 min and analyzed by electrophoresis in a 1-2% agarose gel.

A TaqMan probe-based qPCR analysis can be used. TaqMan probes can be designed for each of the WT target sites and synthesized with fluorescence labeling on the 5′ end and minor groove binder-nonfluorescent quencher (e.g., MGB-NFQ) on the 3′ end. In qPCR analysis, the biallelic mutant will not produce any fluorescent signal, while the WT plant will produce double the signal compared to the monoallelic mutant (e.g., see Li et al., Plant Physiol., 2015. 169(2): 960-70). This TaqMan-qPCR method in the 96-well format used by the StepOnePlus qPCR System (Applied Biosystems) can be used to screen a large number of regenerated plants, produced by the gene gun bombardment with RNP complexes. This method generates gene edited plants that do not carry selectable marker genes.

Mutations from biallelic T₀ mutants are expected to be inherited in the next generations. For transgenic mutant plants produced by Agrobacterium-mediated transformation, gene-specific PCR assays can be used to screen for T₁ plants that have segregated out the Cas9 and Kan^{R} genes. The monoallelic T₀ mutants are expected to segregate according to the Mendelian law with a 1:2:1 ratio.

### VI. Exemplary Methods for Plant Transformation

The disclosed polynucleotides for reducing GROOT1, GROOT2, and/or GROOT3 activity and/or expression of the present disclosure can be transformed into plant cells, plant tissues, plant parts and whole plants by any method disclosed herein or known in the art.

Methods of producing transgenic plants are known. Transgenic plants can now be produced by a variety of transformation methods including, but not limited to, electroporation; microinjection; microprojectile bombardment, also known as particle acceleration or biolistic bombardment; viral-mediated transformation; and Agrobacterium-mediated transformation. See, for example, U.S. Pat. Nos. 5,405,765; 5,472,869; 5,538,877; 5,538,880; 5,550,318; 5,641,664; 5,736,369 and 5,736,369; International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)).

Exemplary methods include Agrobacterium-mediated nucleic acid transfer (e.g., see U.S. Pat. No. 4,536,475, EP0265556, EP0270822, WO8504899, WO8603516, U.S. Pat. No. 5,591,616, EP0604662, EP0672752, WO8603776, WO9209696, WO9419930, WO9967357, U.S. Pat. No. 4,399,216, WO8303259, U.S. Pat. Nos. 5,731,179, 7,250,554, EP068730, WO9516031, U.S. Pat. Nos. 5,693,512, 6,051,757 and EP904362A1), microprojectile bombardment, injection into plant cells or tissues, direct incubation of an exogenous nucleic acid molecule with germinating pollen, and electroporation.

A transgenic plant formed using Agrobacterium transformation methods typically contains a single gene on one chromosome, although multiple copies are possible. Such transgenic plants can be referred to as being hemizygous for the added gene. A more accurate name for such a plant is an independent segregant, because each transformed plant represents a unique T-DNA integration event (U.S. Pat. No. 6,156,953). A transgene locus is generally characterized by the presence and/or absence of the transgene. A heterozygous genotype in which one allele corresponds to the absence of the transgene is also designated hemizygous (U.S. Pat. No. 6,008,437).

For efficient plant transformation, a selection method is used such that whole plants are regenerated from a single transformed cell and every cell of the transformed plant carries the nucleic acid of interest. These methods can employ positive selection, whereby a foreign nucleic acid is supplied to a plant cell that allows it to utilize a substrate present in the medium that it otherwise could not use, such as mannose or xylose (for example, refer U.S. Pat. Nos. 5,767,378; 5,994,629). Negative selection can be used, utilizing selective agents such as herbicides or antibiotics that either kill or inhibit the growth of non-transformed plant cells and reducing the possibility of chimeras. Resistance genes that are effective against negative selective agents are provided on the introduced foreign nucleic acid used for the plant transformation. For example, kanamycin, together with the resistance gene neomycin phosphotransferase (nptII), which confers resistance to kanamycin and related antibiotics (see, for example, Messing & Vierra, Gene 19: 259-268 (1982); Bevan et al., Nature 304:184-187 (1983)) can be used. However, many different antibiotics and antibiotic resistance genes can be used for transformation purposes (refer U.S. Pat. Nos. 5,034,322, 6,174,724 and 6,255,560). In addition, several herbicides and herbicide resistance genes have been used for transformation purposes, including the bar gene, which confers resistance to the herbicide phosphinothricin (White et al., Nucl Acids Res 18: 1062 (1990), Spencer et al., Theor Appl Genet 79: 625-631(1990), U.S. Pat. Nos. 4,795,855, 5,378,824 and 6,107,549). In addition, the dhfr gene, which confers resistance to the anticancer agent methotrexate, has been used for selection (Bourouis et al., EMBO J. 2(7): 1099-1104 (1983).

The expression control elements used to regulate the expression of a given nucleic acid can either be the expression control element that is normally found associated with the coding sequence (homologous expression element) or can be a heterologous expression control element. A variety of homologous and heterologous expression control elements are known and can readily be used to make expression units for use in the present disclosure. Transcription initiation regions, for example, can include any of the various opine initiation regions, such as octopine, mannopine, nopaline and the like that are found in the Ti plasmids of Agrobacterium tumefaciens. Alternatively, plant viral promoters can also be used, such as the cauliflower mosaic virus 19S and 35S promoters (CaMV 19S and CaMV 35S promoters, respectively) to control gene expression in a plant (U.S. Pat. Nos. 5,352,605; 5,530,196 and 5,858,742 for example). Enhancer sequences derived from the CaMV can also be utilized (U.S. Pat. Nos. 5,164,316; 5,196,525; 5,322,938; 5,530,196; 5,352,605; 5,359,142; and 5,858,742 for example). Plant promoters such as prolifera promoter, fruit specific promoters, Ap3 promoter, heat shock promoters, seed specific promoters, etc. can also be used.

A gamete-specific promoter, a constitutive promoter (such as the CaMV or Nos promoter), an organ specific promoter (such as the E8 promoter from tomato), or an inducible promoter can be ligated to the nucleic acid to be expressed. The expression unit may be further optimized by employing supplemental elements such as transcription terminators and/or enhancer elements.

Thus, for expression in plants, the expression units typically contain, in addition to the nucleic acid to be expressed, a plant promoter region, a transcription initiation site and a transcription termination sequence. Unique restriction enzyme sites at the 5′ and 3′ ends of the expression unit are typically included to allow for easy insertion into a pre-existing vector.

In some examples, the promoter is positioned about the same distance from the heterologous transcription start site as it is from the transcription start site in its natural setting. However, some variation in this distance can be accommodated without loss of promoter function.

In addition to a promoter sequence, the expression cassette can also contain a transcription termination region downstream of the nucleic acid to be expressed to provide for efficient termination. The termination region may be obtained from the same gene as the promoter sequence or may be obtained from different genes. If the mRNA encoded by the nucleic acid to be expressed is to be efficiently processed, DNA sequences which direct polyadenylation of the RNA are also commonly added to the vector construct. Polyadenylation sequences include, but are not limited to the Agrobacterium octopine synthase signal (Gielen et al., EMBO J 3:835 846 (1984)) or the nopaline synthase signal (Depicker et al., Mol. and Appl. Genet. 1:561 573 (1982)). The resulting expression unit is ligated into or otherwise constructed to be included in a vector that is appropriate for higher plant transformation. One or more expression units may be included in the same vector. The vector typically contains a selectable marker gene expression unit by which transformed plant cells can be identified in culture. Usually, the marker gene will encode resistance to an antibiotic, such as G418, hygromycin, bleomycin, kanamycin, or gentamicin or to an herbicide, such as glyphosate (Round-Up) or glufosinate (BASTA) or atrazine. Replication sequences, of bacterial or viral origin, can be included to allow the vector to be cloned in a bacterial or phage host; in one example a broad host range for prokaryotic origin of replication is included. A selectable marker for bacteria may also be included to allow selection of bacterial cells bearing the desired construct. Suitable prokaryotic selectable markers include resistance to antibiotics such as ampicillin, kanamycin or tetracycline. Other DNA sequences encoding additional functions may also be present in the vector. For instance, in the case of Agrobacterium transformations, T DNA sequences can be included for subsequent transfer to plant chromosomes.

To introduce a nucleic acid to be expressed by conventional methods requires a sexual cross between two lines, and then repeated back-crossing between hybrid offspring and one of the parents until a plant with the desired characteristics is obtained. This process, however, is restricted to plants that can sexually hybridize, and genes in addition to the desired gene will be transferred.

Recombinant DNA techniques circumvent these limitations by enabling introduction of specific genes for desirable traits, such as improved fatty acid composition, and to introduce these genes into already useful varieties of plants. Once the foreign genes have been introduced into a plant (such as a MYB67 or MYB69 repressor), that plant can then be used in imp plant breeding schemes (e.g., pedigree breeding, single-seed-descent breeding schemes, reciprocal recurrent selection) to produce progeny which also contain the gene of interest.

Genes can be introduced in a site directed fashion using homologous recombination. Homologous recombination permits site-specific modifications in endogenous genes and thus inherited or acquired mutations may be corrected, and/or novel alterations may be engineered into the genome. Homologous recombination and site-directed integration in plants are discussed in, for example, U.S. Pat. Nos. 5,451,513; 5,501,967 and 5,527,695.

An expression construct which includes nucleotide sequences that reduce expression and/or activity of one or more of GROOT1, GROOT2, and GROOT3 can be introduced into embryogenic callus of any plant genus or species and the resulting transformed cells can be regenerated into plants. The transgenic plants are expected to have expression of the exogenous nucleic acid molecule.

The phrase “embryogenic callus cell” used herein refers to an embryogenic cell contained in a cell mass produced in vitro.

Several approaches can be utilized to transform and co-express these polynucleotides in plant cells.

Each nucleic acid molecule to be expressed (e.g., those that decrease expression and/or activity of one or more of GROOT1, GROOT2, and GROOT3) can be separately introduced into a plant cell by using separate nucleic-acid constructs. In some embodiments, two or more nucleic acid molecules to be expressed sequences can be co-introduced and co-expressed in the plant cell using a single nucleic acid construct. Such a construct can be designed with a single promoter sequence, which can transcribe a polycistronic message RNA including the nucleic acid molecules to be expressed. To enable co-translation of multiple nucleic-acid constructs, the polynucleotide sequences can be inter-linked via an internal ribosome entry site (IRES) sequence which facilitates translation of polynucleotide sequences positioned downstream of the IRES sequence. In this case, a transcribed polycistronic RNA molecule encoding the individual nucleic-acid constructs can be translated from both the capped 5′ end and the two internal IRES sequences of the polycistronic RNA molecule to thereby express each nucleic acid molecule to be expressed.

In some examples, the two or more nucleic acid molecules to be expressed are translationally fused via a protease recognition site cleavable by a protease expressed by the cell to be transformed with the nucleic acid construct. In this case, a chimeric polypeptide translated will be cleaved by a cell-expressed protease to thereby generate the plurality of polypeptides.

In other embodiments, a nucleic acid construct includes multiple promoter sequences each capable of directing transcription of a specific polynucleotide sequence.

Suitable promoters which can be used include constitutive, inducible, or tissue-specific promoters.

Exemplary constitutive promoters include, for example, CaMV 35S promoter (Odell et al., Nature 313:810-812, 1985); maize Ubi 1 (Christensen et al., Plant Sol. Biol. 18:675-689, 1992); rice actin (McElroy et al., Plant Cell 2:163-171, 1990); pEMU (Last et al., Theor. Appl. Genet. 81:581-588, 1991); and Synthetic Super MAS (Ni et al., The Plant Journal 7: 661-76, 1995). Other constitutive promoters include those in U.S. Pat. Nos. 5,659,026, 5,608,149; 5,608,144; 5,604,121; 5,569,597: 5,466,785; 5,399,680; 5,268,463; and 5,608,142.

Suitable inducible promoters can be pathogen-inducible promoters such as, for example, the alfalfa PR10 promoter (Coutos-Thevenot et al., Journal of Experimental Botany 52: 901-910, 2001 and the promoters described by Marineau et al., Plant Mol. Biol. 9:335-342, 1987; Matton et al. Molecular Plant-Microbe Interactions 2:325-331, 1989; Somsisch et al., Proc. Natl. Acad. Sci. USA 83:2427-2430, 1986: Somsisch et al., Mol. Gen. Genet. 2:93-98, 1988; and Yang, Proc. Natl. Acad. Sci. USA 93:14972-14977, 1996.

Suitable tissue-specific promoters include, but not limited to, leaf-specific promoters such as described, for example, by Yamamoto et al., Plant J. 12:255-265, 1997; Kwon et al., Plant Physiol. 105:357-67, 1994; Yamamoto et al., Plant Cell Physiol. 35:773-778, 1994; Gotor et al., Plant J. 3:509-18, 1993; Orozco et al., Plant Mol. Biol. 23:1129-1138, 1993; and Matsuoka et al., Proc. Natl. Acad. Sci. USA 90:9586-9590, 1993.

A nucleic acid construct can also include at least one selectable marker such as nptII. In one example, the nucleic acid construct is a shuttle vector, which can propagate both in E. coli (wherein the construct comprises an appropriate selectable marker and origin of replication) and be compatible for propagation in cells. In some examples, a construct can be, for example, a plasmid, a bacmid, a phagemid, a cosmid, a phage, a virus or an artificial chromosome.

Following transformation, the transformed cells can be micropropagated to provide a rapid, consistent reproduction of the transformed material. Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that utilizes alteration of culture medium or growth conditions between stages. The micropropagation process involves four basic stages: stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Integration of an exogenous nucleic acid molecule in the genome of the transformed plants can be determined using standard molecular biology techniques, such as PCR and Southern blot hybridization.

In some examples the transformation is stable. In some examples the transformation is transient.

In one example, transformation is by viral infection. Viruses useful for the transformation of plant hosts include CaMV, TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV), EPA 278,667 (BV); and Gluzman et al. (Communications in Molecular Biology: Viral Vectors, Cold Spring Harbor Laboratory, New York, pp. 172-189, 1988). Pseudovirus particles for use in expressing an exogenous nucleic acid molecule in many hosts, including plants, is described in WO 87/06261.

Suitable modifications can be made to a DNA virus. Alternatively, the virus can first be cloned into a bacterial plasmid for ease of constructing the desired viral vector with the exogenous nucleic acid molecule. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA.

If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

In one embodiment, a plant viral nucleic acid is provided in which the native coat protein coding sequence has been deleted from a viral nucleic acid, a non-native plant viral coat protein coding sequence and a non-native promoter, such as the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the exogenous nucleic acid molecule within it, such that a product is produced. The recombinant plant viral nucleic acid may contain one or more additional non-native subgenomic promoters. Each non-native subgenomic promoter can transcribe or express adjacent genes or nucleic acid sequences in the plant host and incapable of recombination with each other and with native subgenomic promoters. Exogenous nucleic acid molecules can be inserted adjacent the native plant viral subgenomic promoter or the native and a non-native plant viral subgenomic promoters if more than one nucleic acid sequence is included. The exogenous nucleic acid sequences are transcribed or expressed in the host plant under control of the subgenomic promoter to produce the desired products.

In some examples, the native coat protein coding sequence is placed adjacent one of the non-native coat protein subgenomic promoters instead of a non-native coat protein coding sequence.

In some examples, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Exogenous nucleic acid molecules can be inserted adjacent the non-native subgenomic plant viral promoters such that the sequences are transcribed or expressed in the host plant under control of the subgenomic promoters to produce the desired product.

In some examples, a recombinant plant viral nucleic acid is provided in which the native coat protein coding sequence is replaced by a non-native coat protein coding sequence.

The viral vectors can be encapsidated by the coat proteins encoded by the recombinant plant viral nucleic acid to produce a recombinant plant virus. The recombinant plant viral nucleic acid or recombinant plant virus can be used to infect appropriate host plants. The recombinant plant viral nucleic acid can be capable of replication in the host, systemic spread in the host, and transcription or expression of foreign gene(s) (isolated nucleic acid) in the host to produce the desired product.

In some examples, the exogenous nucleic acid sequences can also be introduced into a chloroplast genome thereby enabling chloroplast expression.

### VII. Exemplary Breeding Methods

In one example, open-pollinated methods are used for crops such as rye, many maizes and sugar beets, herbage grasses, legumes such as alfalfa and clover, and tropical tree crops such as cacao, coconuts, oil palm and some rubber.

Population improvement methods fall into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

In one example, a population is changed en masse using a selection procedure. The outcome is an improved population that is indefinitely propagable by random-mating within itself in isolation. Second, the synthetic variety attains the same end result as population improvement but is not itself propagable as such; it has to be reconstructed from parental lines or clones These plant breeding procedures for improving open-pollinated populations are known and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988). For population improvement methods specific for soybean see, e.g., J. R. Wilcox, editor (1987) SOYBEANS: Improvement, Production, and Uses, Second Edition, American Society of Agronomy, Inc., Crop Science Society of America, Inc., and Soil Science Society of America, Inc., publishers, 888 pages.

In one example, mass selection methods are used. In mass selection, desirable individual plants are chosen, harvested, and the seed composited without progeny testing to produce the following generation. Since selection is based on the maternal parent only, and there is no control over pollination, mass selection amounts to a form of random mating with selection. The purpose of mass selection is to increase the proportion of superior genotypes in the population.

In one example, a synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or topcrosses, more generally by polycrosses. Parental seed lines may be deliberately inbred (e.g. by selfing or sib crossing). However, even if the parents are not deliberately inbred, selection within lines during line maintenance ensure that some inbreeding occurs. Clonal parents will, of course, remain unchanged and highly heterozygous.

Whether a synthetic can go straight from the parental seed production plot to the farmer or first undergoes one or two cycles of multiplication depends on seed production and the scale of demand for seed. Generally, grasses and clovers are generally multiplied once or twice and are thus considerably removed from the original synthetic.

In some examples, progeny testing is used for polycrosses, because of their operational simplicity and relevance to the objective, namely exploitation of general combining ability in a synthetic.

The number of parental lines or clones that enters a synthetic can vary. In some examples, numbers of parental lines range from 10 to several hundred, with 100-200 being the average. Broad based synthetics formed from 100 or more clones can be more stable during seed multiplication than narrow based synthetics.

In some examples, hybrids are generated. A hybrid is an individual plant resulting from a cross between parents of differing genotypes. Commercial hybrids are used in many crops, including corn (maize), sorghum, sugar beet, sunflower and broccoli. Hybrids can be formed, for example by crossing two parents directly (single cross hybrids), by crossing a single cross hybrid with another parent (three-way or triple cross hybrids), or by crossing two different hybrids (four-way or double cross hybrids).

Most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids can include the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

In some examples, bulk segregation analysis (BSA) is used. BSA, a.k.a. bulked segregation analysis, or bulk segregant analysis, is described by Michelmore et al. (Michelmore et al., 1991, Proceedings of the National Academy of Sciences, USA, 99:9828-9832) and Quarrie et al. (Quarrie et al., 1999, Journal of Experimental Botany, 50(337):1299-1306). For BSA of a trait of interest, parental lines with certain different phenotypes are chosen and crossed to generate F2, doubled haploid or recombinant inbred populations with QTL analysis. The population is then phenotyped to identify individual plants or lines having high or low expression of the trait. Two DNA bulks are prepared, one from the individuals having one phenotype (e.g., increased phellem size, periderm size, and/or suberin production), and the other from the individuals having reversed phenotype (e.g., average or decreased phellem size, periderm size, and/or suberin production), and analyzed for allele frequency with molecular markers. Only a few individuals are required in each bulk (e.g., 10 plants each) if the markers are dominant (e.g., RAPDs). More individuals are needed when markers are co-dominant (e.g., RFLPs). Markers linked to the phenotype can be identified and used for breeding or QTL mapping.

In some examples, gene pyramiding is used to combine into a single genotype a series of target genes identified in different parents. The first part of a gene pyramiding breeding is called a pedigree and is aimed at cumulating one copy of all target genes in a single genotype (called root genotype). The second part is called the fixation steps and is aimed at fixing the target genes into a homozygous state, that is, to derive the ideal genotype (ideotype) from the root genotype. Gene pyramiding can be combined with marker assisted selection (MAS) or marker based recurrent selection (MBRS).

VIII. Exemplary Plants for Use with the Disclosed Methods

The present disclosure teaches plants transformed with a plant transformation construct or vector. The methods for targeted gene-editing as described herein can be used to confer desired traits on essentially any plant. A wide variety of plants and plant cell systems may be engineered for the desired physiological and agronomic characteristics described herein using the nucleic acid constructs of the present disclosure and the various transformation methods. In some embodiments, the plant for the transformation is a monocotyledonous plant (monocot) or a dicotyledonous plant (dicot).

Monocots are flowering plants having embryos with one cotyledon or seed leaf, parallel leaf veins, and flower parts in multiples of three. Examples of monocots that can be used for transformation, genetic engineering or gene-editing include, but are not limited to turfgrass, corn/maize, rice, oat, annual ryegrass, wheat, barley, sorghum, orchid, iris, lily, onion, and palm. Examples of turfgrass include, but are not limited to Agrostis spp. (bentgrass species including colonial bentgrass and creeping bentgrasses), Poa pratensis (Kentucky bluegrass), Lolium spp. (ryegrass species including annual ryegrass and perennial ryegrass), Festuca arundinacea (tall fescue) Festuca rubra commutata (Chewings fescue), Cynodon dactylon (bermudagrass, Pennisetum clandestinum (kikuyu grass), Stenotaphrum secundatum (St. Augustine grass), Zoysia japonica (zoysia grass), and Dichondra micrantha.

Other exemplary plants that can be used for transformation, genetic engineering or gene-editing include, but are not limited to angiosperm and gymnosperm plants such as acacia, alfalfa, amaranth, apple, apricot, artichoke, ash tree, asparagus, avocado, banana, barley, beans, beet, birch, beech, blackberry, black raspberry, blueberry, broccoli, Brussel's sprouts, cabbage, cane berry, canola, cantaloupe, carrot, cassava, cauliflower, cedar, a cereal, celery, chestnut, cherry, Chinese cabbage, citrus, Clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, peach, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, wild strawberry, yams, yew, and zucchini.

In some aspects, plants and plant cells for transformation, genetic engineering or gene-editing include, but are not limited to, those monocotyledonous and dicotyledonous plants, such as crops including grain crops (e.g., wheat, maize, rice, millet, barley), fruit crops (e.g., tomato, apple, grape, peach, pear, plum, raspberry, black raspberry, blackberry, cane berry, cherry, avocado, strawberry, wild strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). In some embodiments, fruit crops such as tomato, apple, peach, pear, plum, raspberry, black raspberry, blackberry, cane berry, cherry, avocado, strawberry, wild strawberry, grape and orange.

In some aspects, plants and plant cells for transformation, genetic engineering or gene-editing include, but are not limited to, Canola (Brassica napus), Soybean (Glycine max), Cotton (Gossypium hirsutum), Rice (Oryza sativa), Lotus (Lotus japonicus), Radish (Raphanus sativus), Setaria (Setaria italica), Sorghum (Sorghum bicolor), Pennycress (Thlaspi arvense), Southern cattail (Typha domingensis), Wheat (Triticum aestivum), and Maize (Zea mays).

In some aspects, the plant for transformation, genetic engineering or gene-editing is a dicot. In some embodiments, the plant, plant part, or plant cell is a species selected from Arabidopsis genus, Brassica genus, Glycine genus, Gossypium genus, Oryza genus, Raphanus genus, Setaria genus, Sorghum genus, Thlaspi genus, Typha genus, Triticum genus, and Zea genus.

In some aspects, the plant, plant part, or plant cell is from Arabidopsis thaliana.

In some aspects, the plant, plant part, or plant cell is from the Brassica genus, such as Brassica balearica (Mallorca cabbage), Brassica carinata (Abyssinian mustard or Abyssinian cabbage), Brassica elongata (elongated mustard), Brassica fruticulosa (Mediterranean cabbage), Brassica hilarionis (St. Hilarion cabbage), Brassica juncea (Indian mustard, brown and leaf mustards, Sarepta mustard), Brassica napus (rapeseed, canola, rutabaga, Siberian kale), Brassica narinosa (broadbeaked mustard), Brassica nigra (black mustard), Brassica oleracea (kale, cabbage, collard greens, broccoli, cauliflower, kai-lan, brussels sprouts, kohlrabi), Brassica perviridis (tender green, mustard spinach), Brassica rapa (Chinese cabbage, turnip, rapini, komatsuna), Brassica rupestris, Brassica spinescens, or Brassica tournefortii (Asian mustard).

In some aspects, the plant, plant part, or plant cell is from the Thlaspi genus and is Thlaspi alliaceum (roadside penny-cress), Thlaspi arcticum (arctic penny-cress), Thlaspi arvense (field penny-cress), Thlaspi caerulescens (alpine penny-cress), Thlaspi californicum (Kneeland Prairie penny-cress), Thlaspi cyprium (Cyprus penny-cress), Thlaspi fendleri (Fendler's penny-cress), Thlaspi idahoense (Idaho penny-cress), Thlaspi jankae (Slovak penny-cress), Thlaspi montanum (alpine penny-cress), Thlaspi parviflorum (meadow penny-cress), Thlaspi perfoliatum (Cotswold penny-cress), Thlaspi praecox (early penny-cress), or Thlaspi rotundifolium (round-leaved penny-cress).

In some aspects, the plant, plant part, or plant cell is from the Glycine genus and is Glycine albicans, Glycine aphyonota, Glycine arenaria, Glycine argyria, Glycine canescens, Glycine clandestine, Glycine curvata, Glycine cyrtoloba, Glycine falcata, Glycine gracei, Glycine hirticaulis, Glycine hirticaulis subsp., Glycine lactovirens, Glycine latifolia, Glycine latrobeana, Glycine microphylla, Glycine montis-douglas, Glycine peratosa, Glycine pescadrensis, Glycine pindanica, Glycine pullenii, Glycine remota, Glycine rubiginosa, Glycine stenophita, Glycine syndetika, Glycine tabacina, Glycine tomentella, Glycine soja, or Glycine max.

### IX. Exemplary Additional Genes

One or more herbicide resistance genes can be used with the methods and plants provided herein. In particular examples, a herbicide resistance gene confers tolerance to an herbicide, such as glyphosate, sulfonylurea, imidazalinone, dicamba, glufosinate, phenoxy proprionic acid, cyclohexone, triazine, benzonitrile, broxynil, L-phosphinothricin, cyclohexanedione, chlorophenoxy acetic acid, or combinations thereof.

In one example the herbicide resistance gene is a gene that confers resistance to an herbicide that inhibits the growing point or meristem, such as an imidazalinone or a sulfonylurea. Exemplary genes in this category code for mutant ALS and AHAS enzyme as described, for example, by Lee et al. (1988. Embryo J. 7:1241-8) and Miki et al. (1990. Theoret. Appl. Genet. 80:449-458).

Resistance genes for glyphosate (resistance conferred by mutant 5-enolpyruvl-3 phosphikimate synthase (EPSP) and aroA genes, respectively) and other phosphono compounds such as glufosinate (phosphinothricin acetyl transferase (PAT) and Streptomyces hygroscopicus phosphinothricin-acetyl transferase (bar) genes) can be used (e.g., see U.S. Pat. No. 4,940,835). Examples of specific EPSPS transformation events conferring glyphosate resistance are described, for example, in U.S. Pat. No. 6,040,497.

DNA molecules encoding a mutant aroA gene are known (e.g., ATCC accession number 39256 and U.S. Pat. No. 4,769,061), as are sequences for glutamine synthetase genes, which confer resistance to herbicides such as L-phosphinothricin (e.g., U.S. Pat. No. 4,975,374), phosphinothricin-acetyltransferase (e.g., U.S. Pat. No. 5,879,903). DeGreef et al. (1989. Bio/Technology 61-64) describe the production of gene-edited plants that express chimeric bar genes coding for phosphinothricin acetyl transferase activity. Exemplary genes conferring resistance to phenoxy propionic acids and cyclohexones, such as sethoxydim and haloxyfop are the Acct-S1, Accl-S2 and Acct-S3 genes described by Marshall et al. (1992. Theor Appl Genet. 83:435-442).

Exemplary genes conferring resistance to an herbicide that inhibits photosynthesis include triazine (psbA and gs+ genes) and benzonitrile (nitrilase gene) (see Przibilla et al., 1991. Plant Cell. 3:169-174). Nucleotide sequences for nitrilase genes are disclosed in U.S. Pat. No. 4,810,648, and DNA molecules containing these genes are available under ATCC Accession Nos. 53435, 67441, and 67442. Cloning and expression of DNA coding for a glutathione S-transferase is described by Hayes et al. (1992. Biochem. J. 285:173).

U.S. Patent Publication No: 20030135879 describes dicamba monooxygenase (DMO) from Pseudomonas maltophilia, which is involved in the conversion of a herbicidal form of the herbicide dicamba to a non-toxic 3,6-dichlorosalicylic acid and thus can be used for producing plants tolerant to this herbicide.

The metabolism of chlorophenoxyacetic acids, such as, for example 2,4-D herbicide, is well known. Genes or plasmids that contribute to the metabolism of such compounds are described, for example, by Muller et al. (2006. Appl. Environ. Microbiol. 72(7):4853-4861), Don and Pemberton (1981. J Bacteriol 145(2):681-686), Don et al. (1985. J Bacteriol 161(1):85-90) and Evans et al. (1971. Biochem J 122(4):543-551).

### X. Transformed or Modified Plants

Provided are gene-edited plants, plant parts, plant cells, or seeds, comprising one or more deletions of, or one or more loss-of-function mutations in one or more of GROOT1, GROOT2 and GROOT3 genes. In some aspects, the gene-edited plants, plant parts, plant cells, or seeds do not include a transgene used to generate the one or more deletions or loss-of-function mutations, or are transgene-free, while in other aspects include one or more transgenes, which includes an exogenous vector, an inhibitory RNA molecule, a guide nucleic acid, a Cas gene, or combinations thereof.

In some aspects, the GROOT1 gene in the gene-edited plants, plant parts, plant cells, or seeds include (e.g., prior to the one or more deletions or one or more loss-of-function mutations) at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to any of SEQ ID NOs: 1, 3-4 and 6; or encodes a protein coding sequence that includes at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to any of SEQ ID NOs: 2, 5 and 7; or encodes a protein that includes at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to any of SEQ ID NOs: 8-13.

In some aspects, the GROOT2 gene in the gene-edited plants, plant parts, plant cells, or seeds include (e.g., prior to the one or more deletions or one or more loss-of-function mutations) at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to any of SEQ ID NOs: 14 and 16-17; or encodes a protein coding sequence that includes at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to any of SEQ ID NOs: 15 and 18; or encodes a protein that includes at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to any of SEQ ID NOs: 19-22.

In some aspects, the GROOT3 gene in the gene-edited plants, plant parts, plant cells, or seeds include (e.g., prior to the one or more deletions or one or more loss-of-function mutations) at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to any of SEQ ID NOs: 23 and 25-29; or encodes a protein coding sequence that includes at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to SEQ ID NO: 24; or encodes a protein that includes at least about 60%, at least about 65%, at least about 70%, at least about 75%, at least about 80%, at least about 85%, at least about 90%, at least about 95%, at least about 96%, at least about 97%, at least about 98%, or at least about 99%, or 100% sequence identity to any of SEQ ID NOs: 30-35.

In some aspects, the gene-edited plants are, or the plant parts, plant cells, or seeds are from a pennycress, soybean, canola, rice, wheat, corn, or sorghum plant.

In some aspects, the gene-edited plants, or the plants regenerated or grown from the gene-edited plant parts, plant cells, or seeds have increased biomass, particularly shoot biomass and/or root biomass, and/or increased seed size, as compared to a control plant.

In some aspects, the gene-edited plants, or the plants regenerated or grown from the gene-edited plant parts, plant cells, or seeds have at least about 5% more, at least about 10% more, at least about 15% more, at least about 20% more, at least about 25% more, at least about 30% more, at least about 35% more, at least about 40% more, at least about 45% more, at least about 50% more, at least about 55% more, at least about 60% more, at least about 65% more, at least about 70% more, at least about 75% more, at least about 80% more, at least about 85% more, at least about 90% more, at least about 95% more, at least about 100% more, at least about 125% more, at least about 150% more, at least about 175% more, or at least about 200%, or at least about 300%, or at least about 400% more shoot biomass, root biomass, entire biomass, and/or seed size compared to a control plant that does not have the gene edit.

In some aspects, the gene-edited seeds have at least about 5% more, at least about 10% more, at least about 15% more, at least about 20% more, at least about 25% more, at least about 30% more, at least about 35% more, at least about 40% more, at least about 45% more, at least about 50% more, at least about 55% more, at least about 60% more, at least about 65% more, at least about 70% more, at least about 75% more, at least about 80% more, at least about 85% more, at least about 90% more, at least about 95% more, at least about 100% more, at least about 125% more, at least about 150% more, at least about 175% more, or at least about 200% more seed size compared to a control seed that does not have the gene edit.

In some aspects, the gene-edited plants, or the plants regenerated or grown from the gene-edited plant parts, plant cells, or seeds, when grown at an elevated temperature, have at least about 5% more, at least about 10% more, at least about 15% more, at least about 20% more, at least about 25% more, at least about 30% more, at least about 35% more, at least about 40% more, at least about 45% more, at least about 50% more, at least about 55% more, at least about 60% more, at least about 65% more, at least about 70% more, at least about 75% more, at least about 80% more, at least about 85% more, at least about 90% more, at least about 95% more, at least about 100% more, at least about 125% more, at least about 150% more, at least about 175% more, or at least about 200%, or at least about 300%, or at least about 400% more shoot biomass, root biomass, entire biomass, and/or seed size compared to a control plant that does not have the gene edit and grown at the same elevated temperature. Elevated temperature refers to any temperature higher than about 27° C., such as higher than about 28° C., such as about 27° C. to about 40° C., about 27° C. to about 35° C., about 28° C. to about 40° C., about 28° C. to about 35° C., about 27° C., about 28° C., about 29° C., about 30° C., about 31° C., about 32° C., about 33° C., about 34° C., or about 35° C. In some examples, the elevated temperature is about 28° C. to 32° C.

In some aspects, the disclosure teaches a method of producing a plant having increased biomass, particularly root biomass and/or shoot biomass, and/or seed size, including crossing the gene-edited plant of the present disclosure with itself or another plant; and selecting a progeny plant having increased biomass and/or seed size.

In some aspects, the method further comprises using the selected progeny in a breeding method taught herein.

In some aspects, the disclosure teaches a method for increasing biomass, particularly root biomass and/or shoot biomass, and/or seed size, including growing a gene-edited plant of the disclosure, or a progeny thereof, for example in soil or other growth media, including growing at an elevated temperature or at a regular temperature (e.g., about 12° C. to about 27° C.).

### EXAMPLES

Root systems take up water and nutrients from the soil and thereby underpin all essential plant functions. The size of the root system affects the ability of roots to capture resources and determines the amount of carbon that roots transfer into the soil. Understanding the genetic basis of root biomass regulation is therefore very important for enhancing plant resilience and productivity, as well as carbon sequestration capability, especially in the face of climate change.

In the present disclosure, root biomass data of a diverse set of Arabidopsis thaliana accessions mainly derived from Sweden and Spain were catalogued, and GWAS was used to identify loci associated with root biomass in Arabidopsis thaliana. It was found that genetic variants associated with high biomass are enriched in accessions originating from distinct biogeographic regions in Spain. Among the most significant SNPs, one SNP on chromosome 3, was closely linked to three genes for which loss of function mutations caused significant increases in root, shoot, and seed biomass and that were named GROOT genes. These genes act as general growth limiters. Additionally, the present disclosure revealed that the GROOT genes are in strong linkage disequilibrium, implying a potential coordinated function in regulating growth. Furthermore, accessions carrying the non-reference allele at this SNP showed markedly higher biomass under elevated temperatures, indicating that these genes may also play a role in temperature adaptation. The discovery that the GROOT genes can enhance biomass without negative trade-offs across multiple traits is highly valuable, particularly for improving crop resilience and adaptability, as well as carbon sequestration.

Natural variation in the root biomass of Arabidopsis thaliana was studied to identify genes associated with root biomass traits using a genome wide association study (GWAS). By analyzing 52 candidate genes from the top five most significant GWAS hits, three genes (GROOT1, GROOT2, GROOT3) that lead to significantly increased root biomass when mutated were identified. Loss of function of these genes did not only cause a strong increase of root biomass but also influenced other traits, including increasing aboveground biomass and seed size. While being involved in distinct growth-related molecular processes, the natural alleles of the three GROOT genes are genetically linked to one another. Prompted by the geographic distribution of the accessions harboring distinct GROOT alleles, the impact of the genes and alleles on growth responses to temperatures was studied, and significant genotype-by-temperature interactions were found. It is demonstrated herein that GROOT genes and their variants play a critical role in determining the overall growth and fitness of a plant, and can be used to enhance many desirable traits of a plant, including carbon sequestration, crop resilience, seed size, root biomass, aboveground biomass, and productivity.

### Materials and Methods

Plant materials and growth conditions: Seeds of Arabidopsis thaliana were surface sterilized by being placed in open 1.5 mL Eppendorf tubes within a sealed environment with chorine gas. Chlorine gas was generated from a solution comprising 10% sodium hypochlorite (130 mL) and 37% hydrochloric acid (3.5 mL) that was kept in the sealed container with the seeds for an hour. Afterward, for stratification, the seeds underwent water imbibition followed by a 4-day period at 4° C. in darkness. After that the seeds were sown directly on ½ MS agar plates (pH 5.7, comprising 1% (w/v) sucrose and 1% (w/v) phytagel from Sigma, plated within 12 cm×12 cm square plates from Greiner). The plants were grown under long-day conditions (16 hours light followed by 8 hours dark) in a walk-in growth chamber maintained at 21° C., with a light intensity of 50 M and 60% humidity. Nighttime temperatures were reduced to 16° C. Seedlings were allowed to grow for 21 days before imaging. For each line, four plates were grown, with three seedlings (in some experiments five seedlings) per plate.

High-throughput image-based deep learning (UNet⁺⁺) pipeline for root biomass phenotyping: After allowing the seedlings on the plates to grow for 21 days, images of the plates were captured using CCD flatbed scanners (EPSON Perfection V600 Photo, Seiko Epson CO., Nagano, Japan). These images were used to quantify total root pixel counts using a bespoke deep learning-based approach termed the Total Root Pixel (TRP) method. The method operates through several stages, encompassing image pre-processing, UNet⁺⁺ model training, prediction generation, post-processing, and, ultimately, phenotyping. Comprehensive explanations of each step are provided in the following sections, while an overview of the entire procedure is depicted in FIG. 1B.

Each plate image, showing three Arabidopsis thaliana seedlings, was manually cropped to separate individual seedlings. Overlapping roots from different seedlings were carefully delineated and masked to prevent analysis errors. The training dataset was prepared by manually annotating roots and the surrounding background in the images using the LabelMe tool (Russell et al. 2008). To ensure comprehensive detail capture without losing boundary information, high-resolution images and their annotations were split into 512×512 pixel patches with a 16-pixel overlap. The training set, comprising 40 images with cropped seedlings, was randomly selected from the plate images.

The UNet⁺⁺ architecture was then used for root segmentation enhanced by a ResNet18 encoder, known for its efficiency in handling detailed images (Zhou et al. 2018; Ronneberger et al. 2015). The model used a sigmoid activation function, which is particularly effective for binary segmentation tasks. Training was conducted over 40 epochs with an initial learning rate of 0.0001, utilizing the Adam optimizer. The model's efficacy was evaluated using the Intersection over Union (IoU) metric, ensuring that only the best-performing model was retained for future predictions. Additionally, data augmentation strategies were integrated to bolster the model's robustness and adaptability across varied imaging conditions (Shorten and Khoshgoftaar 2019).

In the prediction phase, individual seedling images were cropped, split into patches, and segmented using the trained UNet++ model. Segmented patches were then stitched to reconstruct the original layout of the cropped seedlings. A custom Python script utilizing the OpenCV library was developed to refine segmentation by applying thresholding techniques and identifying connected components. The largest component was targeted as the root, with noise reduction performed by excluding small irrelevant components, ensuring high fidelity in the analyses. The GitHub repository at https://github.com/idelly007/TRP-Total-Root-Pixel-Pipeline contains the scripts developed for the TRP pipelines.

Employing the developed method, the total root pixels (TRP) were quantified for 12 seedlings per accession. Subsequently, to validate the accuracy of the biomass estimate derived from TRP image pixel calculations pipeline, a representative subset of the accessions employed in the estimation process were grown to acquire data on root fresh weight. To achieve this, a total of 72 accessions were randomly selected, and their seedlings cultivated on MS plates for a duration of 21 days. After this growth period, the roots and shoots were carefully separated into distinct collection tubes. After ensuring the removal of any excess moisture from the growth media by employing Kim wipes, the separated plant roots were promptly weighed. The assessment of fresh weight biomass for the root tissue was conducted using a Toledo scale. Then the correlation coefficients (r) were calculated between the root biomass estimated by the TRP method and the root biomass measured from the actual harvest of root tissue.

Broad sense heritability (H²) calculation: The broad-sense heritability (H²=V_{G}/V_{P}) was calculated using the TRP data from 264 accessions. We used h2boot software fitting one-way ANOVA among individual lines with 1000 bootstrap runs (Phillips and Arnold 1999). Broad-sense heritability represents the proportion of phenotypic variation (V_{P}) attributed to genetic variation (V_{G}), estimated from the between-line phenotypic variance.

Genome-Wide Association (GWA) mapping with the TRP data: Genome-Wide Association Study (GWAS) was performed using the mean TRP values of 264 accessions. Genome-Wide Association (GWA) mapping was conducted using fully imputed SNP data from the 1001 Arabidopsis thaliana database (https://1001genomes.org/) with the Efficient Mixed Model Analysis (EMMA) mixed model algorithm as outlined by (Kang et al. 2010), integrated within the PyGWAS software framework. Single nucleotide polymorphisms (SNPs) with minor allele counts (MAC) of 10 or more were considered. The significance of SNP associations was evaluated at a 5% False Discovery Rate (FDR) threshold (P<0.05) computed using the Benjamini-Hochberg method to address multiple testing (Benjamini and Yekutieli 2001).

Analysis of Linkage disequilibrium (LD): To assess Linkage Disequilibrium (LD) (r²) at the GWAS peak, plink 1.9 (Purcell et al. 2007) was used with a window size of 70 kb (“-ld-window-kb 70”). The significance of r² was determined using the 95th percentile (P<0.05) across the window.

T-DNA insertion lines and phenotyping: Based on the GWAS results, T-DNA insertion lines for 51 candidate genes from ABRC (https://abrc.osu.edu/) were acquired. Upon receipt of the lines, they were genotyped to validate their homozygous insertion status. The PCR genotyping primers for each T-DNA line were generated using the Salk T-DNA primer design database (http://signal.salk.edu/tdnaprimers.2.html). Subsequently, phenotypic assessments were conducted on the homozygous T-DNA lines for root biomass and primary root length by growing them on ½ MS plates. Wild-type (WT) (Col-0) samples obtained from three distinct sources were used as a WT control.

The T-DNA lines and WT plants were grown for 21 days under long-day conditions before harvest. The seeds were directly sown onto ½ MS plates and stratified in darkness at 4° C. The growing day count started when the plates were transferred to long-day conditions. Each plate contained 5 seeds, and to minimize potential biases, the plates within the growth chamber were rotated every 3 days. Plates were scanned on days 7, 14, and 21. Primary root length was measured using ImageJ software based on images captured on days 7 and 14. On day 21, seedlings from each plate were harvested and pooled into pre-weighed Eppendorf tubes to ensure accurate fresh and dry weight measurements. Due to the small size and weight of Arabidopsis thaliana seedlings, the unit of replication for biomass weighing was the plate containing 5 seedlings, rather than individual seedlings. Fresh weights were measured using a Mettler Toledo scale, followed by drying the tissues in an oven at 50° C. for four days, to obtain dry weight measurements. Subsequently, Fresh weight and dry weight per plant were calculated for further analysis.

The same methods were used for high-temperature experiments, except that the temperature was maintained at 28° C. during light exposure periods (16 h) and at 21° C. during dark periods (8 h). To ensure consistent sample preparations for both regular and high-temperature conditions, the two experiments were initiated simultaneously.

Expression data and GO enrichment of co-expressed genes: Published datasets were studied to understand the expression variations of the candidate genes. For organ- and development-specific expression patterns, data from Klepikova et al. (2016) were used, and for cell-specific expression, data from Shahan et al. (2022) were used. Next, 200 co-expressed genes were identified using the AttedII database (https://atted.jp/), and GO enrichment analysis was performed on these genes using PlantRegMap (https://plantregmap.gao-lab.org/go.php) with default parameters.

Statistical Analysis: Statistical analyses were conducted using R (https://www.r-project.org/) and JMP 16 from SAS (https://www.jmp.com/en_us/home.html). Inkscape (https://inkscape.org/) was used for image editing. The physical map of accession distribution was created in JMP using the graph builder. Principal Component Analysis (PCA) and correlation analyses were performed using the REML estimation method. Trait value significance was assessed by the Dunnett test, comparing means from multiple experimental groups (T-DNA lines) to a control group (Col-0) to determine significant differences. For multiple comparisons in single-point experiments, significance was determined by one-way or two-way ANOVA with Tukey's HSD test (p<0.05). Each experiment was repeated independently at least twice to ensure consistent results.

### Example 1

### Natural Variation of Root Biomass in Arabidopsis Thaliana

Natural variation of root biomass in Arabidopsis thaliana was assessed by measuring root biomass in 21-day old seedlings of A. thaliana natural accessions. 264 accessions were selected from the 1001 genomes collection (Alonso-Blanco et al. 2016; Weigel and Mott 2009) based on geographic origin. Most of the selected accessions originated from Spain and Sweden.

Twelve seedlings, with three seedlings per plate, were grown on the surface of ½ MS medium agar plates (a total of four plates), for each of the 264 accessions. After 21 days of growth, each plate was scanned using the BRAT scanner system (Slovak et al. 2014). Root growth patterns among the accessions grown for this experiment showed considerable differences. FIG. 1A illustrates a few examples highlighting the variations in root growth patterns.

As weighing A. thaliana roots is technically challenging and error prone due to their low mass, an image-based quantification of the root size was performed. To facilitate this, a deep learning UNet⁺⁺ based pipeline, termed Total Root Pixels (TRP) pipeline, was developed to automatically quantify the number of root pixels from seedlings from a scanned plate (FIG. 1B). The distribution of mean TRP of different accessions spanned a substantial range, from 83,938 pixels to 824,464 pixels, with an average of 278,203 pixels. The distribution of the TRP of the accessions is illustrated in FIG. 1C.

To validate that the pixel count was a good proxy for root weight, a randomly selected subset of 72 accessions were grown for 21 days. The roots were then harvested for mass measuring. Consistent with the assumption that TRP is a good and practical proxy measure for root fresh weight, a strong positive correlation (r=0.879, P<0.001) was observed between fresh weight in grams and the TRP value obtained using the TRP pipeline (FIG. 1D).

Next, to determine whether the phenotypic variation of TRP among the studied lines could be attributed to genetic variation, the broad-sense heritability (H²) of TRP was calculated and found to display a high heritability of 64% (bootstrap-based significance, P<0.001), indicating that a majority of the observed phenotypic variance in TRP data is due to genetic variation. Thus, there is substantial and heritable variation for root biomass in A. thaliana natural accessions.

### Example 2

Genetic Variants Associated with High Biomass are Enriched in Accessions Originating from Distinct Biogeographic Regions in Spain

To identify the associations between genetic variations among the natural accessions and the root biomass phenotype, the TRP data were used to conduct a genome-wide association study (GWAS). For this, a linear mixed model EMMAX (Kang et al. 2010) was used, linking the SNP data extracted from 1001 Genomes data base (full imputed; https://1001genomes.org/). Significantly associated SNPs were then identified by applying a 5% false discovery rate (FDR) threshold, adjusted using the Benjamini-Hochberg (BH) procedure. This analysis revealed 43 significant associations across chromosomes (FIG. 2A). The top five most significant hits, which not only exceeded the 5% FDR threshold but also the more stringent Bonferroni 5% threshold, were selected for candidate gene discovery. All of these hits were located on chromosomes two and three (FIG. 2A).

The most significantly associated SNP (marker SNP) on chromosome 2 (position 7915769) captured a notable increase (103%) in pixel counts for the non-reference allele compared to the reference (Col-0) allele (P<0.0001), as depicted in FIG. 2B. Across four other significant hits from the GWAS dataset, higher pixel counts associated with the non-reference allele (P<0.05) were consistently observed, indicating their contribution to greater pixel counts (FIG. 2B). Notably, at Chromosome 2 at position 7979808 bp, the non-reference allele exhibited a 75% increase in pixel counts compared to the reference allele, while at Chromosome 3 at position 6772287 bp, the SNP association showed an 83% increase (FIG. 2B). Similar trends were observed at Chromosome 3 at position 8040450 bp and Chromosome 3 at position 17969811 bp, with the non-reference alleles displaying 56% and 82% increases, respectively, in pixel counts compared to their reference counterparts (FIG. 2B). Collectively, these findings underscore the diverse impact of allele variation on pixel counts and emphasize the significance of these genomic regions in influencing root biomass. This observation indicates the possibility of natural selection exerting an influence on these loci, leading to the accumulation of pixel counts associated with the non-reference allele. This intricate interplay underscores the complexity of genetic associations and the adaptive potential inherent within these genetic variants.

After SNPs significantly associated with natural variation of root biomass was identified, whether there are geographical patterns of SNP distribution was explored. It was observed that nearly every non-reference allele variant for the top five SNPs, which passed the Bonferroni multiple corrections and was associated with increased root biomass, was found in accessions from Spain (FIG. 2C). This pattern indicates that genetic relatedness and/or geographic or environmental factors within this region played a role in the occurrence of variants associated with higher root biomass. It was first tested whether relatedness explained the biomass accumulation (as opposed to environmental factors). It was found that neither country of origin (P=0.435) nor population structure (P=0.5745) explained TRP difference to a significant extent, while genetic variants for the top five GWAS hits demonstrated a significant association (P<0.0001), highlighting that distinct genetic variants play a role in determining root biomass accumulation patterns.

Next, studies were performed to identify potential environmental factors that are linked to genetic variants associated with increased root biomass accumulation in natural accessions. To achieve this, analysis was focused on populations outside of Sweden, because the top five variants correlated with increased biomass were exclusively found in non-Swedish accessions (FIG. 2C), and individuals from Sweden exhibited distinctiveness within the dataset. Within the subgroup of non-Swedish accessions, the correlation was assessed between biomass data and bioclimatic variables (Bio01-Bio035, Fick and Hijmans 2017; Kriticos et al. 2012). No significant relationship was observed between the biomass data and any of the bioclimatic variables within that subpopulation, after FDR correction was applied to the P-values obtained from statistical tests. As non-reference allele variants were associated with increased root biomass, analysis was then focused only on the accessions with the non-reference allele for the top five GWAS SNPs (FIG. 2A), and correlations between biomass and bioclimatic variables were calculated. One significant correlation between biomass data and Bio024 (Radiation of the wettest quarter (Wm²)) (r=0.47, P<0.05, FIG. 2D) was found. To investigate whether the correlation between the two traits was influenced by population structure, a Principal Component Analysis (PCA) was performed on the data (biomass and Bio24) to capture the main axes of variation that might correspond to population structure. The first two principal components (PC1 explaining 73.4% of the variation, PC2 explaining 26.2%, and together accounting for 99.6% of the total variation) were extracted and included in subsequent analyses. Two linear regression models were then fitted to account for population structure. The first model included population as a categorical variable, while the second model included PC1 and PC2 as covariates. The results showed that Bio24 was significantly associated with biomass after accounting for population structure, whether using population as a categorical variable (P<0.05) or using the principal components (P<0.05). This indicates that the observed correlation between biomass and Bio24 is not solely due to population structure.

In summary, accessions may be selectively adapted to specific climatic conditions where solar radiation during the wettest quarter is highest, favoring allele variants associated with increased root biomass accumulation in their local habitats. These findings underscore the intricate interplay between genetic variation, environmental pressures, and adaptation. They show that local environments, such as those found in Spain, exert a significant influence on the evolutionary trajectories of plant populations, driving the accumulation of beneficial alleles associated with increased biomass accumulation.

### Example 3

### Loss of Function Mutations of GWAS Candidate Genes Result in a Significant Increase of Root and Shoot Biomass, and Seed Size

Based on the several highly significantly associated loci for root biomass, genes were then identified, that underlie the observed variation of root biomass in proximity of the identified loci. Candidate genes within 4,000 base pairs of each of the top five significantly associated SNPs were considered. Due to the number of candidate genes, only those harboring SNPs within their coding regions were further considered, reducing the candidate list to 58 genes. Among these 58 genes, only those with single T-DNA insertion lines available from the stock center were further considered, narrowing the candidate list to 51 genes. For these T-DNA lines, after confirming homozygous mutant insertion lines, phenotyping for root biomass was conducted, comparing biomass of these lines to that of the wildtype (Col-0) collected from three different sources. T-DNA lines were identified, that displayed higher root biomass compared to three Col-0 wildtypes that were obtained from different sources. While the vast majority of the screened T-DNA lines exhibited no significant differences in root biomass compared to the wildtype or produced inconsistent results, T-DNA lines for three distinct genes (AT3G19440, AT3G19590, AT3G19630) consistently displayed significantly increased root dry weight compared to the three wildtype lines. Loss of function mutants of each of these three genes demonstrated not only statistically significant (P<0.05) higher mass for root mass after a 21-day growth period on MS plates, but also significantly higher shoot mass (FIG. 3D). Aside from increased root and shoot biomass, no abnormal growth or any other obvious phenotypes were observed for these lines. Thus, each of these three genes has a function in limiting growth, given that loss of function mutant yielded higher biomass. These previously uncharacterized genes were named GROOT1 to GROOT3 (GRT, based on the finding that mutations of these genes lead to Greater ROOT biomass). All three genes were on chromosome 3 and originated from the GWAS peak at position 6772287.

These genes and their mutant lines were studied further. GROOT1 (AT3G19440) encodes a protein that belongs to the pseudouridine synthase family. Members of this family are enzymes catalyzing the conversion of pseudouridine (Ψ) to uridine (U) (Song et al. 2020). To confirm the initial results obtained with the one T-DNA mutant, three additional, independent T-DNA lines were acquired, in which GROOT1 was disrupted by T-DNA insertions in different parts of the gene including promoter, exons and introns. Biomass changes compared to the wildtype (WT) reference line (Col-0) as measured by root fresh weight and root dry weight were evaluated, after 21 days of growth under long-day conditions on MS plates (FIGS. 3A-3C). Like the first T-DNA line, all T-DNA lines exhibited significantly higher root fresh weight compared to WT (P<0.05). The mean percentage increase in root fresh weight was 61% compared to WT (FIG. 3C). Additionally, it was also evaluated whether the increase in fresh weight accompanied an increase in dry weight. As shown in FIG. 3B, the average increase in root dry weight for these three GROOT1T-DNA lines was 34%, with all lines displaying statistically significant differences at P<0.05 compared to WT. All three T-DNA lines also displayed significantly higher shoot biomass (as measured by shoot dry weight) compared to the WT (P<0.05) (FIG. 3D). Overall, these three GROOT1T-DNA lines exhibited an average of 26% increase in shoot dry weight compared to WT (FIG. 3D). These results show that GROOT1 gene is involved in limiting plant growth.

GROOT2 (AT3G19590) encodes a protein that belongs to the BUB3 (budding uninhibited by benzimidazoles 3) family. Members of this family are involved in the spindle assembly checkpoint and gametophyte development (Lermontova et al. 2008). Multiple T-DNA lines (FIGS. 3A-3C) with disrupted GROOT2 gene were examined, and root biomass, as well as shoot biomass were compared to the WT. As shown in FIGS. 3B and 3C, all T-DNA lines exhibited significant (P<0.05) increases in root biomass compared to WT, as measured by root fresh weight and root dry weight. Particularly, the T-DNA lines exhibited an average increase of 71% in root fresh weight compared to the WT (FIG. 3C), and an average increase of 37% in root dry weight compared to the WT (FIG. 3B). There was also a significant increase in shoot biomass (as measured by shoot dry weight) in the T-DNA lines compared to the WT, with an average increase of 30% (FIG. 3D). These results show that the GROOT2 gene is involved in limiting plant growth.

The GROOT3 gene (AT3G19630) encodes a protein belonging to the radical SAM superfamily. Members of this family encode enzymes that utilize SAM (S-adenosyl-L-methionine) to initiate radical reactions through liberation of the 5′-deoxyadenosyl (5′-dAdo) radical (Holliday et al. 2018; Hoffman et al. 2023). Two distinct T-DNA lines with disrupted GROOT3 gene were studied for the impact of GROOT3 on root fresh and dry weight biomass accumulation. Like the observations with the GROOT1 and GROOT2 T-DNA lines, both GROOT3 T-DNA lines demonstrated significantly higher root biomass, as measured by both fresh weight and dry weight (FIGS. 3A-3C). On average, the GROOT3 T-DNA lines exhibited a 66% increase in root fresh weight (FIG. 3C) and a 38% increase in root dry weight (FIG. 3B) compared to the WT. This underscores the significant role of GROOT3 in limiting biomass accumulation in roots. Like the other GROOT mutant lines, the GROOT3 mutant lines also exhibited significant shoot biomass increase (as measured by shoot dry weight) compared to the WT (FIG. 3D), showing an average of 29% increase in shoot biomass compared to the WT. These results show that the GROOT3 gene is involved in limiting plant growth.

Seeds of the GROOT lines were next studied. The seed area (mm²) of the WT plants and the GROOT1-3 T-DNA lines was measured. It was found that a robust positive correlation existed between seed area and root dry weight (r=0.39, P<0.0001), root fresh weight (r=0.45, P<0.0001), and shoot dry weight (r=0.34, P<0.0001) (FIG. 3E). This result indicates that the increase in biomass accumulation goes along with increases in seed size among the GROOT mutant lines. Overall, these findings show that the three GROOT genes do not determine trade-offs between root and shoot growth and seed size, but rather act as general growth limiters. These findings also indicate at the potential evolutionary significance of altering multiple traits simultaneously, without experiencing negative trade-offs between different aspects of plant growth and development. They reflect an adaptive strategy where plants optimize resource allocation to maximize overall fitness, a concept central to evolutionary biology and ecological adaptation (Grime and Pierce 2012; Ackerly et al. 2000; Anderson et al. 2011; Smith 1978).

### Example 4

GROOT Genes are Associated with Different Growth-Related Processes

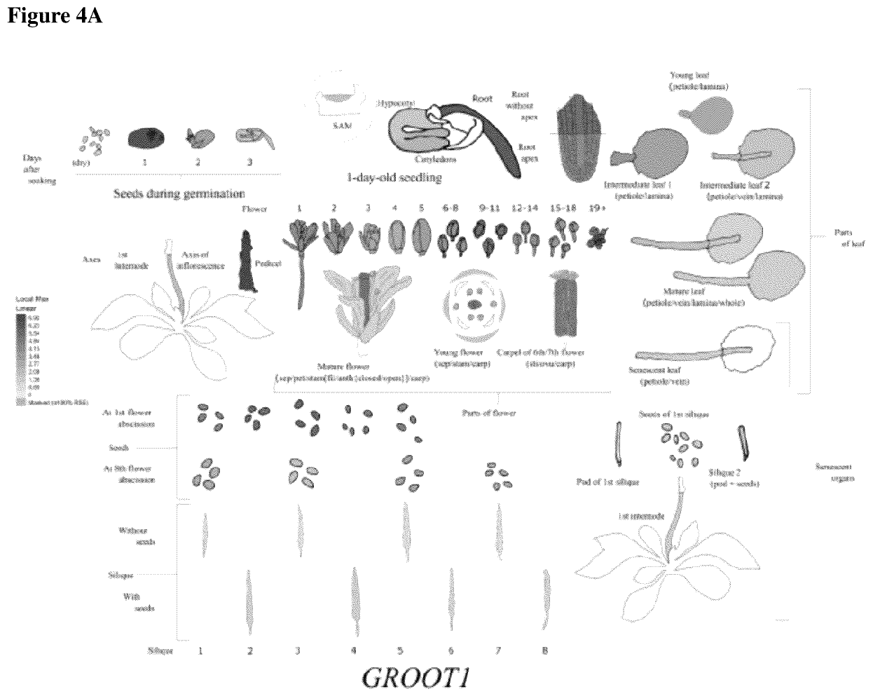

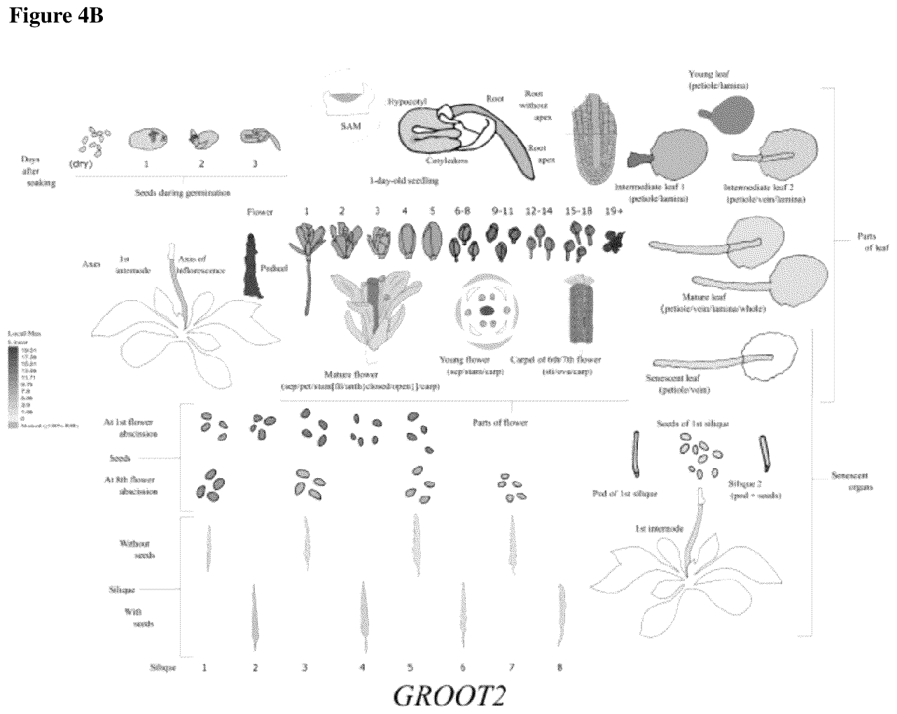

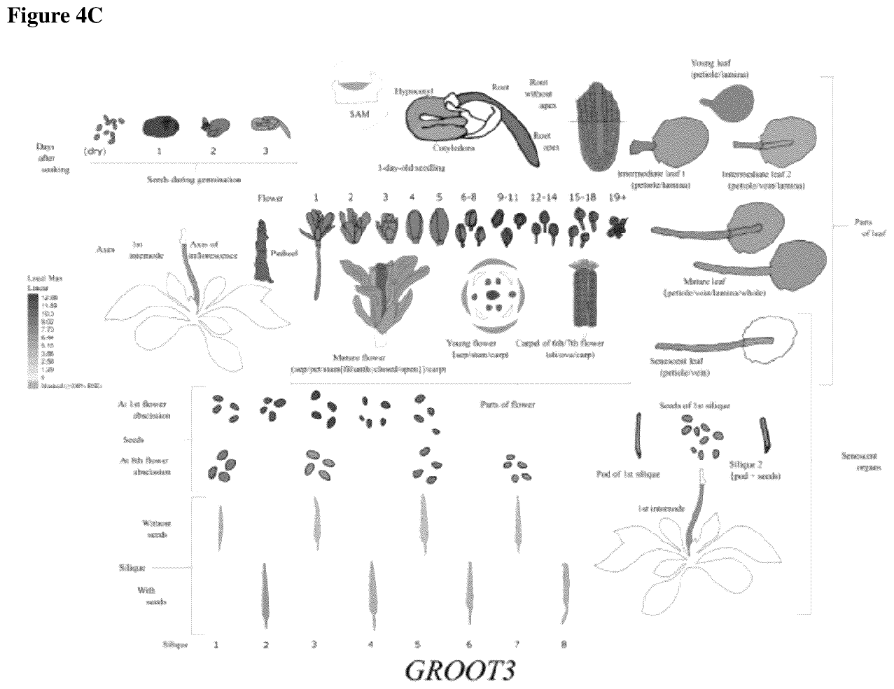

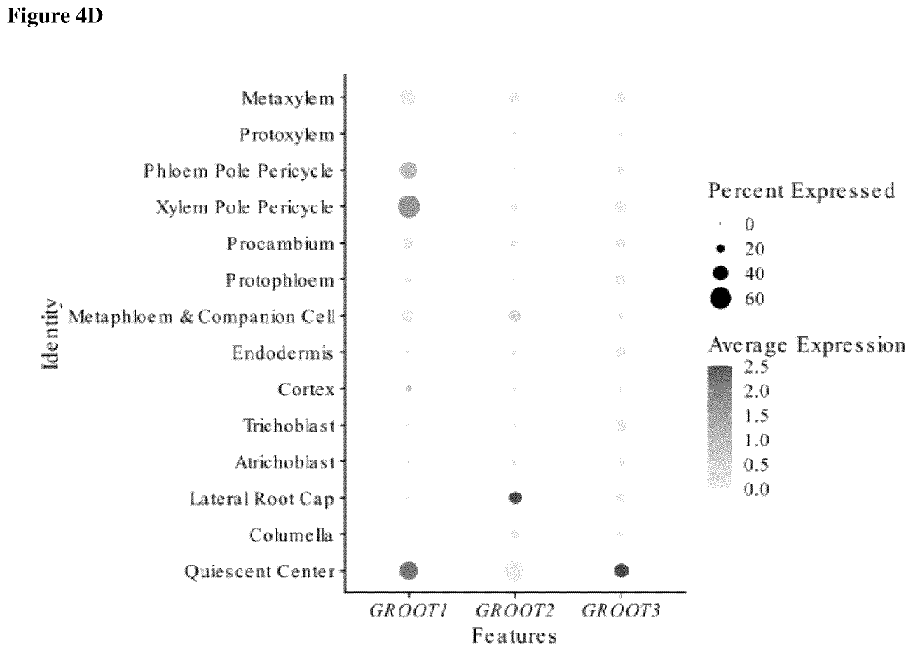

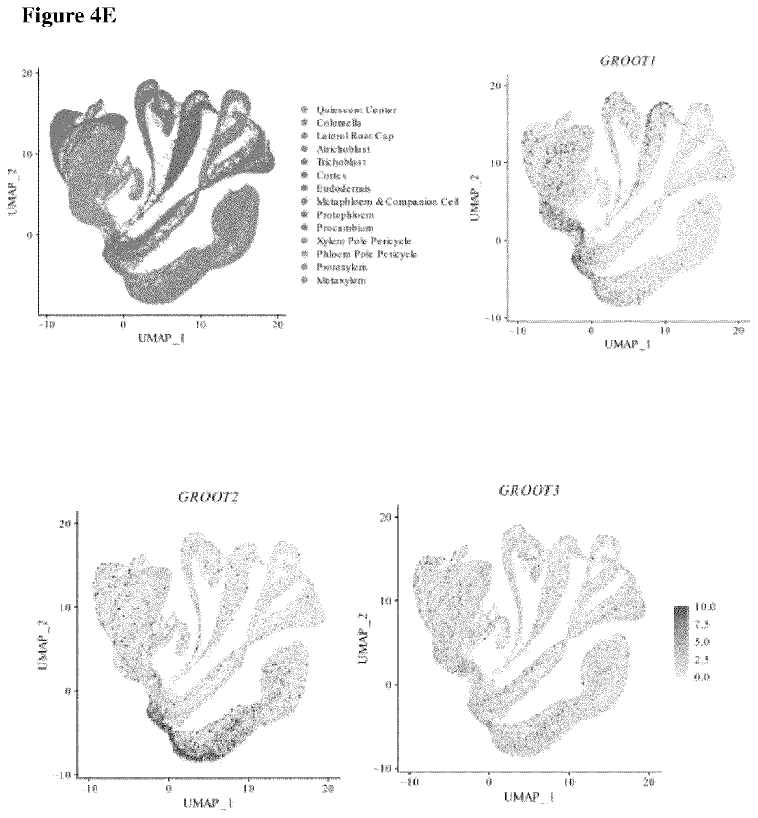

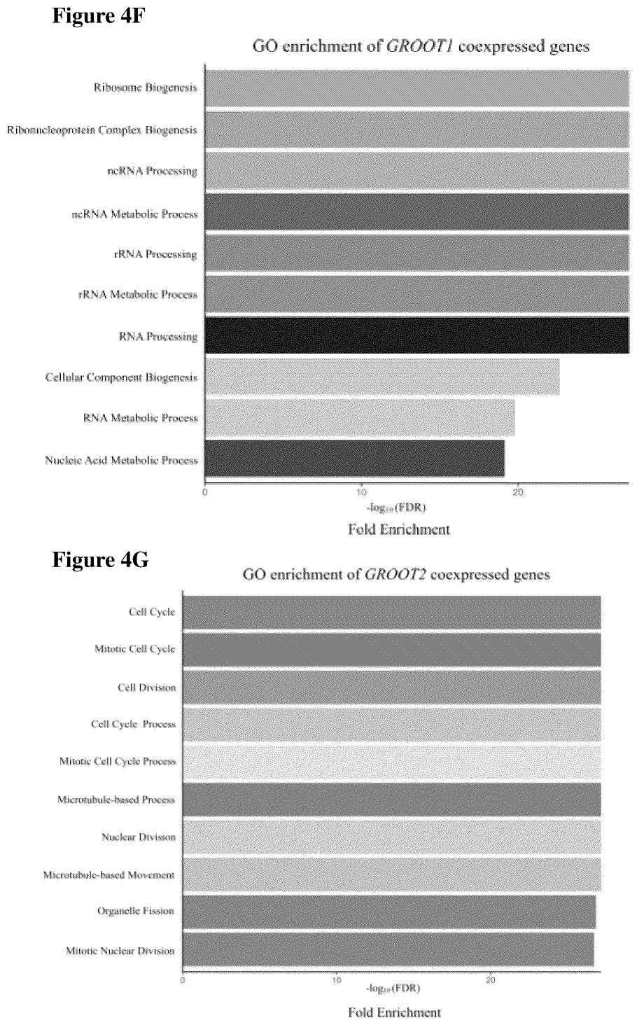

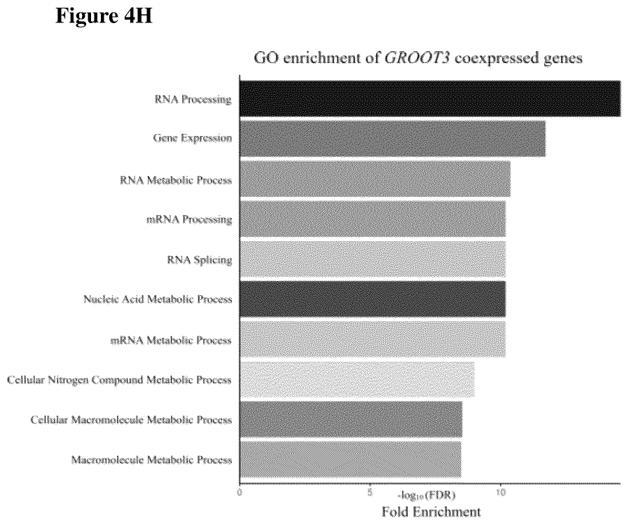

To further understand the role of the GROOT genes, their expression pattern across different cell and tissue types were studied based on available published data. The expression patterns across different organs and developmental stages were first examined using the data from Klepikova et al. (2016). It was found that all three GROOT genes are expressed in various tissue types and developmental stages, though their expression levels varied. Notably, GROOT3 exhibited higher expression levels across all tissue types and developmental stages compared to GROOT1 and GROOT2 (FIGS. 4A-4C). While all three genes were expressed in common developmental stages and tissue types such as the root, seed, shoot apex, and shoot system, each gene displays unique expression characteristics. To further understand their expression patterns in the root at a single-cell resolution, published single-cell root data from Shahan et al. (2022) were analyzed. This analysis revealed additional variations in specific cell type expression patterns (FIG. 4D). For instance, GROOT1 shows higher expression levels in the xylem pole pericycle, phloem pole pericycle, and the quiescent center. In contrast, GROOT2 shows an elevated expression in the lateral root cap, while GROOT3 shows a higher expression in the quiescent center (FIG. 4D). These genes are also expressed in both dividing (meristem) and maturing tissues in the root. However, while all three genes are expressed in the dividing tissues, their expression in maturing tissues varies. For instance, GROOT1 is expressed in the xylem pole pericycle maturing tissue; GROOT3 is expressed in all maturing tissues; GROOT2 is restricted to the lateral root cap maturing tissue (FIG. 4E). These observations show that these three genes might play unique roles at both tissue and cellular levels, indicating diverse functions and regulatory mechanisms.

To further understand their respective biological functions, genes co-expressed with GROOT1, GROOT2, and GROOT3 were studied using gene ontology (GO) enrichment analysis. This analysis aimed to uncover the biological functions and processes the co-expressed genes are involved in at cellular and tissue levels. By identifying enriched GO terms, the roles and interactions of these genes within the broader biological context can be understood, for example, how they contribute to essential processes like cell division, RNA processing, and ribosome biogenesis. This approach also provided insights into the regulatory networks and pathways these genes participate in, for example, with respect to their contributions to growth, development, and overall biomass production. Overall, the GO enrichment analysis revealed distinct functional categories, which might be connected to their association with increased biomass production (FIGS. 4F-4H). Consistent with its annotation as pseudouridine synthase family member, GROOT1 co-expressed genes are enriched for GO categories such as ribosome biogenesis, RNA processing, and RNA metabolic processes, emphasizing its role in ribosome production and RNA metabolism, which are crucial for protein synthesis and overall cellular growth (FIG. 4F). Consistent with its annotation as a cell-cycle related gene, GROOT2 co-expressed genes are enriched in GO categories associated with cell cycle, cell division, and nuclear division, indicating its involvement in regulating cell proliferation and ensuring accurate chromosome segregation during mitosis, which are processes vital for sustaining rapid growth and biomass accumulation (FIG. 4G). Consistent with some members of the radical SAM superfamily having a role in RNA processing, GROOT3 co-expressed genes are enriched in GO categories related to RNA processing, gene expression, and RNA splicing, indicating its role in gene expression and RNA processing, essential for the efficient functioning and growth of cells (FIG. 4H). These functional roles indicate that GROOT3 contributes to the regulation of gene expression, GROOT2 to cell division and growth, and GROOT1 to protein synthesis, all of which are essential processes for biomass production. Therefore, the high expression levels and functional roles of these genes likely underpin their association with increased biomass, as they collectively enhance the cellular and molecular mechanisms that drive growth and development.

### Example 5

### The Linkage Disequilibrium Pattern of GROOT Alleles Indicates a Potential for Coinheritance

The discoveries of all three GROOT genes that are involved in limiting biomass accumulation can be attributed to a single GWAS peak. It was then studied whether natural alleles of these genes are connected to one another. For this, we conducted an analysis of Linkage Disequilibrium (LD) of all three GROOT genes with the top GWAS SNP (6772287) within a 70 kb window. Overall, the LD between the top GWAS SNP and other SNPs in this region was relatively low, with less than 1.1% of SNPs exhibiting an LD r² value over 0.2, indicating a weak association with the top GWAS SNP. However, within the GROOT genes, several SNPs displaying strong LD with the top GWAS SNP were identified. Notably, a SNP (6810046) in the GROOT2 gene exhibited the highest LD (r²=0.66) with the top GWAS SNP (6772287). Similarly, a SNP (6745394) in GROOT1 displayed a high LD (r²=0.62) with the top GWAS SNP, and a SNP (6815838) in GROOT3 displayed a high LD (r²=0.48).

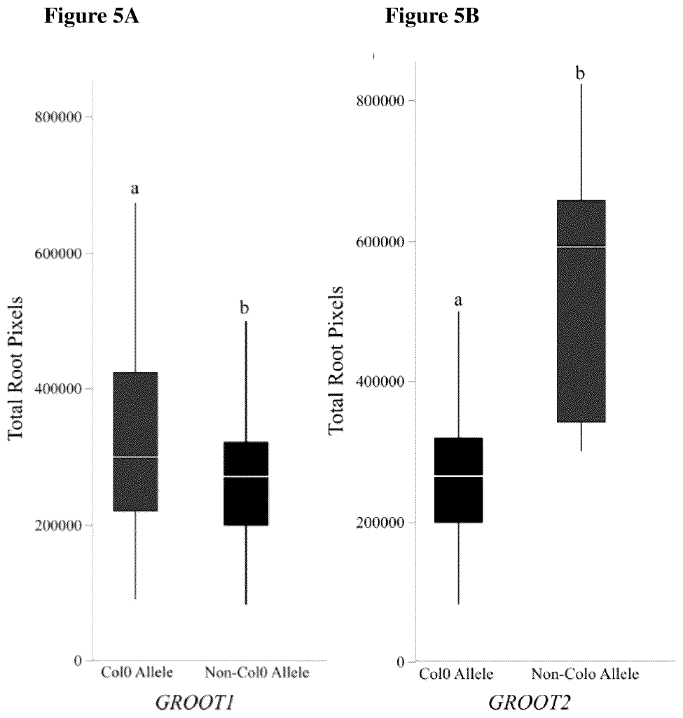

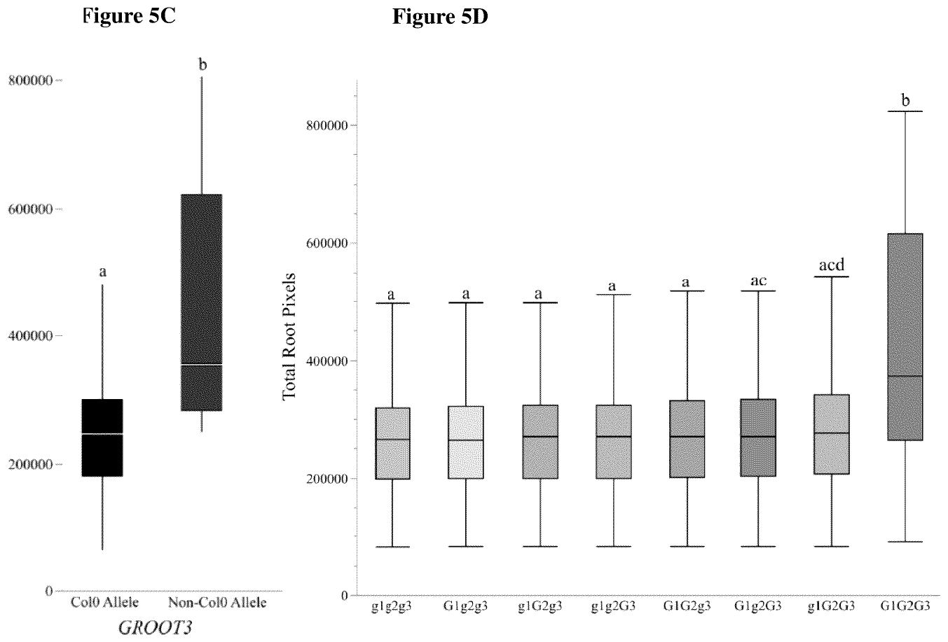

The relation of GROOT variants (as defined by their SNPs in LD with the top GWAS hit) with the root biomass trait was then investigated. The pattern of sequence polymorphism for these gene positions within the natural accessions used herein (https://tools.1001genomes.org/polymorph/) was studied. All natural accessions were grouped based on whether they had reference alleles or non-reference alleles with respect to each position, and the relationship between these SNP patterns and the biomass trait was analyzed. Accessions with reference alleles of GROOT1 were linked to higher biomass values (FIG. 5A). For both GROOT3 and GROOT2, accessions with non-reference alleles exhibited significantly higher biomass values compared to those with reference alleles (FIGS. 5B-5C). Given the distinct and potentially complementary molecular roles of the three GROOT genes, it was then determined whether a combination of biomass increasing alleles would be associated with a non-additive increase compared to single or double allele combination. Accessions were grouped according to their GROOT allele combination. Accessions with a combination of all three biomass increasing alleles showed 55.6% increase of TRP, on average, compared to accessions that did not contain any biomass increasing GROOT alleles (FIG. 5D). Accessions that only contained one or two biomass increasing GROOT alleles did not show significantly higher biomass compared to accessions containing none of the three biomass increasing GROOT alleles (FIG. 5D). These results show a synergistic effect of all three GROOT alleles in increasing root biomass.

Overall, specific SNPs within the GROOT genes exhibit strong LD with the GWAS top SNP, indicating that these loci may have been targets of positive selection. This high LD implies that allelic variants at these SNPs are co-inherited more frequently than would be expected by chance, indicating a selective sweep or historical selection pressure that has preserved these advantageous genetic combinations. Moreover, the SNPs in these genes showing high LD were also associated with high biomass production. Most of the high-biomass-producing accessions possess the SNPs in all three genes. The significant LD observed indicates that these alleles contribute to the adaptive variation in biomass accumulation, reflecting a potential role in the evolutionary fitness of the populations. This interconnectedness indicates the importance of these SNPs in shaping the genetic architecture underlying biomass production, offering insights into the evolutionary dynamics driving trait variation in these genes. These findings underscore the ability of these loci as targets for modifying biomass yield.

### Example 6

### Local Climate Dynamics Drive Interactions Between Root Growth Patterns and Genetic Variants, Shaping Plant Adaptation Strategies

Root growth rate increases with temperature (Gaillochet et al. 2020; Lee et al. 2023). Thus, the link between the allelic variants of the confirmed GWAS hits and local climate conditions was investigated. The relation was studied between local conditions, such as temperature or precipitation, and the allelic variant (SNP Chr3 at 6772287) that was associated with all three GROOT genes. Examined first was the relationship between bioclimatic variables and all studied natural accessions grouped by their allelic variants for the GWAS SNP (6772287). Several environmental factors were identified, that are significantly associated with non-reference allele variants. Accessions with non-reference alleles exhibited higher values for factors related to temperature, including mean diurnal temperature (P=0.001), isothermality (P=0.0002), and mean temperature of the driest quarter (P=0.0030). This shows a significant association between high temperatures and enhanced root biomass. These findings indicate that specific allelic variants may confer an adaptive advantage under certain climatic conditions, particularly higher temperatures, leading to increased root biomass. This adaptation could be useful for plant survival and productivity in varying environmental conditions, indicating the importance of these genetic variants in climate resilience.

Natural accessions with extreme TRP from the TRP distribution graph were selected (marked in red in FIG. 6A), with representation from both reference allele and non-reference allele groups. It was then assessed the performance of the accessions containing the reference allele (Col-0) under high temperature conditions relative to the accessions containing the non-reference allele (non-Col-0). After growing the accessions under both elevated (28° C.) and regular lab-grown temperatures (22° C.) for 21 days, the fresh weight and dry weight of root and shoot tissue were analyzed, showing variable growth patterns at elevated temperatures. For instance, BAY5-1 containing the Col-0 allele and IP-Pie-0 containing non-Col-0 allele displayed distinct growth patterns under elevated temperatures (FIG. 6B).

The effects on biomass of genotype (G), temperature (E), and their interaction (GxE) were analyzed. Within this framework, significant G indicates divergence in biomass traits between lines containing the reference allele and lines containing the non-reference allele; significant E indicates temperature-driven plasticity; significant GxE indicates variation in temperature-driven plastic responses between lines containing different alleles. While accessions containing the non-Col-0 allele showed increased root and shoot biomass compared to accessions containing the Col-0 allele, the rate of biomass increase in response to temperature also significantly differed between the two groups of accessions. The accessions containing the non-Col-0 allele showed a more pronounced growth response to elevated temperature compared to the accessions containing the Col-0 allele (FIG. 6C-6F). Significant effects of genotype (G, P<0.0001) and temperature (E, P<0.0001) were observed for both root fresh weight and dry weight. This indicates that both the genetic makeup of the plants and the environmental conditions (e.g., temperature) play roles in determining root biomass. The genotype-by-environment interaction (GxE) was non-significant for root fresh weight (P=0.6766) but significant for root dry weight (P=0.0132, FIGS. 6C-6D). This shows that the overall root dry weight, which is a more direct measure of growth, was affected by how different genotypes with varying alleles responded to different temperatures. Similar trends were noted for shoot fresh weight and dry weight. Both genotype (G) and temperature (E) had significant effects (P<0.0001) on shoot biomass (FIGS. 6E-6F). Moreover, their interaction effects were also significant for both shoot fresh weight (P=0.0091) and shoot dry weight (P=0.0420) (FIGS. 6E-6F). These findings highlight the intricate nature of genotype-by-environment (GxE) interactions and their vital role in shaping plant growth responses to varying temperatures.

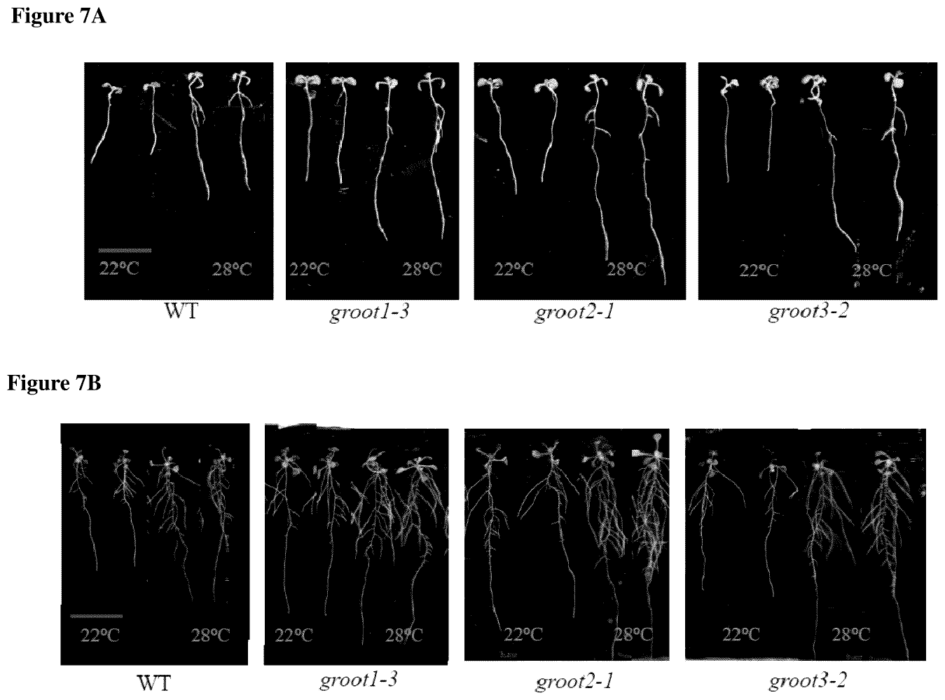

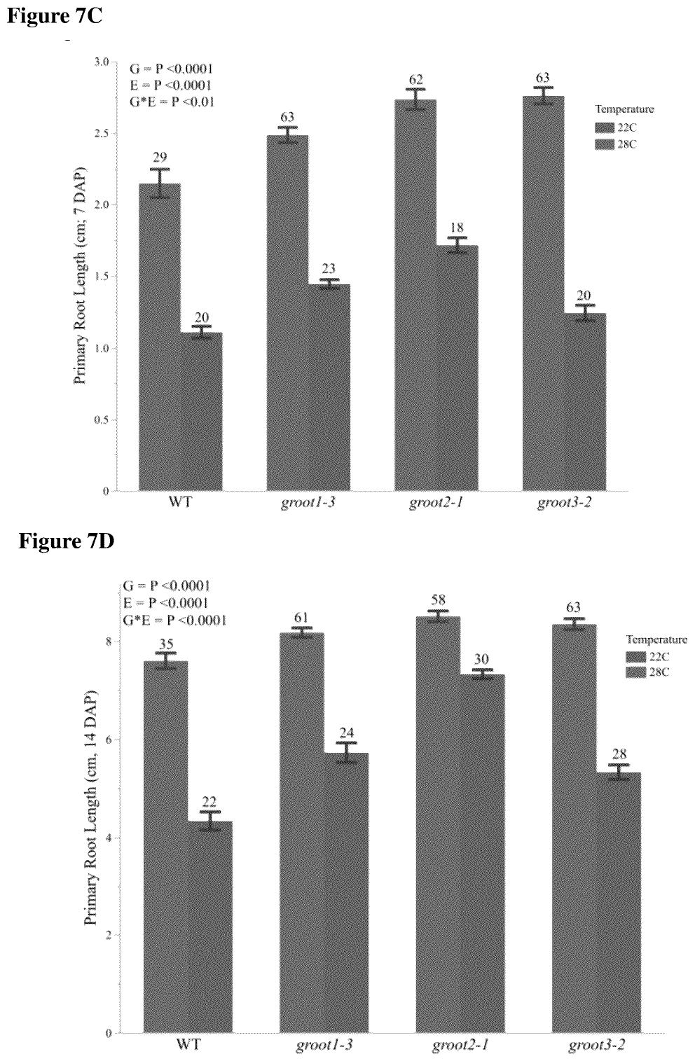

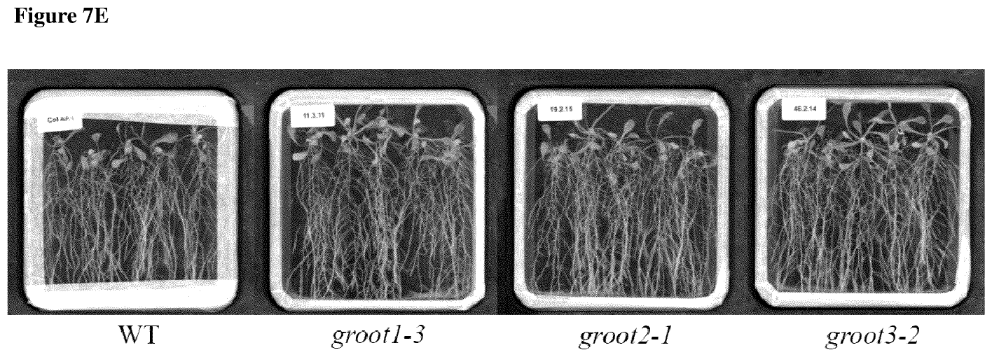

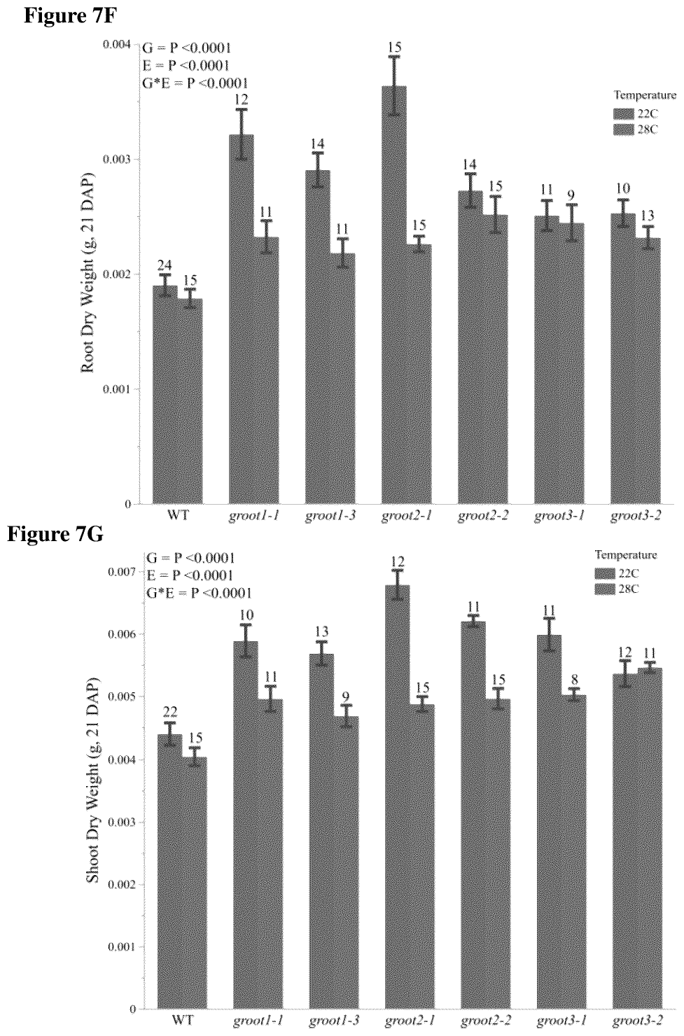

Finally, the effect of elevated temperature on the impact of the three GROOT genes was studied. Like the above experiments, data were gathered from mutant lines and WT plants grown at both 22° C. and 28° C. By day 7, genotype (G, P<0.0001), temperature (E, P<0.0001), and genotype-by-temperature interaction (GxE, P=0.0031) all showed significant effects on primary root length (cm) (FIGS. 7A and 7C). By day 14, accelerated primary root growth at the higher temperature (28° C.) compared to the lower temperature (22° C.) was observed (FIGS. 7B and 7D). Genotype (G, P<0.0001), temperature (E, P<0.0001), and their interaction (GxE, P<0.0001) all showed significant influence on primary root growth by day 14 (FIGS. 7B and 7D).

Furthermore, by day 21, genotype (G, P<0.0001), temperature (E, P<0.0001), and genotype-by-temperature interaction (GxE, P<0.0001) all showed significant effects on both root and shoot biomass (FIGS. 7E-7G). It is worth noting that reduced biomass accumulation was observed at the elevated (28° C.) temperature compared to the regular temperature (22° C.). Specifically, under the elevated temperature, all T-DNA lines, as well as the wild type (WT) plants, began flowering from day 17 on the plate. This shift towards reproductive growth post-flowering could significantly limit root and shoot biomass accumulation post day 17. These findings show that the three GROOT genes play a role in growth limitation under varying temperatures, and highlights the importance of genotype-by-environment (GxE) interactions in understanding how these genes regulate root growth, particularly in response to elevated temperatures.

### Example 7

GROOT Single Mutant Lines Display Increased Biomass Accumulation at Later Developmental Stages without Affecting Flowering Time

To evaluate whether GROOT single mutant lines display enhanced biomass accumulation at later developmental stages, a controlled greenhouse experiment was conducted using Turface growth medium. Three independent Arabidopsis thaliana mutant lines with loss-of-function alleles in the GROOT1, GROOT2, or GROOT3 gene were compared against the Col-0 wildtype control. Each genotype was replicated 15 times to ensure robust statistical power. Seeds were sown in one-gallon pots filled with Turface growth medium, and excess seedlings were thinned after germination to maintain one plant per pot. All plants were grown under uniform greenhouse conditions for a period of nine weeks.

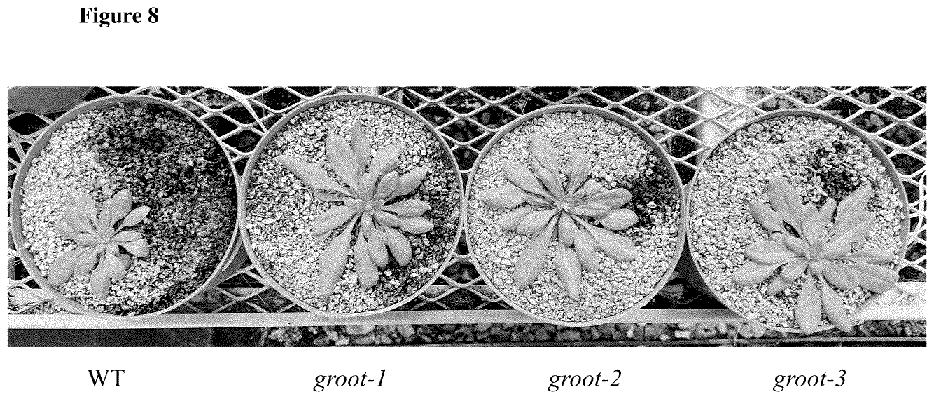

Growth differences between GROOT mutant lines and the Col-0 wildtype became visually apparent after approximately four weeks of above-ground growth in pots. Representative images in FIG. 8 show these early differences, where mutant plants exhibited more robust shoot development compared to wildtype plants.

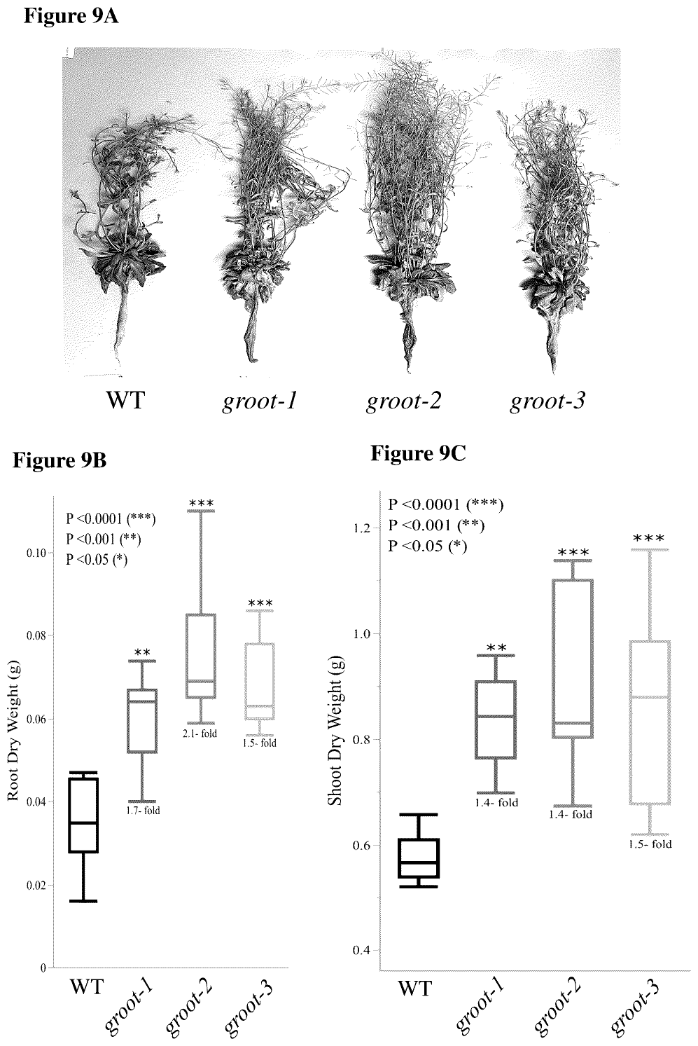

By nine weeks of growth, the differences were even more pronounced, with GROOT mutant lines showing substantially greater vegetative biomass relative to wildtype controls. FIGS. 9A-9C highlight both the visual and quantitative differences in biomass accumulation. At harvest, shoots and roots were separated, dried for four days at 50° C., and weighed to determine dry biomass. Statistical analysis confirmed that the mutants had significantly greater root and shoot dry weights compared to wildtype (FIGS. 9B and 9C).

In addition to biomass, flowering time across genotypes was assessed. No significant differences in flowering onset were detected between wildtype and GROOT mutant lines, indicating that the enhanced vegetative growth did not come at the cost of altered reproductive timing. Together, these results demonstrate that loss-of-function mutations in any of GROOT1-3 promote increased biomass accumulation without affecting flowering time.

### REFERENCES

Ackerly et al., (2000) The evolution of plant ecophysiological traits: recent advances and future directions: new research addresses natural selection, genetic constraints, and the adaptive evolution of plant ecophysiological traits. Bioscience 50 (11):979-995
Alonso-Blanco et al., (2016) 1,135 genomes reveal the global pattern of polymorphism in Arabidopsis thaliana. Cell 166 (2):481-491
Anderson et al., (2011) Evolutionary genetics of plant adaptation. Trends in Genetics 27 (7):258-266
Atwell et al., (2010) Genome-wide association study of 107 phenotypes in Arabidopsis thaliana inbred lines. Nature 465 (7298):627-631
Balasubramanian et al., (2006) Potent induction of Arabidopsis thaliana flowering by elevated growth temperature. PLoS genetics 2 (7):e106
Benjamini Y, Yekutieli D (2001) The control of the false discovery rate in multiple testing under dependency. Annals of statistics:1165-1188
Blázquez M A, Ahn J H, Weigel D (2003) A thermosensory pathway controlling flowering time in Arabidopsis thaliana. Nature genetics 33 (2):168-171
Chen et al., (2016) Drought-up-regulated TaNAC69-1 is a transcriptional repressor of TaSHY2 and TaIAA7, and enhances root length and biomass in wheat. Plant and Cell Physiology 57 (10):2076-2090
Chen W, Wu Y, Fritschi F B, Juenger T E (2021) The genetic basis of the root economics spectrum in a perennial grass. Proceedings of the National Academy of Sciences 118 (47):e2107541118
Courtois et al., (2013) Genome-wide association mapping of root traits in a japonica rice panel. PLoS One 8 (11):e78037
Dwivedi S L, Reynolds M P, Ortiz R (2021) Mitigating tradeoffs in plant breeding. Iscience 24 (9)
Fick S E, Hijmans R J (2017) WorldClim 2: new 1-km spatial resolution climate surfaces for global land areas. International journal of climatology 37 (12):4302-4315
Fitz Gerald et al., (2006) Identification of quantitative trait loci that regulate Arabidopsis root system size and plasticity. Genetics 172 (1):485-498
Fournier-Level et al., (2011) A map of local adaptation in Arabidopsis thaliana. Science 334 (6052):86-89
Gaillochet et al., (2020) HY5 and phytochrome activity modulate shoot-to-root coordination during thermomorphogenesis in Arabidopsis. Development 147 (24):dev192625
Garcia-Palacios P, Maestre F T, Milla R (2013) Community-aggregated plant traits interact with soil nutrient heterogeneity to determine ecosystem functioning. Plant and soil 364:119-129
Gedroc et al., (1996) Plasticity in root/shoot partitioning: optimal, ontogenetic, or both? Functional Ecology: 44-50
Grime J P, Pierce S (2012) The evolutionary strategies that shape ecosystems. John Wiley & Sons,
Gruber B D, Giehl R F, Friedel S, von Wirén N (2013) Plasticity of the Arabidopsis root system under nutrient deficiencies. Plant physiology 163 (1):161-179
Hancock et al., (2011) Adaptation to climate across the Arabidopsis thaliana genome. Science 334 (6052):83-86
He Z, Webster S, He S Y (2022) Growth-defense trade-offs in plants. Curr Biol 32 (12):R634-R639
Hoffman B M, Broderick W E, Broderick J B (2023) Mechanism of radical initiation in the radical SAM enzyme superfamily. Annual review of biochemistry 92:333-349
Holliday et al., (2018) Atlas of the radical SAM superfamily: divergent evolution of function using a “plug and play” domain. In: Methods in enzymology, vol 606. Elsevier, pp 1-71
Jiang et al., (2017) Transcriptional regulatory network of WOX11 is involved in the control of crown root development, cytokinin signals, and redox in rice. Journal of Experimental Botany 68 (11):2787-2798
Kang et al., (2010) Variance component model to account for sample structure in genome-wide association studies. Nature genetics 42 (4):348-354
Karlova et al., (2021) Root plasticity under abiotic stress. Plant Physiology 187 (3):1057-1070
Khandal et al., (2020) Root-specific expression of chickpea cytokinin oxidase/dehydrogenase 6 leads to enhanced root growth, drought tolerance and yield without compromising nodulation. Plant biotechnology journal 18 (11):2225-2240
Klepikova et al., (2016) A high resolution map of the Arabidopsis thaliana developmental transcriptome based on RNA-seq profiling. The Plant Journal 88 (6):1058-1070
Kriticos et al., (2012) CliMond: global high-resolution historical and future scenario climate surfaces for bioclimatic modelling. Methods in Ecology and Evolution 3 (1):53-64
Kumar R, Pandey S, Pandey A (2006) Plant roots and carbon sequestration. Current Science: 885-890
Lasky J R, Des Marais D L, McKAY J K, Richards J H, Juenger T E, Keitt T H (2012) Characterizing genomic variation of Arabidopsis thaliana: the roles of geography and climate. Molecular Ecology 21 (22):5512-5529
Lavorel S, Gamier E (2002) Predicting changes in community composition and ecosystem functioning from plant traits: revisiting the Holy Grail. Functional ecology 16 (5):545-556
Lee S et al., (2023) Nutrient levels control root growth responses to high ambient temperature in plants. bioRxiv:2023.2008. 2004.552051
Lermontova I, Fuchs J, Schubert I (2008) The Arabidopsis checkpoint protein Bub3. 1 is essential for gametophyte development. Front Biosci 13 (13):5202-5211
Lorts C M, Lasky J R (2020) Competitionx drought interactions change phenotypic plasticity and the direction of selection on Arabidopsis traits. New Phytologist 227 (4):1060-1072
Lundgren M R, Des Marais D L (2020) Life history variation as a model for understanding trade-offs in plant-environment interactions. Curr Biol 30 (4):R180-R189
Lynch J P (2022) Harnessing root architecture to address global challenges. The Plant Journal 109 (2):415-431
Maurel C, Nacry P (2020) Root architecture and hydraulics converge for acclimation to changing water availability. Nature plants 6 (7):744-749
Mayjonade et al., Adaptation to plant communities across the genome of Arabidopsis thaliana. In: 18. Congress of International-Society-for-Molecular-Plant-Microbe-Interactions (IS-MPMI), 2019. American Phytopathological Society,
Minervini et al., (2017) Phenotiki: An open software and hardware platform for affordable and easy image-based phenotyping of rosette-shaped plants. The Plant Journal 90 (1):204-216
Ogura et al., (2019) Root system depth in Arabidopsis is shaped by EXOCYST70A3 via the dynamic modulation of auxin transport. Cell 178 (2):400-412. e416
Oren R, Ellsworth D S, Johnsen K H, Phillips N, Ewers B E, Maier C, Schafer K V, McCarthy H, Hendrey G, McNulty S G (2001) Soil fertility limits carbon sequestration by forest ecosystems in a C02-enriched atmosphere. Nature 411 (6836):469-472
Panchal P, Preece C, Penuelas J, Gin J (2022) Soil carbon sequestration by root exudates. Trends in Plant Science 27 (8):749-757
Phillips P C, Arnold S J (1999) Hierarchical comparison of genetic variance-covariance matrices. I. Using the Flury hierarchy. Evolution 53 (5):1506-1515
Purcell S, Neale B, Todd-Brown K, Thomas L, Ferreira M A, Bender D, Maller J, Sklar P, De Bakker P I, Daly M J (2007) PLINK: a tool set for whole-genome association and population-based linkage analyses. The American journal of human genetics 81 (3):559-575
Ramireddy et al., (2021) Root engineering in maize by increasing cytokinin degradation causes enhanced root growth and leaf mineral enrichment. Plant Molecular Biology 106:555-567
Ristova D, Busch W (2014) Natural variation of root traits: from development to nutrient uptake. Plant Physiology 166 (2):518-527
Rockman M V (2012) The QTN program and the alleles that matter for evolution: all that's gold does not glitter. Evolution 66 (1):1-17
Ronneberger O, Fischer P, Brox T U-net: Convolutional networks for biomedical image segmentation. In: Medical image computing and computer-assisted intervention—MICCAI 2015: 18th international conference, Munich, Germany, Oct. 5-9, 2015, proceedings, part III 18, 2015. Springer, pp 234-241
Russell et al., (2008) LabelMe: a database and web-based tool for image annotation. International journal of computer vision 77:157-173
Sang P M, Lamb D, Bonner M, Schmidt S (2013) Carbon sequestration and soil fertility of tropical tree plantations and secondary forest established on degraded land. Plant and Soil 362:187-200
Savolainen O, Lascoux M, Merila J (2013) Ecological genomics of local adaptation. Nature Reviews Genetics 14 (11):807-820
Shahan et al., (2022) A single-cell Arabidopsis root atlas reveals developmental trajectories in wild-type and cell identity mutants. Developmental cell 57 (4):543-560. e549 Shorten C, Khoshgoftaar T M (2019) A survey on image data augmentation for deep learning. Journal of big data 6 (1):1-48
Slovak R, Göschl C, Su X, Shimotani K, Shiina T, Busch W (2014) A scalable open-source pipeline for large-scale root phenotyping of Arabidopsis. The Plant Cell 26 (6):2390-2403
Slovak et al., (2020) Ribosome assembly factor Adenylate Kinase 6 maintains cell proliferation and cell size homeostasis during root growth. New Phytologist 225 (5):2064-2076
Smakowska E, Kong J, Busch W, Belkhadir Y (2016) Organ-specific regulation of growth-defense tradeoffs by plants. Current opinion in plant biology 29:129-137
Smith et al., (2022) RootPainter: deep learning segmentation of biological images with corrective annotation. New Phytologist 236 (2):774-791
Smith J M (1978) Optimization theory in evolution. Annual review of ecology and systematics 9 (1):31-56
Song W, Ressl S, Tracey W D (2020) Loss of pseudouridine synthases in the RluA family causes hypersensitive nociception in Drosophila. G3: Genes, Genomes, Genetics 10 (12):4425-4438
Weigel D, Mott R (2009) The 1001 genomes project for Arabidopsis thaliana. Genome biology 10:1-5
Weigel D, Nordborg M (2005) Natural variation in Arabidopsis. How do we find the causal genes? Plant physiology 138 (2):567-568
Werner T, Motyka V, Strnad M, Schmülling T (2001) Regulation of plant growth by cytokinin. Proceedings of the National Academy of Sciences 98 (18):10487-10492
Ye et al., (2012) Mapping QTL for heat tolerance at flowering stage in rice using SNP markers. Plant Breeding 131 (1):33-41
Zhou Z, Rahman Siddiquee M M, Tajbakhsh N, Liang J Unet++: A nested u-net architecture for medical image segmentation. In: Deep Learning in Medical Image Analysis and Multimodal Learning for Clinical Decision Support: 4th International Workshop, DLMIA 2018, and 8th International Workshop, ML-CDS 2018, Held in Conjunction with MICCAI 2018, Granada, Spain, Sep. 20, 2018, Proceedings 4, 2018. Springer, pp 3-11
Zurek P R, Topp C N, Benfey P N (2015) Quantitative trait locus mapping reveals regions of the maize genome controlling root system architecture. Plant physiology 167 (4):1487-1496

We claim:

1. A method for generating a plant with increased biomass, and/or increased seed size, comprising:
reducing expression and/or activity of one or more of GROOT1, GROOT2, and GROOT3 in a plant, plant part, or plant cell, thereby generating the plant with increased biomass, and/or increased seed size.

2. The method of claim 1, wherein the reducing expression and/or activity comprises introducing one or more exogenous nucleic acid molecules into a plant, plant part, or plant cell, thereby generating a transformed plant, plant part, or plant cell,
wherein the one or more exogenous nucleic acid molecules reduce expression of one or more of GROOT1, GROOT2, and GROOT3, and/or reduce activity of one or more proteins encoded by one or more of GROOT1, GROOT2, and GROOT3.

3. The method of claim 1, wherein the biomass is below-ground biomass, above-ground biomass, or entire biomass.

4. The method of claim 1, wherein the plant with increased biomass, and/or increased seed size has increased productivity, resilience, and/or carbon sequestration capacity.

5. The method of claim 2, wherein the introducing one or more exogenous nucleic acid molecules generates one or more deletions of, or one or more loss-of-function mutations, in the one or more of GROOT1, GROOT2, and GROOT3.

6. The method of claim 2 wherein the one or more exogenous nucleic acid molecules comprise one or more guide nucleic acid molecules that can delete or mutate the one or more of GROOT1, GROOT2, and GROOT3.

7. The method of claim 2, wherein the method further comprises introducing one or more Cas proteins or one or more nucleic acid molecules encoding a Cas protein into the plant, plant cell, or plant part.

8. The method of claim 2, wherein the transformed plant, plant cell, or plant part comprises one or more deletions of, or one or more loss-of-function mutations, in the one or more of GROOT1, GROOT2, and GROOT3.

9. The method of claim 2, wherein the one or more exogenous nucleic acid molecules are one or more RNAi molecules or one or more exogenous nucleic acid molecules that generate one or more RNAi molecules, wherein the one or more RNAi molecules target the mRNAs transcribed from the one or more of GROOT1, GROOT2, and GROOT3.

10. The method of claim 2, wherein
GROOT1 comprises at least 60%, 65%, 70%, 75%, 80%, 85%, 90%, 95%, 96%, 97%, 98%, or 99%, or 100% sequence identity to any of SEQ ID NOs:1, 3-4 and 6; or encodes a coding sequence comprising at least 60%, 65%, 70%, 75%, 80%, 85%, 90%, 95%, 96%, 97%, 98%, or 99%, or 100% sequence identity to any of SEQ ID NOs: 2, 5 and 7; or encodes a protein sequence comprising at least 60%, 65%, 70%, 75%, 80%, 85%, 90%, 95%, 96%, 97%, 98%, or 99%, or 100% sequence identity to any of SEQ ID NOs: 8-13;
GROOT2 comprises at least 60%, 65%, 70%, 75%, 80%, 85%, 90%, 95%, 96%, 97%, 98%, or 99%, or 100% sequence identity to any of SEQ ID NOs: 14 and 16-17; or encodes a coding sequence comprising at least 60%, 65%, 70%, 75%, 80%, 85%, 90%, 95%, 96%, 97%, 98%, or 99%, or 100% sequence identity to any of SEQ ID NOs: 15 and 18; or encodes a protein sequence comprising at least 60%, 65%, 70%, 75%, 80%, 85%, 90%, 95%, 96%, 97%, 98%, or 99%, or 100% sequence identity to any of SEQ ID NOs: 19-22; and/or
GROOT3 comprise at least 60%, 65%, 70%, 75%, 80%, 85%, 90%, 95%, 96%, 97%, 98%, or 99%, or 100% sequence identity to any of SEQ ID NOs: 23 and 25-29; or encodes a coding sequence comprising at least 60%, 65%, 70%, 75%, 80%, 85%, 90%, 95%, 96%, 97%, 98%, or 99%, or 100% sequence identity to SEQ ID NO: 24; or encodes a protein sequence comprising at least 60%, 65%, 70%, 75%, 80%, 85%, 90%, 95%, 96%, 97%, 98%, or 99%, or 100% sequence identity to any of SEQ ID NOs: 30-35.

11. The method of claim 1, wherein the expression and/or activity of the one or more of GROOT1, GROOT2, and GROOT3 is reduced as compared to a control plant, plant cell, or plant part.

12. The method of claim 1, wherein the one or more exogenous nucleic acid molecules are operably linked to a heterologous promoter.

13. The method of claim 12, wherein the heterologous promoter drives expression of the one or more exogenous nucleic acid molecules in a plant cell.

14. The method of claim 1, wherein the plant is, or the plant cell or plant part is from a pennycress, soybean, canola, rice, wheat, corn, or sorghum plant.

15. A transformed plant made by the method of claim 1.

16. A gene-edited plant, plant part, plant cell, or seed, comprising one or more deletions of, or one or more loss-of-function mutations in one or more of GROOT1, GROOT2, and GROOT3.

17. The gene-edited plant, plant part, plant cell, or seed of claim 16, which does not comprise a transgene used to generate the one or more deletions or loss-of-function mutations.

18. The gene-edited plant, plant part, plant cell, or seed of claim 16, which is transgene-free.

19. The gene-edited plant, plant part, plant cell, or seed of claim 16, which comprises one or more transgenes.

20. The gene-edited plant, plant part, plant cell, or seed of claim 19, wherein the one or more transgenes comprise an exogenous vector, an inhibitory RNA molecule, a guide nucleic acid, a Cas gene, or combinations thereof.

21. The gene-edited plant, plant part, plant cell, or seed of claim 16, wherein:
GROOT1, prior to the one or more deletions or loss-of-function mutations, comprises at least 60%, 65%, 70%, 75%, 80%, 85%, 90%, 95%, 96%, 97%, 98%, or 99%, or 100% sequence identity to any of SEQ ID NOs:1, 3-4 and 6; or encodes a coding sequence comprising at least 60%, 65%, 70%, 75%, 80%, 85%, 90%, 95%, 96%, 97%, 98%, or 99%, or 100% sequence identity to any of SEQ ID NOs: 2, 5 and 7; or encodes a protein sequence comprising at least 60%, 65%, 70%, 75%, 80%, 85%, 90%, 95%, 96%, 97%, 98%, or 99%, or 100% sequence identity to any of SEQ ID NOs: 8-13;
GROOT2, prior to the one or more deletions or loss-of-function mutations, comprises at least 60%, 65%, 70%, 75%, 80%, 85%, 90%, 95%, 96%, 97%, 98%, or 99%, or 100% sequence identity to any of SEQ ID NOs: 14 and 16-17; or encodes a coding sequence comprising at least 60%, 65%, 70%, 75%, 80%, 85%, 90%, 95%, 96%, 97%, 98%, or 99%, or 100% sequence identity to any of SEQ ID NOs: 15 and 18; or encodes a protein sequence comprising at least 60%, 65%, 70%, 75%, 80%, 85%, 90%, 95%, 96%, 97%, 98%, or 99%, or 100% sequence identity to any of SEQ ID NOs: 19-22; and/or
GROOT3, prior to the one or more deletions or loss-of-function mutations, comprise at least 60%, 65%, 70%, 75%, 80%, 85%, 90%, 95%, 96%, 97%, 98%, or 99%, or 100% sequence identity to any of SEQ ID NOs: 23 and 25-29; or encodes a coding sequence comprising at least 60%, 65%, 70%, 75%, 80%, 85%, 90%, 95%, 96%, 97%, 98%, or 99%, or 100% sequence identity to SEQ ID NO: 24; or encodes a protein sequence comprising at least 60%, 65%, 70%, 75%, 80%, 85%, 90%, 95%, 96%, 97%, 98%, or 99%, or 100% sequence identity to any of SEQ ID NOs: 30-35.

22. The gene-edited plant, plant part, plant cell, or seed of claim 16, wherein the plant is, or the plant cell, plant part, or seed is from a pennycress, soybean, canola, rice, wheat, corn, or sorghum plant; or the plant cell, plant part, or seed is from a pennycress, soybean, canola, rice, wheat, corn, or sorghum plant.

23. The gene-edited plant or seed of claim 16, wherein biomass, and/or seed size of the plant, or seed size of the seed is increased as compared to a control plant or control seed.

24. The gene-edited plant of claim 23, wherein the biomass is below-ground biomass, above-ground biomass, or entire biomass.

25. A ribonucleoprotein complex, comprising:
an isolated Cas9 protein; and
a gRNA comprising any one of SEQ ID NOs: 39-50.

26. A method of producing a commodity plant product, comprising collecting or producing the commodity plant product from the gene-edited plant, plant part, plant cell, or seed of claim 16.

27. A method of producing plant seed, comprising crossing the gene-edited plant of claim 16 with itself or a second plant.

28. A method for breeding a plant with increased biomass, and/or increased seed size, comprising:
crossing the gene-edited plant of claim 16 with a second plant;
obtaining seeds from the crossing;
planting the seeds and growing the seeds to progeny plants; and
selecting from the progeny plants those with increased biomass, and/or increased seed size when compared to a control plant.


SEQUENCE LISTING (51 sequences)

SEQ ID NO: 1 - DNA, 2604 bp, Arabidopsis thaliana
  source 1..2604: mol_type = genomic DNA
aatgactatcggttccatctttcccaagttcctctgctccaaaacctcttaaaccctaattcgaatttagcctaaaccctaaaatggccaaatggagacttgcgactgcgacgctccgtcgacaacttcaatcctcatcgccaactatctctaccttcaagaatccaaccaaagcattgtctgcggcggctcatcagtctactcgcagctacagtacaactcagaccgatgattcgagagggaaatggttaactttacctcctttttctcccaccatcgatggcaccgccgttggaaaggatctcctttccgacggagactctgtcaaatcttcaacggacaactcaaaaacgacggcgcttaggtggattcttcgttgtcgtcccgatttacccagaactctcgttcagaaactcttccgcttaagacaggttcctagtctcttccaatcttgtattcaacgaattgttgtcagtttcttacattgtaattgattcaggttagaagagaaatgtctatgagtgttgatggtgatgaactacaaagaagccagcttaaaagggtaatgttattttgttatctattgattggaacaagaactgttttgttgaaaggctgctcttttatctgatgatgttgatttctgacccttgaaaggtggcagctaaggagtccttgaatgtaggagatagaatttaccttcctttatcggttgacaatgatacgccgcagacgccacctgctaagaaagaaagctttcaatgtagtgatgaagaacgcaaattcgtttgcagtttggtgttgtataaggttttgaaactgtatactattggtttgcttttttgtttttttattgatgttatatacttatatagattgatagatcttaactgtgtgttaatgtgccaggatccagccattattgttttgaataaacctcatggtttggctgttcaagtgagtcatttatacttcgcattctgttgtattgtgtatcatattcttggagcttgtgaaatgtgatgacatttgctctttaatagggtgggagtgggatcaaaaccagtattgatgaactcgctgcctcttgcttgaaatttgataaatcagaatctccccggctggttagttgcgaatcatagtaaagcactgattaaaactgtatctaaacgaaagtgtatgatttattatgttgtttttgttaatttgaaaagtatgaatctattggcttatgcaagtatgggatatgtattaggtgcacagacttgacagagactgtagtggacttttggtgttggcaagaacacagacggctgcaacagttcttcattccatattccgagagaaaacaactggtgcatccgcatatgtaaatcctctgctttctctccgatctttccattgcttgtcatggatcgtgggaaataaacataagtttctcttccaatgacttttattttacttggcagggtgtcaaaaagaacgtaaaatccttgaaaagaaaatatatggcacttgtgatcgggtgtccaccacgtcaaaggggacagatttcagcgccactcagaaaggtcttcacactctgttttagctagagagttttatccatctgagtttttagtctattttgttttatctaggagttgctttgtttgttcgaattcggtcattgcttttgctgctttactggagtcaaatttgaaggtaaaatatatgttaaatatctgggtaggtggttgtggatgatggaaaatctgaacgtatcactgttaatgacaatggagaactcgtttctactcagcatgctatcaccgaataccgagtgattgaatcttcaccacatggttagtgagactgacttccatttctattcagttaaacttaaagcaaatgattttgccttgagtttttagcacattgttgaattgcaggatacacatggcttgagcttcgccctttaaccgggagaaaacatcaggtctctatagatattcagtttttgtttcaactttctctcttttttatgttctcttaatactaatctgttttcaactgttcttcgattgccacagcttcgtgtacactgcgctgaagtgctaggaacaccgatagtcggggactacaaatacggttggcaagctcataaagcccgggaaccttttgtctcttctgaaaacaacccaaccaagcaatcatcatctccttttggattggatctggatggtggagatgtctcttcgaaacagccacaccttcatctccattcaaagcaaatcgatctgccaaacatatcacagctcttggagaaaatgcaggtctcttcagactctgatatttcggatctcgatagccttaaattcgatgctccattgcctagtcatatgcaactaagctttaatttgttgaaatctagagtcgaaacttgtgacaaaaattagattttttttcttaccgagctttcttctttgtgttcattgaggcccaagtatttgtgtatttggacctgaatattctcatacaaagataaataattataattaaatgatttttcgcatataatcattattgtggtatgattaacacagttggtgtgatgactgattg

SEQ ID NO: 2 - DNA, 1434 bp, Arabidopsis thaliana
  source 1..1434: mol_type = other DNA
atggccaaatggagacttgcgactgcgacgctccgtcgacaacttcaatcctcatcgccaactatctctaccttcaagaatccaaccaaagcattgtctgcggcggctcatcagtctactcgcagctacagtacaactcagaccgatgattcgagagggaaatggttaactttacctcctttttctcccaccatcgatggcaccgccgttggaaaggatctcctttccgacggagactctgtcaaatcttcaacggacaactcaaaaacgacggcgcttaggtggattcttcgttgtcgtcccgatttacccagaactctcgttcagaaactcttccgcttaagacaggttagaagagaaatgtctatgagtgttgatggtgatgaactacaaagaagccagcttaaaagggtggcagctaaggagtccttgaatgtaggagatagaatttaccttcctttatcggttgacaatgatacgccgcagacgccacctgctaagaaagaaagctttcaatgtagtgatgaagaacgcaaattcgtttgcagtttggtgttgtataaggatccagccattattgttttgaataaacctcatggtttggctgttcaaggtgggagtgggatcaaaaccagtattgatgaactcgctgcctcttgcttgaaatttgataaatcagaatctccccggctggtgcacagacttgacagagactgtagtggacttttggtgttggcaagaacacagacggctgcaacagttcttcattccatattccgagagaaaacaactggtgcatccgcatatggtgtcaaaaagaacgtaaaatccttgaaaagaaaatatatggcacttgtgatcgggtgtccaccacgtcaaaggggacagatttcagcgccactcagaaaggtggttgtggatgatggaaaatctgaacgtatcactgttaatgacaatggagaactcgtttctactcagcatgctatcaccgaataccgagtgattgaatcttcaccacatggatacacatggcttgagcttcgccctttaaccgggagaaaacatcagcttcgtgtacactgcgctgaagtgctaggaacaccgatagtcggggactacaaatacggttggcaagctcataaagcccgggaaccttttgtctcttctgaaaacaacccaaccaagcaatcatcatctccttttggattggatctggatggtggagatgtctcttcgaaacagccacaccttcatctccattcaaagcaaatcgatctgccaaacatatcacagctcttggagaaaatgcaggtctcttcagactctgatatttcggatctcgatagccttaaattcgatgctccattgcctagtcatatgcaactaagctttaatttgttgaaatctagagtcgaaacttgtgacaaaaattag

SEQ ID NO: 3 - DNA, 3421 bp, Glycine max
  source 1..3421: mol_type = genomic DNA
aaaaaaacacgaatgggggtggtaaattgaattgggtgagatgagggcggcacaatccatgatgttgagagcgttgaggagtgggcagcgtcagttctcggtggcggtgacaaggccgtgggaggacaaatggcttactctgcctcccgtcagtgcgagttcgagtgcgagtgtggagctgaatcaactctcgtccactccgaccaccgcactcaaatgggttgttcggtgctgcccccaccttcccagagctctggtgcacaagcttttccgtctaagacaggttcgaattcaccctgccactgttatacaacaacaaacattcaaaagggtaagggtatgtgtatgacttttctccctctcctcctcctccctcttcttctaaactaactcattaatggaaggtggcagccaaggacaccttgaacacaggagaccgtatcttacttcctcaatctgttaaagttaaacaaacgcctacacattctcatctcactccccaacaaatcaactttatccgtactcttgttatctataaggttttcaacttttactcccttcttttttctttccgcatttctctttctcattctcattttgtgcttaggatcccgccattctcgtcctcaacaaacctccaggaatgccagtgcaggtacctcatctcaatgtccccattcttattctcggatgctcttccattcattaattccttctcctgctttctaattaattagggtggcattaatatcaaacggagtttagatgctgtagctgctgcatctttaaattatggttactctcaaccccctcgtctggtgagctcattattcacattcactatctcatcttttagttctgtgccaatatgattcgtagtttattgtttttaggtgcatagactagacagagactgttctggcattctggtcatgggaaggacacacaccagtacaacagtcctgcattccatcttccgcgagaaaacttccagggcgtcagatgatgtgagtgataaagcattcagatcattagtctattatggtcacattctcatattgctatatccatttcattgtttttcttgatttggtgccgttagattggcaaagagaagagaatactacaaagaaggtactgggcactggtccttggatgtcctagacgtccaaaggggttggtcactgcttcactgggtaaggttgtatccatattggcacttgttaactatatttaccagaaaatttcagttggttaatcctccctactcctggcatattatacattgtgcatgcttcatccatttcatactcacaaatgtcaaacttataaaacagcaattatttgtatgtaatgtctgcataaatgaaaaaaggtttgcactgaatcatagctttttcttaaaactaaaccatctaacattttaaacaaccaatggtatatgctgtcagtaatcactaaatttaccatgttcttcactatttaaaaaaaagtttgaacaattatattactcagatggtggggaatgttatgtgtggggaatttcccttagtcctttaatactttgatgtttcttctgggtagctagtcttctgataaatccttttttcagttatggtgtttcctttaatgctttacatgtcttgtgaaccaacaaaacaagttcaaaacaaacgtgttagtatccataagatgcagcctcatacacacgaattgccaccaactctttggaattgcgtgacatgaatgcttaggccttacagttacaatataatagcccataacctaaattagcttccagcattaaagtaaatggacattgatttgaggggctttcctcatttattgcaaaataatgttgtaaacttgtagttctttatttgatttaaggtggtggttgacaatgggagatctgatcgaataactatcgttgacaattctacattaatgtcatcacagcatgcaattacagagtaccgagtgattgcatcatcatcacaaggttggtaatgaaacttttaattctaagattaaatctgatcaaatatcttttgagaatgttaagaataaatttcattattttccccattttgcattttattttgtgacattttgaacttattaggaccctagtttcaaccactctcactcacactaattgttaataggatggagtcattaaatgtcattagtgttctgttctagaagcaatatatcaccactgcttataaatttattgcaatgtcaaaaaattcatgcttccaaactatgttaggttacaaattgttttattaaataatgtagttcaacattttcacttgatactggaaaagctgagagtcctagacaaaaagagctaatagttacatatcaagtatgacatcgacttagtttcatgtttgggaaatagaattttacagtctgccaaacagcataagtactcctagcagctggaagatgtctgatttttatcacaacagggcctgtttgcaatacgtttgtgtagaaaatttgtcatgtgtcttttttctccctctttggcatgagttatcctgcaggtgcaaagttcttcccctcagctcttcttaattggcaacccctacttatgtcatctcaaatagtgcaggttacacctggttggagctgacccctttaactggtagaaaacaccaggtaacaagatgatatttgtgtcacaacttaaaatgttaaatcatttcatatgcctcaccatatttctattctaactgcgttgtactggtaccacagcttcgagtccactgtgctgaggtgttaggtacaccaatagtaggagactacaagtatggatggcaagctcacaggaagtggggacattttgatttgcctaatgtggaggactcacgtgaagaacttctcaatgaagaaaaactcccctttggccttaatttgaataaagggagcatctctgagaatcatcctcgtttacatcttcattgcaagcaaatggtcttgcctaatatatctcaagcactgcagaatgtgcaatcagcttccagttgtgatctttcactagttgaagagcttgagttggtagcggatttgcctccatacatgcaaagaagttgggatgtcacaaattattgagttaatgtcatcaaaggcaatttcttccgctcggatttgcctctgtactttactttttctatataaaaaatgaattaaataaattattattaaatattccatatattactatagctctcagtattacatttgttaacgatatcgttcctgatatattggaaactaaaggttcatgtgatttgtttatggactgtacttttttttatgatcattgactgtatttagaattaacggacatgatcaaattaatagtattgaaattatttttcttaatgaaaaagctatagatattttg

SEQ ID NO: 4 - DNA, 2366 bp, Thlaspi arvense
  source 1..2366: mol_type = genomic DNA
atggccaaatggcgacttgcgaccgcgacgctccgtcgccatctccgatcaccatcgcctactatctcttccgtcttcagggatccgaccggagccttgtctgcagctaatcagcgtcgtaggtacaatacacctgaagatccgagagggaaatggctaactctacctcctttttctcccaccgtcgatgccgccgcaatcggaaaggagctatcttccgacagagactccgcaaaaggttcaacggatggctcaacgacggcgattaggtggattcttcgttgccgtcccgatctacctaggaatctcgttcagaaactctttcgtctaagacaggtcctctactctcttcatcatcagccaagagtgtatttcaaaattgaatccaatccctcctcaatggtattgttgttgtcaacttcttacattgtaaatggattcaggttagaagagaaatgtctctgagctgtgatggtgatgagctacaaagaagccaacttaaaagggtaaataatgttcgtctattgatttgaaacaagaaccctttttttagaagctgctgttatctgatgttcttgattctgattctgaccattgaaggtgtcagcaaaggaacccttaaacttaggtgatcggatttaccttcctatatcggtagacaacgatgcgccgccgcagcctgctaagaaagaaagctttcgttgcagtgaagaagagcgcaaattcgtttgcagtttggtgttgtacaaggtttttaagactcttcacacatggcgatgttgttaatattgattggtagatctaaactgtgagttaatattgctccaggatccagccattatcgttttgaataaacctcatggcttggctgttcaagtgagttcatttatactttcttgcattgtaaattgatcatggtcttagagcttgtgacagaaatgattagacttttttgctcttaaacagggtgggactgggatcaaaaccagtatcgatgaactcgctgccacttgcttgacttttgataaatcagaatctcctcggctggttagtagagaatcatagtatgatttaattaagactttattttttgttaaaactagttaatttgaaaaagtatgaatcttttggctaatgcaagtatggtgtatgtataattaggtgcacaggcttgacagagactgtagtggacttttggtgttgggaagaacacaaacggctgcaacagttcttcattctctattccgcgagaaaacatctggtgcatccgcatatgtaaaatcctctcattactctcacgcttgtttcttacttgagatgtttgcttgtcatgattcaagagaaatcttctctccgagtgacttctttttttttatctctttagggtgtaaaaaagaacataaaatccctgaaacgaaaatatttggcactcgtgatcgggtttccaagacgtcaacgtggacagatttcagcgccactcagaaaggtcagaaagcttcaccaatctgagttctagtccatttggttcgatctaagacttgatttgtttgtttacattcagtccattgcttttgctgctttaacatatgtgtgaatgtctgagtaggttgttgtggatgatggaaaatctgatcgtatcacggttaatgacaatggagaactcgtttctactcagcatgctatcactgaataccgagtgattgaatcttctccacatggttagtgtgacaaatgacttcgtttttttcttttcagtcaaaacttaaatcaaacgatttggccttgagtagcacattgttgatttgtaggatacacatggcttgagcttcgtcctttaaccgggagaaaacatcaggtctctataaaaatcttcagtttttttgtttcaactcttgctctgttttctcttctcttatgagttataacaatgttttgtccactgtcttcgattgcgacagcttcgtgtacactgcgcagaagttctaggaacaccgatactcggagactataaatacggttggcaagctcataaagcgcgggaaccatttgtctcatccggaaacagtaacaccccaaccaagccgtcattgtctctttttggcttggatctggatggtggcgatgtgtcttcaaagcagccacaccttcatctccattcgaagcagattgatctgccaaacatctctcagctcttggaaaagttgcaggtttcttcagattctgatatttcggatctcggtggcctcaagttcgatgctccgttgcctgctcatatgcagctaagctttaacttgttaaaatctagagtcgaaacttgtgatattgctaagcccgagttggtatctttaacagatagtaattgttag

SEQ ID NO: 5 - DNA, 1413 bp, Sorghum bicolor
  source 1..1413: mol_type = other DNA
atggcgatcaggacggcagcgctcctcttccgccgccgggccgtcgccgccgccgccgccgccgcagctccaaagccgctacaacagtacttcgctggtctgtccagcgcagtggggcacgttactctagacgatggcggtcgcattggcggtggggaggagaacaagaagcggtgggtggagctccctcccttcgcaccactcgacgccaacgccgccgctcgagccatctatcggggagataatggggaaggttcacgctccaattccacggctatcaggtgggtccggcgctgctgcccacacctgccggcgtcgctcgtgcagaagctgttccgcctccgcaaggtgaagaagaatcttgtgactgctgatacctcttcaacagacagtattgcccagcaactccggctgagaagggtttcagcaaaagatgaacttgtgcctggtgatattctctttctacctgataacattcaagaatcttctgttactgagaagaagaaatttggtaacaagaatgagattgattttctacgcagccttgagatctacaaggatagggccatcatcgtgctcaacaaaccacctggaatgccagtgcaaggtggtgttggcataaaaaatagtattgatatactggccccaatgtttgaggacggttcttctgaagcacctcggctggtccacaggcttgatagggattgcagtggcgtcctagttctgggaagaactcaacttagcacttccattatgcatgctatatttcgtgagaaaactgctgatgctttaggtgatggtactcaacaagtactgcaaagaaaatatgttgcacttgttattggaagacctaggcatcccaagggtttattgtcagctccacttgcaaaggttgtattacaagatggcaggtcggagcgtctgactgtttgcgctggtccaaataccgcttctgttcaagatgctttgacagagtaccgtgtgattgagtcttgtcctcaagggtacacttggttagaattattccctcggactgggaggaagcatcagctccgagtccattgtgcggaggttctgggaacaccaatcgttggggattacaagtacggacggcaagcacaccagaactggacgcctcttcccatgccgcaaacaatcgacgaggaaatgctcaagaaaaggaagcttccctttgggcttgctttgggcggtggaagcgtagctgagcagcagccacagctgcatctacactgcaagcagatgatccttcctgacatctcagcagctatgcagcagctgcagtcttcagatgctgaccacaatttctctgatctggagaagctgagctttgtcgccccattgccatcgcacatgcggttgagctggaggattctgatgtccataggcaagtaa

SEQ ID NO: 6 - DNA, 2229 bp, Brassica napus
  source 1..2229: mol_type = genomic DNA
atggcggccaattggagagttgcgaccgcggccctccgtcgtcatctcctatccccaccgcctattatctttgccgccttcagggagtccaccggagccttatccgcggctaatcagcgtcgtagctacacgacagctctcgctgatgatgatccgagagggaaatggctcacgctacctcctttctctcccaccatcgacgccgccgcggtaggaaaggagctctctttcgacgacggagactctgtcgtcaaaggctcaaatgatggctcaacgacggcgctgaggtggattcttcgttgccgtcctgacctacctagaaacctcgtacagaaactctttcgtttaagacaggtccgtgtaaagattgtatttttttgatgattgaagttacaggggataagatatttgtaaatgttttcaggttagaagacaagtagtagtagtagtgcctatgagttgtgaactacaaagaagccaacttaaaagggtaaattgttcttctgtctatctcttggtgctgttttttttttttttttttttttttttttgagaagctgatgctgtctgatggtactgattgaaggtggcagccaaggagtcattgaatgtgggagatagaatttaccttcctctatctgttggcaatgatgctccccagcctgctaagaaagaacgctttctttgcagtgaagaagagcgcaaattcgtttgcagtttagtgttgtacaaggtttgtttgtttgttttttttttgcaagaaaatgttatatagattgatagatttgtaactgtgggttaatttgccaggatccagccattattgtcttgaataaacctcatggtatggctgttcaagtaagtcatatattctccatgctttctgatgcatcgtattcgtggagctgatagactttttctcttaaacagggagggactggggtgaaaaccagcattgatgaactcgctgccacttgtttgacttttgataagtcagaatctccccggctggttagtagcgaaatcatatagtagtgcaccaaggctttctgttaagtcctagcttatatggaagaagtttatgaatctcttggcttatgcaagtatggtggtgtatgtattaggtgcacagacttgacagagactgtagtgggcttttggtgttgggaagaacacaaacagctgcaacacttcttcattctatattccgcgagaaaacatctggtgcttccgcctatgtaagtcatatgctttctcttagaagtttttcttactttgaaataagttttcttgtcatatgatttctgagaaatcgtaaaaaaatgtctctttcggtgacctttcctttatttctttagggtgtcaagaagaacataaaatccttgaaaagaaaatatctggcactcgtgatcgggtgcccaagacgtcaaaggggacagatctcagcgccactcagaaaggtgattttacactctgttttagctatcagaatgtttcacccgtctaagttttgtccactttggttcgatccaagagatgattagtctgtgcacattcaatccattgcttatgttgttttactggagtaaaattctttgctaaaacatatgtggaatgtctgtgtagcttgttgtggatgatggaaaatctgatcgcatcacagttaatgacaatggagaactagtttccactcagcacgctatcactgaataccgagtggtcgaatcttcaccacatggttagtgtacactgttgctatcgtttcacttaacttagaatcaaatggtttggccttttatagcatactgttgctttataggatacacatggctggagcttcgtcctttaaccggcagaaaacatcagctccgtgtacactgcgcagaagtgctaggaacaccgatactcggggactataaatacggttggcaagctcataaaaccagggaaccttttgtgtctccagaaaacacccggaccaagacatcatctccttttggcctcgatatggaaggtggagatgtatcttcaaaacagccacaccttcatctccattctaagcaaatcgatctgccaaacatctgtcagctcttggagaaattggaggtttcttccgactctgatatctcggatctcgatagccttaaattcgatgctccgttgcctactcatatgcagctaagcttcaacttgttgaaatctagagtcgaaacttgtgactattgttag

SEQ ID NO: 7 - DNA, 1425 bp, Brassica napus
  source 1..1425: mol_type = other DNA
atggcggccaattggagagttgcgaccgcggccctccgtcgtcatctccgatccccaccgcctactatcagggagtccaccggagccttatccgcggctaatcagcgtcgtagctacacgacagctctcgctgacgatgatgatgatccgagagggaaatggctcactctacctcctttctctcccaccatcgacgccgccgcggtaggaaaggagctctctttcggcgacggagactccatcgtcaaaggctcaactgatggctcaacgacggcgctgaggtggattcttcgttgccgccctgacctacctagaaacctcgtacagaaactctttcgtttaagacaggttagaagacaagtagtagtgcctatgagttgtgaactacaaagaagccaacttaaaagggtggcagctaaggagtccttgaatgtaggagatagaatttaccttcctttgtctgtaggcaatgatgcgccgcctcctgctaagaaagaacgctttcgttgcagtgaagaagagcgcaaattcgtttgcagtttggtcttgtacaaggatccagccattattgtcttgaataaacctcatggtatggctgttcaaggagggactggggtgaaaactagcatcgatgaactcgctgccacttgtttgacttttgataagtcagagtctccccgactggtgcacagacttgacagagactgtagtgggcttttggtgttgggaagaacacaaacagctgcaacgcttcttcattctatattccgcgagaaaacatctggtgcttccgcctatggtgtcaagaagaacataaaatccttgaaaagaaaatatctggcactcgtgatcgggtgcccaagacgtcaaaggggacagatctcagcgccactcagaaaggttgttgtggatgatggaaaatctgatcgtatcacagttaatgacaacggagaactcgtttccactcagcatgctatcaccgaataccgagtggtcgaatcttcaccacatggatacacatggctggagcttcgtcctttaaccggcagaaaacatcagctccgtgtacactgcgcagaagtgctaggaacaccgatactcggggactataaatacggttggcaagctcataaaaccagggaaccttttgtctcttctgaaaacaccccgaccaagccatcaccgtctccttttggtctggatatggaaggtggagatgtatcttcaaaacagccacaccttcatctccattctaagcaaatcgatctgccaaacatctgtcagctcttggagaaattggaggtttctccggactctgatatctcggatctcgatggccttaaattcgatgctccgttgcctactcatatgcagctaagcttcaacttgttgaaatctagagtcgaaagtagtgacaattgttag

SEQ ID NO: 8 - protein, 477 aa, Arabidopsis thaliana
MAKWRLATATLRRQLQSSSPTISTFKNPTKALSAAAHQSTRSYSTTQTDDSRGKWLTLPPFSPTIDGTAVGKDLLSDGDSVKSSTDNSKTTALRWILRCRPDLPRTLVQKLFRLRQVRREMSMSVDGDELQRSQLKRVAAKESLNVGDRIYLPLSVDNDTPQTPPAKKESFQCSDEERKFVCSLVLYKDPAIIVLNKPHGLAVQGGSGIKTSIDELAASCLKFDKSESPRLVHRLDRDCSGLLVLARTQTAATVLHSIFREKTTGASAYGVKKNVKSLKRKYMALVIGCPPRQRGQISAPLRKVVVDDGKSERITVNDNGELVSTQHAITEYRVIESSPHGYTWLELRPLTGRKHQLRVHCAEVLGTPIVGDYKYGWQAHKAREPFVSSENNPTKQSSSPFGLDLDGGDVSSKQPHLHLHSKQIDLPNISQLLEKMQVSSDSDISDLDSLKFDAPLPSHMQLSFNLLKSRVETCDKN

SEQ ID NO: 9 - protein, 429 aa, Glycine max
MRAAQSMMLRALRSGQRQFSVAVTRPWEDKWLTLPPVSASSSASVELNQLSSTPTTALKWVVRCCPHLPRALVHKLFRLRQVRIHPATVIQQQTFKRVRVAAKDTLNTGDRILLPQSVKVKQTPTHSHLTPQQINFIRTLVIYKDPAILVLNKPPGMPVQGGINIKRSLDAVAAASLNYGYSQPPRLVHRLDRDCSGILVMGRTHTSTTVLHSIFREKTSRASDDIGKEKRILQRRYWALVLGCPRRPKGLVTASLGKVVVDNGRSDRITIVDNSTLMSSQHAITEYRVIASSSQGYTWLELTPLTGRKHQLRVHCAEVLGTPIVGDYKYGWQAHRKWGHFDLPNVEDSREELLNEEKLPFGLNLNKGSISENHPRLHLHCKQMVLPNISQALQNVQSASSCDLSLVEELELVADLPPYMQRSWDVTNY

SEQ ID NO: 10 - protein, 486 aa, Thlaspi arvense
MAKWRLATATLRRHLRSPSPTISSVFRDPTGALSAANQRRRYNTPEDPRGKWLTLPPFSPTVDAAAIGKELSSDRDSAKGSTDGSTTAIRWILRCRPDLPRNLVQKLFRLRQVRREMSLSCDGDELQRSQLKRVSAKEPLNLGDRIYLPISVDNDAPPQPAKKESFRCSEEERKFVCSLVLYKDPAIIVLNKPHGLAVQGGTGIKTSIDELAATCLTFDKSESPRLVHRLDRDCSGLLVLGRTQTAATVLHSLFREKTSGASAYGVKKNIKSLKRKYLALVIGFPRRQRGQISAPLRKVVVDDGKSDRITVNDNGELVSTQHAITEYRVIESSPHGYTWLELRPLTGRKHQLRVHCAEVLGTPILGDYKYGWQAHKAREPFVSSGNSNTPTKPSLSLFGLDLDGGDVSSKQPHLHLHSKQIDLPNISQLLEKLQVSSDSDISDLGGLKFDAPLPAHMQLSFNLLKSRVETCDIAKPELVSLTDSNC

SEQ ID NO: 11 - protein, 470 aa, Sorghum bicolor
MAIRTAALLFRRRAVAAAAAAAAPKPLQQYFAGLSSAVGHVTLDDGGRIGGGEENKKRWVELPPFAPLDANAAARAIYRGDNGEGSRSNSTAIRWVRRCCPHLPASLVQKLFRLRKVKKNLVTADTSSTDSIAQQLRLRRVSAKDELVPGDILFLPDNIQESSVTEKKKFGNKNEIDFLRSLEIYKDRAIIVLNKPPGMPVQGGVGIKNSIDILAPMFEDGSSEAPRLVHRLDRDCSGVLVLGRTQLSTSIMHAIFREKTADALGDGTQQVLQRKYVALVIGRPRHPKGLLSAPLAKVVLQDGRSERLTVCAGPNTASVQDALTEYRVIESCPQGYTWLELFPRTGRKHQLRVHCAEVLGTPIVGDYKYGRQAHQNWTPLPMPQTIDEEMLKKRKLPFGLALGGGSVAEQQPQLHLHCKQMILPDISAAMQQLQSSDADHNFSDLEKLSFVAPLPSHMRLSWRILMSIGK

SEQ ID NO: 12 - protein, 477 aa, Brassica napus
MAANWRVATAALRRHLLSPPPIIFAAFRESTGALSAANQRRSYTTALADDDPRGKWLTLPPFSPTIDAAAVGKELSFDDGDSVVKGSNDGSTTALRWILRCRPDLPRNLVQKLFRLRQVRRQVVVVVPMSCELQRSQLKRVAAKESLNVGDRIYLPLSVGNDAPQPAKKERFLCSEEERKFVCSLVLYKDPAIIVLNKPHGMAVQGGTGVKTSIDELAATCLTFDKSESPRLVHRLDRDCSGLLVLGRTQTAATLLHSIFREKTSGASAYGVKKNIKSLKRKYLALVIGCPRRQRGQISAPLRKLVVDDGKSDRITVNDNGELVSTQHAITEYRVVESSPHGYTWLELRPLTGRKHQLRVHCAEVLGTPILGDYKYGWQAHKTREPFVSPENTRTKTSSPFGLDMEGGDVSSKQPHLHLHSKQIDLPNICQLLEKLEVSSDSDISDLDSLKFDAPLPTHMQLSFNLLKSRVETCDYC

SEQ ID NO: 13 - protein, 474 aa, Brassica napus
MAANWRVATAALRRHLRSPPPTIRESTGALSAANQRRSYTTALADDDDDPRGKWLTLPPFSPTIDAAAVGKELSFGDGDSIVKGSTDGSTTALRWILRCRPDLPRNLVQKLFRLRQVRRQVVVPMSCELQRSQLKRVAAKESLNVGDRIYLPLSVGNDAPPPAKKERFRCSEEERKFVCSLVLYKDPAIIVLNKPHGMAVQGGTGVKTSIDELAATCLTFDKSESPRLVHRLDRDCSGLLVLGRTQTAATLLHSIFREKTSGASAYGVKKNIKSLKRKYLALVIGCPRRQRGQISAPLRKVVVDDGKSDRITVNDNGELVSTQHAITEYRVVESSPHGYTWLELRPLTGRKHQLRVHCAEVLGTPILGDYKYGWQAHKTREPFVSSENTPTKPSPSPFGLDMEGGDVSSKQPHLHLHSKQIDLPNICQLLEKLEVSPDSDISDLDGLKFDAPLPTHMQLSFNLLKSRVESSDNC

SEQ ID NO: 14 - DNA, 2972 bp, Arabidopsis thaliana
  source 1..2972: mol_type = genomic DNA
ttcgaattcaaataaaataaaattagtttttctgtttggatatgtagagtagatctaatcgacccttgtacttctgcaatttgaattcaaaatttaaaccactctacttttgaatctctctctcactaaacctagaaagtagaaaaccctagcatttgtgatcttcaacgggaaaaatgacgactgtgactccgtccgccggtcgtgagctctcgaatccgccgtccgacggcatttctaatctccgattttctaataacagtgatcatctcctcgtttcttcatgggataaggttagtgaaacacctcatttgctctttgactgatttgattcgagtcctctctttccttcatctgaagtttttttttctgttgttctgcgtttgttgaatctaagcgtgtgagattgtatgatgtgagcaccaattcgttgaaaggagagttcttacatggcggagcagtactcgattgctgttttcacgatgacttctccggcttcagtgttggcgctgattacaaagtccgacggtattgtctctcttttctctcagtcatgtgaagagactgttggcataataatgtttcccctcaggctaatttaacattggctgctcagaagttgttgattttactgcgattttttcttcatattttgtcactgtgacggctatggaatctttctacatttagcaaatgccttgaatccgttgtataatttccttaatttataaggtttcatctggttatactgcaggattgtattcaatgtcggcaaagaggacattttggggacacatgacaaagcagtgcgatgtgttgagtattcttacgctgcgggtaggtagagtggtactctttatgctagtttgaacttcaacactgacgatttctggtgcttttaagttctagaaaactcatagctaaccagtttttgttatatgactctgatgatggatgatttaataccttagctgtcttggacagtgaactttgttccctaatcttttgtaatgtaactatatctggaactcagatattttgatttcagcattttgtttttttacacattatgtttattttgtgatgttacaggacaagtgatcactggatcttgggataaaacagttaaatgttgggatccaagaggcgctagtgggcctgaacgcacccaagtgggaacatatttgcaaccagaacgtgtttactctatgtctcttgttggacatcgtttggtagtggctacagcaggaaggcatgtaaacatctatgatctcagaaatatgtctcagcctgagcaaagaagggagtcttcactgaaataccagacgagatgtgtgcgttgttatcctaatggaacaggttagatgactgacatattgtttctgtccttgctaattttttcttccaattccaaagtatactctcagttcctcttcttccataccaatgaatttcggaaattgaaaatttacaggctatgctcttagctctgttgaaggaagggttgcaatggagttttttgatctgtcagaggctgctcaagctaaaaagtatattccttgtgttcttcttttctcttttccatgactttgactgacaaatgtatttgaacttattctcagatatgctttcaaatgtcatcggaaatcagaggctggaagggacattgtttaccctgtaaattccattgccttccatccaatgtgagttcttgtcttagaaggagcccaactgtttcatagatttctatatttactactttttcaaaacacaagaaggttaagataatactccaactaaataagaaagtaggaaaaatattttcgtgtaaagttgttcagcccttgtctgctgattgatatgttaaagatgaaattctaatgatgaagagaacaaatcgacttggaaagctgcttggatgcttggtctgagaacttatgggatataagagcaaatgaactaatgctaatatgctgtgtctgtaaacagtttctgtaattagacaatatgttagcttatgctttgtctgagaactaatgggaaaaatacaatgctgagaaatatctcctgtatagctactttcctgtgtaatcatctgagatttttaattgcagctatggcacctttgcaactggaggctgtgatggtttcgtcaacatttgggatggtaacaacaagaagaggctatatcaggttagtaatgagcaagatcttttggttattattctttcaatgttagtgtggcacaaaatttatttgcctccttgttccttttgtagtactcaaagtatccaacgagtatctcggcactgtcattcagtcgagatggtcagctgctggctgttgcttcaagttacacatttgaagagggagagaaatcgtaagtaacttgttccctttctaatcctttgaagttttagaatagtatgtctttgaaatctctgatgatttgtgcattggtttgattggttgcttattgagataacatagcagagagtaaccctgtgccttaatcttacagagattatcacaaacaaattaatgctttaaaggcaccctttttgtctttgtcacaaaaccgcacacttaaggatagtaatctctttgtatttggtttcaagttagaattctctttgattgggtctctgtgtgagaggttttgcaggcaagaaccggaggccatctttgtaagaagcgtgaatgaaatcgaagtgaaaccaaaaccgaaagtatacccgaatcctgcggcgtagaaaggaagaaacacaagttatttcctatgttggttgtttttgtatttgctaggagtgttccaaagattgaatcaacgtatgtttgagcttttaaataactagtgaaatatccacattcaccgtatatgtttaaatttctgtagtcaaactctcttgtcaaacatataatataagatgacaaatttccagtgtttaaaagatagaaagagatcattctac

SEQ ID NO: 15 - DNA, 1023 bp, Arabidopsis thaliana
  source 1..1023: mol_type = other DNA
atgacgactgtgactccgtccgccggtcgtgagctctcgaatccgccgtccgacggcatttctaatctccgattttctaataacagtgatcatctcctcgtttcttcatgggataagcgtgtgagattgtatgatgtgagcaccaattcgttgaaaggagagttcttacatggcggagcagtactcgattgctgttttcacgatgacttctccggcttcagtgttggcgctgattacaaagtccgacggattgtattcaatgtcggcaaagaggacattttggggacacatgacaaagcagtgcgatgtgttgagtattcttacgctgcgggacaagtgatcactggatcttgggataaaacagttaaatgttgggatccaagaggcgctagtgggcctgaacgcacccaagtgggaacatatttgcaaccagaacgtgtttactctatgtctcttgttggacatcgtttggtagtggctacagcaggaaggcatgtaaacatctatgatctcagaaatatgtctcagcctgagcaaagaagggagtcttcactgaaataccagacgagatgtgtgcgttgttatcctaatggaacaggctatgctcttagctctgttgaaggaagggttgcaatggagttttttgatctgtcagaggctgctcaagctaaaaaatatgctttcaaatgtcatcggaaatcagaggctggaagggacattgtttaccctgtaaattccattgccttccatccaatctatggcacctttgcaactggaggctgtgatggtttcgtcaacatttgggatggtaacaacaagaagaggctatatcagtactcaaagtatccaacgagtatctcggcactgtcattcagtcgagatggtcagctgctggctgttgcttcaagttacacatttgaagagggagagaaatcgcaagaaccggaggccatctttgtaagaagcgtgaatgaaatcgaagtgaaaccaaaaccgaaagtatacccgaatcctgcggcgtag

SEQ ID NO: 16 - DNA, 2425 bp, Thlaspi arvense
  source 1..2425: mol_type = genomic DNA
atgaatctcgaactctcgaatccaccttccgacggaatttccaatctccgattttccaatgctagtgatcatctcctcgtttcttcatgggataaggttagagacaaaacttctctacatttgtcttgttttccgactgatgtgattaaagtcctcttactgtatctgaagcttgtgaggttgtatgatgtgagcaccaattcgttgaaaggggagttcttacatggcgggccagtactggattgctgttttcacgatgattcctctggcttcagtgttggcgccgacaacaaagtcagacggtaatgtctctttttcctttctctacttcatacttaatgtttagaagtaaaatttgaagtaggcctgcataaaagaatctcaatctcgtgaagatcatacgaataatttcactgaggcttaacatgaaattggcttagaagttctaagtttctgttggcttctatctcggtttttctactcttttagcagaaagtgctcaaaaatgcgttgtaatttttctttatattcacgagattttctctctggttatgtgcttcaggatcgttttcaatgtcggcaaagaggatatcttggggatgcatgaatctccagtgcgatgtgttgagtattcttatgctacaggtaggtggagtggccatcatgctcatgatgctagttcaacagtaacatctctgcaagtatttgctttcagcattttgattctttattattgccttcaaagctatacggacacattatgttttatgtgatgtgatgttgcagggcaagtgatcactggatcttgggataaaacggttaaatgctgggatccaagaggcgcgagtgggccggatcgaactcaggtgggaacgtacttgcaaccagagcgtgtttactctctgtcacttgttgggaaccgtttggttgtggcaacagcaggaagacatgtgaacatctatgatctcagaaacatgtctcagcctgagcaaagaagggagtcttcacttaaataccagacgagatgtgttcgtagttatcctaatggaacaggtttagtgactgacatattgctctcactctggtttttaacctcagttcctcttccacagttttgagtttctgatattgaaatttttacagggtatgctcttagctctgttgaaggaagggttgcaatggagtttttcgatctgtcagaggctgctcaagctaaaaagtattcttccctgtgttatttttttcttttctcttttccttggctttgacttacagatggatttgaacttgttctcagatatgctttcaaatgtcatcggaaatcagaggctggaagggacattgtttactccgtaaataccattgcatatcatccgatgtgagttcttatctttagaaggagtccaactgttttcttgattaatcacttttcagagaacactagtaggttaatactatcgtataactaaagaagggaggaggcaacatattatagtctaaggtgttaattactgattgaaatggtgaagatgaaattctaatgatgaagagaacaaatgggctttgaaggctagatcgaagtttcttggatgctaatatgctatgtttataaatttctgtaactgttttgtttttgtgtttatatattcaactagacaaaggttgacttctgcttggtcatgagaacgaatgagaaaattatgatgctgagaaagtcttcccctgaatcatctgaaatttttaattgcagttatggcacctttgcaaccggaggttgtgatggttttgttaatatttgggatggtaacaacaagaagaggctatatcaggttcagtaaataacaagagctatattgttttcattttctcagtgtatcttagtgtgacacagaagtgtattatttggtccttgtttcgtttgcagtattcaaagtatccaacaagtatctcagcactttcattcagtcgagatggtcagctactagctgttgcttcgagttacacatatgaagagggggagaaatcgtaagtatctttcccctttatcatcatacacagaatgatttaggattgtttctggctgaaatgcttatagggaataatcacatagcatttctcgtataggacaaaactaaactttcggtctatatgaatctttatgttccttagtcataaacaaataaatgcttaaactgcacacttgaggatagtagtcttgtaacgcaaacccgagagtttcataactaaaccaccttcgaaaaaagactgctgtagagatatcttttgtatttgggttaaaaatgatctttaacttgctctgtgtatgaggttttacaggcacgaacctgatgccatctttgtgagaagcgttaatgaaatcgaagtgaaacctaaacccaaagcatatccaaatcatgcagcgtag

SEQ ID NO: 17 - DNA, 2109 bp, Brassica napus
  source 1..2109: mol_type = genomic DNA
atgagtctgcctcctccgtccgccggtcgtgagctagcgaatccaccgtccgacggcatttcgaatctcagattttccaacacgagcgaccatctcctcgtctcttcatgggataaggttagaaacactgatttgatcgaactcccttttccttttaatctgaatcgtttcttcttcttatgtgtctaagcgtgtgagattgtacgatgtgagcaccaattcgttgaagggcgagttcttacacggcggcgcagttctcgattgctgtttccacgacgattcctctggtttcagcgttggcagcgacaacaaagtcagacggtatttgtctcttctgtgaatctggggttggactcaactctgtgaagaaaaataagaaagttttggttatatatgttcaggattgttttcaatgttggcaaagaggatgttctggggatgcatgaaaagccagtgcgttgtgttgagtattcttatgctgcaggtattgttatagttttgatcatcatcatgctctttcatgctagctttgaaacagtgacattgactagttctggtgcttttctttgtctagaaaactcatcatagttaaccaatttatgttataattgaacattaggatagtgaaccttgattgttaatcatgctctttgatgctagctttgaacagaccatttctggtgcttttcatctatagaaaactcttagttaagcaattactatggacactgaactcttgttattgaagtaacgatttctttttagaagtaacttatcatgtttctcactgctccaagcctccaaagtaatgctgatatgttatgttttttttttgttcttatgtgatgttacagggcaagtgattactggatcttgggataaaacagttaagtgttgggatccaagaggtgcaagtgggcccgaacgcacccaggtggggacatacttgcaaccagagcgtgtttactctctgtctcttgttggaaaccgtctcgttgtggcaacagcaggaaggcacgtcaacatctacgatctcaggaatatgtctcagcctgagcaaagaagggagtcttcactcaaataccagaccagatgtgttcgtagttatcctaatggaacaggttatttgactgactggcatatattgcgttctgtggttgctagttaatttgtctttttaagttcccaagtatccctctttcctcagttttgagtatttgatattgaaattttgcaggttatgctctaagctctgttgaaggaagagttgcgatggagttctttgatctgtcagaggctgctcaggccaagaagtatttttcctcagtgttttttttttaattctactcgtctcttttccactactttggttacaaatagatttaaacttgttctcagatatgctttcaaatgccatcggaaatcagaggctggaagagacattgtttaccctgtaaatgccattgcattccatccaatgtgagttcctatcttagcaagattccaactgtttcttagatatctagatttattacatttttacacaacacgagaaggttttaaaataatagtccaactaaaattgtttagtgtaagttgaaggctagttcgaagttactgagatgctaatttgctatgttcggatgttatactataaatgctttttgagtctaacatcttagatacatctaagacaaagggtttcttgtgcctcgtatgataattagtgggaacaattataaacctccgcgtaatcatctgatattttcaattttgcagttatggcacatttgcaacgggaggctgtgatggttttgtcaacatatgggatgggaacaacaagaagaggctgtatcaggtttagcaaatgacttgagatctttttgattttcatcttctgagaatgttccctagtttgggacaagaatgtattataatttggtccttgtttcttttgcagtattcaaaatacccatcaagcatcgcagcactgtcattcagccgagatggtcagctactagctgttgcatcaagctacacgtttgaagagggggagaaatcgtaagtatctttcccttttatagctctctgaagtttggattggagtag

SEQ ID NO: 18 - DNA, 1023 bp, Brassica napus
  source 1..1023: mol_type = other DNA
atgagtcagcctcctccgtccgccggtcgtgagctcgcgaatccaccgtccgacggcatctccaatctcaggttttccaacacgagtgaccatctcctcgtttcttcatgggataagcgtgtgagattgtacgatgtgagcaccaattcgttgaaaggcgagttcttacacggcggcgctgttctcgattgctgtttccacgacgattcctctggtttcagcgttggcagcgacaacaaagtcagacggattgttttcaatgttggcaaagaggatgttctggggatgcatgaaaagcctgtgcgttgtgttgagtattcttatgctgcagggcaagtgattacggtatcatgggacaaaacagttaagtgttgggatccaagaggtgcaagtgggcctgagcggactcaggtggggacatacgtgcaaccggagcgtgtttactctctgtctcttgttggaaaccgtctcgttgtggcaacagcaggaaggcacgtcaacatctacgatctcaggaatatgtctcagcctgagcaaagaagggagtcttcactcaaataccagaccagatgtgttcgtagttatcctaatggaacaggttatgctcttagctctgttgaaggaagagttgcgatggagttctttgatctgtcagaggctgctcaggctaagaaatatgctttcaaatgccatcggaaatcagaggctggaagagacattgtttaccctgtgaatgccattgctttccatccaatttatggcacatttgcaacgggaggctgtgatggttttgtcaacatatgggatgggaacaacaagaagaggctgtatcagtactcaaaatatccatcaagcatcgcagcactgtcattcagccgagatggtcagctactagctgttgcatcaagctacacatttgaagagggagagaaatcgcacgagccagaagccatctttgtaagaaacgtcaatgaaatcgaagtgaagcccaaacccaaggcatatccaaatcctgcggcatag

SEQ ID NO: 19 - protein, 340 aa, Arabidopsis thaliana
MTTVTPSAGRELSNPPSDGISNLRFSNNSDHLLVSSWDKRVRLYDVSTNSLKGEFLHGGAVLDCCFHDDFSGFSVGADYKVRRIVFNVGKEDILGTHDKAVRCVEYSYAAGQVITGSWDKTVKCWDPRGASGPERTQVGTYLQPERVYSMSLVGHRLVVATAGRHVNIYDLRNMSQPEQRRESSLKYQTRCVRCYPNGTGYALSSVEGRVAMEFFDLSEAAQAKKYAFKCHRKSEAGRDIVYPVNSIAFHPIYGTFATGGCDGFVNIWDGNNKKRLYQYSKYPTSISALSFSRDGQLLAVASSYTFEEGEKSQEPEAIFVRSVNEIEVKPKPKVYPNPAA

SEQ ID NO: 20 - protein, 333 aa, Thlaspi arvense
MNLELSNPPSDGISNLRFSNASDHLLVSSWDKLVRLYDVSTNSLKGEFLHGGPVLDCCFHDDSSGFSVGADNKVRRIVFNVGKEDILGMHESPVRCVEYSYATGQVITGSWDKTVKCWDPRGASGPDRTQVGTYLQPERVYSLSLVGNRLVVATAGRHVNIYDLRNMSQPEQRRESSLKYQTRCVRSYPNGTGYALSSVEGRVAMEFFDLSEAAQAKKYAFKCHRKSEAGRDIVYSVNTIAYHPIYGTFATGGCDGFVNIWDGNNKKRLYQYSKYPTSISALSFSRDGQLLAVASSYTYEEGEKSHEPDAIFVRSVNEIEVKPKPKAYPNHAA

SEQ ID NO: 21 - protein, 319 aa, Brassica napus
MSLPPPSAGRELANPPSDGISNLRFSNTSDHLLVSSWDKRVRLYDVSTNSLKGEFLHGGAVLDCCFHDDSSGFSVGSDNKVRRIVFNVGKEDVLGMHEKPVRCVEYSYAAGQVITGSWDKTVKCWDPRGASGPERTQVGTYLQPERVYSLSLVGNRLVVATAGRHVNIYDLRNMSQPEQRRESSLKYQTRCVRSYPNGTGYALSSVEGRVAMEFFDLSEAAQAKKYAFKCHRKSEAGRDIVYPVNAIAFHPIYGTFATGGCDGFVNIWDGNNKKRLYQYSKYPSSIAALSFSRDGQLLAVASSYTFEEGEKSSLKFGLE

SEQ ID NO: 22 - protein, 340 aa, Brassica napus
MSQPPPSAGRELANPPSDGISNLRFSNTSDHLLVSSWDKRVRLYDVSTNSLKGEFLHGGAVLDCCFHDDSSGFSVGSDNKVRRIVFNVGKEDVLGMHEKPVRCVEYSYAAGQVITVSWDKTVKCWDPRGASGPERTQVGTYVQPERVYSLSLVGNRLVVATAGRHVNIYDLRNMSQPEQRRESSLKYQTRCVRSYPNGTGYALSSVEGRVAMEFFDLSEAAQAKKYAFKCHRKSEAGRDIVYPVNAIAFHPIYGTFATGGCDGFVNIWDGNNKKRLYQYSKYPSSIAALSFSRDGQLLAVASSYTFEEGEKSHEPEAIFVRNVNEIEVKPKPKAYPNPAA

SEQ ID NO: 23 - DNA, 2376 bp, Arabidopsis thaliana
  source 1..2376: mol_type = genomic DNA
tttttgttgtgacggaggggatttgctttaaaagtttagggcttttaaaatcggcgacggcgagcggagaattcccaaggaagagtcgatgaagttgaaatcggtgttcgatgcttcggaaatcaaatcggaatttgaatcagcgggaataaaccctaaattcgcgattcaaatctggaagtatgtaattcagaatcctgattgcgtttgggacgagattccttcattgccttccgctgcatactctcttctccattccaagttcaagactcttacttcgtctcttcactcgctatttcattcctccgatggcaccacctcaaagcttctcatcaagctccaggtactctatactctgttactgtatacaacacattgcttttatggattctgaaaaacttgtaatgtctctgtttcctgacgaactcatattggattcttgcttttggttttgaaattgaagaatggagcttttgtggaagctgtggtaatgagatacgatactcggttggggatgttaggagggaaaccacgtcctggaggtataagatctacgttatgtatttcatctcaggtattgatttttatcacgcaatgttatcttggttttatcgaatgaaagacatgtgatgtgaaagtatttatcaataggttggttgtaagatgggttgcacattctgtgctactggtaccatgggatttaaaagcaatttaacatctggagaaattgtggagcagcttgttcacgcctctcgcattgctgatatacgcaacatcgttttcatggtatatttctgcttctgttattgcattttatctacactatgtgcaccttgccttatggttatttattttatatactctgcattgatgaattttattcaccagttgttgccttggtttgatgattgtgatcagggaatgggagaacctttaaataactacaatgctgttgttgaagctgttcgtgtcatgttaaaccaaccatttcagctgtcgcccaaaagaattaccatatcaactgtaagtactcagaaagctaccttgaaacattcgtaaaacagagaaacttattcactgaattttgtaggaatactttagattatggaatttgataaagggaactgatagataaacttcaatccattaaacctcgcagtatgcagcttccaagacatctttcagctttttttgtctagttggttacttttaaattttggtgttttgggattttcttctgatttccttgtccttccgtaatgcttgtaccgtatattccaggcataaatctaaatcatgcaatgctgcctgtgttgacacttttttttactagttagcaaataaatttccctgtatcctaatgtcatttgcataggtcggaattgttcacgcaattaacaagctacacaatgatctacccggtgtaagtttagcagtatccctccatgcaccagttcaagaaatccgctgccagatcatgccagcagctagagcctttcctttacaaaagcttatggatgcacttcaaactttccaaaagaacaggtagttcatgtactcttgactggttcgttgctgttaaatattaaaaccacaatcccttcagttacattgacgattataacattatgccacattgtcattggatttaacagtcaacaaaagatcttcattgagtacataatgcttgatggagtaaatgatcaagagcagcacgctcatctactaggcgaattgctaaagacatttcaagtggtaaatcttcgatgtcttaatactcttaaaggttctgcaattatctgatgatattttttatggaaactctgtgctcttttcttgaacaggtcataaatttgataccatttaatccaattggatccacaagccaattcgaaacaagcagcatacaaggcgtgtcaagattccagaaaatcctgagggaaacatacaagatccggactacaattcgcaaagaaatgggtcaggatattagcggcgcttgcggtcagctagtggtgaaccaaccagacatcaaaaagactcctggaactgtggaacttagagacatagaagatctgcttctctaactttaggaccagagacatgaaacagacatttggaatggttgtgtagtatctttgaagccactcttcaaaacccttttcttaaacttcacattttgtcctctcatcactttgtggaaaacacttggctcactcttatatgcaagacaaacagagatggtccttggtccttccacactatcacatggaggtttaacttctcttataaaatattcaccaacaagtgatgttatgtcatcgttaaagttggaagtaaatgttaaactttaattttaatgaagtaaatagtagc

SEQ ID NO: 24 - DNA, 1119 bp, Arabidopsis thaliana
  source 1..1119: mol_type = other DNA
atgaagttgaaatcggtgttcgatgcttcggaaatcaaatcggaatttgaatcagcgggaataaaccctaaattcgcgattcaaatctggaagtatgtaattcagaatcctgattgcgtttgggacgagattccttcattgccttccgctgcatactctcttctccattccaagttcaagactcttacttcgtctcttcactcgctatttcattcctccgatggcaccacctcaaagcttctcatcaagctccagaatggagcttttgtggaagctgtggtaatgagatacgatactcggttggggatgttaggagggaaaccacgtcctggaggtataagatctacgttatgtatttcatctcaggttggttgtaagatgggttgcacattctgtgctactggtaccatgggatttaaaagcaatttaacatctggagaaattgtggagcagcttgttcacgcctctcgcattgctgatatacgcaacatcgttttcatgggaatgggagaacctttaaataactacaatgctgttgttgaagctgttcgtgtcatgttaaaccaaccatttcagctgtcgcccaaaagaattaccatatcaactgtcggaattgttcacgcaattaacaagctacacaatgatctacccggtgtaagtttagcagtatccctccatgcaccagttcaagaaatccgctgccagatcatgccagcagctagagcctttcctttacaaaagcttatggatgcacttcaaactttccaaaagaacagtcaacaaaagatcttcattgagtacataatgcttgatggagtaaatgatcaagagcagcacgctcatctactaggcgaattgctaaagacatttcaagtggtcataaatttgataccatttaatccaattggatccacaagccaattcgaaacaagcagcatacaaggcgtgtcaagattccagaaaatcctgagggaaacatacaagatccggactacaattcgcaaagaaatgggtcaggatattagcggcgcttgcggtcagctagtggtgaaccaaccagacatcaaaaagactcctggaactgtggaacttagagacatagaagatctgcttctctaa

SEQ ID NO: 25 - DNA, 2162 bp, Thlaspi arvense
  source 1..2162: mol_type = genomic DNA
atgaagttgaaatcggtgtttgacgcttcggaaatcagatcggaattcgagtcagcgggaataaaccctaatttcgtgattcccatctggaagtatgtaattcagaatcctgattgcgtttgggatgagattccttcattgccctccgctgcatacacgctcctccattccaagttcaagactctcacttcgtctcttcactccctcttccactcctccgatggcaccacctcaaaactcctcatcaagctccaggttcctctccactcactctgtttcttagtttgtcatgtaaatattctgggcatgatctgaatgtctatacaaatacgatatgagtctgtttttgacttttacgattgtaaggaagattagtcttgtagataaaaaaatgtacaagaagcaaccattttgtaaatcgattagaatacataggagatttgtctgctgaaaaggtttactttttgattacaactcattggttaatacactacgagacctcgtaattagccttggctttgtaacctctgtgtgagttgtctctatcatctgatgaactcatattccattcttgtttttgtatttgacatttaagaatggagcttttgtggaagctgtgataatgcgatacgatactcggttggggatgtgtggagggaaaccacgtccaggaggtgtaaggtctacattatgcatttcatcccaggtattgatttttatcacccagtgtcatctttgtttcattgaatcgcttttttggagtgagaagagcgtctgatgtaaaagtatttatcaacaggttggctgcaaaatgggctgcacattctgtgcaactggaagcatgggattcaaaagcaatttaacatctggagaaattgtggagcagctcgttcacgcctctcgcctagctgatatacgcaacatcgttttcatggtacattccctattaaatgcatttcacccaaagttctttgtttttgctgcgttttcggttgttgttgttgttcatgtgcacttttccctgtggtaatggtaaggtgtctttctatactctctctatcgatgaagttcattccttggtttgctgattgtgatcagggaatgggagaaccgttaaataactacaatgctgttgttgaagctgtccgcgtcatgttaaaacagccttttcagctttcacccaaaagaatcaccatatcaactgtaagtacagagaaacccaccttgaaacatttagaaaacagagaaatagttactgaaactggtagtaattatcctttagattatgggattccttagggtatcggtgtgaaacagagaaatgataaacttcatcctataaacctctcaatttgcagggctttcatggtttagttggttacgcttagcatttgggactatagtaggatttactttgtgattttctttttctctccactaacccacattctaatttattttgcatataggttggagttattcatgcgattaacaagcttcacaatgatctaccaggtgtaagtttggcggtatctcttcatgcgccagttcaagaaattcgctgtcagatcatgccagcagctagagcctttcctcttcaaaaacttatggatgcacttcaagctttccagaaaaacaggtagctcaaaattgcttctttgctaccaaaatgttaaaaccataatttattaaagatcatataacattttgccacattttccctggatttataatagtcaacagaagatcttcattgagtacatcatgcttgatggagtaaatgatcaagaggagaacgctcatcaactcggcgaattgctaaagacatttcaagtggtaaagtcttcgatttcttaatactacaagagtttctgggactatctgatggaaactctgtgccgtttttctttaaccaggtgataaatttgataccgtttaacccaatcggatccacaagccaattcaagaccagcaccaaacaaagcgtctcaagcttccagaaaatcctgagggaaacatacaaaatccgaaccacaattcgcaaagaaatgggtcaggatattagcggtgcttgtggtcagctagtcgtgaatcaaccggacagcaagagacctcctggaactgtggaaccacttagagacattgaagacctgcatctttaa

SEQ ID NO: 26 - DNA, 5628 bp, Sorghum bicolor
  source 1..5628: mol_type = genomic DNA
agtactccccactcgagccgacgcaatttcgcacccgaagttccaaaccccgccggccgccgcgtcccttccacttcctccttaccccaatggcgtcgtcatcgagggcgacgtcgtcgcgccgttccgtcttcgacgccgcctacatccgctcggagttttctgcggctggcatctccggccacttcatccctctcatctggaagtacgcctctttacgccaaatcacaggcgcatgccacaatccccgcccgcccgtctccctccctttctccaattcccaagttttttttatatatatttctgtttaacctgaggagtgattctctctgcgtgcgcaggtacgtacttcagaaccctaggtgcagcgacctggatggcgtcccgtcgctgccggcggccgcgtacgcgctcctccggcagaagttccggccgaccacgtcgacgctaaccgctgccgcggactccaaggaccgcaccacgaccaagctcctcatctccctgcaggtaatttgcacattcacaaatgtggcatgtggcgtgcgagtccactggtgtttggggattatgcgaaattgggaatatgttgtagtctccccggttatttcggtagctacttttgcatttggttcattgtccgtggacattagaaattttctcttggtgatgtgagcttatgaaacatatgcttgtttgattactccgtcaggaatggaatggttgacacatgcataattgattagcctcatgggcactattttagggattgaattcaccctgattgtctctgatagagtaggcattagaaaacagtacttttgtttcgtgtttatgtggttttccaatgtttgtggaagataatagcataccttgaaatacctaatctgtgtagttttatgttttttttaagatcaattcatcattattgttgtcttggatgtaatagagctgcaatacaaatagggagcaccaagatacagatatgcactatcttgtactaacttctacagattcaacctgcagcacctgaaccatccagttattgaatatttatagcaaagacaagtataactaacaagcaggaccttttgcccctccttccgcgaataaaaaagaaaaaaagagagaagtataacttaaatgcttgacttgttcctaatattaactctaccaactgttgtggaggagcatttattttctgttgctatttggctagatagtaggaggttcgagtcatcttaaaaaatgtacagcttaagccatatatatttgatatttcattctcgttatttggattttatatttcacacttgtatcctttagtttctttgccctttggctctatatctgtagtttttcagtcatcttcaaaatgaatagataaagatataatggtttcagtccttataatatgttatatgacttatggtagcatcttctgtattagatagcctgtaactttgcgatttaaattctactcccttttttttgcatagtgaactccttacaggtggacatcatttcttgcaagctacaattctatttcagaccgcctccttttcttttttaacttggctgttgtttatctaattattttatgtctgtgcattgaagaagtttttaaccacaaatttgccatttttttgttgtaagagttttttcttttcagattttgtattttttaacattgaagaacaacttattggacttcaaatttgtggtgtagaatggagagtctgtagaggcagtggtcatgcgatatgacacacgactggggaagtatgatggaaagcctcggcctggtggactgcgctcaaccctttgtgtgtcatcacaggtgaatatatttctatattggatgtcaggtgctcaagtctggatgaacttgtttctaatggtttaacaccttttaccagaagacctgttcataacttaattctcaccagtgtgttatgattcttggtaaacttgttactctattgttatttggtgactttggctcggtagatagcccagccgggaacgaatcccaagcaatgagaccaagagaatgcagaatgtctactaaagagctctctcaagtctttctttctacaatactaaacaggttgggtgcaagatgggctgtagattctgtgctactggaacaatgggcttcaaaagcaatctgtcttctggagaaattatagagcagctggtccatgcatcccgctattctcagatccgaaatgttgttttcatggtaattttcactcaagtagtagcaattgcctgaaatttctatgtaagaaagaaaaattgtggcatttgaatgcgatgattatgcatcttttgtgatcccttgtagtgttgttttcatcttgattatgctgatcagatttaacttgttacttggttttgtcattgattctgtgaacaaataagctcagtaagatccatatattggaaaatgacatccattacttcattcttttgccaacaagtatcacatgccatcctcacattagattgtattcctaacttgaccattgtgacgatactgagcattattaccctggtagttgtatatactttacattgggggcaatttacttacagacaaaatgcttcattttgcaagtatcaccactgcagaaatgggaaattattcctgcaaagtttacatgaatagaatgaatgccagaattttcttagctagtttgcttgatttatggattgagctggcaatatgttgatctgtttttcttttgctggtgagtggtgacaatggttcttgtacaaattcctgctggtattacagttatcccttgttacctataattctcaaaaactagtggtatgctgtttgagttagtttctaaattattataggtttattctttcatgttcatgttctctggcacttatagcttatgtgctttgctgattctttgatatagaaattagaaacactgctttgctgattgctaaaaaatcgtgggtcgctaaatgtttgtgcatgtttaactttaaaattgcaaattattatctacatatgtgaacatccttggaactgggtggtgtcatgtcattattgagaagaaccatgataaatgaattctatttgatgtttgttataacagtattagtgtcctccagaggttagcgaggtgttatttatatgcagggaatgggggagccgatgaacaactataatgctttggttgaagcaatcggagtgtttacaggatctcctttccagctttcacctaagagaattactgtatctactgtaagcatacttagtcacctttgcatgtgaaatcacattctcaagatttttgttgaagcactatattttgttagtagtgaaatattcttataaactatatgttgtttagtggcggctgttacacttctgaatgacctaatgaatttctgctgtgataaaccacacaatggtgaatccgtcaaaagctttttatttaattgtgcaaatataacttgataaattgtgtagcaggccagttgtatattctttggatgaactgttctattcccccttgttgctagcaagtatatcttgttttagcacttgtaggatttaaacgttaacatttattataaaatacagttattacataagatatgtggtctgatgccaaaccttttttttcacttacgttattgctgcttctaggtttataatagaattcattgtcatgttgtgtggtaaaaacttttctatgcatgttaattttatttatatgtttgctcatttatcaagccaaattttccatattagctttgaagccatggtgtgttttgccactgtattttttgctaaaagtttgttttaagattgaacttttgaattagagaagctgatagattgatttttcttaataattactgcaggtcggaatcattcatggaatcaacaagttcaatgcagatcttccaaaggtgaatttagctgtgtcattgcatgctcctgaccaagatatacgctgtcagataatgcctgctgcacgtgcctttcctttagtaaagttaatgaacgcgctgcagtcctatcaaaatgagaggtgagattacaataacgcatctcttaccagtgtcaaaatgttagcatggagatttgatttctgattaccctttcatgttttatcacagcaaacagaccatctttattgagtacattatgcttgatggagttaacgatcaggaggagcatgctcatcagcttggtaaactgcttgaaacgttcaaagcggtgaggcactattacattacttcattttatcattattactttgctctagttcctttgtagtcacttctttttgcaagatgtctaacgaggcagttttgtgtcgttaacttcttgtgttcattattcaatccattttgtattgcctccaccagtcttacttttccatatttccctatctgtttgcttttgacaattgccatttctgtttagtaatcctggtactccaaatttcttctaggtgtttgtcgtccactcctgtttttatgtgtttcaaaatttttgcaataacttactagtgtaatttttgtacactgagcctattaataccaagtgatggtcaagacatatgtaggtagcagtacttgtacattcacctttgtgtatggaaatggagtgatcaatatgttctctaaactaccaacaggttgtcaatctaataccattcaatccaattgggtcattaagcaatttcaagacaagcagtgaccaaaatgtgaagaagttccaaaaggttctaaaaggcatctaccacatcagaaccactgttcgccagcagatgggtcaagacatagctggtgcttgtggccagttggtggttagcctcccagatgaaagatcagctggtggagcaaccctgttgtcagacatcgaagaccttcggatctgacctctcccaattgttacttctatcttctcaaaaccagatcgtcttactaattagccttacatgatatggtaactagtgctatagtttcagtcaagctttaaaacatggagatgtttagtttgaccattgttgtgtttcccttttgttctgggatgcacaattttgctgtaaacacaatggtgaagactcctgctcgtgtggataggtggccactgaaacggttggtaacttggtattgtaaatgggacaatgttatcgcaacaatgccatggtcagctataatgatatggtatcttctgtctaagacgactttcctgtcagatctcaacgaatatctgatgtttttcctctcttatttgcagcttttcattgccattagcaagttggtcgggttggaattgtgccttcgttttatgctttattcagattccgtgtcggtgggctgtatgtaatggtggtggcatgggcttggtgctgctgcagcgtgttatctttgaaaatgtgaacccaattgctggaaatcatgtgagattgtcaggagcgggacaaggcgaaacatatgagctgtgttgtttggttcttatgccctctttatatccacctagctaactattagctgtgttagctgggaaaaggcaactaatagcttattgtcgagagaaatcagctaataactgctaactgttatctaggtggtaggtaagac

SEQ ID NO: 27 - DNA, 1852 bp, Brassica napus
  source 1..1852: mol_type = genomic DNA
atgaagatgaaatctgtattcgacgctccggagatgaaatcggagttcgagtcagctggaataaaccccaatttcatgatcccgatctggaagtatgtaattcagaatcccgattgcgtttgggacgagatcccttcgttgcccaccgccgcatacactctcctccattcaaagttcaagactttcacttcctctcttcactccctcttccactcctccgatggcaccacctctaaactcctcatcaagctccaggtaactctgctctctctctcgctctctctgtttctgggttttgatgtgagtgtgtatacaatgagagtatgtttttgttattaagattgtatagatgagattagtctttgtagatgagaagctgtccttttaataatacataaaagagatttggctgctgaacattgcttgtagtaagtcctttgttatagtgttatctgatgaactcgtacttgattcttgattttaaagaatggagcttttgtggaagctgtgataatgagatacgatactcgtttggggatgtgtggagggaagccacgccctggaggtgtgagatctactctatgcatttcatcccaggtattgatttttttcatcacgctgtttcactgaatcactgtggagcatctgatacaagtggtttgtcaataggttggttgcaaaatgggctgcacgttctgtgcaactggtagtatgggattcaaaagcaatttaacatctggagaaatcgtggagcagctagtccacgcctctcgcctagctgatatacgcaacatcgtattcatggtaacgcatctcatctaagtttctttttgcctgttgttgttgttactacattatgtatccgtgtcttcacttcttgccttgtggtaattgctagatgtggctttctatagtttctgtatcgatgaagtaaacattcaccagttgttggcttgattttgctgattgtgatcagggaatgggagagcctttaaataactacaatgctgttgttgaatctgttcgtgccatgttaaagcagccttttcagctctcacccaagagaattaccatttcaaccgtaagtactgagaaaactaccttgaaacattcatgaatcagagaagaaaatattctctttcttatttttttttttctccactaacccatatcgtttgcacataggttggagttgttcatgcgattaacaagcttcacaatgatctaccaggtataagtttggcggtatctcttcatgcaccagttcaagaaatccgctgccagatcatgccagcagctagagcctttcctcttcaaaagctcatggatgcacttcaaactttccagaaaaacaggtaactttgctactgcatgttcaaaccataatctcaaaagttgataaagatcataacatacttcgattttcaaacagtcagcagaaaatcttcattgagtacatcatgcttgatggagtaaatgatcaagaggagaacgctcatcaactaggcgaattgctaaagacatttcaagtggtaaaaatcttctgtttctttatactcttaatagtttgagcaattatctgatggaaattctctgctgttttctcaaataataggtgataaatttgataccattcaatccaattggatccacaagccagttcaaaaccagcaccaaagaaagcgtctcaagtttccagaaaatcctgagagaaacctacaatatacgaaccacggttcgcaaagaaatgggtcaggatattagtggggcttgcggacagctagtcgtgaaccaacctgacaacaagagacctgcagaaccacttagagacattgaagatctgcatctttaa

SEQ ID NO: 28 - DNA, 2052 bp, Brassica napus
  source 1..2052: mol_type = genomic DNA
tagcgacggctacggcgaggtgagagtgttcaaggaagatcgatgaagatgaaatctgtattcgatgctccggagatcaaatcggagttcgaatcagcgggaataaaccccaatttcatgattccgatctggaagtatgtaattcagaatcccgattgcgtttgggacgagatcccttcattgcccaccgccgcatacactctcctccattcaaagttcaagactttcacttcgtctctccactccctcttccactcctccgatggcaccacctctaaactcctcatcaagctccaggtactccgctctctctctctgtttctgggttttgatatgagtgtgtatacaaacgcgatatgagtatgtgattgccttttcaattgtataggagattagtctttgtagatgagaagctgtccttttaataatacataaaagagatttggctgctgaacatttgcttgtagtaagtcctttgttatagtgttatctgatgaactcgtacttgattcttgatttaaagaatggagcttttgtggaagctgtgataatgagatacgatactcggttggggatgtgtggagggaagccacgccctggaggtgtgagatctactctatgcatttcatcccaggttttgattttttcatcactcgtgtttcattgagttactgtagagtgatgagagcatctgatttacaagtatttatcaataggttggttgcaaaatgggctgcaccttctgtgcaactggtagcatgggattcaaaagcaatttaacatcaggagaaattgtggagcagcttgtccacgcctctcgcctagctgatatacgcaacatcgtattcatggtaacgcatctcgtctaaagtttctttttggctgttgttgttgttactacattatgtatccgtgtcttcacttcttgccttgcggtgattgctataggtgtctttctatatagtttatgtatcgatgaagtaaacgttcaccggttgttggcttgattttactgattgtgattagggaatgggagagcctttaaataactacaatgctgttgttgaatctgtgcgtgccatgttaaagcagccttttcagctctcacccaagagaattaccatttcaactgtaagtactgagaaaactaccttgaaacattcatgaatcagagcagaaacattctctttcttattttctttttctccactaacccatatcgtttgcacataggttggagttgttcatgcgataaacaagcttcacaatgatctaccaggtataagtttagcggtatctcttcacgcaccagttcaagaaatccgctgccagatcatgccagcggctagagcctttcctcttcaaaagctcatggatgcacttcaagctttccagaaaaacaggtaactttgctactgcatgttcaaaccataatctcagaagttgataattataataacatagtgacatatcttcctttaattttaaacagtcagcagaagatcttcattgagtacatcatgcttgatggagtaaatgatcaagaggagaacgctcatcaactaggcgaattgctaaagacatttcaagtggtaaaaatcttctgtttcttaatactcttaatagtttcagcaattatctgatgtgaatctctgtggcgttttctcaaaaaaaacaggtgataaatttgataccgttcaacccaattgggtccacaagccagttcaaaaccagcaccaaacaaagcgtctcgatcttccagaagatcctgagggaaacctacaatatacgaaccacggttcgcaaagaaatgggtcaggatattagcggtgcttgcggccagctagtcgtgaaccaacctgacaacaagagaccagcagaaccacttagagacattgaagatctgcatctctaactttaggaccaggcatctctgaaattttcctaaagagaccaaagatatatggtttgtattatcttcgaagtcactcttgaaaacctttttcttagttacttgacatattgttcctcattactctggtgaaaacactt

SEQ ID NO: 29 - DNA, 1881 bp, Glycine max
  source 1..1881: mol_type = genomic DNA
gttgagaagaaagagagagagagggagagggcatgggaatccgatcggtattcgatggcggcgagctgagaagggaagtggagaaaagtggaattgacccaaaattcattccaaagatatggaagcatatcctcatctctgccaaagatgaagattgggattgggagaaacaagttccctccttgccctcttcggcctactctctccttcgttccaacttcaaaacccctctctcttcctctattcactccgttttccactctgccgacaacctcaccaccaagctcctcatccagctccaccacaatcatggacctttcgtcgaggctgtcattatgaggtacgatactcgtttgggcaaatatgccggccaacctcgccccggtggtctcagagctactttgtgtatttcttctcaggtaaccatatctttcttttcttttcttttcttttcttttctttttctattgctaacgcttccttttctcttctgcaggttggatgcaaaatgggttgcaatttctgtgccactggatccatgggattcaaaaacaacctatcatccggtgaaattgtggaacagctcgttcatgcctctaccttctcacaaatccgtaatgttgtcttcatgggcatgggagagcctctcaacaactattctgctgtggtagaagccgttcgcatcatgactgggttgccatttcaattgtcatccaaaaggattaccatctcaacggtataaacataaacacacaaatttatgatagccgtttttgagtaaaatcttagaatctgtttgttacatttctctaataggttggcatcattcatgctatcaacaagcttcatgatgacctgcctggtttgaacttggctgtctcactgcatgcgccggcccaagacatccgttgccagataatgcctgctgctcgtgcttttcctttgggaaaactcatggattcactgcaagtctatcaaaggaaaaggtcattcatactttctctcaacaatttctctcatttttttaagccattaatattaatatttcatccttaattatagtctgcagaaaatatttattgaatacataatgcttgatggggtgaacgacgaagagcaccatgcccacctattgggaaaactgttggagacatttcaagtggtaaatactcaactgcttcttcctttctctattcaatattcaatgtctctaccaaacatttatgctctgcccccataattccaatgccactcttctactctaataaaatgtaggcaatcgtgaatgaatagtgttgtttctcgataattcagtgactctcaggaaaatgttcttaaaacttgatcgtcattctctgctcagttacacaatgctgttaagtactaacattttgcatatggtctgaattttgaaacaggttgtgaacttaatacctttcaactctattggtaccttgagtcaattcaaacctaccagtgagcagaaagtctcaaattttcagaaaattcttaggggtacctataatattcgaacaacagttcggaagcagatgggtcaggacataagtggtgcttgtggacaattgctggtgaacatatctgacaagtcccttggcactgcagttcccctaacggacatagaagatattgttatctgatatcaatagcttcaatttaattcttgccctcaattttaatttcgtttcctagtttttctattgtttttatgtttggttctacagaaattagaagtagaactgtagaagacaaggtgcactggctgaaatgcaaccaaaatgctttatatgaatgtgtgatgatcttttagattttgataaaattgaatttcaaagaattgaagaaaaaaaaatcatttgtgag

SEQ ID NO: 30 - protein, 372 aa, Arabidopsis thaliana
MKLKSVFDASEIKSEFESAGINPKFAIQIWKYVIQNPDCVWDEIPSLPSAAYSLLHSKFKTLTSSLHSLFHSSDGTTSKLLIKLQNGAFVEAVVMRYDTRLGMLGGKPRPGGIRSTLCISSQVGCKMGCTFCATGTMGFKSNLTSGEIVEQLVHASRIADIRNIVFMGMGEPLNNYNAVVEAVRVMLNQPFQLSPKRITISTVGIVHAINKLHNDLPGVSLAVSLHAPVQEIRCQIMPAARAFPLQKLMDALQTFQKNSQQKIFIEYIMLDGVNDQEQHAHLLGELLKTFQVVINLIPFNPIGSTSQFETSSIQGVSRFQKILRETYKIRTTIRKEMGQDISGACGQLVVNQPDIKKTPGTVELRDIEDLLL

SEQ ID NO: 31 - protein, 373 aa, Thlaspi arvense
MKLKSVFDASEIRSEFESAGINPNFVIPIWKYVIQNPDCVWDEIPSLPSAAYTLLHSKFKTLTSSLHSLFHSSDGTTSKLLIKLQNGAFVEAVIMRYDTRLGMCGGKPRPGGVRSTLCISSQVGCKMGCTFCATGSMGFKSNLTSGEIVEQLVHASRLADIRNIVFMGMGEPLNNYNAVVEAVRVMLKQPFQLSPKRITISTVGVIHAINKLHNDLPGVSLAVSLHAPVQEIRCQIMPAARAFPLQKLMDALQAFQKNSQQKIFIEYIMLDGVNDQEENAHQLGELLKTFQVVINLIPFNPIGSTSQFKTSTKQSVSSFQKILRETYKIRTTIRKEMGQDISGACGQLVVNQPDSKRPPGTVEPLRDIEDLHL

SEQ ID NO: 32 - protein, 381 aa, Sorghum bicolor
MASSSRATSSRRSVFDAAYIRSEFSAAGISGHFIPLIWKYVLQNPRCSDLDGVPSLPAAAYALLRQKFRPTTSTLTAAADSKDRTTTKLLISLQNGESVEAVVMRYDTRLGKYDGKPRPGGLRSTLCVSSQVGCKMGCRFCATGTMGFKSNLSSGEIIEQLVHASRYSQIRNVVFMGMGEPMNNYNALVEAIGVFTGSPFQLSPKRITVSTVGIIHGINKFNADLPKVNLAVSLHAPDQDIRCQIMPAARAFPLVKLMNALQSYQNESKQTIFIEYIMLDGVNDQEEHAHQLGKLLETFKAVVNLIPFNPIGSLSNFKTSSDQNVKKFQKVLKGIYHIRTTVRQQMGQDIAGACGQLVVSLPDERSAGGATLLSDIEDLRI

SEQ ID NO: 33 - protein, 370 aa, Brassica napus
MKMKSVFDAPEMKSEFESAGINPNFMIPIWKYVIQNPDCVWDEIPSLPTAAYTLLHSKFKTFTSSLHSLFHSSDGTTSKLLIKLQNGAFVEAVIMRYDTRLGMCGGKPRPGGVRSTLCISSQVGCKMGCTFCATGSMGFKSNLTSGEIVEQLVHASRLADIRNIVFMGMGEPLNNYNAVVESVRAMLKQPFQLSPKRITISTVGVVHAINKLHNDLPGISLAVSLHAPVQEIRCQIMPAARAFPLQKLMDALQTFQKNSQQKIFIEYIMLDGVNDQEENAHQLGELLKTFQVVINLIPFNPIGSTSQFKTSTKESVSSFQKILRETYNIRTTVRKEMGQDISGACGQLVVNQPDNKRPAEPLRDIEDLHL

SEQ ID NO: 34 - protein, 370 aa, Brassica napus
MKMKSVFDAPEIKSEFESAGINPNFMIPIWKYVIQNPDCVWDEIPSLPTAAYTLLHSKFKTFTSSLHSLFHSSDGTTSKLLIKLQNGAFVEAVIMRYDTRLGMCGGKPRPGGVRSTLCISSQVGCKMGCTFCATGSMGFKSNLTSGEIVEQLVHASRLADIRNIVFMGMGEPLNNYNAVVESVRAMLKQPFQLSPKRITISTVGVVHAINKLHNDLPGISLAVSLHAPVQEIRCQIMPAARAFPLQKLMDALQAFQKNSQQKIFIEYIMLDGVNDQEENAHQLGELLKTFQVVINLIPFNPIGSTSQFKTSTKQSVSIFQKILRETYNIRTTVRKEMGQDISGACGQLVVNQPDNKRPAEPLRDIEDLHL

SEQ ID NO: 35 - protein, 378 aa, Glycine max
MGIRSVFDGGELRREVEKSGIDPKFIPKIWKHILISAKDEDWDWEKQVPSLPSSAYSLLRSNFKTPLSSSIHSVFHSADNLTTKLLIQLHHNHGPFVEAVIMRYDTRLGKYAGQPRPGGLRATLCISSQVGCKMGCNFCATGSMGFKNNLSSGEIVEQLVHASTFSQIRNVVFMGMGEPLNNYSAVVEAVRIMTGLPFQLSSKRITISTVGIIHAINKLHDDLPGLNLAVSLHAPAQDIRCQIMPAARAFPLGKLMDSLQVYQRKSLQKIFIEYIMLDGVNDEEHHAHLLGKLLETFQVVVNLIPFNSIGTLSQFKPTSEQKVSNFQKILRGTYNIRTTVRKQMGQDISGACGQLLVNISDKSLGTAVPLTDIEDIVI

SEQ ID NO: 36 - DNA, 1989 bp, Arabidopsis thaliana
  source 1..1989: mol_type = other DNA
ctagtctagctcaacagagcttttaacccaaattggtacaatagaatacaactttagatcataattctcaaaagaaagagattccttagctattctatctgccactccatttccttctcggcttgtatgcacaagcataaaatcctcaaacttgctaagtagatactttatgtcttggataattggattgagacttgacaagcataactttcatgtaaccaaagacacaagttgctgagaatccacctcaaaaatgatcttcctataattgaatcgggataatgacagcacagcccatctaagagcctccacttctacttccagcacgcttcttacttttaccacagctcttgcacctaaccataacaccttccctgtatgatcgcgaagcacccaccctaagccacattttaatccttctgttggccatgccccatcaaagttgcacttaacccaagattgtggtggagcttcccatgtttctcgtctgtcccgacggtgttgtggttggtgctttccttacattctgagcctctttccttctaatccactcatctgcatcttcttgtgtccttactaatacctcattggttccaaattccctccctttaagcaccagctcgtttctgttcttccacagcctcccaagtatccaagggactaaagcctccacattcttcagatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacagg

SEQ ID NO: 37 - DNA, 1992 bp, Zea mays
  source 1..1992: mol_type = other DNA
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcacggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcag

SEQ ID NO: 38 - DNA, 1346 bp, synthetic construct
  source 1..1346: mol_type = other DNA
tcgacgaattaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacg

SEQ ID NO: 39 - RNA, 20 nt, synthetic construct
  source 1..20: mol_type = other RNA
acgctgattagctgcagaca

SEQ ID NO: 40 - RNA, 20 nt, synthetic construct
  source 1..20: mol_type = other RNA
ttcaacggatggctcaacga

SEQ ID NO: 41 - RNA, 20 nt, synthetic construct
  source 1..20: mol_type = other RNA
ccttatcccatgaagaaacg

SEQ ID NO: 42 - RNA, 20 nt, synthetic construct
  source 1..20: mol_type = other RNA
tgaaaacagcaatccagtac

SEQ ID NO: 43 - RNA, 20 nt, synthetic construct
  source 1..20: mol_type = other RNA
atggaggagcgtgtatgcag

SEQ ID NO: 44 - RNA, 20 nt, synthetic construct
  source 1..20: mol_type = other RNA
gatactcggttggggatgtg

SEQ ID NO: 45 - RNA, 20 nt, synthetic construct
  source 1..20: mol_type = other RNA
acgctgattagccgcggata

SEQ ID NO: 46 - RNA, 20 nt, synthetic construct
  source 1..20: mol_type = other RNA
tcccaccatcgacgccgccg

SEQ ID NO: 47 - RNA, 20 nt, synthetic construct
  source 1..20: mol_type = other RNA
gcagcgacaacaaagtcaga

SEQ ID NO: 48 - RNA, 20 nt, synthetic construct
  source 1..20: mol_type = other RNA
tgttggcaaagaggatgttc

SEQ ID NO: 49 - RNA, 20 nt, synthetic construct
  source 1..20: mol_type = other RNA
aggagagtgtatgcggcggt

SEQ ID NO: 50 - RNA, 20 nt, synthetic construct
  source 1..20: mol_type = other RNA
tactctatgcatttcatccc

SEQ ID NO: 51 - RNA, 76 nt, synthetic construct
  source 1..76: mol_type = other RNA
gttttagagctagaaatagcaagttaaaataaggctagtccgttatcaacttgaaaaagtggcaccgagtcggtgc